SEQUENCE LISTING (233 sequences)

SEQ ID NO: 1 - protein, 113 aa, synthetic construct
  REGION 1..113: note = Synthetic
EVQLQQSGAELVKPGASVKLSCTASGFNIKDYYMHWVKQRTEQGLEWIGRIDPEDGETKYAPKFQGKATITADTSSNTAYLQLSSLTSEDTAVYYCDRGLAYWGQGTLVTVSA

SEQ ID NO: 2 - protein, 108 aa, synthetic construct
  REGION 1..108: note = Synthetic
QIVLTQSPAIMSASPGEKVTLTCSASSSVSSSYLYWYQQKPGSSPKLWIYSTSNLASGVPARFSGSGSGTSYSLTISSMEAEDAASYFCHQWSSYPYTFGGGTKLEIK

SEQ ID NO: 3 - protein, 113 aa, synthetic construct
  REGION 1..113: note = Synthetic
EVQLVQSGAEVKKPGATVKISCKVSGFNIKDYYMHWVQQAPGKGLEWIGRIDPEDAETKYAPKFQGRVTITADTSTNTAYMELSSLRSEDTAVYYCDRGLAYWGQGTLVTVSS

SEQ ID NO: 4 - protein, 108 aa, synthetic construct
  REGION 1..108: note = Synthetic
EIVLTQSPATLSLSPGERATLSCSASSSVSSSYLYWYQQKPGQAPKLWIYSTSNLASGIPARFSGSGSGTDYTLTISSLEPEDFAVYYCHQWSSYPYTFGQGTKLEIK

SEQ ID NO: 5 - protein, 113 aa, synthetic construct; identical to SEQ ID NO: 3
  REGION 1..113: note = Synthetic

SEQ ID NO: 6 - protein, 108 aa, synthetic construct
  REGION 1..108: note = Synthetic
EIVLTQSPATLSLSPGERATLSCSASSSVSSSYLYWYQQKPGQAPKLWIYSTSNLASGIPARFSGSGSGTDFTLTISSLEPEDFAVYYCHQWSSYPYTFGQGTKLEIK

SEQ ID NO: 7 - protein, 113 aa, synthetic construct
  REGION 1..113: note = Synthetic
EVQLVQSGAEVKKPGATVKISCKASGFNIKDYYMHWVQQAPGKGLEWIGRIDPEDAETKYAPKFQGRVTITADTSTNTAYMELSSLRSEDTAVYYCDRGLAYWGQGTLVTVSS

SEQ ID NO: 8 - protein, 108 aa, synthetic construct; identical to SEQ ID NO: 4
  REGION 1..108: note = Synthetic

SEQ ID NO: 9 - protein, 113 aa, synthetic construct; identical to SEQ ID NO: 7
  REGION 1..113: note = Synthetic

SEQ ID NO: 10 - protein, 108 aa, synthetic construct; identical to SEQ ID NO: 6
  REGION 1..108: note = Synthetic

SEQ ID NO: 11 - protein, 114 aa, synthetic construct
  REGION 1..114: note = Synthetic
EVQLQQSGVEVVQPGASVKLSCTASGFNIEAYYMHWVKQRTEQGLEWIGRIDPEDGESKYAPKFQGKATMTADTSSSTAYLQLSSLTSDDTAVYYCVRGSYEYWGQGTTLTVSS

SEQ ID NO: 12 - protein, 108 aa, synthetic construct
  REGION 1..108: note = Synthetic
QIVLTQSPAIMSASPGEKVTLTCSASSSVSSSYLYWYQQKPGSSPKLWIYSTSNLASGVPPRFSGSGSGTSYSLTISSMQAEDAASYFCYQWSSYPYTFGGGTKLEIK

SEQ ID NO: 13 - protein, 123 aa, synthetic construct
  REGION 1..123: note = Synthetic
QIQLVQSGPELKKPGETVKISCRASGYTFTTYGMSWVKQAPGKGLRWMGWINTYSGVSTCADDFKGRFAFSLETSATTAYLQIHNLTNEDTATYFCARDPHSYGNSPAWFPYWGQGTLVTVSA

SEQ ID NO: 14 - protein, 107 aa, synthetic construct
  REGION 1..107: note = Synthetic
DIVMTQSQKFMSTTIRDRVSITCKASQNVGISVAWYQQKSGQSPKLLIYSASNRYTGVPDRFTGSGSGTDFTLTISNMQSEDLADYFCQQYSSYPLTFGSGTKLAIK

SEQ ID NO: 15 - protein, 120 aa, synthetic construct
  REGION 1..120: note = Synthetic
QVQLQQSGPELVKPGASVKMSCKASGYTFSEYVLSWVKQRTGQGLEWIGEIYPGTITTYYNEKFKGKATLTADKSSNTAYIQLTSLTSEDSAVYFCGRFYDYDGGWFAYWGQGTLLTVSA

SEQ ID NO: 16 - protein, 108 aa, synthetic construct
  REGION 1..108: note = Synthetic
ENVLTQSPEKMAVSLGQKVTMTCSASSSVSSSDLHWYQQKSGASPKPLIHGTSNLASGVPARFSGSGSGTSYSLTISSVEAEDAATYYCQQWSGYPWTFGGGTNLEIK

SEQ ID NO: 17 - protein, 123 aa, synthetic construct
  REGION 1..123: note = Synthetic
QIQLVQSGPELKKPGETVKISCKASGYTFTTYGMSWVKQAPGKGLKWMVWINTYSGVPTYADDFKGRFAFSLETSASTSYLQINNLKNEDTATYFCARDPHYYGSSPAWFVYWGQGTLVTVSA

SEQ ID NO: 18 - protein, 107 aa, synthetic construct
  REGION 1..107: note = Synthetic
DIVMTQSQKFMSTTIGDRVSITCEASQIVGIAVAWYQQKPGQSPKLLIYSASNRYTGVPDRFTGSGSGTDFTLTISNMQSEDLANYFCQQYSAYPFTFGSGTKLEVK

SEQ ID NO: 19 - protein, 123 aa, synthetic construct
  REGION 1..123: note = Synthetic
QIQLVQSGPELRKPGETVKISCKASGYSFTNYAMNWVKQAPGKVLKWMGFINTYTGEPTYADDFKGRFAFSLETSASTAYLQINNLKNEDTATYFCTRTRGYYDFDGGAFDYWGQGTSLTVSS

SEQ ID NO: 20 - protein, 107 aa, synthetic construct
  REGION 1..107: note = Synthetic
DIVMTQSQKFMSTSIGDRVSVTCKASQNVGTHLAWYQQKPGQSPKALIFSASYRYIGVPDRFTGSGSGTDFTLTITNVQSEDLAEYFCQQYNTYPLTFGAGTKLELK

SEQ ID NO: 21 - protein, 117 aa, synthetic construct
  REGION 1..117: note = Synthetic
QIQLVQSGPELKKPGETVKISCKASGYTFTHYSMHWVKQAPGKGLKWMGWINTETAEPTYVDDFKGRFAFSLEASASTAFFQINNLKNEDTATYFCARGGLRQGDYWGQGTTLTVSS

SEQ ID NO: 22 - protein, 108 aa, synthetic construct
  REGION 1..108: note = Synthetic
QIVLTQSPPIMSASPGEKVTLTCSATSSVSASYLYWFQQKPGSSPKLWIYSTSNLASGVPARFSGSGSGTSYSLTISNMEPADAASYFCHQWSSYPYTFGGGTKLEIK

SEQ ID NO: 23 - protein, 5 aa, synthetic construct
  REGION 1..5: note = Synthetic
DYYMH

SEQ ID NO: 24 - protein, 17 aa, synthetic construct
  REGION 1..17: note = Synthetic
RIDPEDGETKYAPKFQG

SEQ ID NO: 25 - protein, 4 aa, synthetic construct
  REGION 1..4: note = Synthetic
GLAY

SEQ ID NO: 26 - protein, 12 aa, synthetic construct
  REGION 1..12: note = Synthetic
SASSSVSSSYLY

SEQ ID NO: 27 - protein, 7 aa, synthetic construct
  REGION 1..7: note = Synthetic
STSNLAS

SEQ ID NO: 28 - protein, 9 aa, synthetic construct
  REGION 1..9: note = Synthetic
HQWSSYPYT

SEQ ID NO: 29 - protein, 5 aa, synthetic construct
  REGION 1..5: note = Synthetic
AYYMH

SEQ ID NO: 30 - protein, 17 aa, synthetic construct
  REGION 1..17: note = Synthetic
RIDPEDGESKYAPKFQG

SEQ ID NO: 31 - protein, 5 aa, synthetic construct
  REGION 1..5: note = Synthetic
GSYEY

SEQ ID NO: 32 - protein, 12 aa, synthetic construct
  REGION 1..12: note = Synthetic
SASSSVSSSYLY

SEQ ID NO: 33 - protein, 7 aa, synthetic construct
  REGION 1..7: note = Synthetic
STSNLAS

SEQ ID NO: 34 - protein, 9 aa, synthetic construct
  REGION 1..9: note = Synthetic
YQWSSYPYT

SEQ ID NO: 35 - protein, 5 aa, synthetic construct
  REGION 1..5: note = Synthetic
TYGMS

SEQ ID NO: 36 - protein, 17 aa, synthetic construct
  REGION 1..17: note = Synthetic
WINTYSGVSTCADDFKG

SEQ ID NO: 37 - protein, 14 aa, synthetic construct
  REGION 1..14: note = Synthetic
DPHSYGNSPAWFPY

SEQ ID NO: 38 - protein, 11 aa, synthetic construct
  REGION 1..11: note = Synthetic
KASQNVGISVA

SEQ ID NO: 39 - protein, 7 aa, synthetic construct
  REGION 1..7: note = Synthetic
SASNRYT

SEQ ID NO: 40 - protein, 9 aa, synthetic construct
  REGION 1..9: note = Synthetic
QQYSSYPLT

SEQ ID NO: 41 - protein, 5 aa, synthetic construct
  REGION 1..5: note = Synthetic
EYVLS

SEQ ID NO: 42 - protein, 17 aa, synthetic construct
  REGION 1..17: note = Synthetic
EIYPGTITTYYNEKFKG

SEQ ID NO: 43 - protein, 11 aa, synthetic construct
  REGION 1..11: note = Synthetic
FYDYDGGWFAY

SEQ ID NO: 44 - protein, 12 aa, synthetic construct
  REGION 1..12: note = Synthetic
SASSSVSSSDLH

SEQ ID NO: 45 - protein, 7 aa, synthetic construct
  REGION 1..7: note = Synthetic
GTSNLAS

SEQ ID NO: 46 - protein, 9 aa, synthetic construct
  REGION 1..9: note = Synthetic
QQWSGYPWT

SEQ ID NO: 47 - protein, 5 aa, synthetic construct
  REGION 1..5: note = Synthetic
TYGMS

SEQ ID NO: 48 - protein, 17 aa, synthetic construct
  REGION 1..17: note = Synthetic
WINTYSGVPTYADDFKG

SEQ ID NO: 49 - protein, 14 aa, synthetic construct
  REGION 1..14: note = Synthetic
DPHYYGSSPAWFVY

SEQ ID NO: 50 - protein, 11 aa, synthetic construct
  REGION 1..11: note = Synthetic
EASQIVGIAVA

SEQ ID NO: 51 - protein, 7 aa, synthetic construct
  REGION 1..7: note = Synthetic
SASNRYT

SEQ ID NO: 52 - protein, 9 aa, synthetic construct
  REGION 1..9: note = Synthetic
QQYSAYPFT

SEQ ID NO: 53 - protein, 11 aa, synthetic construct
  REGION 1..11: note = Synthetic
SGYSFTNYAMN

SEQ ID NO: 54 - protein, 17 aa, synthetic construct
  REGION 1..17: note = Synthetic
FINTYTGEPTYADDFKG

SEQ ID NO: 55 - protein, 14 aa, synthetic construct
  REGION 1..14: note = Synthetic
TRGYYDFDGGAFDY

SEQ ID NO: 56 - protein, 11 aa, synthetic construct
  REGION 1..11: note = Synthetic
KASQNVGTHLA

SEQ ID NO: 57 - protein, 7 aa, synthetic construct
  REGION 1..7: note = Synthetic
SASYRYI

SEQ ID NO: 58 - protein, 9 aa, synthetic construct
  REGION 1..9: note = Synthetic
QQYNTYPLT

SEQ ID NO: 59 - protein, 11 aa, synthetic construct
  REGION 1..11: note = Synthetic
SGYTFTHYSMH

SEQ ID NO: 60 - protein, 17 aa, synthetic construct
  REGION 1..17: note = Synthetic
WINTETAEPTYVDDFKG

SEQ ID NO: 61 - protein, 8 aa, synthetic construct
  REGION 1..8: note = Synthetic
GGLRQGDY

SEQ ID NO: 62 - protein, 12 aa, synthetic construct
  REGION 1..12: note = Synthetic
SATSSVSASYLY

SEQ ID NO: 63 - protein, 7 aa, synthetic construct
  REGION 1..7: note = Synthetic
STSNLAS

SEQ ID NO: 64 - protein, 9 aa, synthetic construct
  REGION 1..9: note = Synthetic
HQWSSYPYT

SEQ ID NO: 65 - protein, 119 aa, synthetic construct
  REGION 1..119: note = Synthetic
EVQLLESGGGLVQPGGSLRLSCAASGFTLSSYALSWVRQAPGKGLEWVSYISNLGGSTFYPDTVKGRFTISRDNSKNTLYLQMNSLRAEDTAVYYCAKHLYNYDAFASWGQGTLVTVSS

SEQ ID NO: 66 - protein, 106 aa, synthetic construct
  REGION 1..106: note = Synthetic
DIQLTQSPSFLSASVGDRVTITCRASSSVNYIHWYQQKPGKAPKLLIYATSNLASGVPSRFSGSGSGTEFTLTISSLQPEDFATYYCQQWNSNPLTFGQGTKLEIK

SEQ ID NO: 67 - protein, 106 aa, synthetic construct
  REGION 1..106: note = Synthetic
DIQLTQSPSFLSASVGDRVTITCRASSSVNYIHWYQQKPGKAPKALIYATSNLASGVPSRFSGSGSGTEYTLTISSLQPEDFATYYCQQWNSNPLTFGQGTKLEIK

SEQ ID NO: 68 - protein, 119 aa, synthetic construct
  REGION 1..119: note = Synthetic
EVQLLESGGGLVQPGGSLRLSCAASGFTLSSYALSWVRQAPGKGLEWVAYISNLGGSTFYPDTVKGRFTISRDNSKNTLYLQMNSLRAEDTAVYYCATHLYNYDAFASWGQGTLVTVSS

SEQ ID NO: 69 - protein, 117 aa, synthetic construct
  REGION 1..117: note = Synthetic
QVQLVQSGAEVKKPGASVKVSCKASGYTFTNWVHWVRQAPGQGLEWMGEINPTNARSNYNEKFKKRVTMTRDTSTSTVYMELSSLRSEDTAVYYCARIYYGNSFAHWGQGTLVTVSS

SEQ ID NO: 70 - protein, 113 aa, synthetic construct
  REGION 1..113: note = Synthetic
DIVMTQSPDSLAVSLGERATINCKSSQSLLNAGNQKNYLTWYQQKPGQPPKLLIYWSSTRESGVPDRFSGSGSGTDFTLTISSLQAEDVAVYHCQNNYYYPLTFGGGTKLEIK

SEQ ID NO: 71 - protein, 118 aa, synthetic construct
  REGION 1..118: note = Synthetic
EVQLQQSGPELVKPGSSVKMSCKASGYTFTDYNMHWLKQSHGKSLEWIGYINPKNGGTRYNQKFKGKATLTVNKSSSTAYMELRSLTSEDSAVYYCARLYYGNSFDYWGQGTTLTVSS

SEQ ID NO: 72 - protein, 113 aa, synthetic construct
  REGION 1..113: note = Synthetic
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYFCQNDYSFPFTFGSGTKLEIK

SEQ ID NO: 73 - protein, 119 aa, synthetic construct
  REGION 1..119: note = Synthetic
DVQLVESGGGLVQPGGSRKLSCAASGFTFSSFGMHWVRQAPEKGLEWVAYISSGSSSIYYVDTVKGRFTISRDNPKNTLFLQMTSLRSEDTAMYYCARNAYYGNAFDYWGQGTTLTVSS

SEQ ID NO: 74 - protein, 113 aa, synthetic construct
  REGION 1..113: note = Synthetic
DIVMTQSPSSLTVTAGEKVTMNCKSSQSLLNSGNQKNYLTWYQQKPGQPPKLLIYWASTRTSGVPDRFTGSGSGTDFTLTISSVQAEDLAVYCCQNGYTYPLTFGAGTKLELK

SEQ ID NO: 75 - protein, 118 aa, synthetic construct
  REGION 1..118: note = Synthetic
QVQLQQSGPDLVKPGASVKLSCKASGYTFTNYDINWVKQRPGQGLEWIGGIHPRDGNTKYNEKFKDKATLTIDTSANTAYMEFHSLTSEDSAVYFCARGYYGNSFAYWGQGTLVTVSA

SEQ ID NO: 76 - protein, 113 aa, synthetic construct
  REGION 1..113: note = Synthetic
DIVMTQSPSSLTVTAGEKVTMTCKSGQSLLNSGNQRNYLTWYQQKPGQSPKLLIYWASTRESGVPDRFTGSGSGADFTLTISSVQAEDLALYYCQNAYFYPYTFGGGTKLEIK

SEQ ID NO: 77 - protein, 5 aa, synthetic construct
  REGION 1..5: note = Synthetic
SYALS

SEQ ID NO: 78 - protein, 17 aa, synthetic construct
  REGION 1..17: note = Synthetic
YISNLGGSTFYPDTVKG

SEQ ID NO: 79 - protein, 10 aa, synthetic construct
  REGION 1..10: note = Synthetic
HLYNYDAFAS

SEQ ID NO: 80 - protein, 10 aa, synthetic construct
  REGION 1..10: note = Synthetic
RASSSVNYIH

SEQ ID NO: 81 - protein, 7 aa, synthetic construct
  REGION 1..7: note = Synthetic
ATSNLAS

SEQ ID NO: 82 - protein, 9 aa, synthetic construct
  REGION 1..9: note = Synthetic
QQWNSNPLT

SEQ ID NO: 83 - protein, 4 aa, synthetic construct
  REGION 1..4: note = Synthetic
NWVH

SEQ ID NO: 84 - protein, 17 aa, synthetic construct
  REGION 1..17: note = Synthetic
EINPTNARSNYNEKFKK

SEQ ID NO: 85 - protein, 9 aa, synthetic construct
  REGION 1..9: note = Synthetic
IYYGNSFAH

SEQ ID NO: 86 - protein, 17 aa, synthetic construct
  REGION 1..17: note = Synthetic
KSSQSLLNAGNQKNYLT

SEQ ID NO: 87 - protein, 7 aa, synthetic construct
  REGION 1..7: note = Synthetic
WSSTRES

SEQ ID NO: 88 - protein, 9 aa, synthetic construct
  REGION 1..9: note = Synthetic
QNNYYYPLT

SEQ ID NO: 89 - protein, 5 aa, synthetic construct
  REGION 1..5: note = Synthetic
DYNMH

SEQ ID NO: 90 - protein, 17 aa, synthetic construct
  REGION 1..17: note = Synthetic
YINPKNGGTRYNQKFKG

SEQ ID NO: 91 - protein, 9 aa, synthetic construct
  REGION 1..9: note = Synthetic
LYYGNSFDY

SEQ ID NO: 92 - protein, 17 aa, synthetic construct
  REGION 1..17: note = Synthetic
KSSQSLLNSGNQKNYLT

SEQ ID NO: 93 - protein, 7 aa, synthetic construct
  REGION 1..7: note = Synthetic
WASTRES

SEQ ID NO: 94 - protein, 9 aa, synthetic construct
  REGION 1..9: note = Synthetic
QNDYSFPFT

SEQ ID NO: 95 - protein, 5 aa, synthetic construct
  REGION 1..5: note = Synthetic
SFGMH

SEQ ID NO: 96 - protein, 17 aa, synthetic construct
  REGION 1..17: note = Synthetic
YISSGSSSIYYVDTVKG

SEQ ID NO: 97 - protein, 10 aa, synthetic construct
  REGION 1..10: note = Synthetic
NAYYGNAFDY

SEQ ID NO: 98 - protein, 17 aa, synthetic construct
  REGION 1..17: note = Synthetic
KSSQSLLNSGNQKNYLT

SEQ ID NO: 99 - protein, 7 aa, synthetic construct
  REGION 1..7: note = Synthetic
WASTRTS

SEQ ID NO: 100 - protein, 9 aa, synthetic construct
  REGION 1..9: note = Synthetic
QNGYTYPLT

SEQ ID NO: 101 - protein, 5 aa, synthetic construct
  REGION 1..5: note = Synthetic
NYDIN

SEQ ID NO: 102 - protein, 17 aa, synthetic construct
  REGION 1..17: note = Synthetic
GIHPRDGNTKYNEKFKD

SEQ ID NO: 103 - protein, 9 aa, synthetic construct
  REGION 1..9: note = Synthetic
GYYGNSFAY

SEQ ID NO: 104 - protein, 17 aa, synthetic construct
  REGION 1..17: note = Synthetic
KSGQSLLNSGNQRNYLT

SEQ ID NO: 105 - protein, 7 aa, synthetic construct
  REGION 1..7: note = Synthetic
WASTRES

SEQ ID NO: 106 - protein, 9 aa, synthetic construct
  REGION 1..9: note = Synthetic
QNAYFYPYT

SEQ ID NO: 107 - protein, 112 aa, synthetic construct
  REGION 1..112: note = Synthetic
EVQVVESGGGLVQSGGSLKLSCAGSGFTESAGFMVWHRQVPGKERELVALIATPSGSTNYADSVKGRFTISRDNGKNTVYLQMNSLKPEDTAVYYCNIRGYWGQGTLVTVSS

SEQ ID NO: 108 - protein, 112 aa, synthetic construct
  REGION 1..112: note = Synthetic
EVQVVESGGGLVQSGGSLRLSCAGSGFTESAGFMVWHRQVPGKERELVALIATPSGSTQYADSVKGRFTISRDNGKNTVYLQMNSLKPEDTAVYYCNIRGYWGQGTLVTVSS

SEQ ID NO: 109 - protein, 119 aa, synthetic construct
  REGION 1..119: note = Synthetic
EVQVVESGGGLVQPGGSLRLSCAASGFTFNYFAMSWVRQAPGKGLEWVADIDTGGTDTDYADSVKGRFSISRDNAKNTLYLQMNSLKPEDTAVYYCAKGPREATDIRSWGQGTQVTVSS

SEQ ID NO: 110 - protein, 119 aa, synthetic construct
  REGION 1..119: note = Synthetic
EVQLVESGGDLVQTGGSLRLSCSASGFTFNYFAMSWVRQAPGKGLEWVADIDTGGTDTDYADSVKGRFSISRDNAKNTLYLQMNSLKPEDTAVYYCAKGPREATDIRSWGQGTQVTVSS

SEQ ID NO: 111 - protein, 119 aa, synthetic construct
  REGION 1..119: note = Synthetic
EVQLVESGGGLVQAGGSLRLSCAASGFTFSSHAMSWVRQAPGKGLEWVSDIDTSGTTTDYADSVKGRFTISRDNAKNTLYLQMNSLKPEDTAVYYCAKGPREATDIRSWGQGTQVTVSS

SEQ ID NO: 112 - protein, 114 aa, synthetic construct
  REGION 1..114: note = Synthetic
QVQLVESGGDLVQPGGSLRLSCKASGIIFSSYLLFWYRQPPGKERELVATISSAGSSIDYADSVKGRFTISRDNAKNTVYLQMNSLKPEDTAVYYCRTWPYNYWGQGTLVTVSS

SEQ ID NO: 113 - protein, 123 aa, synthetic construct
  REGION 1..123: note = Synthetic
QVQLVESGGDLVQPGGSLRLSCAASGRTFADAAWGWFRQVPGRDREWVASISRSGDTYYEDSVKGRFTISRDNAKNTVCLQMNSLRAEDTAVYHCAARNSGAPSSIVIAYEYWGQGTQVTVSS

SEQ ID NO: 114 - protein, 123 aa, synthetic construct
  REGION 1..123: note = Synthetic
QVQLVESGGGLVQAGGSLRLTCAASGRTFADAAWGWFRQVPGRDREWVASISRSGDTYYEDSVKGRFTISRDNAKNTVYLQMNSLRAEDTAVYHCAARNSGAPSSIVIAYEYWGQGTQVTVSS

SEQ ID NO: 115 - protein, 123 aa, synthetic construct
  REGION 1..123: note = Synthetic
QVQLVESGGGLVQAGGSLRLTCAASGRAFADAAWGWFRQVPGRDREWVASISRSGDTYYEDSVKGRFTISRDNAKNTVYLQMNSLRAEDTAVYHCAARNSGAPSSIVIAYEYWGQGTQVTVSS

SEQ ID NO: 116 - protein, 123 aa, synthetic construct
  REGION 1..123: note = Synthetic
QVQLEESGGGLAQAGDSLRLTCAASGRTFADAAWGWFRQVPGRNREWVASISRSGDTYYEDSVKGRFTISRDNAKNTVYLQMNSLRAEDTAVYHCAARNSGAPSSIVIAYEYWGQGTQVTVSS

SEQ ID NO: 117 - protein, 123 aa, synthetic construct
  REGION 1..123: note = Synthetic
AVQLMESGGGLVQAGGSLRLTCAASGRTFADAAWGWFRQVPGRDREWVASISRSGDTYYEDSVKGRFTISRDYAKNTVYLQMNSLRAEDTAVYHCAARNSGAPSSIVIAYEYWGHGTQVIVSS

SEQ ID NO: 118 - protein, 123 aa, synthetic construct
  REGION 1..123: note = Synthetic
QVQLVESGGGLVQAGGSLRLTCAASGRTFADAAWGWFRQVPGRDREWVASISRSGDTYYEDSVKGRFTISRDNAKNTVYLQMNSLKPEDTAVYHCAARNSGAPSSIVIAYEYWGQGTQVTVSS

SEQ ID NO: 119 - protein, 10 aa, synthetic construct
  REGION 1..10: note = Synthetic
GFTESAGFMV

SEQ ID NO: 120 - protein, 13 aa, synthetic construct
  REGION 1..13: note = Synthetic
LVALIATPSGSTN

SEQ ID NO: 121 - protein, 7 aa, synthetic construct
  REGION 1..7: note = Synthetic
NIRGYWG

SEQ ID NO: 122 - protein, 10 aa, synthetic construct
  REGION 1..10: note = Synthetic
GFTESAGFMV

SEQ ID NO: 123 - protein, 12 aa, synthetic construct
  REGION 1..12: note = Synthetic
VALIATPSGSTQ

SEQ ID NO: 124 - protein, 7 aa, synthetic construct
  REGION 1..7: note = Synthetic
NIRGYWG

SEQ ID NO: 125 - protein, 10 aa, synthetic construct
  REGION 1..10: note = Synthetic
GFTFNYFAMS

SEQ ID NO: 126 - protein, 8 aa, synthetic construct
  REGION 1..8: note = Synthetic
IDTGGTDT

SEQ ID NO: 127 - protein, 12 aa, synthetic construct
  REGION 1..12: note = Synthetic
AKGPREATDIRS

SEQ ID NO: 128 - protein, 10 aa, synthetic construct
  REGION 1..10: note = Synthetic
GFTFNYFAMS

SEQ ID NO: 129 - protein, 8 aa, synthetic construct
  REGION 1..8: note = Synthetic
IDTGGTDT

SEQ ID NO: 130 - protein, 12 aa, synthetic construct
  REGION 1..12: note = Synthetic
AKGPREATDIRS

SEQ ID NO: 131 - protein, 10 aa, synthetic construct
  REGION 1..10: note = Synthetic
GFTFSSHAMS

SEQ ID NO: 132 - protein, 8 aa, synthetic construct
  REGION 1..8: note = Synthetic
IDTSGTTT

SEQ ID NO: 133 - protein, 12 aa, synthetic construct
  REGION 1..12: note = Synthetic
AKGPREATDIRS

SEQ ID NO: 134 - protein, 10 aa, synthetic construct
  REGION 1..10: note = Synthetic
GIIFSSYLLF

SEQ ID NO: 135 - protein, 8 aa, synthetic construct
  REGION 1..8: note = Synthetic
ISSAGSSI

SEQ ID NO: 136 - protein, 7 aa, synthetic construct
  REGION 1..7: note = Synthetic
RTWPYNY

SEQ ID NO: 137 - protein, 10 aa, synthetic construct
  REGION 1..10: note = Synthetic
GRTFADAAWG

SEQ ID NO: 138 - protein, 7 aa, synthetic construct
  REGION 1..7: note = Synthetic
ISRSGDT

SEQ ID NO: 139 - protein, 17 aa, synthetic construct
  REGION 1..17: note = Synthetic
AARNSGAPSSIVIAYEY

SEQ ID NO: 140 - protein, 10 aa, synthetic construct
  REGION 1..10: note = Synthetic
GRTFADAAWG

SEQ ID NO: 141 - protein, 7 aa, synthetic construct
  REGION 1..7: note = Synthetic
ISRSGDT

SEQ ID NO: 142 - protein, 17 aa, synthetic construct
  REGION 1..17: note = Synthetic
AARNSGAPSSIVIAYEY

SEQ ID NO: 143 - protein, 10 aa, synthetic construct
  REGION 1..10: note = Synthetic
GRAFADAAWG

SEQ ID NO: 144 - protein, 7 aa, synthetic construct
  REGION 1..7: note = Synthetic
ISRSGDT

SEQ ID NO: 145 - protein, 17 aa, synthetic construct
  REGION 1..17: note = Synthetic
AARNSGAPSSIVIAYEY

SEQ ID NO: 146 - protein, 10 aa, synthetic construct
  REGION 1..10: note = Synthetic
GRTFADAAWG

SEQ ID NO: 147 - protein, 7 aa, synthetic construct
  REGION 1..7: note = Synthetic
ISRSGDT

SEQ ID NO: 148 - protein, 17 aa, synthetic construct
  REGION 1..17: note = Synthetic
AARNSGAPSSIVIAYEY

SEQ ID NO: 149 - protein, 10 aa, synthetic construct
  REGION 1..10: note = Synthetic
GRTFADAAWG

SEQ ID NO: 150 - protein, 7 aa, synthetic construct
  REGION 1..7: note = Synthetic
ISRSGDT

SEQ ID NO: 151 - protein, 17 aa, synthetic construct
  REGION 1..17: note = Synthetic
AARNSGAPSSIVIAYEY

SEQ ID NO: 152 - protein, 10 aa, synthetic construct
  REGION 1..10: note = Synthetic
GRTFADAAWG

SEQ ID NO: 153 - protein, 7 aa, synthetic construct
  REGION 1..7: note = Synthetic
ISRSGDT

SEQ ID NO: 154 - protein, 17 aa, synthetic construct
  REGION 1..17: note = Synthetic
AARNSGAPSSIVIAYEY

SEQ ID NO: 155 - protein, 327 aa, synthetic construct
  REGION 1..327: note = Synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 156 - protein, 330 aa, synthetic construct [sequence omitted]

SEQ ID NO: 157 - protein, 107 aa, synthetic construct [sequence omitted]

SEQ ID NO: 158 - protein, 106 aa, synthetic construct
  REGION 1..106: note = Synthetic
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 159 - protein, 113 aa, synthetic construct
  REGION 1..113: note = Synthetic
EVQLVQSGAEVKKPGATVKISCKVSGFNIKDYYMHWVQQAPGKGLEWIGRIDPEDAETKYAPKFQGRVTITADTSTDTAYMELSSLRSEDTAVYYCDRGLAYWGQGTLVTVSS

SEQ ID NO: 160 - protein, 108 aa, synthetic construct; identical to SEQ ID NO: 4
  REGION 1..108: note = Synthetic

SEQ ID NO: 161 - protein, 5 aa, synthetic construct
  REGION 1..5: note = Synthetic
  SITE 1: note = MISC_FEATURE - X is A or D
XYYMH

SEQ ID NO: 162 - protein, 17 aa, synthetic construct
  REGION 1..17: note = Synthetic
  SITE 7: note = MISC_FEATURE - X is G or A
  SITE 9: note = MISC_FEATURE - X is T or S
RIDPEDXEXKYAPKFQG

SEQ ID NO: 163 - skipped

SEQ ID NO: 164 - protein, 9 aa, synthetic construct
  REGION 1..9: note = Synthetic
  SITE 1: note = MISC_FEATURE - X is Y or H
XQWSSYPYT

SEQ ID NO: 165 - protein, 17 aa, synthetic construct
  REGION 1..17: note = Synthetic
  SITE 9: note = MISC_FEATURE - X is S or P
  SITE 11: note = MISC_FEATURE - X is Y or C
WINTYSGVXTXADDFKG

SEQ ID NO: 166 - protein, 14 aa, synthetic construct
  REGION 1..14: note = Synthetic
  SITE 4: note = MISC_FEATURE - X is Y or S
  SITE 7: note = MISC_FEATURE - X is N or S
  SITE 13: note = MISC_FEATURE - X is P or V
DPHXYGXSPAWFXY

SEQ ID NO: 167 - protein, 30 aa, synthetic construct
  REGION 1..30: note = Synthetic
EVQLLESGGGLVQPGGSLRLSCAASGFTLS

SEQ ID NO: 168 - protein, 30 aa, synthetic construct
  REGION 1..30: note = Synthetic
QVQLVQSGAEVKKPGASVKVSCKASGYTFT

SEQ ID NO: 169 - protein, 14 aa, synthetic construct
  REGION 1..14: note = Synthetic
  SITE 14: note = MISC_FEATURE - X is S or A
WVRQAPGKGLEWVX

SEQ ID NO: 170 - protein, 14 aa, synthetic construct
  REGION 1..14: note = Synthetic
WVRQAPGQGLEWMG

SEQ ID NO: 171 - protein, 32 aa, synthetic construct
  REGION 1..32: note = Synthetic
  SITE 32: note = MISC_FEATURE - X is T or K
RFTISRDNSKNTLYLQMNSLRAEDTAVYYCAX

SEQ ID NO: 172 - protein, 32 aa, synthetic construct
  REGION 1..32: note = Synthetic
RVTMTRDTSTSTVYMELSSLRSEDTAVYYCAR

SEQ ID NO: 173 - protein, 11 aa, synthetic construct
  REGION 1..11: note = Synthetic
WGQGTLVTVSS

SEQ ID NO: 174 - protein, 23 aa, synthetic construct
  REGION 1..23: note = Synthetic
DIQLTQSPSFLSASVGDRVTITC

SEQ ID NO: 175 - protein, 23 aa, synthetic construct
  REGION 1..23: note = Synthetic
DIVMTQSPDSLAVSLGERATINC

SEQ ID NO: 176 - protein, 15 aa, synthetic construct
  REGION 1..15: note = Synthetic
  SITE 8: note = MISC_FEATURE - X is Q or K
  SITE 9: note = MISC_FEATURE - X is P or A
  SITE 12: note = MISC_FEATURE - X is L or A
WYQQKPGXXPKXLIY

SEQ ID NO: 177 - protein, 15 aa, synthetic construct
  REGION 1..15: note = Synthetic
WYQQKPGQPPKLLIY

SEQ ID NO: 178 - protein, 32 aa, synthetic construct
  REGION 1..32: note = Synthetic
  SITE 15: note = MISC_FEATURE - X is Y or F
GVPSRFSGSGSGTEXTLTISSLQPEDFATYYC

SEQ ID NO: 179 - protein, 32 aa, synthetic construct
  REGION 1..32: note = Synthetic
GVPDRFSGSGSGTDFTLTISSLQAEDVAVYHC

SEQ ID NO: 180 - protein, 10 aa, synthetic construct
  REGION 1..10: note = Synthetic
  SITE 3: note = MISC_FEATURE - X is Q or G
FGXGTKLEIK

SEQ ID NO: 181 - protein, 10 aa, synthetic construct
  REGION 1..10: note = Synthetic
FGQGTKLEIK

SEQ ID NO: 182 - protein, 10 aa, synthetic construct
  REGION 1..10: note = Synthetic
FGGGTKLEIK

SEQ ID NO: 183 - protein, 5 aa, synthetic construct
  REGION 1..5: note = Synthetic
TGGGG

SEQ ID NO: 184 - protein, 5 aa, synthetic construct
  REGION 1..5: note = Synthetic
GGGGS

SEQ ID NO: 185 - protein, 10 aa, synthetic construct
  REGION 1..10: note = Synthetic
GGGGSGGGGS

SEQ ID NO: 186 - protein, 15 aa, synthetic construct
  REGION 1..15: note = Synthetic
GGGGSGGGGSGGGGS

SEQ ID NO: 187 - protein, 5 aa, synthetic construct
  REGION 1..5: note = Synthetic
SGGGG

SEQ ID NO: 188 - protein, 11 aa, synthetic construct
  REGION 1..11: note = Synthetic
  SITE 1: note = MISC_FEATURE - X is E or K
  SITE 5: note = MISC_FEATURE - X is N or I
  SITE 9: note = MISC_FEATURE - X is S or A
XASQXVGIXVA

SEQ ID NO: 189 - protein, 9 aa, synthetic construct
  REGION 1..9: note = Synthetic
  SITE 5: note = MISC_FEATURE - X is S or A
  SITE 8: note = MISC_FEATURE - X is F or L
QQYSXYPXT

SEQ ID NO: 190 - protein, 30 aa, synthetic construct
  REGION 1..30: note = Synthetic
  SITE 24: note = MISC_FEATURE - X is A or V
EVQLVQSGAEVKKPGATVKISCKXSGFNIK

SEQ ID NO: 191 - protein, 14 aa, synthetic construct
  REGION 1..14: note = Synthetic
WVQQAPGKGLEWIG

SEQ ID NO: 192 - protein, 32 aa, synthetic construct
  REGION 1..32: note = Synthetic
  SITE 11: note = MISC_FEATURE - X is N or D
RVTITADTSTXTAYMELSSLRSEDTAVYYCDR

SEQ ID NO: 193 - protein, 11 aa, synthetic construct
  REGION 1..11: note = Synthetic
WGQGTLVTVSS

SEQ ID NO: 194 - protein, 23 aa, synthetic construct
  REGION 1..23: note = Synthetic
EIVLTQSPATLSLSPGERATLSC

SEQ ID NO: 195 - protein, 15 aa, synthetic construct
  REGION 1..15: note = Synthetic
WYQQKPGQAPKLWIY

SEQ ID NO: 196 - protein, 32 aa, synthetic construct
  REGION 1..32: note = Synthetic
  SITE 15: note = MISC_FEATURE - X is Y or F
GIPARFSGSGSGTDXTLTISSLEPEDFAVYYC

SEQ ID NO: 197 - protein, 10 aa, synthetic construct
  REGION 1..10: note = Synthetic
FGQGTKLEIK

SEQ ID NO: 198 - protein, 17 aa, synthetic construct
  REGION 1..17: note = Synthetic
RIDPEDAETKYAPKFQG

SEQ ID NO: 199 - protein, 14 aa, synthetic construct
  REGION 1..14: note = Synthetic
WVRQAPGKGLEWVS

SEQ ID NO: 200 - protein, 32 aa, synthetic construct
  REGION 1..32: note = Synthetic
RFTISRDNSKNTLYLQMNSLRAEDTAVYYCAK

SEQ ID NO: 201 - protein, 15 aa, synthetic construct
  REGION 1..15: note = Synthetic
WYQQKPGKAPKLLIY

SEQ ID NO: 202 - protein, 32 aa, synthetic construct
  REGION 1..32: note = Synthetic
GVPSRFSGSGSGTEFTLTISSLQPEDFATYYC

SEQ ID NO: 203 - protein, 15 aa, synthetic construct
  REGION 1..15: note = Synthetic
WYQQKPGKAPKALIY

SEQ ID NO: 204 - protein, 32 aa, synthetic construct
  REGION 1..32: note = Synthetic
GVPSRFSGSGSGTEYTLTISSLQPEDFATYYC

SEQ ID NO: 205 - protein, 32 aa, synthetic construct
  REGION 1..32: note = Synthetic
RFTISRDNSKNTLYLQMNSLRAEDTAVYYCAT

SEQ ID NO: 206 - protein, 15 aa, synthetic construct
  REGION 1..15: note = Synthetic
  SITE 12: note = MISC_FEATURE - X is L or A
WYQQKPGKAPKXLIY

SEQ ID NO: 207 - protein, 30 aa, synthetic construct
  REGION 1..30: note = Synthetic
EVQLVQSGAEVKKPGATVKISCKVSGFNIK

SEQ ID NO: 208 - protein, 32 aa, synthetic construct
  REGION 1..32: note = Synthetic
RVTITADTSTNTAYMELSSLRSEDTAVYYCDR

SEQ ID NO: 209 - protein, 32 aa, synthetic construct
  REGION 1..32: note = Synthetic
GIPARFSGSGSGTDYTLTISSLEPEDFAVYYC

SEQ ID NO: 210 - protein, 32 aa, synthetic construct
  REGION 1..32: note = Synthetic
RVTITADTSTDTAYMELSSLRSEDTAVYYCDR

SEQ ID NO: 211 - protein, 30 aa, synthetic construct
  REGION 1..30: note = Synthetic
EVQLVQSGAEVKKPGATVKISCKASGFNIK

SEQ ID NO: 212 - protein, 32 aa, synthetic construct
  REGION 1..32: note = Synthetic
GIPARFSGSGSGTDFTLTISSLEPEDFAVYYC

SEQ ID NO: 213 - protein, 14 aa, synthetic construct
  REGION 1..14: note = Synthetic
WVRQAPGKGLEWVA

SEQ ID NO: 214 - protein, 120 aa, synthetic construct
  REGION 1..120: note = Synthetic
EVQLVQSGAEVKKPGESLRISCKASGYSFTSYWVHWVRQMPGKGLEWMGNIDPSDSDTHYSPSFQGHVTLSVDKSISTAYLQLSSLKASDTAMYYCVRGGTGTLAYFAYWGQGTLVTVSS

SEQ ID NO: 215 - protein, 112 aa, synthetic construct
  REGION 1..112: note = Synthetic
DVVMTQSPLSLPVTLGQPASISCRSSQSLVHSYGNTYLYWFQQRPGQSPRLLIYRVSNRFSGVPDRFSGSGSGTDFTLKISRVEAEDVGVYYCFQGTHVPYTFGGGTKVEIK

SEQ ID NO: 216 - protein, 120 aa, synthetic construct
  REGION 1..120: note = Synthetic
QVQLVQSGAEVKKPGASVKVSCKASGYTFTSYWITWVKQAPGQGLEWIGDIYPGSGSTNHIEKFKSKATLTVDTSISTAYMELSRLRSDDTAVYYCATGYGSSYGYFDYWGQGTLVTVSS

SEQ ID NO: 217 - protein, 107 aa, synthetic construct
  REGION 1..107: note = Synthetic
DIQMTQSPSSLSASVGDRVTITCRASENIYSYLAWYQQKPGKAPKLLIYTAKTLAEGVPSRFSGSGSGTDFTLTISSLQPEDFATYYCQHQYGPPFTFGQGTKLELK

SEQ ID NO: 218 - protein, 17 aa, synthetic construct
  REGION 1..17: note = Synthetic
YNQKEGHFPRVTTVSDL

SEQ ID NO: 219 - protein, 7 aa, synthetic construct
  REGION 1..7: note = Synthetic
SGAGTEL

SEQ ID NO: 220 - protein, 11 aa, synthetic construct
  REGION 1..11: note = Synthetic
TNVDPVGESVS

SEQ ID NO: 221 - protein, 12 aa, synthetic construct
  REGION 1..12: note = Synthetic
TNVDPVGESVSY

SEQ ID NO: 222 - protein, 6 aa, synthetic construct
  REGION 1..6: note = Synthetic
VGPIQW

SEQ ID NO: 223 - protein, 119 aa, synthetic construct
  REGION 1..119: note = Synthetic
EVQLVESGGGLVQPGGSLRLSCAASGFTFSSHAMSWVRQAPGKGLEWVSDIDTSGTTTDYADSVKGRFTISRDNSKNTLYLQMNSLRAEDTAVYYCAKGPREATDIRSWGQGTTVTVSS

SEQ ID NO: 224 - protein, 106 aa, synthetic construct
  REGION 1..106: note = Synthetic
DIQLTQSPSFLSASVGDRVTITCRASSSVNYIHWYQQKPGKAPKALIYATSNLASGVPSRFSGSGSGTEYTLTISSLQPEDFATYYCQQWNANPLTFGQGTKLEIK

SEQ ID NO: 225 - protein, 9 aa, synthetic construct
  REGION 1..9: note = Synthetic
QQWNANPLT

SEQ ID NO: 226 - protein, 241 aa, synthetic construct
  REGION 1..241: note = Synthetic
QIVLTQSPAIMSASPGEKVTLTCSASSSVSSSYLYWYQQKPGSSPKLWIYSTSNLASGVPARFSGSGSGTSYSLTISSMEAEDAASYFCHQWSSYPYTFGGGTKLEIKGGGGSGGGGSGGGGSGGGGSEVQLQQSGAELVKPGASVKLSCTASGFNIKDYYMHWVKQRTEQGLEWIGRIDPEDGETKYAPKFQGKATITADTSSNTAYLQLSSLTSEDTAVYYCDRGLAYWGQGTLVTVSA

SEQ ID NO: 227 - protein, 250 aa, synthetic construct
  REGION 1..250: note = Synthetic
DIVMTQSPDSLAVSLGERATINCKSSQSLLNAGNQKNYLTWYQQKPGQPPKLLIYWSSTRESGVPDRFSGSGSGTDFTLTISSLQAEDVAVYHCQNNYYYPLTFGGGTKLEIKGGGGSGGGGSGGGGSGGGGSQVQLVQSGAEVKKPGASVKVSCKASGYTFTNWVHWVRQAPGQGLEWMGEINPTNARSNYNEKFKKRVTMTRDTSTSTVYMELSSLRSEDTAVYYCARIYYGNSFAHWGQGTLVTVSS

SEQ ID NO: 228 - protein, 98 aa, synthetic construct
  REGION 1..98: note = Synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 229 - protein, 12 aa, synthetic construct
  REGION 1..12: note = Synthetic
ESKYGPPCPPCP

SEQ ID NO: 230 - protein, 217 aa, synthetic construct
  REGION 1..217: note = Synthetic
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 231 - protein, 98 aa, synthetic construct
  REGION 1..98: note = Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 232 - protein, 12 aa, synthetic construct
  REGION 1..12: note = Synthetic
EPKSCDKTHTCP

SEQ ID NO: 233 - protein, 220 aa, synthetic construct
  REGION 1..220: note = Synthetic
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2024/0336686 A1
(43) Pub. Date: Oct. 10, 2024

(54) NOVEL MULTI-SPECIFIC MOLECULES

(71) Applicants: ELPISCIENCE (SUZHOU) BIOPHARMA, LTD., Suzhou, Jiangsu (CN); ELPISCIENCE BIOPHARMA, LTD., Shanghai (CN)

(72) Inventors: Hongtao LU, Shanghai (CN); Dawei SUN, Shanghai (CN); Yanan GENG, Shanghai (CN); Jing WANG, Shanghai (CN); Haixia JIANG, Shanghai (CN); Zhihao WU, Shanghai (CN); Rui GAO, Shanghai (CN); Xiaofeng NIU, Shanghai (CN); Yangsheng QIU, Shanghai (CN)

(21) Appl. No.: 18/292,921

(22) PCT Filed: Jul. 28, 2022
(86) PCT No.: PCT/US2022/074284

§ 371 Date: Jan. 28, 2024

(30) Foreign Application Priority Data
Jul. 28, 2021 (WO) PCT/CN2021/109028
Jul. 4, 2022 (WO) PCT/CN2022/103725

(51) Int. Cl. C07K 16/28; A61K 39/00; A61P 35/00

(52) U.S. Cl. CPC C07K 16/2827; A61P 35/00; A61K 2039/505; C07K 2317/24; C07K 2317/31; C07K 2317/52; C07K 2317/55; C07K 2317/565; C07K 2317/569; C07K 2317/622; C07K 2317/73; C07K 2317/76; C07K 2317/92

(57) ABSTRACT

The present disclosure provides multi-specific molecules specific for SIPR-alpha and one or more target antigens, isolated polynucleotide encoding the same, pharmaceutical compositions comprising the same, and the uses thereof. The present disclosure provides the multi-specific molecule provided comprises a SIRP-alpha binding domain, an activating receptor-binding domain comprising an Fc domain, and a target antigen binding domain.

### FIELD OF THE INVENTION

The present disclosure generally relates to novel multi-specific molecules specific for SIRP-α and tumor associated antigen.

### BACKGROUND

Signal-regulatory protein alpha (SIRPα), is an inhibitory receptor expressed primarily on myeloid cells and dendritic cells. SIRPα comprises an immunoreceptor tyrosine-based inhibition motif (ITIM) cytoplasmic domain. The immunoregulatory activity of SIRPα on myeloid cells is activated by binding to its ligand CD47, which induces tyrosine phosphorylation of the ITIM cytoplasmic domain of SIRPα, with subsequent recruitment of SH2-containing tyrosine phosphatase (SHP-1/2). SHP-1/2 then mediates inhibitory signaling events through protein dephosphorylation, ultimately leading to the inhibition of phagocytosis in macrophages (Barclay A N, Van den Berg T K. The interaction between signal regulatory protein alpha (SIRPα) and CD47: structure, function, and therapeutic target. Annu Rev Immunol. 2014; 32:25-50; Oldenborg P A, et al. Role of CD47 as a marker of self on red blood cells. Science. 2000; 288(5473):2051-2054.). As such, binding of CD47 to SIRPα delivers a “don't eat me” signal to suppress phagocytosis.

CD47 is ubiquitously expressed on normal cells and upregulated on many cancer cells. High CD47 expression is a mechanism used by cancer cells to evade the immune system that correlates with poor clinical outcomes (Willingham S B, et al. The CD47-signal regulatory protein alpha (SIRPα) interaction is a therapeutic target for human solid tumors. Proc Natl Acad Sci USA. 2012; 109(17):6662-6667; Zhao X W, et al. CD47-signal regulatory protein—a (SIRPα) interactions form a barrier for antibody-mediated tumor cell destruction. Proc Natl Acad Sci USA. 2011; 108(45):18342-18347; Majeti R, et al. CD47 is an adverse prognostic factor and therapeutic antibody target on human acute myeloid leukemia stem cells. Cell. 2009; 138(2):286-299.). Bi-specific macrophage enhancing (BiME) antibodies specific for SIRPα and a second antigen (e.g., a target antigen) have been designed to provide enhanced phagocytotic properties on cancer cells, see, for examples, WO2015138600A2, which are incorporated herein by reference. And more BiME molecules constructed with different properties of SIRPα antibodies and formats need to be more studied and compared.

Therefore, there is need for developing an improved BiME antibody or multi-specific molecule specific for SIRPα with reduced side effects and high safety.

### BRIEF SUMMARY OF THE INVENTION

Throughout the present disclosure, the articles “a,” “an,” and “the” are used herein to refer to one or to more than one (i.e., to at least one) of the grammatical object of the article. By way of example, “an antibody” means one antibody or more than one antibody.

The present disclosure provides novel multi-specific molecules specific for SIRP-α and tumor associated antigen, amino acid and nucleotide sequences thereof, and uses thereof.

In one aspect, the present disclosure provides a multi-specific molecule comprising:
(a) a SIRP-alpha binding domain,
(b) an activating receptor-binding domain, and
(c) a target antigen binding domain that binds to a target antigen expressed on a target cell co-expressing the target antigen and CD47,
wherein the multi-specific molecule selectively induces effector function of an immune effector cell in the presence of the target antigen, and wherein the immune effector cell co-expresses SIRP-alpha and the activating receptor.

In certain embodiments, the multi-specific molecule provided herein comprises a SIRP-alpha binding domain provided herein, an activating receptor-binding domain provided herein comprising an Fc domain, and a target antigen binding domain provided herein.

In certain of these embodiments, the target binding domain is a Claudin18.2 binding domain or a PD-L1-binding domain.

In certain embodiments, the target cell co-expressing the target antigen and CD47 is a cancer cell, an infected cell, or a disease cell of interest for elimination by the effector function of the immune effector cell.

In certain embodiments, the multi-specific molecule induces minimal effector function of the immune effector cell in the absence of the target antigen.

In certain embodiments, the effector function induced by the multi-specific molecule in the absence of the target antigen is no more than 10% of that induced in the presence of the target antigen.

In certain embodiments, the immune effector cell is a myeloid cell, optionally, the immune effector cell is a macrophage cell, monocytes, neutrophils, eosinophil, phagocyte or basophil, optionally the immune effector cell is macrophage cell.

In certain embodiments, the effector function comprises phagocytosis of the cell co-expressing the antigen and CD47 by the immune effector cell.

In certain embodiments, the activating receptor is fragment crystallizable γ receptors (FcγRs), TREM2, lectin, scavenger receptor A1 (SRA1), MARCO, CD36, CD163, CD68, CD205, CD206, FcDR1, CD207, CD209, RAGE, CD14, CD64, F4/80, CD64, CD32a, CD16a, CD89, CD19, CD28, CSFR, PDGFR, MSR1, SCARA3, COLEC12, SCARA5, SCARB1, SCARB2, dectin 1, RAGE (SR-E1), LRP1, LRP2, ASGP, SR-PSOX, CXCL16, OLR1, SCARF1, SCARF2, CXCL16, STAB1, STAB2, SRCRB4D, SSC5D, CCR2, CX3CR1, CSF1R, Tie2, HuCRIg(L), and CD169 receptor or complement receptors (such as CR1 and CR3), PI3K, FcγRI, FcγR2A, FcγR2B2, FcγR2C, FcγR3A, BAH. Tyro3, Axl, Traf6, Syk, MyD88, Zap70, FcεR1, FcαR1, BAFF-R, DAP 12, NFAM1, MRC1, ItgB5, MERTK, ELMO, and CD79b; optionally, the activating receptor is FcγR.

In certain embodiments, the activating receptor-binding domain comprises an Fc domain, optionally the Fc domain is derived from IgG1 or IgG4.

In certain embodiments, the SIRP-alpha binding domain is capable of substantially blocking interaction between SIRP-alpha and CD47.

In certain embodiments, the SIRP-alpha binding domain is capable of completely blocking interaction between SIRP-alpha and CD47.

In certain embodiments, the SIRP-alpha binding domain is capable of substantially blocking SHP-1 recruitment mediated by interaction between SIRP-alpha and CD47.

In certain embodiments, the SIRP-alpha binding domain is capable of completely blocking SHP-1 recruitment mediated by interaction between SIRP-alpha and CD47.

In certain embodiments, the SIRP-alpha binding domain has minimal intrinsic activity to induce the effector function of the immune effector cell.

In certain embodiments, the SIRP-alpha binding domain and the activating receptor-binding domain are in close proximity to permit binding of the multi-specific molecule to both SIRP-alpha and the activating receptor co-expressed on the same immune effector cell.

In certain embodiments, the SIRP-alpha binding domain and/or the target antigen binding domain comprises an antibody domain or an antibody mimetic domain; optionally the antibody mimetic domain comprises a fibronectin domain, Z domain of protein A (Affibody), gamma-B crystalline domain, ubiquitin domain, cystatin domain, Sac7d domain, triple helix coiled coil domain, lipocalins domain, A domains of a membrane receptor, Ankyrin repeat motif, SH3 domain of Fyn, Kunitz domain of a protease inhibitor, type III domain of fibronectin (Minibody), carbohydrae binding module 32-2.

In certain embodiments, the antibody domain comprises a Fab, a VHH, a single chain Fv (scFv), diabody, a Fab′, a F(ab′)₂, a Fd, an Fv fragment, a disulfide stabilized Fv fragment (dsFv), a (dsFv)₂, a bispecific dsFv (dsFv-dsFv′), a disulfide stabilized diabody (ds diabody), F(ab)₂, an scFv dimer (bivalent diabody), a camelized single domain antibody, a nanobody, a Tetrabody, a domain antibody, or a bivalent domain antibody.

In certain embodiments, the multi-specific molecule provided herein comprises a multi-specific antibody comprising a target antigen binding antibody domain, a SIRP-a binding antibody domain, and an Fc domain.

In certain embodiments, the target antigen binding antibody domain is linked to N-terminus of the Fc domain.

In certain embodiments, the target antigen binding antibody domain comprises a Fab domain, optionally, the Fab domain comprises a heavy chain linked to one of the N-termini of the Fc domains.

In certain embodiments, the multi-specific molecule comprises two target antigen binding antibody domains, each of which comprises a Fab domain, optionally, each of the Fab domains comprises a heavy chain linked to each N-terminus of the Fc domain, respectively.

In certain embodiments, the SIRP-alpha binding domain is linked to the Fc domain or to the target antigen binding antibody domain.

In certain embodiments, the SIRP-alpha binding domain is linked to C-terminus of the Fc domain.

In certain embodiments, the SIRP-alpha binding domain is linked to N-terminus of the Fc domain, with the proviso that the SIRP-alpha binding domain and the target antigen binding antibody domain are not linked to the same N-terminus of the Fc domain.

In certain embodiments, the SIRP-alpha binding domain is linked to the C-terminus of light chain of the target antigen binding Fab domain.

In certain embodiments, the SIRP-alpha binding antibody domain is linked to N-terminus of the Fc domain.

In certain embodiments, the SIRP-alpha binding antibody domain comprises a Fab domain, optionally, the Fab domain comprises a heavy chain linked to one of the N-termini of the Fc domains.

In certain embodiments, the antibody comprises two SIRP-alpha binding antibody domains, each of which comprises a Fab domain, optionally, each of the Fab domains comprises a heavy chain linked to each N-terminus of the Fc domain, respectively.

In certain embodiments, the target antigen binding domain is linked to the Fc domain or to the SIRP-alpha binding antibody domain.

In certain embodiments, the target antigen binding domain is linked to the N-terminus of the Fc domain, with the provision that the target antigen binding domain and the SIRP-alpha binding domain are not linked to the same N-terminus of the Fc domain.

In certain embodiments, the target antigen binding domain is linked to the C-terminus of the light chain of the SIRP-alpha binding Fab domain.

In certain embodiments, the target antigen comprises a tumor surface antigen.

In certain embodiments, the tumor surface antigen is PD-L1, claudin 18.2, BCMA, CD19, CD20, CD22, CD24, CD25, CD30, CD33, CD38, CD44, CD52, CD56, CD70, CD96, CD97, CD99, CD123, EGFR, HER2, HER3, CD117, C-Met, EGFR, EGFRvIII, ERBB3, ERBB4, VEGFR1, VEGFR2, ROR1, PTHR2, B7-H1(PD-L1), B7-H2, B7-H3, B7-H4, B7-H5, B7-H6, B7-H7, Trop-2, GPC-3, EPCAM, DLL-3, Nectin-4, Claudin6, Muc-1, PSMA, GD3, FAP, CEA, or EphA2.

The multi-specific molecule provided herein can be in any suitable format. Illustrative examples are provided as follows. In certain embodiments, the multi-specific molecule provided herein comprises a target antigen-binding antibody comprising two heavy chains and two light chains, wherein the C-terminus of each of the light chain is fused to an anti-SIRPα scFv (i.e. SIRPα binding domain). The target antigen-binding antibody comprises the target antigen binding domain and Fc domain. Illustrative example is shown in FIG. 2A.

In certain embodiments, the multi-specific molecule provided herein comprises a target antigen-binding antibody comprising two heavy chains and two light chains, wherein the C-terminus of each of the heavy chain is fused to an anti-SIRPα scFv (i.e. SIRPα binding domain). The target antigen-binding antibody comprises the target antigen binding domain and Fc domain. Illustrative example is shown in FIG. 2B.

In certain embodiments, the multi-specific molecule provided herein comprises an anti-SIRPα antibody comprising two heavy chains and two light chains, wherein the C-terminus of each of the light chain is fused to an scFv capable of binding to a target antigen (i.e. target antigen binding domain). The anti-SIRPα antibody comprises the SIRPα binding domain and Fc domain. Illustrative example is shown in FIG. 2C.

In certain embodiments, the multi-specific molecule provided herein comprises an anti-SIRPα antibody comprising two heavy chains and two light chains, wherein the C-terminus of each of the heavy chain is fused to an scFv capable of binding to a target antigen (i.e. target antigen binding domain). The anti-SIRPα antibody comprises the SIRPα binding domain and Fc domain. Illustrative example is shown in FIG. 2D.

In certain embodiments, the multi-specific molecule provided herein comprises an anti-SIRPα antibody comprising two heavy chains and two light chains, wherein the C-terminus of each of the light chain is fused to a single domain antibody (sdAb) capable of binding to a target antigen (i.e. target antigen binding domain). The anti-SIRPα antibody comprises the SIRPα binding domain and Fc domain. Illustrative example is shown in FIG. 6A.

In certain embodiments, the multi-specific molecule provided herein comprises an anti-SIRPα antibody comprising two heavy chains and two light chains, wherein the C-terminus of each of the heavy chain is fused to a single domain antibody (sdAb) capable of binding to a target antigen (i.e. target antigen binding domain). The anti-SIRPα antibody comprises the SIRPα binding domain and Fc domain. Illustrative example is shown in FIG. 6B.

In certain embodiments, the multi-specific molecule provided herein comprises an anti-SIRPα antibody comprising two heavy chains and two light chains, wherein the N-terminus of each of the heavy chain is fused to a single domain antibody (sdAb) capable of binding to a target antigen (i.e. target antigen binding domain). The anti-SIRPα antibody comprises the SIRPα binding domain and Fc domain. Illustrative example is shown in FIG. 6C.

In certain embodiments, the multi-specific molecule provided herein comprises an anti-SIRPα antibody comprising two heavy chains and two light chains, wherein the N-terminus of each of the light chain is fused to a single domain antibody (sdAb) capable of binding to a target antigen (i.e. target antigen binding domain). The anti-SIRPα antibody comprises the SIRPα binding domain and Fc domain. Illustrative example is shown in FIG. 6D.

In certain embodiments, the multi-specific molecule provided herein comprises an anti-SIRPα binding domain (e.g. Fab) and a single domain antibody (sdAb) capable of binding to a target antigen, respectively, each being fused to N terminus of a polypeptide chain of the Fc domain. Illustrative example is shown in FIG. 6E.

In certain embodiments, the multi-specific molecule provided herein comprises two heavy chains, each comprising a single domain antibody (sdAb) capable of binding to a target antigen fused to N terminus of a polypeptide chain of the Fc domain, and further comprises at least one anti-SIRPα binding domain fused to the C terminus of one of the polypeptide chains of the Fc domain. In certain embodiments, the multi-specific molecule provided herein comprises one anti-SIRPα binding domain fused to the C terminus of one of the polypeptide chains of the Fc domain. Illustrative example is shown in FIG. 6F.

In certain embodiments, the multi-specific molecule provided herein comprises two anti-SIRPα binding domains, each being fused to the C terminus of one of the polypeptide chains of the Fc domain. Illustrative example is shown in FIG. 6G.

In certain embodiments, the SIRP-alpha binding domain comprises:
a) a HCDR1 comprising the sequence of X₁YYMH (SEQ ID NO: 161), a HCDR2 comprising the sequence of RIDPEDX₂EX₃KYAPKFQG (SEQ ID NO: 162), and a HCDR3 comprising the sequence of GX₁₅X₄X₅Y (SEQ ID NO: 163); and/or a LCDR1 comprising the sequence of SASSSVSSSYLY (SEQ ID NO: 26), a LCDR2 comprising the sequence of STSNLAS (SEQ ID NO: 27), and a LCDR3 comprising the sequence of X₆QWSSYPYT (SEQ ID NO: 164); or
b) a HCDR1 comprising the sequence of TYGMS (SEQ ID NO: 35), a HCDR2 comprising the sequence of WINTYSGVX₇TX₈ADDFKG (SEQ ID NO: 165), and a HCDR3 comprising the sequence of DPHX₉YGX₁₀SPAWFX₁₁Y (SEQ ID NO: 166); and/or a LCDR1 comprising the sequence of X₁₂ASQX₁₃VGIX₁₄VA (SEQ ID NO: 188), a LCDR2 comprising the sequence of SASNRYT (SEQ ID NO: 39), and a LCDR3 comprising the sequence of QQYSX₁₆YPX₁₇T (SEQ ID NO: 189); or
c) a HCDR1 comprising the sequence of EYVLS (SEQ ID NO: 41), a HCDR2 comprising the sequence of EIYPGTITTYYNEKFKG (SEQ ID NO: 42), and a HCDR3 comprising the sequence of FYDYDGGWFAY (SEQ ID NO: 43); and/or a LCDR1 comprising the sequence of SASSSVSSSDLH (SEQ ID NO: 44), a LCDR2 comprising the sequence of GTSNLAS (SEQ ID NO: 45), and a LCDR3 comprising the sequence of QQWSGYPWT (SEQ ID NO: 46),
   wherein
   X₁ is A or D; X₂ is G or A; X₃ is T or S; X₄ is L or Y; X₅ is E or A; X₆ is Y or H; X₇ is S or P; X₈ is Y or C; X₉ is Y or S; X₁₀ is N or S; X₁₁ is P or V; X₁₂ is E or K; X₁₃ is N or I; X₁₄ is S or A; X₁₅ is S or absent; X₁₆ is S or A; X₁₇ is F or L.

In certain embodiments, the SIRP-alpha binding domain comprises:
a) a HCDR1 comprising the sequence of SEQ ID NO: 23, a HCDR2 comprising the sequence of SEQ ID NO: 24 or SEQ ID NO: 198, and a HCDR3 comprising the sequence of SEQ ID NO: 25; and/or a LCDR1 comprising the sequence of SEQ ID NO: 26, a LCDR2 comprising the sequence of SEQ ID NO: 27, and a LCDR3 comprising the sequence of SEQ ID NO: 28; or
b) a HCDR1 comprising the sequence of SEQ ID NO: 29, a HCDR2 comprising the sequence of SEQ ID NO: 30, and a HCDR3 comprising the sequence of SEQ ID NO: 31; and/or a LCDR1 comprising the sequence of SEQ ID NO: 32, a LCDR2 comprising the sequence of SEQ ID NO: 33, and a LCDR3 comprising the sequence of SEQ ID NO: 34; or
c) a HCDR1 comprising the sequence of SEQ ID NO: 35, a HCDR2 comprising the sequence of SEQ ID NO: 36, and a HCDR3 comprising the sequence of SEQ ID NO: 37; and/or a LCDR1 comprising the sequence of SEQ ID NO: 38, a LCDR2 comprising the sequence of SEQ ID NO: 39, and a LCDR3 comprising the sequence of SEQ ID NO: 40; or
d) a HCDR1 comprising the sequence of SEQ ID NO: 47, a HCDR2 comprising the sequence of SEQ ID NOs: 48, and a HCDR3 comprising the sequence of SEQ ID NOs: 49; and/or a LCDR1 comprising the sequence of SEQ ID NOs: 50, a LCDR2 comprising the sequence of SEQ ID NOs: 51, and a LCDR3 comprising the sequence of SEQ ID NOs: 52.

In certain embodiments, the SIRP-alpha binding domain comprises the same HCDRs and LCDRs as anti-SIRP-alpha antibody selected from the group consisting of C25, C15, C42, C59 and C73, wherein:
a) the C25 comprises a heavy chain variable region comprising the sequence of SEQ ID NO: 1, and/or a light chain variable region comprising the sequence of SEQ ID NO: 2,
b) the C15 comprises a heavy chain variable region comprising the sequence of SEQ ID NO: 11, and/or a light chain variable region comprising the sequence of SEQ ID NO: 12,
c) the C42 comprises a heavy chain variable region comprising the sequence of SEQ ID NO: 13, and/or a light chain variable region comprising the sequence of SEQ ID NO: 14,
d) the C59 comprises a heavy chain variable region comprising the sequence of SEQ ID NO: 15, and/or a light chain variable region comprising the sequence of SEQ ID NO: 16, and
e) the C73 comprises a heavy chain variable region comprising the sequence of SEQ ID NO: 17, and/or a light chain variable region comprising the sequence of SEQ ID NO: 18.

In certain embodiments, the SIRP-alpha binding domain further comprises one or more of heavy chain HFR1, HFR2, HFR3 and HFR4, and/or one or more of light chain LFR1, LFR2, LFR3 and LFR4, wherein:
a) the HFR1 comprises EVQLVQSGAEVKKPGATVKISCKX₂₀SGFNIK (SEQ ID NO: 190) or a homologous sequence of at least 80% sequence identity thereof, and/or
b) the HFR2 comprises WVQQAPGKGLEWIG (SEQ ID NO: 191) or a homologous sequence of at least 80% sequence identity thereof, and/or
c) the HFR3 sequence comprises RVTITADTSTX₂₁TAYMELSSLRSEDTAVYYCDR (SEQ ID NO: 192) or a homologous sequence of at least 80% sequence identity thereof, and/or
d) the HFR4 comprises WGQGTLVTVSS (SEQ ID NO: 193) or a homologous sequence of at least 80% sequence identity thereof, and/or
e) the LFR1 comprises EIVLTQSPATLSLSPGERATLSC (SEQ ID NO: 194) or a homologous sequence of at least 80% sequence identity thereof, and/or
f) the LFR2 comprises WYQQKPGQAPKLWIY (SEQ ID NO: 195) or a homologous sequence of at least 80% sequence identity thereof, and/or
g) the LFR3 comprises GIPARFSGSGSGTDX₂₂TLTISSLEPEDFAVYYC (SEQ ID NO: 196) or a homologous sequence of at least 80% sequence identity thereof, and/or
h) the LFR4 comprises FGQGTKLEIK (SEQ ID NO: 197) or a homologous sequence of at least 80% sequence identity thereof,
   wherein X₂₀ is A or V; X₂₁ is N or D; X₂₂ is Y or F.

In certain embodiments, the SIRP-alpha binding domain comprises
f) the heavy chain variable region comprises the sequence of SEQ ID NO: 1 and/or the light chain variable region comprises the sequence of SEQ ID NO: 2; or
g) the heavy chain variable region comprises the sequence of SEQ ID NO: 3 and/or the light chain variable region comprises the sequence of SEQ ID NO: 4; or
h) the heavy chain variable region comprises the sequence of SEQ ID NO: 5 and/or the light chain variable region comprises the sequence of SEQ ID NO: 6; or
i) the heavy chain variable region comprises the sequence of SEQ ID NO: 7 and/or the light chain variable region comprises the sequence of SEQ ID NO: 8; or
j) the heavy chain variable region comprises the sequence of SEQ ID NO: 9 and/or the light chain variable region comprises the sequence of SEQ ID NO: 10; or
k) the heavy chain variable region comprises the sequence of SEQ ID NO: 11 and/or the light chain variable region comprises the sequence of SEQ ID NO: 12; or
l) the heavy chain variable region comprises the sequence of SEQ ID NO: 13 and/or the light chain variable region comprises the sequence of SEQ ID NO: 14; or
m) the heavy chain variable region comprises the sequence of SEQ ID NO: 15 and/or the light chain variable region comprises the sequence of SEQ ID NO: 16; or
n) the heavy chain variable region comprises the sequence of SEQ ID NO: 17 and/or the light chain variable region comprises the sequence of SEQ ID NO: 18 or
o) the heavy chain variable region comprises the sequence of SEQ ID NO: 159 and/or the light chain variable region comprises the sequence of SEQ ID NO: 160.

In certain embodiments, the target antigen binding domain comprises a claudin 18.2 binding domain.

In certain embodiments, the claudin 18.2 binding domain comprises:
p) a HCDR1 comprising the sequence of SEQ ID NO: 77, a HCDR2 comprising the sequence of SEQ ID NO: 78, and a HCDR3 comprising the sequence of SEQ ID NO: 79; and/or a LCDR1 comprising the sequence of SEQ ID NO: 80, a LCDR2 comprising the sequence of SEQ ID NO: 81, and a LCDR3 comprising the sequence of SEQ ID NO: 82 or SEQ ID NO: 225; or
q) a HCDR1 comprising the sequence of SEQ ID NO: 83, a HCDR2 comprising the sequence of SEQ ID NO: 84, and a HCDR3 comprising the sequence of SEQ ID NO: 85; and/or a LCDR1 comprising the sequence of SEQ ID NO: 86, a LCDR2 comprising the sequence of SEQ ID NO: 87, and a LCDR3 comprising the sequence of SEQ ID NO: 88; or
r) a HCDR1 comprising the sequence of SEQ ID NO: 89, a HCDR2 comprising the sequence of SEQ ID NO: 90, and a HCDR3 comprising the sequence of SEQ ID NO: 91; and/or a LCDR1 comprising the sequence of SEQ ID NO: 92, a LCDR2 comprising the sequence of SEQ ID NO: 93, and a LCDR3 comprising the sequence of SEQ ID NO: 94; or
s) a HCDR1 comprising the sequence of SEQ ID NO: 95, a HCDR2 comprising the sequence of SEQ ID NO: 96, and a HCDR3 comprising the sequence of SEQ ID NO: 97; and/or a LCDR1 comprising the sequence of SEQ ID NO: 98, a LCDR2 comprising the sequence of SEQ ID NO: 99, and a LCDR3 comprising the sequence of SEQ ID NO: 100; or
t) a HCDR1 comprising the sequence of SEQ ID NO: 101, a HCDR2 comprising the sequence of SEQ ID NO: 102, and a HCDR3 comprising the sequence of SEQ ID NO: 103; and/or a LCDR1 comprising the sequence of SEQ ID NO: 104, a LCDR2 comprising the sequence of SEQ ID NO: 105, and a LCDR3 comprising the sequence of SEQ ID NO: 106.

In certain embodiments, the claudin 18.2 binding domain comprises the same HCDRs and LCDRs as anti-claudin 18.2 antibody selected from the group consisting of hu26.H1L1, hu26.H1L2 (S92A), hu28.H1L2, C10, C29 and C30,
u) wherein the hu26.H1L1 comprises a heavy chain variable region comprising the sequence of SEQ ID NO: 65, and/or a light chain variable region comprising the sequence of SEQ ID NO: 66,
v) wherein the hu26.H1L2 (S92A) comprises a heavy chain variable region comprising the sequence of SEQ ID NO: 65, and/or a light chain variable region comprising the sequence of SEQ ID NO: 224
w) the hu28.H1L2 comprises a heavy chain variable region comprising the sequence of SEQ ID NO: 69, and/or a light chain variable region comprising the sequence of SEQ ID NO: 70,
x) the C10 comprises a heavy chain variable region comprising the sequence of SEQ ID NO: 71, and/or a light chain variable region comprising the sequence of SEQ ID NO: 72,
y) the C29 comprises a heavy chain variable region comprising the sequence of SEQ ID NO: 73, and/or a light chain variable region comprising the sequence of SEQ ID NO: 74, and
z) the C30 comprises a heavy chain variable region comprising the sequence of SEQ ID NO: 75, and/or a light chain variable region comprising the sequence of SEQ ID NO: 76.

In certain embodiments, the claudin 18.2 binding domain further comprises one or more of heavy chain HFR1, HFR2, HFR3 and HFR4, and/or one or more of light chain LFR1, LFR2, LFR3 and LFR4, wherein:
a) the HFR1 comprises an amino acid sequence selected from the group consisting of EVQLLESGGGLVQPGGSLRLSCAASGFTLS (SEQ ID NO: 167) and QVQLVQSGAEVKKPGASVKVSCKASGYTFT (SEQ ID NO: 168) or a homologous sequence of at least 80% sequence identity thereof,
b) the HFR2 comprises an amino acid sequence selected from the group consisting of WVRQAPGKGLEWVX₁₈ (SEQ ID NO: 169) and WVRQAPGQGLEWMG (SEQ ID NO: 170) or a homologous sequence of at least 80% sequence identity thereof,
c) the HFR3 sequence comprises an amino acid sequence selected from the group consisting of RFTISRDNSKNTLYLQMNSLRAEDTAVYYCAX₂₃ (SEQ ID NO: 171) and RVTMTRDTSTSTVYMELSSLRSEDTAVYYCAR (SEQ ID NO: 172) or a homologous sequence of at least 80% sequence identity thereof,
d) the HFR4 comprises WGQGTLVTVSS (SEQ ID NO: 173) or a homologous sequence of at least 80% sequence identity thereof,
e) the LFR1 comprises an amino acid sequence selected from the group consisting of DIQLTQSPSFLSASVGDRVTITC (SEQ ID NO: 174) and DIVMTQSPDSLAVSLGERATINC (SEQ ID NO: 175) or a homologous sequence of at least 80% sequence identity thereof,
f) the LFR2 comprises an amino acid sequence selected from the group consisting of WYQQKPGX₂₆X₂₇PKX₁₉LIY (SEQ ID NO: 176) or a homologous sequence of at least 80% sequence identity thereof,
g) the LFR3 comprises an amino acid sequence selected from the group consisting of GVPSRFSGSGSGTEX₂₄TLTISSLQPEDFATYYC (SEQ ID NO: 178) and GVPDRFSGSGSGTDFTLTISSLQAEDVAVYHC (SEQ ID NO: 179) or a homologous sequence of at least 80% sequence identity thereof, and
h) the LFR4 comprises FGX₂₅GTKLEIK (SEQ ID NO: 180) or a homologous sequence of at least 80% sequence identity thereof,
   wherein X₈ is S or A, X₁₉ is L or A, X₂₃ is T or K, X₂₄ is Y or F, X₂₅ is Q or G, X₂₆ is Q or K, X₂₇ is P or A.

In certain embodiments, the claudin 18.2 binding domain comprises:
aa) the heavy chain variable region comprises the sequence of SEQ ID NO: 65 or 68, and/or the light chain variable region comprises the sequence of SEQ ID NO: 66 or 67 or 224; or
bb) the heavy chain variable region comprises the sequence of SEQ ID NO: 69 and/or the light chain variable region comprises the sequence of SEQ ID NO: 70; or
cc) the heavy chain variable region comprises the sequence of SEQ ID NO: 71 and/or the light chain variable region comprises the sequence of SEQ ID NO: 72; or
dd) the heavy chain variable region comprises the sequence of SEQ ID NO: 73 and/or the light chain variable region comprises the sequence of SEQ ID NO: 74; or
ee) the heavy chain variable region comprises the sequence of SEQ ID NO: 75 and/or the light chain variable region comprises the sequence of SEQ ID NO: 76.

In certain embodiments, the target antigen binding domain comprises a PD-L1 binding domain.

In certain embodiments, the PD-L1 binding domain comprises:
ff) a HCDR1 comprising the sequence of SEQ ID NO: 119, a HCDR2 comprising the sequence of SEQ ID NO: 120, and a HCDR3 comprising the sequence of SEQ ID NO: 121; or
gg) a HCDR1 comprising the sequence of SEQ ID NO: 122, a HCDR2 comprising the sequence of SEQ ID NO: 123, and a HCDR3 comprising the sequence of SEQ ID NO: 124; or
hh) a HCDR1 comprising the sequence of SEQ ID NO: 125, a HCDR2 comprising the sequence of SEQ ID NO: 126, and a HCDR3 comprising the sequence of SEQ ID NO: 127; or
ii) a HCDR1 comprising the sequence of SEQ ID NO: 128, a HCDR2 comprising the sequence of SEQ ID NO: 129, and a HCDR3 comprising the sequence of SEQ ID NO: 130; or
jj) a HCDR1 comprising the sequence of SEQ ID NO: 131, a HCDR2 comprising the sequence of SEQ ID NO: 132, and a HCDR3 comprising the sequence of SEQ ID NO: 133; or
kk) a HCDR1 comprising the sequence of SEQ ID NO: 134, a HCDR2 comprising the sequence of SEQ ID NO: 135, and a HCDR3 comprising the sequence of SEQ ID NO: 136; or
ll) a HCDR1 comprising the sequence of SEQ ID NO: 137, a HCDR2 comprising the sequence of SEQ ID NO: 138, and a HCDR3 comprising the sequence of SEQ ID NO: 139; or
mm) a HCDR1 comprising the sequence of SEQ ID NO: 140, a HCDR2 comprising the sequence of SEQ ID NO: 141, and a HCDR3 comprising the sequence of SEQ ID NO: 142; or
nn) a HCDR1 comprising the sequence of SEQ ID NO: 143, a HCDR2 comprising the sequence of SEQ ID NO: 144, and a HCDR3 comprising the sequence of SEQ ID NO: 145; or
oo) a HCDR1 comprising the sequence of SEQ ID NO: 146, a HCDR2 comprising the sequence of SEQ ID NO: 147, and a HCDR3 comprising the sequence of SEQ ID NO: 148; or
pp) a HCDR1 comprising the sequence of SEQ ID NO: 149, a HCDR2 comprising the sequence of SEQ ID NO: 150, and a HCDR3 comprising the sequence of SEQ ID NO: 151; or
   a HCDR1 comprising the sequence of SEQ ID NO: 152, a HCDR2 comprising the sequence of SEQ ID NO: 153, and a HCDR3 comprising the sequence of SEQ ID NO: 154.

In certain embodiments, the PD-L1 binding domain comprises the same HCDRs as anti-PD-L1 antibody selected from the group consisting of C71, C71v38, C239, C492, C570, 570h3, C446, C2811, C1778, C1793, C2855, C2713 and C2719,
qq) wherein the C71 comprises a heavy chain variable region comprising the sequence of SEQ ID NO: 107,
rr) the C71v38 comprises a heavy chain variable region comprising the sequence of SEQ ID NO: 108,
ss) the C239 comprises a heavy chain variable region comprising the sequence of SEQ ID NO: 109,
tt) the C492 comprises a heavy chain variable region comprising the sequence of SEQ ID NO: 110,
uu) the C570 comprises a heavy chain variable region comprising the sequence of SEQ ID NO: 111,
vv) the 570h3 comprises a heavy chain variable region comprising the sequence of SEQ ID NO: 223,
ww) the C446 comprises a heavy chain variable region comprising the sequence of SEQ ID NO: 112,
xx) the C2811 comprises a heavy chain variable region comprising the sequence of SEQ ID NO: 113,
yy) the C1778 comprises a heavy chain variable region comprising the sequence of SEQ ID NO: 114,
zz) the C1793 comprises a heavy chain variable region comprising the sequence of SEQ ID NO: 115,
aaa) the C2855 comprises a heavy chain variable region comprising the sequence of SEQ ID NO: 116,
bbb) the C2713 comprises a heavy chain variable region comprising the sequence of SEQ ID NO: 117, and
ccc) the C2719 comprises a heavy chain variable region comprising the sequence of SEQ ID NO: 118.

In certain embodiments, the PD-L1 binding domain comprises: the heavy chain variable region comprises the sequence selected from the group consisting of SEQ ID NOs: 107-118 and 223.

In certain embodiments,
ddd) the SIRP-alpha binding domain further comprises one or more amino acid residue substitutions or modifications yet retains specific binding to human SIRPα; and/or
eee) the claudin 18.2 binding domain further comprises one or more amino acid residue substitutions or modifications yet retains specific binding to claudin 18.2, and/or
fff) the PD-L1 binding domain further comprises one or more amino acid residue substitutions or modifications yet retains specific binding to PD-L1.

In certain embodiments, at least one of the substitutions or modifications is in one or more of the CDR sequences, and/or in one or more of the non-CDR sequences of the heavy chain variable region or light chain variable region.

In certain embodiments, the multi-specific molecule provided herein is humanized.

In certain embodiments, the multi-specific molecule provided herein is linked to one or more conjugate moieties.

In certain embodiments, the conjugate moiety comprises a clearance-modifying agent, a chemotherapeutic agent, a toxin, a radioactive isotope, a lanthanide, a luminescent label, a fluorescent label, an enzyme-substrate label, a DNA-alkylator, a topoisomerase inhibitor, a tubulin-binder, a purification moiety, or other anticancer drugs.

In another aspect, the present disclosure provides a pharmaceutical composition comprising the multi-specific molecule provided herein, and one or more pharmaceutically acceptable carriers.

In another aspect, the present disclosure provides an isolated polynucleotide encoding the multi-specific molecule provided herein.

In another aspect, the present disclosure provides a vector comprising the isolated polynucleotide provided herein.

In another aspect, the present disclosure provides a host cell comprising the vector provided herein.

In another aspect, the present disclosure provides a kit comprising the multi-specific molecule provided herein and/or the pharmaceutical composition provided herein, and a second therapeutic agent.

In another aspect, the present disclosure provides a method of expressing the multi-specific molecule provided herein, comprising culturing the host cell provided herein under the condition at which the vector provided herein is expressed.

In another aspect, the present disclosure provides a method of treating a disease, disorder or condition that can be benefited from induced phagocytosis of a target cell in a subject, comprising administering to the subject a therapeutically effective amount of the multi-specific molecule provided herein.

In another aspect, the present disclosure provides a method of treating a target antigen related disease, disorder or condition in a subject, comprising administering to the subject a therapeutically effective amount of the multi-specific molecule provided herein.

In another aspect, the present disclosure provides a method of treating a SIRPα related disease, disorder or condition in a subject, comprising administering to the subject a therapeutically effective amount of the multi-specific molecule provided herein.

In another aspect, the present disclosure provides a method of treating a CD47 related disease, disorder or condition in a subject, comprising administering to the subject a therapeutically effective amount of the multi-specific molecule provided herein.

In certain embodiments, the subject is human.

In certain embodiments, the subject has been diagnosed with or is at risk for a disease, disorder or condition selected from the group consisting of immune related disease or disorder, tumors and cancers, autoimmune diseases, and infectious disease.

In certain embodiments, the immune related disease or disorder is selected from the group consisting of systemic lupus erythematosus, acute respiratory distress syndrome (ARDS), vasculitis, myasthenia gravis, idiopathic pulmonary fibrosis, Crohn's Disease, asthma, rheumatoid arthritis, graft versus host disease, a spondyloarthropathy (e.g., ankylosing spondylitis, psoriatic arthritis, isolated acute enteropathic arthritis associated with inflammatory bowel disease, reactive arthritis, Behcet's syndrome, undifferentiated spondyloarthropathy, anterior uveitis, and juvenile idiopathic arthritis.), multiple sclerosis, endometriosis, glomerulonephritis, sepsis, diabetes, acute coronary syndrome, ischemic reperfusion, psoriasis, progressive systemic sclerosis, atherosclerosis, Sjogren's syndrome, scleroderma, or inflammatory autoimmune myositis.

In certain embodiments, the tumors and cancers are solid tumor or hematologic malignancy, optionally selected from the group consisting of non-small cell lung cancer, small cell lung cancer, renal cell cancer, colorectal cancer, ovarian cancer, breast cancer, pancreatic cancer, gastric carcinoma, bladder cancer, esophageal cancer, mesothelioma, melanoma, head and neck cancer, thyroid cancer, sarcoma, prostate cancer, glioblastoma, cervical cancer, thymic carcinoma, leukemia, lymphomas, myelomas, mycoses fungoids, merkel cell cancer, and other hematologic malignancies, such as classical Hodgkin lymphoma (CHL), primary mediastinal large B-cell lymphoma, T-cell/histiocyte-rich B-cell lymphoma, EBV-positive and -negative PTLD, and EBV-associated diffuse large B-cell lymphoma (DLBCL), plasmablastic lymphoma, extranodal NK/T-cell lymphoma, nasopharyngeal carcinoma, and HHV8-associated primary effusion lymphoma, Hodgkin's lymphoma, neoplasm of the central nervous system (CNS), such as primary CNS lymphoma, spinal axis tumor, brain stem glioma, anal cancer, appendix cancer, astrocytoma, basal cell carcinoma, gallbladder cancer, gastric cancer, lung cancer, bronchial cancer, bone cancer, liver and bile duct cancer, pancreatic cancer, breast cancer, liver cancer, ovarian cancer, testicle cancer, kidney cancer, renal pelvis and ureter cancer, salivary gland cancer, small intestine cancer, urethral cancer, bladder cancer, head and neck cancer, spine cancer, brain cancer, cervix cancer, uterine cancer, endometrial cancer, colon cancer, colorectal cancer, rectal cancer, esophageal cancer, gastrointestinal cancer, skin cancer, prostate cancer, pituitary cancer, vagina cancer, thyroid cancer, throat cancer, glioblastoma, melanoma, myelodysplastic syndrome, sarcoma, teratoma, chronic lymphocytic leukemia (CLL), chronic myeloid leukemia (CIL), acute lymphocytic leukemia (ALL), acute myeloid leukemia (AMIL), Hodgkin lymphoma, non-Hodgkin lymphoma, multiple myeloma, T or B cell lymphoma, GI organ interstitialoma, soft tissue tumor, hepatocellular carcinoma, and adenocarcinoma, or the metastases thereof.

In certain embodiments, the administration is via oral, nasal, intravenous, subcutaneous, sublingual, or intramuscular administration.

In certain embodiments, the method provided herein further comprises administering a therapeutically effective amount of a second therapeutic agent.

In certain embodiments, the second therapeutic agent is selected from the group consisting of a chemotherapeutic agent, an anti-cancer drug, a radiation therapy agent, an immunotherapy agent, an anti-angiogenesis agent, a targeted therapy agent, a cellular therapy agent, a gene therapy agent, a hormonal therapy agent, an antiviral agent, an antibiotic, an analgesics, an antioxidant, a metal chelator, and cytokines.

In another aspect, the present disclosure provides use of the multi-specific molecule provided herein and/or the pharmaceutical composition provided herein in the manufacture of a medicament for treating, preventing or alleviating a disease, disorder or condition that can be benefited from induced phagocytosis of a target cell in a subject.

In another aspect, the present disclosure provides a method of inducing phagocytosis of a target cell in a subject, comprising administering to the subject the multi-specific molecule provided herein and/or the pharmaceutical composition provided herein in a dose effective to induce phagocytosis of the target cell.

In certain embodiments, the subject is human.

In certain embodiments, the subject has been diagnosed with or is at risk for a disease, disorder or condition selected from the group consisting of immune related disease or disorder, tumors and cancers, autoimmune diseases, and infectious disease.

In another aspect, the present disclosure provides a method of inducing phagocytosis of a target cell in vitro, comprising contacting the target cell with a SIRPα positive phagocytic cell sample in the presence of the multi-specific molecule provided herein and/or the pharmaceutical composition provided herein, thereby inducing the phagocytosis of the target cell by the SIRPα positive phagocytic cell.

In certain embodiments, the target cell is a cell expressing the target antigen.

In another aspect, the present disclosure provides a method of inducing elimination of a target cell co-expressing a target antigen and CD47 by phagocytosis, comprising contacting the target cell with the multi-specific molecule provided herein in presence of a phagocytic immune cell.

In another aspect, the present disclosure provides a method of inducing phagocytic effect selectively against a target cell co-expressing a target antigen and CD47 over a cell that does not express the target antigen in a subject, comprising administering to the subject a therapeutically effective amount of the multi-specific molecule provided herein.

In another aspect, the present disclosure provides a method of increasing the level of M1 macrophage in a tumor microenvironment of a subject in need thereof, comprising administering to the subject a therapeutically effective amount of the multi-specific molecule provided herein.

### BRIEF DESCRIPTION OF FIGURES

FIG. 1 shows a schematic drawing of a bispecific macrophage engager (BiME) antibody enhancing macrophage phagocytotic activities against a cancer cell expressing tumor associated antigen (TAA).

FIG. 2A shows a schematic drawing of an anti-Claudin18.2/SIRPα bispecific molecule (ES028-001) comprising an antibody targeting Claudin18.2, which is fused at the C terminus of the light chain (LC) with two anti-SIRPα scFv.

FIG. 2B shows a schematic drawing of an anti-Claudin18.2/SIRPα bispecific molecule (ES028-005) comprising an antibody targeting Claudin18.2, which is fused at the C terminus of the heavy chain (HC) with two anti-SIRPα scFv.

FIG. 2C shows a schematic drawing of an anti-Claudin18.2/SIRPα bispecific molecule (ES028-009) comprising an antibody targeting SIRPα, which is fused at the C terminus of the light chain (LC) with two anti-Claudin18.2 scFv.

FIG. 2D shows a schematic drawing of an anti-Claudin18.2/SIRPα bispecific molecule (ES028-013) comprising an antibody targeting SIRPα, which is fused at the C terminus of the heavy chain (LC) with two anti-Claudin18.2 scFv.

FIG. 3A shows that the representative bispecific antibodies could bind to Raji/hClaudin18.2 cells by FACS detection. Anti-Claudin18.2 antibody was used as control.

FIG. 3B shows that the representative bispecific antibodies could bind to CHO-K1/SIRPα cells by FACS detection. Anti-SIRPα antibody was used as control.

FIG. 4A shows that the anti-Claudin18.2/SIRPα bispecific antibodies stimulate mouse BMDM phagocytosis against MC38/hCD47/hClaudin18.2 cell better than single or combination treatment. Anti-CD47 antibody is used as control.

FIG. 4B shows that the anti-Claudin18.2/SIRPα bispecific antibodies do not stimulate mouse BMDM phagocytosis against MC38/hCD47 cell that does not express Cluadin18.2. Anti-CD47 antibody is used as control.

FIG. 5 shows that the comparison of anti-Claudin18.2/SIRPα bispecific antibody isotypes on the phagocytosis of mouse BMDM phagocytosis against MC38/hCD47/hClaudin18.2 cell. Anti-CD47 antibody is used as control.

FIG. 6A is a schematic drawing of an anti-PDL1/SIRPα bispecific molecule (ES019-020) comprising one antibody targeting SIRPα, which is fused at the C terminus of the light chain (LC) with two anti-PDL1 sdAb.

FIG. 6B is a schematic drawing of an anti-PDL1/SIRPα bispecific molecule (ES019-024) comprising one antibody targeting SIRPα, which is fused at the C terminus of the heavy chain (HC) with two anti-PDL1 sdAb.

FIG. 6C is a schematic drawing of an anti-PDL1/SIRPα bispecific molecule (ES019-025) comprising one antibody targeting SIRPα, which is fused at the N terminus of the heavy chain (HC) with two anti-PDL1 sdAb.

FIG. 6D is a schematic drawing of an anti-PDL1/SIRPα bispecific molecule (ES019-026) comprising one antibody targeting SIRPα, which is fused at the N terminus of the light chain (LC) with two anti-PDL1 sdAb.

FIG. 6E is a schematic drawing of an anti-PDL1/SIRPα bispecific molecule (ES019-029) comprising one asymmetric antibody with one Fab arm targeting SIRPα, and the other arm containing two anti-PDL1 sdAb; the heterodimer is connected by knob-in hole mutations in Fc region.

FIG. 6F is a schematic drawing of an anti-PDL1/SIRPα bispecific molecule (ES019-072) comprising one asymmetric antibody with two anti-PDL1 sdAb are fused to N-terminus of Fc and one anti-SIRPα Fab is fused to C-terminus of Fc; the heterodimer is connected by knob-in hole mutations in Fc region.

FIG. 6G is a schematic drawing of an anti-PDL1/SIRPα bispecific molecule (ES019-073 or ES019-079) comprising one asymmetric antibody with two anti-PDL1 sdAb are fused to N-terminus of Fc and two anti-SIRPα Fabs are fused to C-terminus of Fc.

FIG. 7A shows that the representative bispecific antibodies ES019-020, ES019-024, ES019-025 and ES019-026 could bind to Raji/hPDL1 cells by FACS detection. Anti-PDL1 antibody was used as control.

FIG. 7B shows that the representative bispecific antibodies ES019-020, ES019-024, ES019-025 and ES019-026 could bind to CHO-K1/SIRPα cells by FACS detection. Anti-SIRPα antibody was used as control.

FIG. 8 shows that the representative bispecific antibodies ES019-020, ES019-024, ES019-025 and ES019-026 could activate Jurkat T cell by a Jurat/PD1 reporter cell assay. Anti-PDL1 antibody was used as control.

FIG. 9A shows that the anti-PDL1/SIRPα bispecific antibodies ES019-020, ES019-024, ES019-025, ES019-026 and ES019-029 stimulate human monocyte derived macrophage phagocytosis against K562/hPDL1 cell better than single or similar to combination treatment. Anti-CD47 antibody is used as control.

FIG. 9B shows that the anti-PDL1/SIRPα bispecific antibodies do not stimulate human monocyte derived macrophage phagocytosis against K562(PDL1 negative) cell similar to single or combination treatment. Anti-CD47 antibody is used as control.

FIG. 9C shows that the anti-PDL1/SIRPα bispecific antibodies do not stimulate human monocyte derived macrophage phagocytosis against Jurkat cell (SIRPγ positive) similar to single or combination treatment. Anti-CD47 antibody is used as control.

FIG. 10 shows that in vivo anti-tumor efficacy of ES028 BiME (e.g., ES028-001, ES028-005 and ES028-009) in MC38/hClaudin18.2/hSIRPα syngeneic model.

FIG. 11A shows that different SIRPα antibodies based combination or SIRPα bispecific antibodies induced phagocytosis against Raji/hPDL1 cell.

FIG. 11B shows that different SIRPα antibodies based combination or PDL1/SIRPα bispecific antibodies induced phagocytosis against Raji (PDL1 negative) cell.

FIG. 12 shows binding affinity to CHOK1-hSIRPα v1 and CHOK1-hSIRPα v2 of the chimeric antibodies C15, C25, C42, C59, C73 and hu1H9G4.

FIG. 13 shows IC50 value and blocking percentage for human CD47/SIRPα v1 interaction and human CD47/SIRPα v2 interaction by the chimeric antibodies C15, C25, C42, C59, C73 and hu1H9G4.

FIG. 14 shows IC50 value and blocking percentage for SHP-1 recruitment by the chimeric antibodies C15, C25, C42, C59, C73 and hu1H9G4.

FIG. 15 shows binding affinity to CHOK1-hSIRPα v1 and CHOK1-hSIRPα v2 of the humanized antibodies hu025.021, hu025.023, hu025.033, hu025.059 and hu025.060 and C25.

FIG. 16 shows binding kinetics to human SIRPα v1 and human SIRPα v2 of the humanized antibodies hu025.021, hu025.023, hu025.033, hu025.059 and hu025.060 and C25.

FIG. 17 shows IC50 value and blocking percentage for human CD47/SIRPα v1 interaction and human CD47/SIRPα v2 interaction by the humanized antibodies hu025.021, hu025.023, hu025.033, hu025.059 and hu025.060, and C25 as measured by competitive ELISA assay.

FIG. 18 shows IC50 value and blocking percentage for human CD47/SIRPα v1 interaction and human CD47/SIRPα v2 interaction by the humanized antibodies hu025.023, hu025.060, and C25 as measured by competitive FACS assay.

FIG. 19 shows IC50 value and blocking percentage for SHP-1 recruitment by the humanized antibodies hu025.021, hu025.023, hu025.033, hu025.059 and hu025.060, and C25.

FIG. 20A shows phagocytic index of Raji cells by human macrophages expressing hSIRPα v1/v2 in presence of anti-SIRPα antibodies 025c, 015c, 042c, 059c, or 073c alone or in combination with Rituximab.

FIG. 20B shows phagocytic index of Raji cells by human macrophages expressing hSIRPα v1/v1 in presence of anti-SIRPα antibodies 025c or 042c alone or in combination with anti-PD-L1 antibodies with different concentrations.

FIG. 20C shows phagocytic index of Raji cells by human macrophages expressing hSIRPα v2/v2 in presence of anti-SIRPα antibodies 025c, 042c or 073c alone or in combination with anti-PD-L1 antibodies with different concentrations.

FIG. 21A shows phagocytic index of Raji cells by human macrophages expressing hSIRPα v1/v1 in presence of anti-SIRPα antibodies 025c, hu025.023 or hu025.060 alone or in combination with anti-PD-L1 antibodies with different concentrations.

FIG. 21B shows phagocytic index of Raji cells by human macrophages expressing hSIRPα v1/v1 in presence of anti-SIRPα antibodies 025c, hu025.023 or hu025.060 alone or in combination with Rituximab with different concentrations.

FIG. 21C shows phagocytic index of Raji cells by human macrophages expressing hSIRPα v2/v2 in presence of anti-SIRPα antibodies 025c, hu025.023 or hu025.060 alone or in combination with anti-PD-L1 antibodies with different concentrations.

FIG. 21D shows phagocytic index of Raji cells by human macrophages expressing hSIRPα v2/v2 in presence of anti-SIRPα antibodies 025c, hu025.023 or hu025.060 alone or in combination with Rituximab with different concentrations.

FIG. 22 shows blocking percentage (% Blocking) of anti-SIRPα antibodies 035, 050 and 025 at different concentrations for blocking SIRPα and CD47 interaction.

FIG. 23 shows potential binding epitopes of anti-SIRPα antibodies 025c (FIG. 23A), 042c (FIG. 23B), 073c (FIG. 23C), hu1H9G4 (FIG. 23D), HEFLB (FIG. 23E) as measured by HDX-MS. All antibodies are human IgG4 chimeric antibodies with S228P mutation.

### DETAILED DESCRIPTION OF THE INVENTION

The following description of the disclosure is merely intended to illustrate various embodiments of the disclosure. As such, the specific modifications discussed are not to be construed as limitations on the scope of the disclosure. It will be apparent to one skilled in the art that various equivalents, changes, and modifications may be made without departing from the scope of the disclosure, and it is understood that such equivalent embodiments are to be included herein. All references cited herein, including publications, patents and patent applications are incorporated herein by reference in their entirety.

### Definitions

The term “antibody” as used herein includes any immunoglobulin, monoclonal antibody, polyclonal antibody, multivalent antibody, bivalent antibody, monovalent antibody, single domain antibody, multi-specific antibody, or bispecific antibody that binds to a specific antigen. A native intact IgG antibody comprises two heavy (H) chains and two light (L) chains. Mammalian heavy chains are classified as alpha, delta, epsilon, gamma, and mu, each heavy chain consists of a variable region (V_{H}) and a first, second, and third constant region (C_{H1}, C_{H2}, C_{H3}, respectively); mammalian light chains are classified as λ or κ, while each light chain consists of a variable region (V_{L}) and a constant region. The antibody has a “Y” shape, with the stem of the Y consisting of the second and third constant regions of two heavy chains bound together via disulfide bonding. Each arm of the Y includes the variable region and first constant region of a single heavy chain bound to the variable and constant regions of a single light chain. The variable regions of the light and heavy chains are responsible for antigen binding. The variable regions in both chains generally contain three highly variable loops called the complementarity determining regions (CDRs) (light chain CDRs including LCDR1, LCDR2, and LCDR3, heavy chain CDRs including HCDR1, HCDR2, HCDR3). CDR boundaries for the antibodies and antigen-binding domains disclosed herein may be defined or identified by the conventions of Kabat, IMGT, AbM, Chothia, or Al-Lazikani (Al-Lazikani, B., Chothia, C., Lesk, A. M., J. Mol. Biol., 273(4), 927 (1997); Chothia, C. et al., J Mol Biol. December 5; 186(3):651-63 (1985); Chothia, C. and Lesk, A. M., J. Mol.Biol., 196,901 (1987); N. R. Whitelegg et al, Protein Engineering, v13(12), 819-824 (2000); Chothia, C. et al., Nature. December 21-28; 342(6252):877-83 (1989); Kabat E. A. et al., National Institutes of Health, Bethesda, Md. (1991); Marie-Paule Lefranc et al, Developmental and Comparative Immunology, 27: 55-77 (2003); Marie-Paule Lefranc et al, Immunome Research, 1(3), (2005); Marie-Paule Lefranc, Molecular Biology of B cells (second edition), chapter 26, 481-514, (2015)). The three CDRs are interposed between flanking stretches known as framework regions (FRs), which are more highly conserved than the CDRs and form a scaffold to support the hypervariable loops. The constant regions of the heavy and light chains are not involved in antigen-binding, but exhibit various effector functions. Antibodies are assigned to classes based on the amino acid sequence of the constant region of their heavy chain. The five major classes or isotypes of antibodies are IgA, IgD, IgE, IgG, and IgM, which are characterized by the presence of alpha, delta, epsilon, gamma, and mu heavy chains, respectively. Several of the major antibody classes are divided into subclasses such as IgG1 (gamma1 heavy chain), IgG2 (gamma2 heavy chain), IgG3 (gamma3 heavy chain), IgG4 (gamma4 heavy chain), IgA1 (alpha1 heavy chain), or IgA2 (alpha2 heavy chain).

The term “antibody” as used herein, can also encompass a single domain antibody such as a heavy chain antibody. “Heavy chain antibody” or “HCAb” refers to an antibody that contains two VH domains and no light chains (Riechmann L. and Muyldermans S., J Immunol Methods. December 10; 231(1-2):25-38 (1999); Muyldermans S., J Biotechnol. June; 74(4):277-302 (2001); WO94/04678; WO94/25591; U.S. Pat. No. 6,005,079). Heavy chain antibodies were originally derived from Camelidae (camels, dromedaries, and llamas). Although devoid of light chains, camelized antibodies have an authentic antigen-binding repertoire (Hamers-Casterman C. et al., Nature. June 3; 363(6428):446-8 (1993); Nguyen V K. et al. “Heavy-chain antibodies in Camelidae; a case of evolutionary innovation,” Immunogenetics. April; 54(l):39-47 (2002); Nguyen V K. et al. Immunology. May; 109(1):93-101 (2003)). The variable domain of a heavy chain antibody (VHH domain) represents the smallest known antigen-binding unit generated by adaptive immune responses (Koch-Nolte F. et al., FASEB J November; 21(13):3490-8. Epub 2007 June 15 (2007)).

The term “antigen-binding domain” as used herein refers to an antibody fragment formed from a portion of an antibody comprising one or more CDRs, or any other antibody fragment that binds to an antigen but does not comprise an intact native antibody structure. Examples of antigen-binding domain include, without limitation, a diabody, a Fab, a Fab′, a F(ab′)₂, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a bispecific antibody, a multispecific antibody, a camelized single domain antibody, a nanobody, a domain antibody, and a bivalent domain antibody. An antigen-binding domain is capable of binding to the same antigen to which the parent antibody binds. In certain embodiments, an antigen-binding domain may comprise one or more CDRs from a particular human antibody grafted to a framework region from one or more different human antibodies. For more and detailed formats of antigen-binding domain are described in Spiess et al, 2015 (Supra), and Brinkman et al., mAbs, 9(2), pp. 182-212 (2017), which are incorporated herein by entirety reference.

As used herein, the term “phagocytosis” refers to the process of cellular uptake of particulates (≥0.5 Dm) within a plasmamembrane envelope. Phagocytosis includes different variants, such as efferocytosis that involves the uptake of apoptotic cells, necroptosis and pyroptosis that involve update of necrotic cells arising from infection and inflammation, and heterophagy that involve the uptake of exogenous particles.

An “antigen” as used herein refers to a compound, composition, peptide, polypeptide, protein or substance that can stimulate the production of antibodies or an immune cell (e.g., T cell or myeoid cell) response in cell culture or in an animal, including compositions (such as one that includes a cancer-specific protein) that are added to a cell culture (such as a hybridoma), or injected or absorbed into an animal, or expressed on a cell surface. An antigen reacts with the products of specific humoral or cellular immunity (such as an antibody).

“Fab” with regard to an antibody refers to that portion of the antibody consisting of a single light chain (both variable and constant regions) bound to the variable region and first constant region of a single heavy chain by a disulfide bond. “F(ab)₂” refers to a dimer of Fab.

“Fab′” refers to a Fab fragment that includes a portion of the hinge region.

“F(ab′)₂” refers to a dimer of Fab′.

A “fragment difficult (Fd)” with regard to an antibody refers to the amino-terminal half of the heavy chain fragment that can be combined with the light chain to form a Fab. For example, Fd fragment may consists of the VH and CH1 domains

“Fv” with regard to an antibody refers to the smallest fragment of the antibody to bear the complete antigen-binding site. An Fv fragment consists of the variable region of a single light chain bound to the variable region of a single heavy chain. A number of Fv designs have been provided, including dsFvs, in which the association between the two domains is enhanced by an introduced disulphide bond; and scFvs can be formed using a peptide linker to bind the two domains together as a single polypeptide. Fvs constructs containing a variable domain of a heavy or light immunoglobulin chain associated to the variable and constant domain of the corresponding immunoglobulin heavy or light chain have also been produced. Fvs have also been multimerised to form diabodies and triabodies (Maynard et al., Annu Rev Biomed Eng 2 339-376 (2000)).

“Single-chain Fv antibody” or “scFv” refers to an engineered antibody consisting of a light chain variable region and a heavy chain variable region connected to one another directly or via a peptide linker sequence (Huston J S et al. Proc Natl Acad Sci USA, 85:5879(1988)). ScFv can also serve as basic modules for the development of multimeric structures (dimeric: “diabody”, trimeric: “triabody”, tetrameric: “tetrabody”).

A “diabody” or “dAb” includes small antibody fragments with two antigen-binding sites, wherein the fragments comprise a V_{H} domain connected to a V_{L} domain in the same polypeptide chain (V_{H}-V_{L} or V_{L}-V_{H}) (see, e.g. Holliger P. et al., Proc Natl Acad Sci USA. July 15; 90(14):6444-8 (1993); EP404097; WO93/11161). By using a linker that is too short to allow pairing between the two domains on the same chain, the domains are forced to pair with the complementary domains of another chain, thereby creating two antigen-binding sites. The antigen-binding sites may target the same or different antigens (or epitopes). In certain embodiments, a “bispecific ds diabody” is a diabody target two different antigens (or epitopes).

A “dsFv” refers to a disulfide-stabilized Fv fragment that the linkage between the variable region of a single light chain and the variable region of a single heavy chain is a disulfide bond. In some embodiments, a “(dsFv)₂” or “(dsFv-dsFv′)” comprises three peptide chains: two V_{H} moieties linked by a peptide linker (e.g. a long flexible linker) and bound to two V_{L} moieties, respectively, via disulfide bridges. In some embodiments, dsFv-dsFv′ is bispecific in which each disulfide paired heavy and light chain has a different antigen specificity.

The term “valent” as used herein refers to the presence of a specified number of antigen binding sites in a given molecule. The term “monovalent” refers to an antibody or an antigen-binding fragment having only one single antigen-binding site; and the term “multivalent” refers to an antibody or an antigen-binding fragment having multiple (i.e. more than one) antigen-binding sites. As such, the terms “bivalent”, “tetravalent”, and “hexavalent” denote the presence of two binding sites, four binding sites, and six binding sites, respectively, in an antigen-binding molecule. In some embodiments, the antibody or antigen-binding fragment thereof is bivalent.

A “nanobody” refers to an antibody fragment that consists of a VHH domain from a heavy chain antibody and two constant domains, CH2 and CH3.

A “domain antibody” or “single domain antibody” or “sdAb” refers to an antibody fragment containing only the variable region of a heavy chain or the variable region of a light chain. In certain instances, two or more VH domains are covalently joined with a peptide linker to create a bivalent or multivalent domain antibody. The two VH domains of a bivalent domain antibody may target the same or different antigens.

“Fc” with regard to an antibody refers to that portion of the antibody consisting of the second and third constant regions of a first heavy chain bound to the second and third constant regions of a second heavy chain via disulfide bonding. The Fc portion of the antibody is responsible for various effector functions such as antibody-dependent cell-mediated cytotoxicity (ADCC), complement dependent cytotoxicity (CDC), and phagocytosis.

The term “chimeric” as used herein, means an antibody or antigen-binding domain, having a portion of heavy and/or light chain derived from one species, and the rest of the heavy and/or light chain derived from a different species. In an illustrative example, a chimeric antibody may comprise a constant region derived from human and a variable region from a non-human animal, such as from mouse. In another illustrative example, a chimeric antibody may comprise FR regions derived from human and CDR regions from a non-human animal, such as from mouse. In some embodiments, the non-human animal is a mammal, for example, a mouse, a rat, a rabbit, a goat, a sheep, a guinea pig, or a hamster.

The term “humanized” as used herein means that the antibody or antigen-binding domain comprises CDRs derived from non-human animals, FR regions derived from human, and when applicable, the constant regions derived from human.

The term “operably link” or “operably linked” refers to a juxtaposition, with or without a spacer or a linker or an intervening sequence, of two or more biological sequences of interest in such a way that they are in a relationship permitting them to function in an intended manner. When used with respect to polypeptides, it is intended to mean that the polypeptide sequences are linked in such a way that permits the linked product to have the intended biological function. For example, an antibody variable region may be operably linked to a constant region so as to provide for a stable product with antigen-binding activity. For another example, an antigen-binding domain can be operably linked to another antigen-binding domain with an intervening sequence there between, and such intervening sequence can be a spacer or can comprise a much longer sequence such as a constant region of an antibody. The term may also be used with respect to polynucleotides. For one instance, when a polynucleotide encoding a polypeptide is operably linked to a regulatory sequence (e.g., promoter, enhancer, silencer sequence, etc.), it is intended to mean that the polynucleotide sequences are linked in such a way that permits regulated expression of the polypeptide from the polynucleotide.

The term “fusion” or “fused” when used with respect to amino acid sequences (e.g. peptide, polypeptide or protein) refers to combination of two or more amino acid sequences, for example by chemical bonding or recombinant means, into a single amino acid sequence. A fusion amino acid sequence may be produced by genetic recombination of two encoding polynucleotide sequences, and can be expressed by a method of introducing a construct containing the recombinant polynucleotides into a host cell.

“SIRPα” used interchangeably with “SIRPalpha” or “SIRP-alpha” as used herein, refers to a regulatory membrane glycoprotein from signal regulatory protein (SIRP) family expressed mainly by myeloid cells (e.g., macrophages, granulocytes, myeloid dendritic cells, mast cells and their precursors, such as hematopoietic stem cells (HSCs)), dendritic cells and also by stem cells or neurons. The structure of SIRPα includes an extracellular domain and a cytoplasmic domain. The extracellular domain of SIRPα consists of a membrane-distal Ig variable-like (IgV) fold, and two membrane-proximal Ig constant-like (IgC) folds. The IgV domain of SIRPα is responsible for the binding of the extracellular Ig-domain of CD47. In certain embodiments, the SIRPα is human SIRPα. The gene coding for human SIRPα is a polymorphic gene and several variants were described in human population. The most common protein variants are SIRPα v1 and SIRPα v2 (accession numbers NP_542970 (P78324) and CAA71403). SIRPα as used herein may be from other animal species, such as from mouse, and cynomolgus, among others. Exemplary sequence of Mus musculus (mouse) SIRPα protein is disclosed in NCBI Ref Seq No. NP_031573, or BAA20376.1, or BAA13521.1. Exemplary sequence of Cynomolgus (monkey) SIRPα protein is disclosed in NCBI Ref Seq No. NP_001271679.

“PD-L1” as used herein refers to programmed cell death ligand 1 (PD-L1, see, for example, Freeman et al. (2000) J. Exp. Med. 192:1027). Representative amino acid sequence of human PD-L1 is disclosed under the NCBI accession number: NP_054862.1, and the representative nucleic acid sequence encoding the human PD-L1 is shown under the NCBI accession number: NM_014143.3. PD-L1 is expressed in placenta, spleen, lymph nodes, thymus, heart, fetal liver, and is also found on many tumor or cancer cells. PD-L1 binds to its receptor PD-1 or B7-1, which is expressed on activated T cells, B cells and myeloid cells. The binding of PD-L1 and its receptor induces signal transduction to suppress TCR-mediated activation of cytokine production and T cell proliferation. Accordingly, PD-L1 plays a major role in suppressing immune system during particular events such as pregnancy, autoimmune diseases, tissue allografts, and is believed to allow tumor or cancer cells to circumvent the immunological checkpoint and evade the immune response.

“CLDN18” as used herein refers to Claudin18 and includes any variants thereof, including CLDN18.1 and CLDN18.2, conformations, isoforms and species homologs of CLDN18 which are naturally expressed by cells or are expressed by cells transfected with the CLDN18 gene. In certain embodiments, the CLDN18 is human CLDN18. CLDN18 as used herein may be from other animal species, such as from human, mouse, and cynomolgus, among others. The terms “CLDN18”, “CLDN-18”, “CLDN 18”, “Claudin18”, “Claudin-18”, or “Claudin 18” may be used interchangeably in the present disclosure. Unless otherwise specified, CLDN18 used herein refers to CLDN18 protein.

“CLDN18.1” is a splice variant of CLDN18, and includes post-translationally modified variants, isoforms and species homologs of CLDN18.1 which are naturally expressed by cells or are expressed on cells transfected with the CLDN18.1 gene. The terms “CLDN18.1”, “CLDN-18.1”, “CLDN 18.1”, “Claudin18.1”, “Claudin-18.1”, or “Claudin 18.1” may be used interchangeably in the present disclosure. Unless otherwise specified, CLDN18.1 used herein refers to CLDN18.1 protein. Exemplary sequence of human CLDN18.1 protein is disclosed in NCBI Ref Seq No. NP_057453.1.

“CLDN18.2” is a splice variant of CLDN18, and includes post-translationally modified variants, isoforms and species homologs of CLDN18.2 which are naturally expressed by cells or are expressed on cells transfected with the CLDN18.2 gene. The terms “CLDN18.2”, “CLDN-18.2”, “CLDN 18.2”, “Claudin18.2”, “Claudin-18.2”, or “Claudin 18.2” may be used interchangeably in the present disclosure. Unless otherwise specified, CLDN18.2 used herein refers to CLDN18.2 protein. Exemplary sequence of human CLDN18.2 protein is disclosed in NCBI Ref Seq No. NP_001002026.1.

The term “specific binding” or “specifically binds” as used herein refers to a non-random binding reaction between two molecules, such as for example between an antibody or antigen-binding domain thereof and an antigen. In certain embodiments, the antibody molecules or antigen-binding domains provided herein specifically bind to human SIRPα, human Claudin18.2 and/or human PD-L1 with a binding affinity (K_{D}) of ≤10⁻⁶ M (e.g., ≤5×10⁻⁷M, ≤2×10⁻⁷ M, ≤10⁻⁷ M, ≤5×10⁻⁸ M, ≤2×10⁻⁸ M, ≤10⁻⁸ M, ≤5×10⁻⁹ M, ≤4×10⁻⁹M). K_{D} used herein refers to the ratio of the dissociation rate to the association rate (k_{off}/kₒₙ), which may be determined by using any conventional method known in the art, including but are not limited to surface plasmon resonance method, microscale thermophoresis method, HPLC-MS method and flow cytometry (such as FACS) method. In certain embodiments, the K_{D} value can be appropriately determined by using flow cytometry.

The term “epitope” as used herein refers to the specific group of atoms or amino acids on an antigen to which an antibody binds. Epitopes can be formed both from contiguous amino acids (also called linear or sequential epitope) or noncontiguous amino acids juxtaposed by tertiary folding of a protein (also called configurational or conformational epitope). Epitopes formed from contiguous amino acids are typically arranged linearly along the primary amino acid residues on the protein and the small segments of the contiguous amino acids can be digested from an antigen binding with major histocompatibility complex (MHC) molecules or retained on exposure to denaturing solvents whereas epitopes formed by tertiary folding are typically lost on treatment with denaturing solvents. An epitope typically includes at least 3, and more usually, at least 5, about 7, or about 8-10 amino acids in a unique spatial conformation. Two antibodies may bind the same or a closely related epitope within an antigen if they exhibit competitive binding for the antigen. For example, if an antibody or antigen-binding domain blocks binding of a reference antibody to the antigen by at least 85%, or at least 90%, or at least 95%, then the antibody or antigen-binding domain may be considered to bind the same/closely related epitope as the reference antibody.

The term “amino acid” as used herein refers to an organic compound containing amine (—NH₂) and carboxyl (—COOH) functional groups, along with a side chain specific to each amino acid. The names of amino acids are also represented as standard single letter or three-letter codes in the present disclosure, which are summarized as follows.

| Name of Amino Acid | Three-letter Code | Single-letter Code |
|---|---|---|
| Alanine | Ala | A |
| Arginine | Arg | R |
| Asparagine | Asn | N |
| Aspartic acid | Asp | D |
| Cysteine | Cys | C |
| Glutamic acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

A “conservative substitution” with reference to amino acid sequence refers to replacing an amino acid residue with a different amino acid residue having a side chain with similar physiochemical properties. For example, conservative substitutions can be made among amino acid residues with hydrophobic side chains (e.g. Met, Ala, Val, Leu, and Ile), among residues with neutral hydrophilic side chains (e.g. Cys, Ser, Thr, Asn and Gln), among residues with acidic side chains (e.g. Asp, Glu), among amino acids with basic side chains (e.g. His, Lys, and Arg), or among residues with aromatic side chains (e.g. Trp, Tyr, and Phe). As known in the art, conservative substitution usually does not cause significant change in the protein conformational structure, and therefore could retain the biological activity of a protein.

The term “homolog” and “homologous” as used herein are interchangeable and refer to nucleic acid sequences (or its complementary strand) or amino acid sequences that have sequence identity of at least 80% (e.g., at least 85%, 88%, 90%, 91%, 92%, 93%, 94%, 95%, 96%, 97%, 98%, 99%) to another sequences when optimally aligned.

“Percent (%) sequence identity” with respect to amino acid sequence (or nucleic acid sequence) is defined as the percentage of amino acid (or nucleic acid) residues in a candidate sequence that are identical to the amino acid (or nucleic acid) residues in a reference sequence, after aligning the sequences and, if necessary, introducing gaps, to achieve the maximum number of identical amino acids (or nucleic acids). Conservative substitution of the amino acid residues may or may not be considered as identical residues. Alignment for purposes of determining percent amino acid (or nucleic acid) sequence identity can be achieved, for example, using publicly available tools such as BLASTN, BLASTp (available on the website of U.S. National Center for Biotechnology Information (NCBI), see also, Altschul S. F. et al, J. Mol. Biol., 215:403-410 (1990); Stephen F. et al, Nucleic Acids Res., 25:3389-3402 (1997)), ClustalW2 (available on the website of European Bioinformatics Institute, see also, Higgins D. G. et al, Methods in Enzymology, 266:383-402 (1996); Larkin M. A. et al, Bioinformatics (Oxford, England), 23(21): 2947-8 (2007)), and ALIGN or Megalign (DNASTAR) software. Those skilled in the art may use the default parameters provided by the tool, or may customize the parameters as appropriate for the alignment, such as for example, by selecting a suitable algorithm.

As used herein, the term “myeloid cell” refers to normal or neoplastic cells found in the blood, bone marrow, other hematopoietic or other non-hematopoietic compartments of the body. In particular, the term “myeloid cells” is used herein to mean the cell lineage originating from the bone marrow that includes monocytes (which gives rise to macrophages and dendritic cells), polymorphonuclear neutrophils, eosinophils, basophils, and mast cells, as well as the monocyte/macrophage lineage and different dendritic cell lineages. The term refers to cells of the myeloid lineages in all stages of their differentiation and therefore includes hematopoietic blast cells, i.e., hematopoietic cells that are committed to the myeloid cell lineage, but that are in early stages of differentiation. Examples are inter alia myeloblasts. The term “myeloid cells” also includes myeloid progenitor cells, i.e., cell lineages, e.g., in the bone marrow, that are capable of differentiating in cells such as myelomonocytic progenitor cells, proerythroblasts or immature megakaryoblasts. “Treating” or “treatment” of a condition as used herein includes preventing or alleviating a condition, slowing the onset or rate of development of a condition, reducing the risk of developing a condition, preventing or delaying the development of symptoms associated with a condition, reducing or ending symptoms associated with a condition, generating a complete or partial regression of a condition, curing a condition, or some combination thereof.

The term “subject” or “individual” or “animal” or “patient” as used herein refers to human or non-human animal, including a mammal or a primate, in need of diagnosis, prognosis, amelioration, prevention and/or treatment of a disease or disorder. Mammalian subjects include humans, domestic animals, farm animals, and zoo, sports, or pet animals such as dogs, cats, guinea pigs, rabbits, rats, mice, horses, swine, cows, bears, and so on.

The term “vector” as used herein refers to a vehicle into which a polynucleotide encoding a protein may be operably inserted so as to bring about the expression of that protein. A vector may be used to transform, transduce, or transfect a host cell so as to bring about expression of the genetic element it carries within the host cell. Examples of vectors include plasmids, phagemids, cosmids, and artificial chromosomes such as yeast artificial chromosome (YAC), bacterial artificial chromosome (BAC), or P1-derived artificial chromosome (PAC), bacteriophages such as lambda phage or M13 phage, and animal viruses. Categories of animal viruses used as vectors include retrovirus (including lentivirus), adenovirus, adeno-associated virus, herpesvirus (e.g., herpes simplex virus), poxvirus, baculovirus, papillomavirus, and papovavirus (e.g., SV40). A vector may contain a variety of elements for controlling expression, including promoter sequences, transcription initiation sequences, enhancer sequences, selectable elements, and reporter genes. In addition, the vector may contain an origin of replication. A vector may also include materials to aid in its entry into the cell, including but not limited to a viral particle, a liposome, or a protein coating. A vector can be an expression vector or a cloning vector.

The phrase “host cell” as used herein refers to a cell into which an exogenous polynucleotide and/or a vector has been introduced.

“Cancer” as used herein interchangeably with “tumor”, refers to any medical condition characterized by malignant cell growth or neoplasm, abnormal proliferation, infiltration or metastasis, and includes both solid tumors and non-solid cancers (hematologic malignancies) such as leukemia. As used herein “solid tumor” refers to a solid mass of neoplastic and/or malignant cells. Examples of cancer or tumors include hematological malignancies, oral carcinomas (for example of the lip, tongue or pharynx), digestive organs (for example esophagus, stomach, small intestine, colon, large intestine, or rectum), peritoneum, liver and biliary passages, pancreas, respiratory system such as larynx or lung (small cell and non-small cell), bone, connective tissue, skin (e.g., melanoma), breast, reproductive organs (fallopian tube, uterus, cervix, testicles, ovary, or prostate), urinary tract (e.g., bladder or kidney), brain and endocrine glands such as the thyroid. In certain embodiments, the cancer is selected from ovarian cancer, breast cancer, head and neck cancer, renal cancer, bladder cancer, hepatocellular cancer, and colorectal cancer. In certain embodiments, the cancer is selected from a lymphoma, Hodgkin's lymphoma, non-Hodgkin's lymphoma and B-cell lymphoma.

The term “pharmaceutically acceptable” indicates that the designated carrier, vehicle, diluent, excipient(s), and/or salt is generally chemically and/or physically compatible with the other ingredients comprising the formulation, and physiologically compatible with the recipient thereof.

### A. Multi-Specific Molecule

In one aspect, the present disclosure provides a multi-specific molecule comprising: a SIRP-alpha binding domain, an activating receptor-binding domain, and a target antigen binding domain. The multi-specific molecules provided herein are configured to be able to bind to 1) an activating receptor (for example, FcγR expressed on a phagocytic cell), 2) a target antigen expressed on a target cell, and 3) SIRPalpha expressed on an immune effector cell (e.g., a phagocytic cell, such as monocyte or a macrophage). The target antigen can be, for example, a tumor surface antigen, an inflammatory antigen, or an antigen of an infectious microorganism.

Without wishing to be bound by any theory, it is believed that SIRP-alpha binding domain and the activating receptor-binding domain could engage and provide multiple activation signals by crosslinking with SIRP-alpha and the activating receptor on the immune effector cell (e.g., phagocytic cell) and then crosslinking with the target cell via the interaction between the target-antigen binding domain and the target antigen on the target cell. Such design allows the activation of the immune effector cell and engagement or recruitment of the activated immune effector cell to a target microenvironment to trigger phagocytosis and killing of the target cell (e.g., cancer cell, an infected cell, or a damaged or disease cell). Accordingly, the multi-specific molecules of the present disclosure are also called multi-specific macrophage engagers. The engagers provided herein are advantageous, especially in selectively eliminating unwanted cells (such as cancer cells), while normal cells even for those expressing CD47 would not be targeted by the phagocytosis of the macrophages. Therefore, the multi-specific molecules provided herein show high selectivity in eliminating abnormal cells over normal cells, and thus have reduced side effect, lower toxicity and high safety in, for example, cancer treatment.

Without wishing to be bound by any theory, in cancer treatment, the activation of SIRP-alpha and/or the activating receptors (e.g., FcγR) redirects tumor associated macrophages (TAMs) from a pro-tumorigenic state to an anti-tumorigenic state. Accordingly, the design of the multi-specific molecules or engagers provided herein also allows redirecting tumor associated macrophages (TAMs) previously present in and/or newly recruited to the tumor microenvironment to enhance cancer killing. In other words, the multi-specific molecules/multi-specific macrophage engagers provided herein can promote or maintain monocytes or macrophages that can effectively and specifically kill cancer cells, as opposed to becoming immunesuppressive and TAMs. As such, the present disclosure also provides methods of redirecting TAMs into anti-tumor macrophages to enhance phagocytosis of the cancer cells.

In cancer, there is a tumor microenvironment which contribute to establishment of an immunosuppressive environment. The tumor microenvironment is a complex ecology of cells that evolves with and provides support to tumor cells during the transition to malignancy (Noy et al., Tumor-associated macrophages: from mechanisms to therapy, Immunity. 2014 Jul. 17; 41(1): 49-61.). Factors like IL-10, glucocorticoid hormones, apoptotic cells, and immune complexes can interfere with innate immune cell function.

Monocytes that give rise to mature macrophages can be attracted by numerous factors and migrate into the tumor microenvironment, where a majority of these monocytes can differentiat into TAMs (Zhou et al., (2020) Tumor-Associated Macrophages: Recent Insights and Therapies. Front. Oncol. 10:188). Macrophages residing within the tumor microenvironment may also be re-polarized by the various factors in the tumor microenvironment so that these macrophages become differentiated into tumor-promoting TAMs.

TAMs comprise mainly alternatively activated macrophages (M2 phenotype) and a small fraction of classically activated macrophages (M1 phenotype) (Zhou et al., (2020) Tumor-Associated Macrophages: Recent Insights and Therapies. Front. Oncol. 10:188). Macrophages with M1 phenotype are potent and capable of killing pathogens or cancer cells. Macrophages with M2 phenotype lack the function of phagocytizing tumor cells and also help these tumor cells escape from being killed and help them spread to other tissues and organs (Zhou et al., (2020) Tumor-Associated Macrophages: Recent Insights and Therapies. Front. Oncol. 10:188).

The SIRP-alpha binding domain of the engagers provided herein can inhibit downregulation of the phagocytosis of a macrophage mediated by CD47-SIRPalpha axis signaling. Cancer cells typically overexpress CD47, which binds to SIRPalpha expressed on an immune cell (e.g., monocyte or macrophage) to elicit the “don't eat me” signal that prevents the cancer cell from being eliminated by the immune cell (e.g., monocyte or macrophage). Inhibition of the CD47-SIRPalpha axis can therefore counteract the cancer cell mediated anti-phagocytotic activity.

The SIRP-alpha binding domain provided herein can inhibit the CD47-SIRPalpha axis signaling by blocking the CD47-SIRPalpha interaction and/or blocking downstream signaling (e.g., SHP-1 signaling) mediated by the CD47-SIRPalpha interaction. In certain embodiments, the SIRP-alpha binding domain can comprise a SIRPalpha blocker, such as the the extracellular domain (ECD) of CD47 that recognizes and binds to SIRPalpha, or an antibody or its antigen-binding domain that recognizes and binds to SIRPalpha.

In a more preferred embodiments, the antibody or its antigen-binding domain that recognizes and binds to SIRPalpha has one or more of the following properties: 1) capable of substantially or completely blocking interaction between SIRP-alpha and CD47; 2) capable of substantially or completely blocking SHP-1 recruitment mediated by interaction between SIRP-alpha and CD47; 3) has minimal intrinsic activity to induce the phagocytosis of monocyte or macrophage; and 4) capable of binding to an epitope outside the IgV domain of SIRPα.

The multi-specific molecule provided herein further comprises a target antigen binding domain. As such, the term “target antigen binding domain” as used herein encompasses any binding domain for the target antigen. The term “target antigen” refers to any cell surface marker that can distinguish the cell expressing the target antigen from other cells, which includes but not limited to tumor antigen, or antigens presented on infected cells.

In certain embodiments, the multi-specific molecule provided herein selectively induce effector function of an immune effector cell (that co-expresses SIRP-alpha and the activating receptor) in the presence of the target antigen.

The multi-specific molecule provided herein is capable of binding to and activating an immune effector cell to mount a selective response to a target cell over a non-target cell.

In certain embodiments, the target cell expresses the target antigen. In certain embodiments, the target cell co-expresses the target antigen and CD47.

In certain embodiments, the multi-specific molecule provided herein induces minimal effector function of the immune effector cell in the absence of the target antigen. As used herein, the term “minimal” with respect to effector function refers to the level of effector function comparable to that induced by an isotype control. In certain embodiments, the effector function induced by the multi-specific molecule provided herein in the absence of the target antigen is no more than 10%, 20%, 30%, 40%, 50% of that induced in the presence of the target antigen.

The multi-specific molecules or the engagers of the present disclosure also comprise additional structures to assist the engagers to modularly and concomitantly engage with multiple targets. The additional structures are, for example, connecting elements such as linkers, cognate peptides, or chemical binding, for two or more binding domains to be linked and separated to realize the spatial proximity and flexibility.

The incorporation of the additional structures described above into the higher order multi-specific macrophage engagers provided herein assists in the production, folding, stability, function and tissue availability of the engagers.

i. SIRPα-Binding Domain

The SIRPα-binding domain of the multi-specific molecule provided herein is capable of substantially blocking interaction between SIRP-alpha and CD47.

By “substantially block interaction” between two interacting molecules, it is meant that an antibody is capable of inhibiting at least 50% binding between the two interacting molecules, or capable of inhibiting at least 40% signal transduction induced by interaction of the two molecules. The signal transduction induced by interaction between SIRP-alpha and CD47 can be characterized by SHP1 recruitment to intracellular portion (e.g. C-terminal tail) of SIRP-alpha.

In certain embodiments, the SIRPα-binding domain of the multi-specific molecule provided herein are capable of completely blocking interaction between SIRP-alpha and CD47. The phrase “completely block” with respect to two interacting molecules, means inhibition of at least 80% binding between the two interacting molecules, or inhibition of at least 50% signal transduction induced by interaction of the two molecules.

In certain embodiments, the SIRPα-binding domain of the multi-specific molecule provided herein can completely block the “don't eat me” signal delivered by binding of CD47 to SIRPα that suppresses phagocytosis, either by completely blocking SIRPα-CD47 interaction or by completely blocking SIRPα-CD47 interaction mediated downstream signaling (e.g., SHP-1 recruitment), regardless its blocking effect on the SIRPα-CD47 interaction. The SIRP-alpha antibodies that are capable of completely blocking interaction between SIRP-alpha and CD47 are also referred to herein as complete blocker.

Blocking of binding interaction between SIRP-alpha and CD47 can be determined by any suitable assay, for example, competitive ELISA or competitive FACS assay. In brief, for competitive ELISA, soluble extracellular domain (ECD) of SIRP-alpha can be immobilized on a substrate, a test SIRP-alpha antibody can be tested at different concentrations for its capability to block binding of a certain concentration of soluble ECD of CD47 to the immobilized ECD of SIRP-alpha. Binding of ECD of CD47 to the immobilized ECD of SIRP-alpha can be determined in the absence of and in the presence of a test SIRP-alpha binding domain, respectively. Reduction in binding of CD47 and SIPR-alpha in the presence of the test SIRP-alpha binding domain can be determined, and accordingly the percentage of blocking can be determined.

In certain embodiments, certain anti-SIRPα binding domains provided herein have maximal blocking percentage of more than 90% as measured by competitive ELISA assay.

The assay conditions can be similar to those provided in Example 12 of the present disclosure (the concentration of soluble ECD of CD47 is 25 nM, and the concentration of soluble ECD of SIRPα is 20 nM). Exemplary complete blockers as provided herein are anti-SIRPα binding domains derived from C15, C25, C42, C59, C73 and humanized antibodies thereof.

The term “maximal blocking percentage” used interchangeably with “top blocking percentage” refers to the plateau of blocking percentage of the interaction between two proteins (e.g., SIRP-alpha and CD47) in presence of a blocker (e.g., SIRPα-binding domain) at an increasing concentration. In general, the percentage of blocking may rise with the increase in concentration of the SIRP-alpha binding domains, it could, however, reach a plateau where no more blocking can be achieved despite of further increase in the concentration of the SIRP-alpha binding domains. The maximal blocking percentage may vary depending on different assays, such as competitive ELISA assay and competitive FACS assay.

In certain embodiments, certain anti-SIRPα binding domains provided herein have maximal blocking percentage of no more than 10% maximal blocking percentage as measured by competitive ELISA assay. The assay conditions can be similar to those provide in Example 12 of the present disclosure (the concentration of soluble ECD of CD47 is 25 nM, and the concentration of soluble ECD of SIRPα is 20 nM). Exemplary non-blocker as provided herein is C50 and humanized antibody thereof. Such SIRP-alpha antibodies that are also referred to herein as non-blocker.

In certain embodiments, certain anti-SIRPα antibodies provided herein has maximal blocking percentage of no more than 50% maximal blocking percentage as measured by competitive ELISA assay. The assay conditions are similar to those provide in Example 12 of the present disclosure. Such SIRP-alpha antibodies that are also referred to herein as partial-blocker. Exemplary partial blocker as provided herein is C35 and humanized antibody thereof.

In certain embodiments, the SIRPα-binding domain of the multi-specific molecule provided herein has minimal intrinsic activity to induce the effector function of the immune effector cell to a target cell. In certain embodiments, the target cell is a tumor cell or a cancerous cell, an infected cell, or a specific disease cell type that needs to be eliminated by SIRP-alpha mediated effector functions, such as phagocytosis. As used herein, the term “intrinsic activity” with respect to the SIRPα-binding domain refers to the capability of the SIRPα-binding domain, in the absence of a target antigen binding domain, to induce phagocytosis of the immune effector cell on the target cell co-expressing certain target antigen and CD47. In certain embodiments, the SIRPα-binding domain induces no more than 20%, no more than 15%, no more than 10% phagocytosis to a target cell. As used herein, the term “minimal” with respect to intrinsic activity to induce phagocytosis refers to the level of phagocytosis induced by intrinsic activity of SIRP-alpha binding domain comparable to that induced by an isotype control. In certain embodiments, the phagocytosis induced by the SIRP-alpha binding domain alone or molecules comprising the SIRP-alpha binding domain alone is no more than 10%, 20%, 30%, 40%, 50% of that induced by the engagers provided herein.

In certain embodiments, the SIRPα-binding domain of the multi-specific molecule provided herein comprises an antibody domain or an antibody mimetic domain.

As used herein, the term “antibody domain” refers to an antigen-binding domain derived from an antibody and comprises at least one antibody fragment (such as CDR, and/or variable region sequence). An antibody domain includes, for example, a monoclonal antibody, an antibody fragment or domain, a fusion protein comprising an antibody fragment or domain, a polypeptide complex comprising an antibody fragment or domain, and so on. In certain embodiments, the antibody domain comprises a Fab, a VHH, a single chain Fv (scFv), diabody, a Fab′, a F(ab′)₂, a Fd, an Fv fragment, a disulfide stabilized Fv fragment (dsFv), a (dsFv)₂, a bispecific dsFv (dsFv-dsFv′), a disulfide stabilized diabody (ds diabody), F(ab)₂, an scFv dimer (bivalent diabody), a camelized single domain antibody, a nanobody, a Tetrabody, a domain antibody, or a bivalent domain antibody.

As used herein, the term “antibody mimetic domain” refers to artificial peptides or compounds that behave in a similar fashion to antibodies (e.g., can specifically bind to antigens similar to antibodies), but that are not generated by the immune system nor structurally related to antibodies. The term “antibody mimetic domain” may also refer to unrelated protein scaffolds comprising α-helices, β-sheets, or random coils that can bind to specific targets and could be designed to incorporate new binding sites through protein engineering strategies.

In certain embodiments, the SIRPα-binding domain is the antibody mimetic domain comprising an intrabody (e.g., fibronectin domain), a monobody, a linear peptide, Z domain of protein A (Affibody), gamma-B crystalline domain, ubiquitin domain, cystatin domain, Sac7d domain, triple helix coiled coil domain, lipocalins domain, A domains of a membrane receptor, Ankyrin repeat motif, SH3 domain of Fyn, Kunitz domain of a protease inhibitor, type III domain of fibronectin (Minibody), a DARPin domain, or carbohydrae binding module 32-2.

In certain embodiments, the SIRPα-binding domain comprises one or more (e.g. 1, 2, 3, 4, 5, or 6) CDR sequences of an anti-SIRPα antibody selected from the group consisting of: C25, hu025.021, hu025.033, hu025.023, hu025.059, hu025.060, C15, C42, C59 and C73.

“C25” or “025c” as used herein refers to a mouse antibody having a heavy chain variable region of SEQ ID NO: 1, and a light chain variable region of SEQ ID NO: 2.

“hu025.021” as used herein refers to a humanized antibody based on C25 that comprises a heavy chain variable region of SEQ ID NO: 3, and a light chain variable region of SEQ ID NO: 4.

“hu025.023” as used herein refers to a humanized antibody based on C25 that comprises a heavy chain variable region of SEQ ID NO: 5, and a light chain variable region of SEQ ID NO: 6.

“hu025.033” as used herein refers to a humanized antibody based on C25 that comprises a heavy chain variable region of SEQ ID NO: 159, and a light chain variable region of SEQ ID NO: 160.

“hu025.059” as used herein refers to a humanized antibody based on C25 that comprises a heavy chain variable region of SEQ ID NO: 7, and a light chain variable region of SEQ ID NO: 8.

“hu025.060” as used herein refers to a humanized antibody based on C25 that comprises a heavy chain variable region of SEQ ID NO: 9, and a light chain variable region of SEQ ID NO: 10.

“C15” or “015c” as used herein refers to a mouse antibody having a heavy chain variable region of SEQ ID NO: 11, and a light chain variable region of SEQ ID NO: 12.

“C42” or “042c” as used herein refers to a mouse antibody having a heavy chain variable region of SEQ ID NO: 13, and a light chain variable region of SEQ ID NO: 14.

“C59” or “059c” as used herein refers to a mouse antibody having a heavy chain variable region of SEQ ID NO: 15, and a light chain variable region of SEQ D NO: 16.

“C73” or “073C” as used herein refers to a mouse antibody having a heavy chain variable region of SEQ ID NO: 17, and a light chain variable region of SEQ TD NO: 18.

The SIRPalpha binding domain of the multi-specific molecules provided herein can be obtained from anti-SIRPalpha antibodies, amino acid sequences of the variable regions and CDRs of which are shown in Tables 1-2 below.

TABLE 1
Amino acid sequences of the variable regions for anti-SIRPα antibodies
(CDRs are underlined).

| Antibody | VH | VL |
|---|---|---|
| C25 | C25.VH, SEQ ID NO: 1: | C25.VL, SEQ ID NO: 2: |
| | EVQLQQSGAELVKPGASVKL | QIVLTQSPAIMSASPGEKVTL |
| | SCTASGFNIKDYYMHWVKQR | TCSASSSVSSSYLYWYQQKP |
| | TEQGLEWIGRIDPEDGETKYA | GSSPKLWIYSTSNLASGVPA |
| | PKFQGKATITADTSSNTAYLQ | RFSGSGSGTSYSLTISSMEAE |
| | LSSLTSEDTAVYYCDRGLAY | DAASYFCHQWSSYPYTFGG |
| | WGQGTLVTVSA | GTKLEIK |
| hu025.021 | hu025.021.VH, SEQ ID NO: 3: | hu025.021.VL, SEQ ID NO: 4: |
| | EVQLVQSGAEVKKPGATVKIS | EIVLTQSPATLSLSPGERATL |
| | CKVSGFNIKDYYMHWVQQA | SCSASSSVSSSYLYWYQQKP |
| | PGKGLEWIGRIDPEDAETKYA | GQAPKLWIYSTSNLASGIPA |
| | PKFQGRVTITADTSTNTAYME | RFSGSGSGTDYTLTISSLEPE |
| | LSSLRSEDTAVYYCDRGLAY | DFAVYYCHQWSSYPYTFGQ |
| | WGQGTLVTVSS | GTKLEIK |
| hu025.023 | hu025.023.VH, SEQ ID NO: 5: | hu025.023.VL, SEQ ID NO: 6: |
| | EVQLVQSGAEVKKPGATVKIS | EIVLTQSPATLSLSPGERATL |
| | CKVSGFNIKDYYMHWVQQA | SCSASSSVSSSYLYWYQQKP |
| | PGKGLEWIGRIDPEDAETKYA | GQAPKLWIYSTSNLASGIPA |
| | PKFQGRVTITADTSTNTAYME | RFSGSGSGTDFTLTISSLEPE |
| | LSSLRSEDTAVYYCDRGLAY | DFAVYYCHQWSSYPYTFGQ |
| | WGQGTLVTVSS | GTKLEIK |
| hu025.033 | SEQ ID NO: 159 | SEQ ID NO: 160 |
| | EVQLVQSGAEVKKPGATVKIS | EIVLTQSPATLSLSPGERATL |
| | CKVSGFNIKDYYMHWVQQA | SCSASSSVSSSYLYWYQQKP |
| | PGKGLEWIGRIDPEDAETKYA | GQAPKLWIYSTSNLASGIPA |
| | PKFQGRVTITADTSTDTAYME | RFSGSGSGTDYTLTISSLEPE |
| | LSSLRSEDTAVYYCDRGLAY | DFAVYYCHQWSSYPYTFGQ |
| | WGQGTLVTVSS | GTKLEIK |
| hu025.059 | hu025.059.VH, SEQ ID NO: 7: | hu025.059.VL, SEQ ID NO: 8: |
| | EVQLVQSGAEVKKPGATVKIS | EIVLTQSPATLSLSPGERATL |
| | CKASGFNIKDYYMHWVQQA | SCSASSSVSSSYLYWYQQKP |
| | PGKGLEWIGRIDPEDAETKYA | GQAPKLWIYSTSNLASGIPA |
| | PKFQGRVTITADTSTNTAYME | RFSGSGSGTDYTLTISSLEPE |
| | LSSLRSEDTAVYYCDRGLAY | DFAVYYCHQWSSYPYTFGQ |
| | WGQGTLVTVSS | GTKLEIK |
| hu025.060 | hu025.060.VH, SEQ ID NO: 9: | hu025.060.VL, SEQ ID NO: 10: |
| | EVQLVQSGAEVKKPGATVKIS | EIVLTQSPATLSLSPGERATL |
| | CKASGFNIKDYYMHWVQQA | SCSASSSVSSSYLYWYQQKP |
| | PGKGLEWIGRIDPEDAETKYA | GQAPKLWIYSTSNLASGIPA |
| | PKFQGRVTITADTSTNTAYME | RFSGSGSGTDFTLTISSLEPE |
| | LSSLRSEDTAVYYCDRGLAY | DFAVYYCHQWSSYPYTFGQ |
| | WGQGTLVTVSS | GTKLEIK |
| C15 | C15.VH, SEQ ID NO: 11: | C15.VL, SEQ ID NO: 12: |
| | EVQLQQSGVEVVQPGASVKL | QIVLTQSPAIMSASPGEKVTL |
| | SCTASGFNIEAYYMHWVKQR | TCSASSSVSSSYLYWYQQKP |
| | TEQGLEWIGRIDPEDGESKYA | GSSPKLWIYSTSNLASGVPPR |
| | PKFQGKATMTADTSSSTAYL | FSGSGSGTSYSLTISSMQAED |
| | QLSSLTSDDTAVYYCVRGSYE | AASYFCYQWSSYPYTFGGG |
| | YWGQGTTLTVSS | TKLEIK |
| C42 | C42.VH, SEQ ID NO: 13: | C42.VL, SEQ ID NO: 14: |
| | QIQLVQSGPELKKPGETVKISC | DIVMTQSQKFMSTTIRDRVSI |
| | RASGYTFTTYGMSWVKQAPG | TCKASQNVGISVAWYQQKS |
| | KGLRWMGWINTYSGVSTCA | GQSPKLLIYSASNRYTGVPD |
| | DDFKGRFAFSLETSATTAYLQ | RFTGSGSGTDFTLTISNMQSE |
| | IHNLTNEDTATYFCARDPHSY | DLADYFCQQYSSYPLTFGSG |
| | GNSPAWFPYWGQGTLVTVSA | TKLAIK |
| C59 | C59.VH, SEQ ID NO: 15: | C59.VL, SEQ ID NO: 16: |
| | QVQLQQSGPELVKPGASVKM | ENVLTQSPEKMAVSLGQKV |
| | SCKASGYTFSEYVLSWVKQR | TMTCSASSSVSSSDLHWYQQ |
| | TGQGLEWIGEIYPGTITTYYN | KSGASPKPLIHGTSNLASGVP |
| | EKFKGKATLTADKSSNTAYIQ | ARFSGSGSGTSYSLTISSVEA |
| | LTSLTSEDSAVYFCGRFYDYD | EDAATYYCQQWSGYPWTFG |
| | GGWFAYWGQGTLLTVSA | GGTNLEIK |
| C73 | C73.VH, SEQ ID NO: 17: | C73. VL, SEQ ID NO: 18: |
| | QIQLVQSGPELKKPGETVKISC | DIVMTQSQKFMSTTIGDRVSI |
| | KASGYTFTTYGMSWVKQAPG | TCEASQIVGIAVAWYQQKPG |
| | KGLKWMVWINTYSGVPTYA | QSPKLLIYSASNRYTGVPDR |
| | DDFKGRFAFSLETSASTSYLQI | FTGSGSGTDFTLTISNMQSED |
| | NNLKNEDTATYFCARDPHYY | LANYFCQQYSAYPFTFGSGT |
| | GSSPAWFVYWGQGTLVTVSA | KLEVK |
| C35 | C35.VH, SEQ ID NO: 19: | C35.VL, SEQ ID NO: 20: |
| | QIQLVQSGPELRKPGETVKISC | DIVMTQSQKFMSTSIGDRVS |
| | KASGYSFTNYAMNWVKQAP | VTCKASQNVGTHLAWYQQ |
| | GKVLKWMGFINTYTGEPTYA | KPGQSPKALIFSASYRYIGVP |
| | DDFKGRFAFSLETSASTAYLQ | DRFTGSGSGTDFTLTITNVQS |
| | INNLKNEDTATYFCTRTRGYY | EDLAEYFCQQYNTYPLTFGA |
| | DFDGGAFDYWGQGTSLTVSS | GTKLELK |
| C50 | C50.VH, SEQ ID NO: 21: | C50.VL, SEQ ID NO: 22: |
| | QIQLVQSGPELKKPGETVKISC | QIVLTQSPPIMSASPGEKVTL |
| | KASGYTFTHYSMHWVKQAP | TCSATSSVSASYLYWFQQKP |
| | GKGLKWMGWINTETAEPTYV | GSSPKLWIYSTSNLASGVPA |
| | DDFKGRFAFSLEASASTAFFQI | RFSGSGSGTSYSLTISNMEPA |
| | NNLKNEDTATYFCARGGLRQ | DAASYFCHQWSSYPYTFGG |
| | GDYWGQGTTLTVSS | GTKLEIK |

| | |
|---|---|
| C25.scFv | SEQ ID NO: 226 |
| | QIVLTQSPAIMSASPGEKVILTCSASSSVSSSYLYWYQQKPGSS |
| | PKLWIYSTSNLASGVPARFSGSGSGTSYSLTISSMEAEDAASYF |
| | CHQWSSYPYTFGGGTKLEIKGGGGSGGGGSGGGGSGGGGSE |
| | VQLQQSGAELVKPGASVKLSCTASGFNIKDYYMHWVKQRTE |
| | QGLEWIGRIDPEDGETKYAPKFQGKATITADTSSNTAYLQLSS |
| | LTSEDTAVYYCDRGLAYWGQGTLVTVSA |

TABLE 2
CDRs of the anti-SIRPa antibodies provided herein

| Anti- | | | | | | |
|---|---|---|---|---|---|---|
| body | | | | | | |
| ID | HCDR1 | HCDR2 | HCDR3 | LCDR1 | LCDR2 | LCDR3 |
| C15 | SEQ ID | SEQ ID | SEQ ID | SEQ ID | SEQ ID | SEQ ID |
| | NO: 29 | NO: 30 | NO: 31 | NO: 32 | NO: 33 | NO: 34 |
| | AYYMH | RIDPEDGE | GSYEY | SASSSVSSS | STSNLAS | YQWSSYP |
| | | SKYAPKFQ | | YLY | | YT |
| | | G | | | | |
| C25 | SEQ ID | SEQ ID | SEQ ID | SEQ ID | SEQ ID | SEQ ID |
| | NO: 23 | NO: 24 | NO: 25 | NO: 26 | NO: 27 | NO: 28 |
| | DYYMH | RIDPEDGE | GLAY | SASSSVSSS | STSNLAS | HQWSSYP |
| | | TKYAPKFQ | | YLY | | YT |
| | | G | | | | |
| | | SEQ ID | | | | |
| | | NO: 198 | | | | |
| | | RIDPEDAE | | | | |
| | | TKYAPKFQ | | | | |
| | | G | | | | |
| | SEQ ID | SEQ ID | SEQ ID | | | SEQ ID |
| | NO: 161 | NO: 162 | NO: 163 | | | NO: 164 |
| | X₁YYMH | RIDPEDX₂E | GX₁₅X₄X₅Y | | | X₆QWSSY |
| | | X₃KYAPKF | | | | PYT |
| | | QG | | | | |
| C42 | SEQ ID | SEQ ID | SEQ ID | SEQ ID | SEQ ID | SEQ ID |
| | NO: 35 | NO: 36 | NO: 37 | NO: 38 | NO: 39 | NO: 40 |
| | TYGMS | WINTYSG | DPHSYGN | KASQNVGI | SASNRYT | QQYSSYP |
| | | VSTCADDF | SPAWFPY | SVA | | LT |
| | | KG | | | | |
| C73 | SEQ ID | SEQ ID | SEQ ID | SEQ ID | SEQ ID | SEQ ID |
| | NO: 47 | NO: 48 | NO: 49 | NO: 50 | NO: 51 | NO: 52 |
| | TYGMS | WINTYSG | DPHYYGSS | EASQIVGI | SASNRYT | QQYSAYP |
| | | VPTYADDF | PAWFVY | AVA | | FT |
| | | KG | | | | |
| | | SEQ ID | SEQ ID | SEQ ID | | SEQ ID |
| | | NO: 165 | NO: 166 | NO: 188 | | NO: 189 |
| | | WINTYSG | DPHX₉YG | X₁₂ASQX₁₃ | | QQYSX₁₆ |
| | | VX₇TX₈AD | X₁₀SPAWF | VGIX₁₄V | | YPX₁₇T |
| | | DFKG | X₁₁Y | A | | |
| C59 | SEQ ID | SEQ ID | SEQ ID | SEQ ID | SEQ ID | SEQ ID |
| | NO: 41 | NO: 42 | NO: 43 | NO: 44 | NO: 45 | NO: 46 |
| | EYVLS | EIYPGTITT | FYDYDGG | SASSSVSSS | GTSNLAS | QQWSGY |
| | | YYNEKFKG | WFAY | DLH | | PWT |
| C35 | SEQ ID | SEQ ID | SEQ ID | SEQ ID | SEQ ID | SEQ ID |
| | NO: 53 | NO: 54 | NO: 55 | NO: 56 | NO: 57 | NO: 58 |
| | SGYSFTNY | FINTYTGE | TRGYYDFD | KASQNVG | SASYRYI | QQYNTYP |
| | AMN | PTYADDFK | GGAFDY | THLA | | LT |
| | | G | | | | |
| C50 | SEQ ID | SEQ ID | SEQ ID | SEQ ID | SEQ ID | SEQ ID |
| | NO: 59 | NO: 60 | NO: 61 | NO: 62 | NO: 63 | NO: 64 |
| | SGYTFTHY | WINTETAE | GGLRQGD | SATSSVSA | STSNLAS | HQWSSYP |
| | SMH | PTYVDDFK | Y | SYLY | | YT |
| | | G | | | | |

X₁ is A or D; X₂ is G or A; X₃ is T or S; X₄ is L or Y; X₅ is E or A; X₆ is Y or H; X₇ is S or P; X₈ is Y or C; X₉ is Y or S; X₁₀ is N or S, X₁₁ is P or V; X₁₂ is E or K; X₁₃ is N or I; X₁₄ is S or A; X₁₅ is S or absent; X₁₆ is S or A; X₁₇ is For L.

CDRs are known to be responsible for antigen binding, however, it has been found that not all of the 6 CDRs are indispensable or unchangeable. In other words, it is possible to replace or change or modify one or more CDRs provided herein for SIRPα-binding domains, yet substantially retain the specific binding affinity to SIRPα.

Heavy chain CDR3 regions are located at the center of the antigen-binding site, and therefore are believed to make the most contact with antigen and provide the most free energy to the affinity of antibody to antigen. It is also believed that the heavy chain CDR3 is by far the most diverse CDR of the antigen-binding site in terms of length, amino acid composition and conformation by multiple diversification mechanisms (Tonegawa S. Nature. 302:575-81). The diversity in the heavy chain CDR3 is sufficient to produce most antibody specificities (Xu J L, Davis M M. Immunity. 13:37-45) as well as desirable antigen-binding affinity (Schier R, etc. J Mol Biol. 263:551-67).

In certain embodiments, the SIRP-alpha binding domain comprises:
a) a HCDR1 comprising the sequence of X₁YYMH (SEQ ID NO: 161), a HCDR2 comprising the sequence of RIDPEDX₂EX₃KYAPKFQG (SEQ ID NO: 162), and a HCDR3 comprising the sequence of GX₁₅X₄X₅Y (SEQ ID NO: 163); and/or a LCDR1 comprising the sequence of SASSSVSSSYLY (SEQ ID NO: 26), a LCDR2 comprising the sequence of STSNLAS (SEQ ID NO: 27), and a LCDR3 comprising the sequence of X₆QWSSYPYT (SEQ ID NO: 164); or
b) a HCDR1 comprising the sequence of TYGMS (SEQ ID NO: 35), a HCDR2 comprising the sequence of WINTYSGVX₇TX₈ADDFKG (SEQ ID NO: 165), and a HCDR3 comprising the sequence of DPHX₉YGX₁₀SPAWFX₁₁Y (SEQ ID NO: 166); and/or a LCDR1 comprising the sequence of X₁₂ASQX₁₃VGIX₁₄VA (SEQ ID NO: 188), a LCDR2 comprising the sequence of SASNRYT (SEQ ID NO: 39), and a LCDR3 comprising the sequence of QQYSX₁₆YPX₁₇T (SEQ ID NO: 189); or
c) a HCDR1 comprising the sequence of EYVLS (SEQ ID NO: 41), a HCDR2 comprising the sequence of EIYPGTITTYYNEKFKG (SEQ ID NO: 42), and a HCDR3 comprising the sequence of FYDYDGGWFAY (SEQ ID NO: 43); and/or a LCDR1 comprising the sequence of SASSSVSSSDLH (SEQ ID NO: 44), a LCDR2 comprising the sequence of GTSNLAS (SEQ ID NO: 45), and a LCDR3 comprising the sequence of QQWSGYPWT (SEQ ID NO: 46),
wherein X₁ is A or D; X₂ is G or A; X₃ is T or S; X₄ is L or Y; X₅ is E or A; X₆ is Y or H; X₇ is S or P; X₈ is Y or C; X₉ is Y or S; X₁₀ is N or S; X₁₁ is P or V; X₁₂ is E or K; X₁₃ is N or I; X₁₄ is S or A; X₁₅ is S or absent; X₁₆ is S or A; X₁₇ is F or L.

In certain embodiments, the SIRP-alpha binding domain comprises:
a) a HCDR1 comprising the sequence of SEQ ID NO: 23, a HCDR2 comprising the sequence of SEQ ID NO: 24 or SEQ ID NO: 198, and a HCDR3 comprising the sequence of SEQ ID NO: 25; and/or a LCDR1 comprising the sequence of SEQ ID NO: 26, a LCDR2 comprising the sequence of SEQ ID NO: 27, and a LCDR3 comprising the sequence of SEQ ID NO: 28; or
b) a HCDR1 comprising the sequence of SEQ ID NO: 29, a HCDR2 comprising the sequence of SEQ ID NO: 30, and a HCDR3 comprising the sequence of SEQ ID NO: 31; and/or a LCDR1 comprising the sequence of SEQ ID NO: 32, a LCDR2 comprising the sequence of SEQ ID NO: 33, and a LCDR3 comprising the sequence of SEQ ID NO: 34; or
c) a HCDR1 comprising the sequence of SEQ ID NO: 35, a HCDR2 comprising the sequence of SEQ ID NO: 36, and a HCDR3 comprising the sequence of SEQ ID NO: 37; and/or a LCDR1 comprising the sequence of SEQ ID NO: 38, a LCDR2 comprising the sequence of SEQ ID NO: 39, and a LCDR3 comprising the sequence of SEQ ID NO: 40; or
d) a HCDR1 comprising the sequence of SEQ ID NO: 47, a HCDR2 comprising the sequence of SEQ ID NOs: 48, and a HCDR3 comprising the sequence of SEQ ID NOs: 49; and/or a LCDR1 comprising the sequence of SEQ ID NOs: 50, a LCDR2 comprising the sequence of SEQ ID NOs: 51, and a LCDR3 comprising the sequence of SEQ ID NOs: 52.

In certain embodiments, the SIRP-alpha binding domain comprises the same HCDRs and LCDRs as anti-SIRP-alpha antibody selected from the group consisting of C25, C15, C42, C59 and C73, wherein:
a) the C25 comprises a heavy chain variable region comprising the sequence of SEQ ID NO: 1, and/or a light chain variable region comprising the sequence of SEQ ID NO: 2,
b) the C15 comprises a heavy chain variable region comprising the sequence of SEQ ID NO: 11, and/or a light chain variable region comprising the sequence of SEQ ID NO: 12,
c) the C42 comprises a heavy chain variable region comprising the sequence of SEQ ID NO: 13, and/or a light chain variable region comprising the sequence of SEQ ID NO: 14,
d) the C59 comprises a heavy chain variable region comprising the sequence of SEQ ID NO: 15, and/or a light chain variable region comprising the sequence of SEQ ID NO: 16, and
e) the C73 comprises a heavy chain variable region comprising the sequence of SEQ ID NO: 17, and/or a light chain variable region comprising the sequence of SEQ ID NO: 18.

In certain embodiments, the SIRPα-binding domains provided herein comprise any suitable framework region (FR) sequences, as long as the antigen-binding domains can specifically bind to SIRPα. In certain embodiments, the CDR sequences of hu025.021, hu025.033, hu025.023, hu025.059 and hu025.060 are obtained from mouse antibodies C25, but they can be grafted to any suitable FR sequences of any suitable species such as mouse, human, rat, rabbit, among others, using suitable methods known in the art such as recombinant techniques.

In certain embodiments, the SIRPα-binding domains provided herein are humanized. A humanized antigen-binding domain is desirable in its reduced immunogenicity in human. A humanized antigen-binding domain is chimeric in its variable regions, as non-human CDR sequences are grafted to human or substantially human FR sequences. Humanization of an antigen-binding domain can be essentially performed by substituting the non-human (such as murine) CDR genes for the corresponding human CDR genes in a human immunoglobulin gene (see, for example, Jones et al. (1986) Nature 321:522-525; Riechmann et al. (1988) Nature 332:323-327; Verhoeyen et al. (1988) Science 239:1534-1536).

Suitable human heavy chain and light chain variable domains can be selected to achieve this purpose using methods known in the art. In an illustrative example, “best-fit” approach can be used, where a non-human (e.g. rodent) antibody variable domain sequence is screened or BLASTed against a database of known human variable domain sequences, and the human sequence closest to the non-human query sequence is identified and used as the human scaffold for grafting the non-human CDR sequences (see, for example, Sims et al, (1993) J. Immunol. 151:2296; Chothia et al. (1987) J. Mot. Biol. 196:901). Alternatively, a framework derived from the consensus sequence of all human antibodies may be used for the grafting of the non-human CDRs (see, for example, Carter et at. (1992) Proc. Natl. Acad. Sci. USA, 89:4285; Presta et al. (1993) J. Immunol., 151:2623).

In certain embodiments, the humanized antigen-binding domains provided herein are composed of substantially all human sequences except for the CDR sequences which are non-human. In some embodiments, the variable region FRs, and constant regions if present, are entirely or substantially from human immunoglobulin sequences. The human FR sequences and human constant region sequences may be derived from different human immunoglobulin genes, for example, FR sequences derived from one human antibody and constant region from another human antibody. In some embodiments, the humanized antigen-binding domain comprise human FR1-4.

In some embodiments, the FR regions derived from human may comprise the same amino acid sequence as the human immunoglobulin from which it is derived. In some embodiments, one or more amino acid residues of the human FR are substituted with the corresponding residues from the parent non-human antibody. This may be desirable in certain embodiments to make the humanized antibody or its fragment closely approximate the non-human parent antibody structure. In certain embodiments, the humanized SIRPα-binding domain provided herein comprises no more than 10, 9, 8, 7, 6, 5, 4, 3, 2, or 1 amino acid residue substitutions in each of the human FR sequences, or no more than 10, 9, 8, 7, 6, 5, 4, 3, 2, or 1 amino acid residue substitutions in all the FRs of a heavy or a light chain variable domain. In some embodiments, such change in amino acid residue could be present in heavy chain FR regions only, in light chain FR regions only, or in both chains. Table 2.1 shows the FR sequences of the humanized antibodies hu025.021, hu025.023, hu025.033, hu025.059 and hu025.060.

TABLE 2.1
FR amino acid sequences of 5 humanized antibodies

| Antibody | | FR1 | FR2 | FR3 | FR4 |
|---|---|---|---|---|---|
| hu025.021 | HFR | SEQ ID NO: 207 | SEQ ID NO: 191 | SEQ ID NO: 208 | SEQ ID NO: |
| | | EVQLVQSGAEVK | WVQQAPGKGLE | RVTITADTSTNTA | 193 |
| | | KPGATVKISCKVS | WIG | YMELSSLRSEDTA | WGQGTLVTV |
| | | GFNIK | | VYYCDR | SS |
| | LFR | SEQ ID NO: 194 | SEQ ID NO: 195 | SEQ ID NO: 209 | SEQ ID NO: |
| | | EIVLTQSPATLSLS | WYQQKPGQAPK | GIPARFSGSGSGT | 197 |
| | | PGERATLSC | LWIY | DYTLTISSLEPEDF | FGQGTKLEIK |
| | | | | AVYYC | |
| hu025.023 | HFR | SEQ ID NO: 207 | SEQ ID NO: 191 | SEQ ID NO: 208 | SEQ ID NO: |
| | | EVQLVQSGAEVK | WVQQAPGKGLE | RVTITADTSTNTA | 193 |
| | | KPGATVKISCKVS | WIG | YMELSSLRSEDTA | WGQGTLVTV |
| | | GFNIK | | VYYCDR | SS |
| | LFR | SEQ ID NO: 194 | SEQ ID NO: 195 | SEQ ID NO: 212 | SEQ ID NO: |
| | | EIVLTQSPATLSLS | WYQQKPGQAPK | GIPARFSGSGSGT | 197 |
| | | PGERATLSC | LWIY | DFTLTISSLEPEDF | FGQGTKLEIK |
| | | | | AVYYC | |
| hu025.033 | HFR | SEQ ID NO: 207 | SEQ ID NO: 191 | SEQ ID NO: 210 | SEQ ID NO: |
| | | EVQLVQSGAEVK | WVQQAPGKGLE | RVTITADTSTDTA | 193 |
| | | KPGATVKISCKVS | WIG | YMELSSLRSEDTA | WGQGTLVTV |
| | | GFNIK | | VYYCDR | SS |
| | LFR | SEQ ID NO: 194 | SEQ ID NO: 195 | SEQ ID NO: 209 | SEQ ID NO: |
| | | EIVLTQSPATLSLS | WYQQKPGQAPK | GIPARFSGSGSGT | 197 |
| | | PGERATLSC | LWIY | DYTLTISSLEPEDF | FGQGTKLEIK |
| | | | | AVYYC | |
| hu025.059 | HFR | SEQ ID NO: 211 | SEQ ID NO: 191 | SEQ ID NO: 208 | SEQ ID NO: |
| | | EVQLVQSGAEVK | WVQQAPGKGLE | RVTITADTSTNTA | 193 |
| | | KPGATVKISCKAS | WIG | YMELSSLRSEDTA | WGQGTLVTV |
| | | GFNIK | | VYYCDR | SS |
| | LFR | SEQ ID NO: 194 | SEQ ID NO: 195 | SEQ ID NO: 209 | SEQ ID NO: |
| | | EIVLTQSPATLSLS | WYQQKPGQAPK | GIPARFSGSGSGT | 197 |
| | | PGERATLSC | LWIY | DYTLTISSLEPEDF | FGQGTKLEIK |
| | | | | AVYYC | |
| hu025.060 | HFR | SEQ ID NO: 211 | SEQ ID NO: 191 | SEQ ID NO: 208 | SEQ ID NO: |
| | | EVQLVQSGAEVK | WVQQAPGKGLE | RVTITADTSTNTA | 193 |
| | | KPGATVKISCKAS | WIG | YMELSSLRSEDTA | WGQGTLVTV |
| | | GFNIK | | VYYCDR | SS |
| | LFR | SEQ ID NO: 194 | SEQ ID NO: 195 | SEQ ID NO: 212 | SEQ ID |
| | | EIVLTQSPATLSLS | WYQQKPGQAPK | GIPARFSGSGSGT | NO:197 |
| | | PGERATLSC | LWIY | DFTLTISSLEPEDF | FGQGTKLEIK |
| | | | | AVYYC | |
| | HFR | SEQ ID NO: 190 | | SEQ ID NO: 192 | |
| | | EVQLVQSGAEVK | | RVTITADTSTX₂₁T | |
| | | KPGATVKISCKX₂₀ | | AYMELSSLRSEDT | |
| | | SGFNIK | | AVYYCDR | |
| | LFR | | | SEQ ID NO: 196 | |
| | | | | GIPARFSGSGSGT | |
| | | | | DX₂₂TLTISSLEPE | |
| | | | | DFAVYYC | |

wherein X₂₀ is A or V; X₂₁ is N or D; X₂₂ is Y or F.

In certain embodiments, the SIRP-alpha binding domain further comprises one or more of heavy chain HFR1, HFR2, HFR3 and HFR4, and/or one or more of light chain LFR1, LFR2, LFR3 and LFR4, wherein:
a) the HFR1 comprises EVQLVQSGAEVKKPGATVKISCKX₂₀SGFNIK (SEQ ID NO: 190) or a homologous sequence of at least 800% sequence identity thereof, and/or
b) the HFR2 comprises WVQQAPGKGLEWIG (SEQ ID NO: 191) or a homologous sequence of at least 800% sequence identity thereof, and/or
c) the HFR3 sequence comprises RVTITADTSTX₂₁TAYMELSSLRSEDTAVYYCDR (SEQ ID NO: 192) or a homologous sequence of at least 800% sequence identity thereof, and/or
d) the HFR4 comprises WGQGTLVTVSS (SEQ ID NO: 193) or a homologous sequence of at least 800% sequence identity thereof, and/or
e) the LFR1 comprises EIVLTQSPATLSLSPGERATLSC (SEQ ID NO: 194) or a homologous sequence of at least 80% sequence identity thereof, and/or
f) the LFR2 comprises WYQQKPGQAPKLWIY (SEQ ID NO: 195) or a homologous sequence of at least 80% sequence identity thereof, and/or
g) the LFR3 comprises GIPARFSGSGSGTDX₂₂TLTISSLEPEDFAVYYC (SEQ ID NO: 196) or a homologous sequence of at least 80% sequence identity thereof, and/or
h) the LFR4 comprises FGQGTKLEIK (SEQ ID NO: 197) or a homologous sequence of at least 80% sequence identity thereof, wherein X₂₀ is A or V; X₂₁ is N or D; X₂₂ is Y or F.

In certain embodiments, the SIRP-alpha binding domain further comprises one or more of heavy chain HFR1, HFR2, HFR3 and HFR4, and/or one or more of light chain LFR1, LFR2, LFR3 and LFR4, wherein:
i) the HFR1 comprises EVQLVQSGAEVKKPGATVKISCKVSGFNIK (SEQ ID NO: 207) or a homologous sequence of at least 80% sequence identity thereof, and/or
j) the HFR2 comprises WVQQAPGKGLEWIG (SEQ ID NO: 191) or a homologous sequence of at least 80% sequence identity thereof, and/or
k) the HFR3 sequence comprises RVTITADTSTNTAYMELSSLRSEDTAVYYCDR (SEQ ID NO: 208) or a homologous sequence of at least 80% sequence identity thereof, and/or
l) the HFR4 comprises WGQGTLVTVSS (SEQ ID NO: 193) or a homologous sequence of at least 80% sequence identity thereof, and/or
m) the LFR1 comprises EIVLTQSPATLSLSPGERATLSC (SEQ ID NO: 194) or a homologous sequence of at least 80% sequence identity thereof, and/or
n) the LFR2 comprises WYQQKPGQAPKLWIY (SEQ ID NO: 195) or a homologous sequence of at least 80% sequence identity thereof, and/or
o) the LFR3 comprises GIPARFSGSGSGTDYTLTISSLEPEDFAVYYC (SEQ ID NO: 209) or a homologous sequence of at least 80% sequence identity thereof, and/or
p) the LFR4 comprises FGQGTKLEIK (SEQ ID NO: 197) or a homologous sequence of at least 80% sequence identity thereof.

In certain embodiments, the SIRP-alpha binding domain further comprises one or more of heavy chain HFR1, HFR2, HFR3 and HFR4, and/or one or more of light chain LFR1, LFR2, LFR3 and LFR4, wherein:
q) the HFR1 comprises EVQLVQSGAEVKKPGATVKISCKVSGFNIK (SEQ ID NO: 207) or a homologous sequence of at least 80% sequence identity thereof, and/or
r) the HFR2 comprises WVQQAPGKGLEWIG (SEQ ID NO: 191) or a homologous sequence of at least 80% sequence identity thereof, and/or
s) the HFR3 sequence comprises RVTITADTSTNTAYMELSSLRSEDTAVYYCDR (SEQ ID NO: 208) or a homologous sequence of at least 80% sequence identity thereof, and/or
t) the HFR4 comprises WGQGTLVTVSS (SEQ ID NO: 193) or a homologous sequence of at least 80% sequence identity thereof, and/or
u) the LFR1 comprises EIVLTQSPATLSLSPGERATLSC (SEQ ID NO: 194) or a homologous sequence of at least 80% sequence identity thereof, and/or
v) the LFR2 comprises WYQQKPGQAPKLWIY (SEQ ID NO: 195) or a homologous sequence of at least 80% sequence identity thereof, and/or
w) the LFR3 comprises GIPARFSGSGSGTDFTLTISSLEPEDFAVYYC (SEQ ID NO: 212) or a homologous sequence of at least 80% sequence identity thereof, and/or
x) the LFR4 comprises FGQGTKLEIK (SEQ ID NO: 197) or a homologous sequence of at least 80% sequence identity thereof.

In certain embodiments, the SIRP-alpha binding domain further comprises one or more of heavy chain HFR1, HFR2, HFR3 and HFR4, and/or one or more of light chain LFR1, LFR2, LFR3 and LFR4, wherein:
y) the HFR1 comprises EVQLVQSGAEVKKPGATVKISCKVSGFNIK (SEQ ID NO: 207) or a homologous sequence of at least 80% sequence identity thereof, and/or
z) the HFR2 comprises WVQQAPGKGLEWIG (SEQ ID NO: 191) or a homologous sequence of at least 80% sequence identity thereof, and/or
aa) the HFR3 sequence comprises RVTITADTSTDTAYMELSSLRSEDTAVYYCDR (SEQ ID NO: 210) or a homologous sequence of at least 80% sequence identity thereof, and/or
bb) the HFR4 comprises WGQGTLVTVSS (SEQ ID NO: 193) or a homologous sequence of at least 80% sequence identity thereof, and/or
cc) the LFR1 comprises EIVLTQSPATLSLSPGERATLSC (SEQ ID NO: 194) or a homologous sequence of at least 80% sequence identity thereof, and/or
dd) the LFR2 comprises WYQQKPGQAPKLWIY (SEQ ID NO: 195) or a homologous sequence of at least 80% sequence identity thereof, and/or
ee) the LFR3 comprises GIPARFSGSGSGTDYTLTISSLEPEDFAVYYC (SEQ ID NO: 209) or a homologous sequence of at least 80% sequence identity thereof, and/or
ff) the LFR4 comprises FGQGTKLEIK (SEQ ID NO: 197) or a homologous sequence of at least 80% sequence identity thereof.

In certain embodiments, the SIRP-alpha binding domain further comprises one or more of heavy chain HFR1, HFR2, HFR3 and HFR4, and/or one or more of light chain LFR1, LFR2, LFR3 and LFR4, wherein:
gg) the HFR1 comprises EVQLVQSGAEVKKPGATVKISCKASGFNIK (SEQ ID NO: 211) or a homologous sequence of at least 80% sequence identity thereof, and/or
hh) the HFR2 comprises WVQQAPGKGLEWIG (SEQ ID NO: 191) or a homologous sequence of at least 80% sequence identity thereof, and/or
ii) the HFR3 sequence comprises RVTITADTSTNTAYMELSSLRSEDTAVYYCDR (SEQ ID NO: 208) or a homologous sequence of at least 80% sequence identity thereof, and/or
jj) the HFR4 comprises WGQGTLVTVSS (SEQ ID NO: 193) or a homologous sequence of at least 80% sequence identity thereof, and/or
kk) the LFR1 comprises EIVLTQSPATLSLSPGERATLSC (SEQ ID NO: 194) or a homologous sequence of at least 80% sequence identity thereof, and/or
ll) the LFR2 comprises WYQQKPGQAPKLWIY (SEQ ID NO: 195) or a homologous sequence of at least 80% sequence identity thereof, and/or
mm) the LFR3 comprises GIPARFSGSGSGTDYTLTISSLEPEDFAVYYC (SEQ ID NO: 209) or a homologous sequence of at least 80% sequence identity thereof, and/or
nn) the LFR4 comprises FGQGTKLEIK (SEQ ID NO: 197) or a homologous sequence of at least 80% sequence identity thereof.

In certain embodiments, the SIRP-alpha binding domain further comprises one or more of heavy chain HFR1, HFR2, HFR3 and HFR4, and/or one or more of light chain LFR1, LFR2, LFR3 and LFR4, wherein:
oo) the HFR1 comprises EVQLVQSGAEVKKPGATVKISCKASGFNIK (SEQ ID NO: 211) or a homologous sequence of at least 80% sequence identity thereof, and/or
pp) the HFR2 comprises WVQQAPGKGLEWIG (SEQ ID NO: 191) or a homologous sequence of at least 80% sequence identity thereof, and/or
qq) the HFR3 sequence comprises RVTITADTSTNTAYMELSSLRSEDTAVYYCDR (SEQ ID NO: 208) or a homologous sequence of at least 80% sequence identity thereof, and/or
rr) the HFR4 comprises WGQGTLVTVSS (SEQ ID NO: 193) or a homologous sequence of at least 80% sequence identity thereof, and/or
ss) the LFR1 comprises EIVLTQSPATLSLSPGERATLSC (SEQ ID NO: 194) or a homologous sequence of at least 80% sequence identity thereof, and/or
tt) the LFR2 comprises WYQQKPGQAPKLWIY (SEQ ID NO: 195) or a homologous sequence of at least 80% sequence identity thereof, and/or
uu) the LFR3 comprises GIPARFSGSGSGTDFTLTISSLEPEDFAVYYC (SEQ ID NO: 212) or a homologous sequence of at least 80% sequence identity thereof, and/or
vv) the LFR4 comprises FGQGTKLEIK (SEQ ID NO: 197) or a homologous sequence of at least 80% sequence identity thereof.

In certain embodiments, the SIRPα-binding domains provided herein comprise a heavy chain variable domain sequence selected from the group consisting of SEQ ID NOs: 1, 3, 5, 7, 9, 11, 13, 15, 17 and 159. In certain embodiments, SIRPα-binding domains provided herein comprise a light chain variable domain sequence selected from the group consisting of SEQ ID NOs: 2, 4, 6, 8, 10, 12, 16, 18 and 160.

In certain embodiments, the SIRP-alpha binding domain comprises
f) the heavy chain variable region comprises the sequence of SEQ ID NO: 1 and/or the light chain variable region comprises the sequence of SEQ ID NO: 2; or
g) the heavy chain variable region comprises the sequence of SEQ ID NO: 3 and/or the light chain variable region comprises the sequence of SEQ ID NO: 4; or
h) the heavy chain variable region comprises the sequence of SEQ ID NO: 5 and/or the light chain variable region comprises the sequence of SEQ ID NO: 6; or
i) the heavy chain variable region comprises the sequence of SEQ ID NO: 7 and/or the light chain variable region comprises the sequence of SEQ ID NO: 8; or
j) the heavy chain variable region comprises the sequence of SEQ ID NO: 9 and/or the light chain variable region comprises the sequence of SEQ ID NO: 10; or
k) the heavy chain variable region comprises the sequence of SEQ ID NO: 11 and/or the light chain variable region comprises the sequence of SEQ ID NO: 12; or
l) the heavy chain variable region comprises the sequence of SEQ ID NO: 13 and/or the light chain variable region comprises the sequence of SEQ ID NO: 14; or
m) the heavy chain variable region comprises the sequence of SEQ ID NO: 15 and/or the light chain variable region comprises the sequence of SEQ ID NO: 16; or
n) the heavy chain variable region comprises the sequence of SEQ ID NO: 17 and/or the light chain variable region comprises the sequence of SEQ ID NO: 18 and/or
o) the heavy chain variable region comprises the sequence of SEQ ID NO: 159 and/or the light chain variable region comprises the sequence of SEQ ID NO: 160.

In some embodiments, the SIRPα-binding domains provided herein comprise all or a portion of the heavy chain variable domain and/or all or a portion of the light chain variable domain. In one embodiment, the SIRPα-binding domains provided herein are a single domain antibody which consists of all or a portion of the heavy chain variable domain provided herein. More information of such a single domain antibody is available in the art (see, e.g., U.S. Pat. No. 6,248,516).

In certain embodiments, the SIRPα binding domain further comprises one or more amino acid residue substitutions or modifications yet retains specific binding to SIRPα. In certain embodiments, the at least one of the substitutions or modifications is in one or more of the CDR sequences, and/or in one or more of the non-CDR sequences of the heavy chain variable region or light chain variable region.

ii. Target Antigen Binding Domain

As used herein, the term “target antigen binding domain” refers to an antigen binding domain that targets a cell co-expressing the antigen and CD47. The target antigen binding domain of the multi-specific molecule provided herein can be a tumor antigen binding domain. In certain embodiments, the target antigen comprises a tumor surface antigen. As used herein, the term “tumor surface antigen” refers to an antigen predominantly presented by tumor cells to differentiate from non-malignant tissue, and is preferably located on the cell membrane of a tumor cell. The tumor surface antigens can be in various forms, for example, polypeptides (in particular glycosylated proteins), or glycosylation patterns of polypeptides, glycolipids (e.g., gangliosides, such as GM2), or even changes in the composition of lipids of the cell membrane which may be characteristic of cancer cells. The tumor surface antigens may be an antigen specifically expressed on a cancer cell that elicits an immune response; and/or binds to a T cell receptor (e.g., when presented by an MHC molecule) or to an antibody. In some embodiments, a tumor surface antigen elicits a humoral response (e.g., including production of antigen-specific antibodies). In some embodiments, a tumor surface antigen elicits a cellular response (e.g., involving T-cells whose receptors specifically interact with the tumor surface antigen). In some embodiments, a tumor surface antigen binds to an antibody and may or may not induce a particular physiological response in an organism.

In certain embodiments, the tumor surface antigen is, for example, PD-L1, claudin 18.2, BCMA, CD19, CD20, CD22, CD24, CD25, CD30, CD33, CD38, CD44, CD52, CD56, CD70, CD96, CD97, CD99, CD123, EGFR, HER2, HER3, CD 117, C-Met, EGFR, EGFRvIII, ERBB3, ERBB4, VEGFR1, VEGFR2, PTHR2, B7-H1(PD-L1), B7-H2, B7-H3, B7-H4, B7-H5, B7-H6, B7-H7, Trop-2, GPC-3, EPCAM, DLL-3, Nectin-4, Claudin6, Claudin18.2, Muc-1, PSMA, GD3, FAP, CEA, or EphA2.

### Claudin18.2-Binding Domain

In certain embodiments, the tumor surface antigen is claudin18.2. In certain embodiments, the claudin18.2-binding domain is capable of specifically binding to claudin18.2 (such as human claudin18.2).

In certain embodiments, the claudin18.2-binding domain comprises one or more (e.g. 1, 2, 3, 4, 5, or 6) CDR sequences of an anti-claudin18.2 antibody selected from the group consisting of hu26.H1L1, hu26.H1L2, hu26.H1L2 (S92A), hu26.H3L1, hu26.H3L2, hu28.H1L2, C10, C29 and C30.

“hu26.H1L1” as used herein refers to a humanized monoclonal antibody having a heavy chain variable region of SEQ ID NO: 65, and a light chain variable region of SEQ ID NO: 66.

“hu26.H1L2” as used herein refers to a humanized monoclonal antibody having a heavy chain variable region of SEQ ID NO: 65, and a light chain variable region of SEQ ID NO: 67.

“hu26.H1L2 (S92A)” as used herein refers to a humanized monoclonal antibody having a heavy chain variable region of SEQ ID NO: 65, and a light chain variable region of SEQ ID NO: 224.

“hu26.H3L1” as used herein refers to a humanized monoclonal antibody having a heavy chain variable region of SEQ ID NO: 68, and a light chain variable region of SEQ ID NO: 66.

“hu26.H3L2” as used herein refers to a humanized monoclonal antibody having a heavy chain variable region of SEQ ID NO: 68, and a light chain variable region of SEQ ID NO: 67.

“hu28.H1L2” as used herein refers to a humanized monoclonal antibody having a heavy chain variable region of SEQ ID NO: 69, and a light chain variable region of SEQ ID NO: 70.

“C10” as used herein refers to a humanized monoclonal antibody having a heavy chain variable region of SEQ ID NO: 71, and a light chain variable region of SEQ ID NO: 72.

“C29” as used herein refers to a humanized monoclonal antibody having a heavy chain variable region of SEQ ID NO: 73, and a light chain variable region of SEQ ID NO: 74.

“C30” as used herein refers to a humanized monoclonal antibody having a heavy chain variable region of SEQ ID NO: 75, and a light chain variable region of SEQ ID NO: 76.

Table 3 shows the CDR sequences of the anti-claudin18.2 antibody. The heavy chain and light chain variable region sequences are also provided below in Table 4 and Table 5.

TABLE 3
Amino acid sequences of the variable regions for anti-Claudin18.2
antibodies (CDRs are underlined).

| Antibody | VH | VL |
|---|---|---|
| hu26.HIL1 | hu26.VH_1, SEQ ID NO: 65: | hu26.VL_1, SEQ ID NO: 66: |
| | EVQLLESGGGLVQPGGSLRLS | DIQLTQSPSFLSASVGDRVTI |
| | CAASGFTLSSYALSWVRQAP | TCRASSSVNYIHWYQQKPG |
| | GKGLEWVSYISNLGGSTFYPD | KAPKLLIYATSNLASGVPSRF |
| | TVKGRFTISRDNSKNTLYLQM | SGSGSGTEFTLTISSLQPEDF |
| | NSLRAEDTAVYYCAKHLYNY | ATYYCQQWNSNPLTFGQGT |
| | DAFASWGQGTLVTVSS | KLEIK |
| hu26.H1L2 | hu26.VH_1, SEQ ID NO: 65: | hu26.VL_2, SEQ ID NO: 67: |
| | EVQLLESGGGLVQPGGSLRLS | DIQLTQSPSFLSASVGDRVTI |
| | CAASGFTLSSYALSWVRQAP | TCRASSSVNYIHWYQQKPG |
| | GKGLEWVSYISNLGGSTFYPD | KAPKALIYATSNLASGVPSR |
| | TVKGRFTISRDNSKNTLYLQM | FSGSGSGTEYTLTISSLQPED |
| | NSLRAEDTAVYYCAKHLYNY | FATYYCQQWNSNPLTFGQG |
| | DAFASWGQGTLVTVSS | TKLEIK |
| hu26.HIL2 | hu26.VH_1, SEQ ID NO: 65: | hu26.VL_2, SEQ ID NO: 224: |
| (S92A) | EVQLLESGGGLVQPGGSLRLS | DIQLTQSPSFLSASVGDRVTI |
| | CAASGFTLSSYALSWVRQAP | TCRASSSVNYIHWYQQKPG |
| | GKGLEWVSYISNLGGSTFYPD | KAPKALIYATSNLASGVPSR |
| | TVKGRFTISRDNSKNTLYLQM | FSGSGSGTEYTLTISSLQPED |
| | NSLRAEDTAVYYCAKHLYNY | FATYYCQQWNANPLTFGQG |
| | DAFASWGQGTLVTVSS | TKLEIK |
| hu26.H3L1 | hu26.VH_3, SEQ ID NO: 68: | hu26.VL_1, SEQ ID NO: 66: |
| | EVQLLESGGGLVQPGGSLRLS | DIQLTQSPSFLSASVGDRVTI |
| | CAASGFTLSSYALSWVRQAP | TCRASSSVNYIHWYQQKPG |
| | GKGLEWVAYISNLGGSTFYP | KAPKLLIYATSNLASGVPSRF |
| | DTVKGRFTISRDNSKNTLYLQ | SGSGSGTEFTLTISSLQPEDF |
| | MNSLRAEDTAVYYCATHLYN | ATYYCQQWNSNPLTFGQGT |
| | YDAFASWGQGTLVTVSS | KLEIK |
| hu26.H3L2 | hu26.VH_3, SEQ ID NO: 68: | hu26.VL_2, SEQ ID NO: 67: |
| | EVQLLESGGGLVQPGGSLRLS | DIQLTQSPSFLSASVGDRVTI |
| | CAASGFTLSSYALSWVRQAP | TCRASSSVNYIHWYQQKPG |
| | GKGLEWVAYISNLGGSTFYP | KAPKALIYATSNLASGVPSR |
| | DTVKGRFTISRDNSKNTLYLQ | FSGSGSGTEYTLTISSLQPED |
| | MNSLRAEDTAVYYCATHLYN | FATYYCQQWNSNPLTFGQG |
| | YDAFASWGQGTLVTVSS | TKLEIK |
| hu28.HIL2 | hu28.VH_1, SEQ ID NO: 69: | hu28.VL_1, SEQ ID NO: 70: |
| | QVQLVQSGAEVKKPGASVKV | DIVMTQSPDSLAVSLGERATI |
| | SCKASGYTFTNWVHWVRQA | NCKSSQSLLNAGNQKNYLT |
| | PGQGLEWMGEINPTNARSNY | WYQQKPGQPPKLLIYWSSTR |
| | NEKFKKRVTMTRDTSTSTVY | ESGVPDRFSGSGSGTDFTLTI |
| | MELSSLRSEDTAVYYCARIYY | SSLQAEDVAVYHCQNNYYY |
| | GNSFAHWGQGTLVTVSS | PLTFGGGTKLEIK |
| C10 | C10.VH, SEQ ID NO: 71: | C10.VL, SEQ ID NO: 72: |
| | EVQLQQSGPELVKPGSSVKM | DIVMTQSPSSLTVTAGEKVT |
| | SCKASGYTFTDYNMHWLKQS | MSCKSSQSLLNSGNQKNYLT |
| | HGKSLEWIGYINPKNGGTRY | WYQQKPGQPPKLLIYWAST |
| | NQKFKGKATLTVNKSSSTAY | RESGVPDRFTGSGSGTDFTL |
| | MELRSLTSEDSAVYYCARLY | TISSVQAEDLAVYFCQNDYS |
| | YGNSFDYWGQGTTLTVSS | FPFTFGSGTKLEIK |
| C29 | C29.VH, SEQ ID NO: 73: | C29.VL, SEQ ID NO: 74: |
| | DVQLVESGGGLVQPGGSRKL | DIVMTQSPSSLTVTAGEKVT |
| | SCAASGFTFSSFGMHWVRQA | MNCKSSQSLLNSGNQKNYL |
| | PEKGLEWVAYISSGSSSIYYV | TWYQQKPGQPPKLLIYWAS |
| | DTVKGRFTISRDNPKNTLFLQ | TRTSGVPDRFTGSGSGTDFT |
| | MTSLRSEDTAMYYCARNAYY | LTISSVQAEDLAVYCCQNGY |
| | GNAFDYWGQGTTLTVSS | TYPLTFGAGTKLELK |
| C30 | C30.VH, SEQ ID NO: 75: | C30.VL, SEQ ID NO: 76: |
| | QVQLQQSGPDLVKPGASVKL | DIVMTQSPSSLTVTAGEKVT |
| | SCKASGYTFTNYDINWVKQR | MTCKSGQSLLNSGNQRNYL |
| | PGQGLEWIGGIHPRDGNTKY | TWYQQKPGQSPKLLIYWAS |
| | NEKFKDKATLTIDTSANTAY | TRESGVPDRFTGSGSGADFT |
| | MEFHSLTSEDSAVYFCARGY | LTISSVQAEDLALYYCQNAY |
| | YGNSFAYWGQGTLVTVSA | FYPYTFGGGTKLEIK |

| | |
|---|---|
| hu28.HIL2. | SEQ ID NO: 227 |
| scFv | DIVMTQSPDSLAVSLGERATINCKSSQSLLNAGNQKNYLTWY |
| | QQKPGQPPKLLIYWSSTRESGVPDRFSGSGSGTDFTLTISSLQA |
| | EDVAVYHCQNNYYYPLTFGGGTKLEIKGGGGSGGGGSGGGG |
| | SGGGGSQVQLVQSGAEVKKPGASVKVSCKASGYTFTNWVH |
| | WVRQAPGQGLEWMGEINPTNARSNYNEKFKKRVTMTRDTST |
| | STVYMELSSLRSEDTAVYYCARIYYGNSFAHWGQGTLVTVSS |

TABLE 4
CDRs of the anti-Claudin18.2 antibodies provided herein

| Antibody | VH | VL |
|---|---|---|
| hu26.H1L1 | CDR1 (SEQ ID NO: 77) | CDR1 (SEQ ID NO: 80) |
| hu26.H1L2 | SYALS | RASSSVNYIH |
| hu26.H3L1 | CDR2 (SEQ ID NO: 78) | CDR2 (SEQ ID NO: 81) |
| hu26.H3L2 | YISNLGGSTFYPDTVKG | ATSNLAS |
| | CDR3 (SEQ ID NO: 79) | CDR3 (SEQ ID NO: 82) |
| | HLYNYDAFAS | QQWNSNPLT |
| hu26.H1L2 | CDR1 (SEQ ID NO: 77) | CDR1 (SEQ ID NO: 80) |
| (S92A) | SYALS | RASSSVNYIH |
| | CDR2 (SEQ ID NO: 78) | CDR2 (SEQ ID NO: 81) |
| | YISNLGGSTFYPDTVKG | ATSNLAS |
| | CDR3 (SEQ ID NO: 79) | CDR3 (SEQ ID NO: 225) |
| | HLYNYDAFAS | QQWNANPLT |
| hu28.H1L2 | CDR1 (SEQ ID NO: 83) | CDR1 (SEQ ID NO: 86) |
| | NWVH | KSSQSLLNAGNQKNYLT |
| | CDR2 (SEQ ID NO: 84) | CDR2 (SEQ ID NO: 87) |
| | EINPTNARSNYNEKFKK | WSSTRES |
| | CDR3 (SEQ ID NO: 85) | CDR3 (SEQ ID NO: 88) |
| | IYYGNSFAH | QNNYYYPLT |
| C10 | CDR1 (SEQ ID NO: 89) | CDR1 (SEQ ID NO: 92) |
| | DYNMH | KSSQSLLNSGNQKNYLT |
| | CDR2 (SEQ ID NO: 90) | CDR2 (SEQ ID NO: 93) |
| | YINPKNGGTRYNQKFKG | WASTRES |
| | CDR3 (SEQ ID NO: 91) | CDR3 (SEQ ID NO: 94) |
| | LYYGNSFDY | QNDYSFPFT |
| C29 | CDR1 (SEQ ID NO: 95) | CDR1 (SEQ ID NO: 98) |
| | SFGMH | KSSQSLLNSGNQKNYLT |
| | CDR2 (SEQ ID NO: 96) | CDR2 (SEQ ID NO: 99) |
| | YISSGSSSIYYVDTVKG | WASTRTS |
| | CDR3 (SEQ ID NO: 97) | CDR3 (SEQ ID NO: 100) |
| | NAYYGNAFDY | QNGYTYPLT |
| C30 | CDR1 (SEQ ID NO: 101) | CDR1 (SEQ ID NO: 104) |
| | NYDIN | KSGQSLLNSGNQRNYLT |
| | CDR2 (SEQ ID NO: 102) | CDR2 (SEQ ID NO: 105) |
| | GIHPRDGNTKYNEKFKD | WASTRES |
| | CDR3 (SEQ ID NO: 103) | CDR3 (SEQ ID NO: 106) |
| | GYYGNSFAY | QNAYFYPYT |

CDRs are known to be responsible for antigen binding, however, it has been found that not all of the 6 CDRs are indispensable or unchangeable. In other words, it is possible to replace or change or modify one or more CDRs provided herein for Claudin18.2-binding domain, yet substantially retain the specific binding affinity to PD-1 (e.g. human Claudin18.2).

In certain embodiments, the claudin 18.2 binding domain comprises:
a) a HCDR1 comprising the sequence of SEQ ID NO: 77, a HCDR2 comprising the sequence of SEQ ID NO: 78, and a HCDR3 comprising the sequence of SEQ ID NO: 79; and/or a LCDR1 comprising the sequence of SEQ ID NO: 80, a LCDR2 comprising the sequence of SEQ ID NO: 81, and a LCDR3 comprising the sequence of SEQ ID NO: 82 or SEQ ID NO: 225; or
b) a HCDR1 comprising the sequence of SEQ ID NO: 83, a HCDR2 comprising the sequence of SEQ ID NO: 84, and a HCDR3 comprising the sequence of SEQ ID NO: 85; and/or a LCDR1 comprising the sequence of SEQ ID NO: 86, a LCDR2 comprising the sequence of SEQ ID NO: 87, and a LCDR3 comprising the sequence of SEQ ID NO: 88; or
c) a HCDR1 comprising the sequence of SEQ ID NO: 89, a HCDR2 comprising the sequence of SEQ ID NO: 90, and a HCDR3 comprising the sequence of SEQ ID NO: 91; and/or a LCDR1 comprising the sequence of SEQ ID NO: 92, a LCDR2 comprising the sequence of SEQ ID NO: 93, and a LCDR3 comprising the sequence of SEQ ID NO: 94; or
d) a HCDR1 comprising the sequence of SEQ ID NO: 95, a HCDR2 comprising the sequence of SEQ ID NO: 96, and a HCDR3 comprising the sequence of SEQ ID NO: 97; and/or a LCDR1 comprising the sequence of SEQ ID NO: 98, a LCDR2 comprising the sequence of SEQ ID NO: 99, and a LCDR3 comprising the sequence of SEQ ID NO: 100; or
e) a HCDR1 comprising the sequence of SEQ ID NO: 101, a HCDR2 comprising the sequence of SEQ ID NO: 102, and a HCDR3 comprising the sequence of SEQ ID NO: 103; and/or a LCDR1 comprising the sequence of SEQ ID NO: 104, a LCDR2 comprising the sequence of SEQ ID NO: 105, and a LCDR3 comprising the sequence of SEQ ID NO: 106.

In certain embodiments, the claudin 18.2 binding domain comprises the same HCDRs and LCDRs as anti-claudin 18.2 antibody selected from the group consisting of hu26.H1L1, hu26.H1L2 (S92A), hu28.H1L2, C10, C29 and C30,
a) wherein the hu26.H1L1 comprises a heavy chain variable region comprising the sequence of SEQ ID NO: 65, and/or a light chain variable region comprising the sequence of SEQ ID NO: 66,
b) wherein the hu26.H1L2 (S92A) comprises a heavy chain variable region comprising the sequence of SEQ ID NO: 65, and/or a light chain variable region comprising the sequence of SEQ ID NO: 224,
c) the hu28.H1L2 comprises a heavy chain variable region comprising the sequence of SEQ ID NO: 69, and/or a light chain variable region comprising the sequence of SEQ ID NO: 70,
d) the C10 comprises a heavy chain variable region comprising the sequence of SEQ ID NO: 71, and/or a light chain variable region comprising the sequence of SEQ ID NO: 72,
e) the C29 comprises a heavy chain variable region comprising the sequence of SEQ ID NO: 73, and/or a light chain variable region comprising the sequence of SEQ ID NO: 74, and
f) the C30 comprises a heavy chain variable region comprising the sequence of SEQ ID NO: 75, and/or a light chain variable region comprising the sequence of SEQ ID NO: 76.

In certain embodiments, the Claudin18.2-binding domain of the multi-specific molecule provided herein comprises any suitable framework region (FR) sequences, as long as the antigen-binding domains can specifically bind to Claudin18.2.

In certain embodiments, the Claudin18.2-binding domains of the multi-specific molecule provided herein are humanized. A humanized antigen-binding domain is desirable in its reduced immunogenicity in human. A humanized antigen-binding domain is chimeric in its variable regions, as non-human CDR sequences are grafted to human or substantially human FR sequences. Humanization of an antigen-binding domain can be essentially performed by substituting the non-human (such as murine) CDR genes for the corresponding human CDR genes in a human immunoglobulin gene (see, for example, Jones et al. (1986) Nature 321:522-525; Riechmann et al. (1988) Nature 332:323-327; Verhoeyen et al. (1988) Science 239:1534-1536). As described above, suitable human heavy chain and light chain variable domains can be selected to achieve this purpose using methods known in the art.

In certain embodiments, the humanized antigen-binding domains provided herein are composed of substantially all human sequences except for the CDR sequences which are non-human. In some embodiments, the variable region FRs, and constant regions if present, are entirely or substantially from human immunoglobulin sequences. The human FR sequences and human constant region sequences may be derived from different human immunoglobulin genes, for example, FR sequences derived from one human antibody and constant region from another human antibody. In some embodiments, the humanized antigen-binding domain comprise human FR1-4.

In some embodiments, the FR regions derived from human may comprise the same amino acid sequence as the human immunoglobulin from which it is derived. In some embodiments, one or more amino acid residues of the human FR are substituted with the corresponding residues from the parent non-human antibody. This may be desirable in certain embodiments to make the humanized antibody or its fragment closely approximate the non-human parent antibody structure. In certain embodiments, the humanized Claudin18.2-binding domain provided herein comprises no more than 10, 9, 8, 7, 6, 5, 4, 3, 2, or 1 amino acid residue substitutions in each of the human FR sequences, or no more than 10, 9, 8, 7, 6, 5, 4, 3, 2, or 1 amino acid residue substitutions in all the FRs of a heavy or a light chain variable domain. In some embodiments, such change in amino acid residue could be present in heavy chain FR regions only, in light chain FR regions only, or in both chains. Table 4.1 shows the FR sequences of the humanized antibodies hu26.H1L1, hu26.H1L2, hu26.H3L1, hu26.H3L2, hu28.H1L2.

TABLE 4.1
FR amino acid sequences of 5 humanized antibodies

| Antibody | | FR1 | FR2 | FR3 | FR4 |
|---|---|---|---|---|---|
| hu26.H1L1 | HFR | SEQ ID NO: 167 | SEQ ID NO: 199 | SEQ ID NO: 200 | SEQ ID NO: |
| | | EVQLLESGGG | WVRQAPGKG | RFTISRDNSK | 173 |
| | | LVQPGGSLRL | LEWVS | NTLYLQMNSL | WGQGTLV |
| | | SCAASGFTLS | | RAEDTAVYY | TVSS |
| | | | | CAK | |
| | LFR | SEQ ID NO: 174 | SEQ ID NO:201 | SEQ ID NO: 202 | SEQ ID NO: |
| | | DIQLTQSPSFL | WYQQKPGKA | GVPSRFSGSG | 181 |
| | | SASVGDRVTI | PKLLIY | SGTEFTLTISS | FGQGTKLE |
| | | TC | | LQPEDFATYY | IK |
| | | | | C | |
| hu26.H1L2/ | HFR | SEQ ID NO: 167 | SEQ ID NO: 199 | SEQ ID NO: 200 | SEQ ID NO: |
| hu26.H1L2 | | EVQLLESGGG | WVRQAPGKG | RFTISRDNSK | 173 |
| (S92A) | | LVQPGGSLRL | LEWVS | NTLYLQMNSL | WGQGTLV |
| | | SCAASGFTLS | | RAEDTAVYY | TVSS |
| | | | | CAK | |
| | LFR | SEQ ID NO: 174 | SEQ ID NO: 203 | SEQ ID NO: 204 | SEQ ID NO: |
| | | DIQLTQSPSFL | WYQQKPGKA | GVPSRFSGSG | 181 |
| | | SASVGDRVTI | PKALIY | SGTEYTLTISS | FGQGTKLE |
| | | TC | | LQPEDFATYY | IK |
| | | | | C | |
| hu26.H3L1 | HFR | SEQ ID NO: 167 | SEQ ID NO: 213 | SEQ ID NO: 205 | SEQ ID NO: |
| | | EVQLLESGGG | WVRQAPGKG | RFTISRDNSK | 173 |
| | | LVQPGGSLRL | LEWVA | NTLYLQMNSL | WGQGTLV |
| | | SCAASGFTLS | | RAEDTAVYY | TVSS |
| | | | | CAT | |
| | LFR | SEQ ID NO: 174 | SEQ ID NO: 201 | SEQ ID NO: 202 | SEQ ID NO: |
| | | DIQLTQSPSFL | WYQQKPGKA | GVPSRFSGSG | 181 |
| | | SASVGDRVTI | PKLLIY | SGTEFTLTISS | FGQGTKLE |
| | | TC | | LQPEDFATYY | IK |
| | | | | C | |
| hu26.H3L2 | HFR | SEQ ID NO: 167 | SEQ ID NO:213 | SEQ ID NO: 205 | SEQ ID NO: |
| | | EVQLLESGGG | WVRQAPGKG | RFTISRDNSK | 173 |
| | | LVQPGGSLRL | LEWVA | NTLYLQMNSL | WGQGTLV |
| | | SCAASGFTLS | | RAEDTAVYY | TVSS |
| | | | | CAT | |
| | LFR | SEQ ID NO: 174 | SEQ ID NO: 203 | SEQ ID NO: 204 | SEQ ID NO: |
| | | DIQLTQSPSFL | WYQQKPGKA | GVPSRFSGSG | 181 |
| | | SASVGDRVTI | PKALIY | SGTEYTLTISS | FGQGTKLE |
| | | TC | | LQPEDFATYY | IK |
| | | | | C | |
| | LFR | | SEQ ID NO: | | |
| | | | 206 | | |
| | | | WYQQKPGK | | |
| | | | APKX₁₉LIY | | |
| hu28.H1L2 | HFR | SEQ ID NO: 168 | SEQ ID NO: 170 | SEQ ID NO: 172 | SEQ ID NO: |
| | | QVQLVQSGA | WVRQAPGQG | RVTMTRDTST | 173 |
| | | EVKKPGASVK | LEWMG | STVYMELSSL | WGQGTLV |
| | | VSCKASGYTF | | RSEDTAVYYC | TVSS |
| | | T | | AR | |
| | LFR | SEQ ID NO: 175 | SEQ ID NO: 177 | SEQ ID NO: 179 | SEQ ID NO: |
| | | DIVMTQSPDS | WYQQKPGQP | GVPDRFSGSG | 182 |
| | | LAVSLGERAT | PKLLIY | SGTDFTLTISS | FGGGTKLE |
| | | INC | | LQAEDVAVY | IK |
| | | | | HC | |
| | HFR | | SEQ ID NO: | SEQ ID NO: | |
| | | | 169 | 171 | |
| | | | WVRQAPGK | RFTISRDNSK | |
| | | | GLEWVX₁₈ | NTLYLQMNS | |
| | | | | LRAEDTAVY | |
| | | | | YCAX₂₃ | |
| | LFR | | SEQ ID NO: | SEQ ID NO: | SEQ ID NO: |
| | | | 176 | 178 | 180 |
| | | | WYQQKPGX₂₆ | GVPSRFSGSG | FGX₂₅GTK |
| | | | X₂₇PKX₁₉LIY | SGTEX₂₄TLTI | LEIK |
| | | | | SSLQPEDFAT | |
| | | | | YYC | |

wherein X₁₈ is S or A, X₁₉ is L or A, X₂₃ is T or K, X₂₄ is Y or F, X₂₅ is Q or G, X₂₆ is Q or K, X₂₇ is P or A.

In some embodiments, the claudin 18.2 binding domain further comprises one or more of heavy chain HFR1, HFR2, HFR3 and HFR4, and/or one or more of light chain LFR1, LFR2, LFR3 and LFR4, wherein:
a) the HFR1 comprises an amino acid sequence selected from the group consisting of EVQLLESGGGLVQPGGSLRLSCAASGFTLS (SEQ ID NO: 167) and QVQLVQSGAEVKKPGASVKVSCKASGYTFT (SEQ ID NO: 168) or a homologous sequence of at least 80% sequence identity thereof,
b) the HFR2 comprises an amino acid sequence selected from the group consisting of WVRQAPGKGLEWVX₁₈ (SEQ ID NO: 169) and WVRQAPGQGLEWMG (SEQ ID NO: 170) or a homologous sequence of at least 80% sequence identity thereof,
c) the HFR3 sequence comprises an amino acid sequence selected from the group consisting of RFTISRDNSKNTLYLQMNSLRAEDTAVYYCAX₂₃ (SEQ ID NO: 171) and RVTMTRDTSTSTVYMELSSLRSEDTAVYYCAR (SEQ ID NO: 172) or a homologous sequence of at least 80% sequence identity thereof,
d) the HFR4 comprises WGQGTLVTVSS (SEQ ID NO: 173) or a homologous sequence of at least 80% sequence identity thereof,
e) the LFR1 comprises an amino acid sequence selected from the group consisting of DIQLTQSPSFLSASVGDRVTITC (SEQ ID NO: 174) and DIVMTQSPDSLAVSLGERATINC (SEQ ID NO: 175) or a homologous sequence of at least 80% sequence identity thereof,
f) the LFR2 comprises an amino acid sequence selected from the group consisting of WYQQKPGX₂₆X₂₇PKX₁₉LIY (SEQ ID NO: 176) or a homologous sequence of at least 80% sequence identity thereof,
g) the LFR3 comprises an amino acid sequence selected from the group consisting of GVPSRFSGSGSGTEX₂₄TLTISSLQPEDFATYYC (SEQ ID NO: 178) and GVPDRFSGSGSGTDFTLTISSLQAEDVAVYHC (SEQ ID NO: 179) or a homologous sequence of at least 80% sequence identity thereof, and
h) the LFR4 comprises FGX₂₅GTKLEIK (SEQ ID NO: 180) or a homologous sequence of at least 80% sequence identity thereof,
   wherein X₁₈ is S or A, X₁₉ is L or A, X₂₃ is T or K, X₂₄ is Y or F, X₂₅ is Q or G, X₂₆ is Q or K, X₂₇ is P or A.

In some embodiments, the claudin 18.2 binding domain further comprises one or more of heavy chain HFR1, HFR2, HFR3 and HFR4, and/or one or more of light chain LFR1, LFR2, LFR3 and LFR4, wherein:
a) the HFR1 comprises EVQLLESGGGLVQPGGSLRLSCAASGFTLS (SEQ ID NO: 167) or a homologous sequence of at least 80% sequence identity thereof,
b) the HFR2 comprises WVRQAPGKGLEWVX₁₈ (SEQ ID NO: 169) or a homologous sequence of at least 80% sequence identity thereof,
c) the HFR3 sequence comprises RFTISRDNSKNTLYLQMNSLRAEDTAVYYCAX₂₃ (SEQ ID NO: 171) or a homologous sequence of at least 80% sequence identity thereof,
d) the HFR4 comprises WGQGTLVTVSS (SEQ ID NO: 173) or a homologous sequence of at least 80% sequence identity thereof,
e) the LFR1 comprises DIQLTQSPSFLSASVGDRVTITC (SEQ ID NO: 174) or a homologous sequence of at least 80% sequence identity thereof,
f) the LFR2 comprises WYQQKPGKAPKX₁₉LIY (SEQ ID NO: 206) or a homologous sequence of at least 80% sequence identity thereof,
g) the LFR3 comprises GVPSRFSGSGSGTEX₂₄TLTISSLQPEDFATYYC (SEQ ID NO: 178) or a homologous sequence of at least 80% sequence identity thereof, and h) the LFR4 comprises FGQGTKLEIK (SEQ ID NO: 181) or a homologous sequence of at least 80% sequence identity thereof,
wherein X₁₈ is S or A, X₁₉ is L or A, X₂₃ is T or K, X₂₄ is Y or F.

In some embodiments, the claudin 18.2 binding domain further comprises one or more of heavy chain HFR1, HFR2, HFR3 and HFR4, and/or one or more of light chain LFR1, LFR2, LFR3 and LFR4, wherein:
a) the HFR1 comprises EVQLLESGGGLVQPGGSLRLSCAASGFTLS (SEQ ID NO: 167) or a homologous sequence of at least 80% sequence identity thereof,
b) the HFR2 comprises WVRQAPGKGLEWVS (SEQ ID NO: 199) or a homologous sequence of at least 80% sequence identity thereof,
c) the HFR3 sequence comprises RFTISRDNSKNTLYLQMNSLRAEDTAVYYCAK (SEQ ID NO: 200) or a homologous sequence of at least 80% sequence identity thereof,
d) the HFR4 comprises WGQGTLVTVSS (SEQ ID NO: 173) or a homologous sequence of at least 80% sequence identity thereof,
e) the LFR1 comprises DIQLTQSPSFLSASVGDRVTITC (SEQ ID NO: 174) or a homologous sequence of at least 80% sequence identity thereof,
f) the LFR2 comprises WYQQKPGKAPKLLIY (SEQ ID NO: 201) or a homologous sequence of at least 80% sequence identity thereof,
g) the LFR3 comprises GVPSRFSGSGSGTEFTLTISSLQPEDFATYYC (SEQ ID NO: 202) or a homologous sequence of at least 80% sequence identity thereof, and
h) the LFR4 comprises FGQGTKLEIK (SEQ ID NO: 181) or a homologous sequence of at least 80% sequence identity thereof.

In some embodiments, the claudin 18.2 binding domain further comprises one or more of heavy chain HFR1, HFR2, HFR3 and HFR4, and/or one or more of light chain LFR1, LFR2, LFR3 and LFR4, wherein:
a) the HFR1 comprises EVQLLESGGGLVQPGGSLRLSCAASGFTLS (SEQ ID NO: 167) or a homologous sequence of at least 80% sequence identity thereof,
b) the HFR2 comprises WVRQAPGKGLEWVS (SEQ ID NO: 199) or a homologous sequence of at least 80% sequence identity thereof,
c) the HFR3 sequence comprises RFTISRDNSKNTLYLQMNSLRAEDTAVYYCAK (SEQ ID NO: 200) or a homologous sequence of at least 80% sequence identity thereof,
d) the HFR4 comprises WGQGTLVTVSS (SEQ ID NO: 173) or a homologous sequence of at least 80% sequence identity thereof,
e) the LFR1 comprises DIQLTQSPSFLSASVGDRVTITC (SEQ ID NO: 174) or a homologous sequence of at least 80% sequence identity thereof,
f) the LFR2 comprises WYQQKPGKAPKALIY (SEQ ID NO: 203) or a homologous sequence of at least 80% sequence identity thereof,
g) the LFR3 comprises GVPSRFSGSGSGTEYTLTISSLQPEDFATYYC (SEQ ID NO: 204) or a homologous sequence of at least 80% sequence identity thereof, and
h) the LFR4 comprises FGQGTKLEIK (SEQ ID NO: 181) or a homologous sequence of at least 80% sequence identity thereof.

In some embodiments, the claudin 18.2 binding domain further comprises one or more of heavy chain HFR1, HFR2, HFR3 and HFR4, and/or one or more of light chain LFR1, LFR2, LFR3 and LFR4, wherein:
a) the HFR1 comprises EVQLLESGGGLVQPGGSLRLSCAASGFTLS (SEQ ID NO: 167) or a homologous sequence of at least 80% sequence identity thereof,
b) the HFR2 comprises WVRQAPGKGLEWVA (SEQ ID NO: 213) or a homologous sequence of at least 80% sequence identity thereof,
c) the HFR3 sequence comprises RFTISRDNSKNTLYLQMNSLRAEDTAVYYCAT (SEQ ID NO: 205) or a homologous sequence of at least 80% sequence identity thereof,
d) the HFR4 comprises WGQGTLVTVSS (SEQ ID NO: 173) or a homologous sequence of at least 80% sequence identity thereof,
e) the LFR1 comprises DIQLTQSPSFLSASVGDRVTITC (SEQ ID NO: 174) or a homologous sequence of at least 80% sequence identity thereof,
f) the LFR2 comprises WYQQKPGKAPKLLIY (SEQ ID NO: 201) or a homologous sequence of at least 80% sequence identity thereof,
g) the LFR3 comprises GVPSRFSGSGSGTEFTLTISSLQPEDFATYYC (SEQ ID NO: 202) or a homologous sequence of at least 80% sequence identity thereof, and
h) the LFR4 comprises FGQGTKLEIK (SEQ ID NO: 181) or a homologous sequence of at least 80% sequence identity thereof.

In some embodiments, the claudin 18.2 binding domain further comprises one or more of heavy chain HFR1, HFR2, HFR3 and HFR4, and/or one or more of light chain LFR1, LFR2, LFR3 and LFR4, wherein:
a) the HFR1 comprises EVQLLESGGGLVQPGGSLRLSCAASGFTLS (SEQ ID NO: 167) or a homologous sequence of at least 80% sequence identity thereof,
b) the HFR2 comprises WVRQAPGKGLEWVA (SEQ ID NO: 213) or a homologous sequence of at least 80% sequence identity thereof,
c) the HFR3 sequence comprises RFTISRDNSKNTLYLQMNSLRAEDTAVYYCAT (SEQ ID NO: 205) or a homologous sequence of at least 80% sequence identity thereof,
d) the HFR4 comprises WGQGTLVTVSS (SEQ ID NO: 173) or a homologous sequence of at least 80% sequence identity thereof,
e) the LFR1 comprises DIQLTQSPSFLSASVGDRVTITC (SEQ ID NO: 174) or a homologous sequence of at least 80% sequence identity thereof,
f) the LFR2 comprises WYQQKPGKAPKALIY (SEQ ID NO: 203) or a homologous sequence of at least 80% sequence identity thereof,
g) the LFR3 comprises GVPSRFSGSGSGTEYTLTISSLQPEDFATYYC (SEQ ID NO: 204) or a homologous sequence of at least 80% sequence identity thereof, and
h) the LFR4 comprises FGQGTKLEIK (SEQ ID NO: 181) or a homologous sequence of at least 80% sequence identity thereof.

In some embodiments, the claudin 18.2 binding domain further comprises one or more of heavy chain HFR1, HFR2, HFR3 and HFR4, and/or one or more of light chain LFR1, LFR2, LFR3 and LFR4, wherein:
a) the HFR1 comprises QVQLVQSGAEVKKPGASVKVSCKASGYTFT (SEQ ID NO: 168) or a homologous sequence of at least 80% sequence identity thereof,
b) the HFR2 comprises WVRQAPGQGLEWMG (SEQ ID NO: 170) or a homologous sequence of at least 80% sequence identity thereof,
c) the HFR3 sequence comprises RVTMTRDTSTSTVYMELSSLRSEDTAVYYCAR (SEQ ID NO: 172) or a homologous sequence of at least 80% sequence identity thereof,
d) the HFR4 comprises WGQGTLVTVSS (SEQ ID NO: 173) or a homologous sequence of at least 80% sequence identity thereof,
e) the LFR1 comprises DIVMTQSPDSLAVSLGERATINC (SEQ ID NO: 175) or a homologous sequence of at least 80% sequence identity thereof,
f) the LFR2 comprises WYQQKPGQPPKLLIY (SEQ ID NO: 177) or a homologous sequence of at least 80% sequence identity thereof,
g) the LFR3 comprises GVPDRFSGSGSGTDFTLTISSLQAEDVAVYHC (SEQ ID NO: 179) or a homologous sequence of at least 80% sequence identity thereof, and
h) the LFR4 comprises FGGGTKLEIK (SEQ ID NO: 182) or a homologous sequence of at least 80% sequence identity thereof.

In some embodiments, the claudin 18.2 binding domain comprises:
a) the heavy chain variable region comprises the sequence of SEQ ID NO: 65 or 68, and/or the light chain variable region comprises the sequence of SEQ ID NO: 66 or 67 or 224; or
b) the heavy chain variable region comprises the sequence of SEQ ID NO: 69 and/or the light chain variable region comprises the sequence of SEQ ID NO: 70; or
c) the heavy chain variable region comprises the sequence of SEQ ID NO: 71 and/or the light chain variable region comprises the sequence of SEQ ID NO: 72; or
d) the heavy chain variable region comprises the sequence of SEQ ID NO: 73 and/or the light chain variable region comprises the sequence of SEQ ID NO: 74; or
e) the heavy chain variable region comprises the sequence of SEQ ID NO: 75 and/or the light chain variable region comprises the sequence of SEQ ID NO: 76.

In some embodiments, the Claudin18.2-binding domains provided herein comprise all or a portion of the heavy chain variable domain and/or all or a portion of the light chain variable domain. In one embodiment, the Claudin18.2-binding domains provided herein are a single domain antibody which consists of all or a portion of the heavy chain variable domain provided herein. More information of such a single domain antibody is available in the art (see, e.g., U.S. Pat. No. 6,248,516).

In certain embodiments, the Claudin18.2 binding domain further comprises one or more amino acid residue substitutions or modifications yet retains specific binding to Claudin18.2. In certain embodiments, the at least one of the substitutions or modifications is in one or more of the CDR sequences, and/or in one or more of the non-CDR sequences of the heavy chain variable region or light chain variable region. In certain embodiments, the at least one of the substitutions or modifications is in one or more of the CDR sequences of, for example, hu26.H1L2 (S92A)

### PD-L1-Binding Domain

In certain embodiments, the tumor surface antigen is PD-L1. In certain embodiments, the PD-L1-binding domain is capable of specifically binding to PD-L1 (such as human PD-L1).

In certain embodiments, the PD-L1-binding domain comprises one or more (e.g. 1, 2, or 3) CDR sequences of an anti-PD-L1 antibody selected from the group consisting of C71, C71v38, C239, C492, C570, C446, C2811, C1778, C1793, C2855, C2713 and C2719.

“C71” as used herein refers to a humanized heavy chain antibody having a heavy chain variable region of SEQ ID NO: 107.

“C71v38” as used herein refers to a humanized heavy chain antibody having a heavy chain variable region of SEQ ID NO: 108.

“C239” as used herein refers to a humanized heavy chain antibody having a heavy chain variable region of SEQ ID NO: 109.

“C492” as used herein refers to a humanized heavy chain antibody having a heavy chain variable region of SEQ ID NO: 110.

“C570” as used herein refers to a humanized heavy chain antibody having a heavy chain variable region of SEQ ID NO: 111.

“570h3” as used herein refers to a humanized heavy chain antibody having a heavy chain variable region of SEQ ID NO: 223.

“C446” as used herein refers to a humanized heavy chain antibody having a heavy chain variable region of SEQ ID NO: 112.

“C2811” as used herein refers to a humanized heavy chain antibody having a heavy chain variable region of SEQ ID NO: 113.

“C1778” as used herein refers to a humanized heavy chain antibody having a heavy chain variable region of SEQ ID NO: 114.

“C1793” as used herein refers to a humanized heavy chain antibody having a heavy chain variable region of SEQ ID NO: 115.

“C2855” as used herein refers to a humanized heavy chain antibody having a heavy chain variable region of SEQ ID NO: 116.

“C2713” as used herein refers to a humanized heavy chain antibody having a heavy chain variable region of SEQ ID NO: 117.

“C2719” as used herein refers to a humanized heavy chain antibody having a heavy chain variable region of SEQ ID NO: 118.

In certain embodiments, the PD-L1-binding domain comprises heavy chain CDR1 comprising the sequence of SEQ ID NO: 119, heavy chain CDR2 comprising the sequence of SEQ ID NO: 120, and heavy chain CDR3 comprising the sequence of SEQ ID NO: 121.

In certain embodiments, the PD-L1-binding domain comprises heavy chain CDR1 comprising the sequence of SEQ ID NO: 122, heavy chain CDR2 comprising the sequence of SEQ ID NO: 123, and heavy chain CDR3 comprising the sequence of SEQ ID NO: 124.

In certain embodiments, the PD-L1-binding domain comprises heavy chain CDR1 comprising the sequence of SEQ ID NO: 125, heavy chain CDR2 comprising the sequence of SEQ ID NO: 126, and heavy chain CDR3 comprising the sequence of SEQ ID NO: 127.

In certain embodiments, the PD-L1-binding domain comprises heavy chain CDR1 comprising the sequence of SEQ ID NO: 128, heavy chain CDR2 comprising the sequence of SEQ ID NO: 129, and heavy chain CDR3 comprising the sequence of SEQ ID NO: 130.

In certain embodiments, the PD-L1-binding domain comprises heavy chain CDR1 comprising the sequence of SEQ ID NO: 131, heavy chain CDR2 comprising the sequence of SEQ ID NO: 132, and heavy chain CDR3 comprising the sequence of SEQ ID NO: 133.

In certain embodiments, the PD-L1-binding domain comprises heavy chain CDR1 comprising the sequence of SEQ ID NO: 134, heavy chain CDR2 comprising the sequence of SEQ ID NO: 135, and heavy chain CDR3 comprising the sequence of SEQ ID NO: 136.

In certain embodiments, the PD-L1-binding domain comprises heavy chain CDR1 comprising the sequence of SEQ ID NO: 137, heavy chain CDR2 comprising the sequence of SEQ ID NO: 138, and heavy chain CDR3 comprising the sequence of SEQ ID NO: 139.

In certain embodiments, the PD-L1-binding domain comprises heavy chain CDR1 comprising the sequence of SEQ ID NO: 140, heavy chain CDR2 comprising the sequence of SEQ ID NO: 141, and heavy chain CDR3 comprising the sequence of SEQ ID NO: 142.

In certain embodiments, the PD-L1-binding domain comprises heavy chain CDR1 comprising the sequence of SEQ ID NO: 143, heavy chain CDR2 comprising the sequence of SEQ ID NO: 144, and heavy chain CDR3 comprising the sequence of SEQ ID NO: 145.

In certain embodiments, the PD-L1-binding domain comprises heavy chain CDR1 comprising the sequence of SEQ ID NO: 146, heavy chain CDR2 comprising the sequence of SEQ ID NO: 147, and heavy chain CDR3 comprising the sequence of SEQ ID NO: 148.

In certain embodiments, the PD-L1-binding domain comprises heavy chain CDR1 comprising the sequence of SEQ ID NO: 149, heavy chain CDR2 comprising the sequence of SEQ ID NO: 150, and heavy chain CDR3 comprising the sequence of SEQ ID NO: 151.

In certain embodiments, the PD-L1-binding domain comprises heavy chain CDR1 comprising the sequence of SEQ ID NO: 152, heavy chain CDR2 comprising the sequence of SEQ ID NO: 153, and heavy chain CDR3 comprising the sequence of SEQ ID NO: 154.

In certain embodiments, the PD-L1 binding domain comprises the same HCDRs as anti-PD-L1 antibody selected from the group consisting of C71, C71v38, C239, C492, C570, 570h3, C446, C2811, C1778, C1793, C2855, C2713 and C2719.

In certain embodiments, the PD-L1binding domain comprises: the heavy chain variable region comprises the sequence selected from the group consisting of SEQ ID NOs: 107-118 and 223.

Table 5 shows the heavy chain variable region sequences of the anti-PD-L1 antibodies. The CDR sequences are also provided below in Table 6.

TABLE 5
Amino acid sequences of the variable regions for anti-PD-L1 antibodies
(CDRs are underlined).

| Antibody | VH |
|---|---|
| C71 | C71.VH, SEQ ID NO: 107: |
| | EVQVVESGGGLVQSGGSLKLSCAGSGFTESAGFMVWHRQVPGKE |
| | RELVALIATPSGSTNYADSVKGRFTISRDNGKNTVYLQMNSLKPE |
| | DTAVYYCNIRGYWGQGTLVTVSS |
| C71v38 | C71v38.VH, SEQ ID NO: 108: |
| | EVQVVESGGGLVQSGGSLRLSCAGSGFTESAGFMVWHRQVPGKE |
| | RELVALIATPSGSTQYADSVKGRFTISRDNGKNTVYLQMNSLKPE |
| | DTAVYYCNIRGYWGQGTLVTVSS |
| C239 | C239.VH, SEQ ID NO: 109: |
| | EVQVVESGGGLVQPGGSLRLSCAASGFTFNYFAMSWVRQAPGKG |
| | LEWVADIDTGGTDTDYADSVKGRFSISRDNAKNTLYLQMNSLKP |
| | EDTAVYYCAKGPREATDIRSWGQGTQVTVSS |
| C492 | C492.VH, SEQ ID NO: 110: |
| | EVOLVESGGDLVQTGGSLRLSCSASGFTFNYFAMSWVRQAPGKG |
| | LEWVADIDTGGTDTDYADSVKGRFSISRDNAKNTLYLQMNSLKP |
| | EDTAVYYCAKGPREATDIRSWGQGTQVTVSS |
| C570 | C570.VH, SEQ ID NO: 111: |
| | EVOLVESGGGLVQAGGSLRLSCAASGFTFSSHAMSWVRQAPGKG |
| | LEWVSDIDTSGTTTDYADSVKGRFTISRDNAKNTLYLQMNSLKPE |
| | DTAVYYCAKGPREATDIRSWGQGTQVTVSS |
| 570h3 | 570h3.VH, SEQ ID NO 223: |
| | EVOLVESGGGLVQPGGSLRLSCAASGFTFSSHAMSWVRQAPGKG |
| | LEWVSDIDTSGTTTDYADSVKGRFTISRDNSKNTLYLQMNSLRAE |
| | DTAVYYCAKGPREATDIRSWGQGTTVTVSS |
| C446 | C446.VH, SEQ ID NO: 112: |
| | QVQLVESGGDLVQPGGSLRLSCKASGIIFSSYLLFWYRQPPGKERE |
| | LVATISSAGSSIDYADSVKGRFTISRDNAKNTVYLQMNSLKPEDTA |
| | VYYCRTWPYNYWGQGTLVTVSS |
| C2811 | C2811.VH, SEQ ID NO: 113: |
| | QVQLVESGGDLVQPGGSLRLSCAASGRTFADAAWGWFRQVPGR |
| | DREWVASISRSGDTYYEDSVKGRFTISRDNAKNTVCLQMNSLRAE |
| | DTAVYHCAARNSGAPSSIVIAYEYWGQGTQVTVSS |
| C1778 | C1778.VH, SEQ ID NO: 114: |
| | QVQLVESGGGLVQAGGSLRLTCAASGRTFADAAWGWFRQVPGR |
| | DREWVASISRSGDTYYEDSVKGRFTISRDNAKNTVYLQMNSLRAE |
| | DTAVYHCAARNSGAPSSIVIAYEYWGQGTQVTVSS |
| C1793 | C1793.VH, SEQ ID NO: 115: |
| | QVQLVESGGGLVQAGGSLRLTCAASGRAFADAAWGWFRQVPGR |
| | DREWVASISRSGDTYYEDSVKGRFTISRDNAKNTVYLQMNSLRAE |
| | DTAVYHCAARNSGAPSSIVIAYEYWGQGTQVTVSS |
| C2855 | C2855.VH, SEQ ID NO: 116: |
| | QVQLEESGGGLAQAGDSLRLTCAASGRTFADAAWGWFRQVPGR |
| | NREWVASISRSGDTYYEDSVKGRFTISRDNAKNTVYLQMNSLRAE |
| | DTAVYHCAARNSGAPSSIVIAYEYWGQGTQVTVSS |
| C2713 | C2713.VH, SEQ ID NO: 117: |
| | AVQLMESGGGLVQAGGSLRLTCAASGRTFADAAWGWFRQVPGR |
| | DREWVASISRSGDTYYEDSVKGRFTISRDYAKNTVYLQMNSLRAE |
| | DTAVYHCAARNSGAPSSIVIAYEYWGHGTQVIVSS |
| C2719 | C2719.VH, SEQ ID NO: 118: |
| | QVQLVESGGGLVQAGGSLRLTCAASGRTFADAAWGWFRQVPGR |
| | DREWVASISRSGDTYYEDSVKGRFTISRDNAKNTVYLQMNSLKPE |
| | DTAVYHCAARNSGAPSSIVIAYEYWGQGTQVTVSS |

TABLE 6
CDRs of the anti-PDL1 single domain antibodies
provided herein

| Antibody | VH |
|---|---|
| C71 | CDR1 (SEQ ID NO: 119) |
| | GFTESAGFMV |
| | CDR2 (SEQ ID NO: 120) |
| | LVALIATPSGSTN |
| | CDR3 (SEQ ID NO: 121) |
| | NIRGYWG |
| C71v38 | CDR1 (SEQ ID NO: 122) |
| | GFTESAGFMV |
| | CDR2 (SEQ ID NO: 123) |
| | VALIATPSGSTQ |
| | CDR3 (SEQ ID NO: 124) |
| | NIRGYWG |
| C239 | CDR1 (SEQ ID NO: 125) |
| | GFTFNYFAMS |
| | CDR2 (SEQ ID NO: 126) |
| | IDTGGTDT |
| | CDR3 (SEQ ID NO: 127) |
| | AKGPREATDIRS |
| C492 | CDR1 (SEQ ID NO: 128) |
| | GFTFNYFAMS |
| | CDR2 (SEQ ID NO: 129) |
| | IDTGGTDT |
| | CDR3 (SEQ ID NO: 130) |
| | AKGPREATDIRS |
| C570/570h3 | CDR1 (SEQ ID NO: 131) |
| | GFTFSSHAMS |
| | CDR2 (SEQ ID NO: 132) |
| | IDTSGTTT |
| | CDR3 (SEQ ID NO: 133) |
| | AKGPREATDIRS |
| C446 | CDR1 (SEQ ID NO: 134) |
| | GIIFSSYLLF |
| | CDR2 (SEQ ID NO: 135) |
| | ISSAGSSI |
| | CDR3 (SEQ ID NO: 136) |
| | RTWPYNY |
| C2811 | CDR1 (SEQ ID NO: 137) |
| | GRTFADAAWG |
| | CDR2 (SEQ ID NO: 138) |
| | ISRSGDT |
| | CDR3 (SEQ ID NO: 139) |
| | AARNSGAPSSIVIAYEY |
| C1778 | CDR1 (SEQ ID NO: 140) |
| | GRTFADAAWG |
| | CDR2 (SEQ ID NO: 141) |
| | ISRSGDT |
| | CDR3 (SEQ ID NO: 142) |
| | AARNSGAPSSIVIAYEY |
| C1793 | CDR1 (SEQ ID NO: 143) |
| | GRAFADAAWG |
| | CDR2 (SEQ ID NO: 144) |
| | ISRSGDT |
| | CDR3 (SEQ ID NO: 145) |
| | AARNSGAPSSIVIAYEY |
| C2855 | CDR1 (SEQ ID NO: 146) |
| | GRTFADAAWG |
| | CDR2 (SEQ ID NO: 147) |
| | ISRSGDT |
| | CDR3 (SEQ ID NO: 148) |
| | AARNSGAPSSIVIAYEY |
| C2713 | CDR1 (SEQ ID NO: 149) |
| | GRTFADAAWG |
| | CDR2 (SEQ ID NO: 150) |
| | ISRSGDT |
| | CDR3 (SEQ ID NO: 151) |
| | AARNSGAPSSIVIAYEY |
| C2719 | CDR1 (SEQ ID NO: 152) |
| | GRTFADAAWG |
| | CDR2 (SEQ ID NO: 153) |
| | ISRSGDT |
| | CDR3 (SEQ ID NO: 154) |
| | AARNSGAPSSIVIAYEY |

In certain embodiments, the PD-L1 binding domain comprises: the heavy chain variable region comprises the sequence selected from the group consisting of SEQ ID NOs: 107-118 and 223.

In certain embodiments, the PD-L1 binding domain further comprises one or more amino acid residue substitutions or modifications yet retains specific binding to PD-L1. In certain embodiments, the at least one of the substitutions or modifications is in one or more of the CDR sequences, and/or in one or more of the non-CDR sequences of the heavy chain variable region or light chain variable region.

iv. Activating Receptor-Binding Domain

The multi-specific molecule further comprises an activating receptor-binding domain. As used herein, the term “activating receptor” refers to receptors (e.g., FcγR) expressed on an immune effector cell (e.g., a phagocytic cell, such as a macrophage), which upon activation by binding to, for example, Fc domain, mediates at least one effector functions of the immune effector cell (e.g., a phagocytic cell, such as a macrophage) or pro-inflammatory response. In certain embodiments, the immune effector cell provided herein co-expresses SIRP-alpha and the activating receptor.

In certain embodiments, the activating receptor is an FcγR, and the activating receptor-binding domain is an Fc domain. Fc domain can activate Fc receptors (FcRs) on macrophages to drive a phosphorylation cascade propagated by the receptors' immunoreceptor tyrosine-based activation motifs (ITAMs). ITAMs are conserved sequences present in the cytoplasmic tails of several activating receptors on immune effector cells, such as FcRs, T cell receptors and immunoglobulins (Ig). ITAMs can be featured with a conserved amino acid sequence motif, which consists of paired YXXL/I motifs (wherein Y, L and I refer to Tyrosine, Lysine and Isoleucine respectively) and separated by a defined interval of six to eight amino acids. Phosphorylation of residues within the ITAM recruits several signaling molecules for phagocytosis activation. Accordingly, the activating receptor can be any receptor expressed on an immune effector cell that can be bound and activated to induce phagocytosis via an ITAM-comprising intracellular phagocytosis signaling domain.

In other embodiments, the activating receptor is a receptor involved in a different phagocytic signaling or mechanism, such as, Akt mediated signaling cascade (via CD19, CD28, CSFR or PDGFR receptor), clustering of a group of receptors on an immune effector cell (e.g., macrophage) that potentiates phagocytosis (via, for example, integrins or selectins), or antigen mediated cytotoxicity (via FcDR1 (CD89) receptor or CD206).

For example, activating receptors that are relevant to effector functions such as phagocytosis can be fragment crystallizable γ receptors (FcγRs), TREM2, lectin, scavenger receptor A1 (SRA1), MARCO, CD36, CD163, CD68, CD205, CD206, FcDR1, CD207, CD209, RAGE, CD14, CD64, F4/80, CD64, CD32a, CD16a, CD89, CD19, CD28, CSFR, PDGFR, MSR1, SCARA3, COLEC12, SCARA5, SCARB1, SCARB2, dectin 1, RAGE (SR-E1), LRP1, LRP2, ASGP, SR-PSOX, CXCL16, OLR1, SCARF1, SCARF2, CXCL16, STAB1, STAB2, SRCRB4D, SSC5D, CCR2, CX3CR1, CSF1R, Tie2, HuCRIg(L), and CD169 receptor or complement receptors, such as CR1 and CR3. In certain embodiments, the activating receptor is FcγR.

In certain embodiments, the activating receptor that can generate pro-inflammatory signals upon activation, include without limitation, PI3K, FcγR1, FcγR2A, FcγR2B2, FcγR2C, FcγR3A, BAH. Tyro3, Axl, Traf6, Syk, MyD88, Zap70, FcεR1, Fe a RI, BAFF-R, DAP 12, NFAM1, MRC1, ItgB5, MERTK, ELMO, and CD79b.

The term “activating receptor-binding domain”, as used herein, refers to domain (e.g., a portion of an antibody) that is capable of specifically binding to an activating receptor on an immune effector cell and such binding leads to activation of the receptor as well as downstream signaling thereof (e.g, effector function or pro-inflammatory response of the immune cell). For example, the activating receptor-binding domain comprises an Fc domain of an antibody or a variant thereof. In certain embodiments, the Fc domain can be derived from IgG1 or IgG4.

In certain embodiments, the activating receptor-binding domain of the multi-specific molecules provided herein binds and activates activating receptor selected from the group consisting of fragment crystallizable γ receptors (FcγRs), TREM2, lectin, scavenger receptor A1 (SRA1), MARCO, CD36, CD163, CD68, CD205, CD206, FcDR1, CD207, CD209, RAGE, CD14, CD64, F4/80, CD64, CD32a, CD16a, CD89, CD19, CD28, CSFR, PDGFR, MSR1, SCARA3, COLEC12, SCARA5, SCARB1, SCARB2, dectin 1, RAGE (SR-E1), LRP1, LRP2, ASGP, SR-PSOX, CXCL16, OLR1, SCARF1, SCARF2, CXCL16, STAB1, STAB2, SRCRB4D, SSC5D, CCR2, CX3CR1, CSF1R, Tie2, HuCRIg(L), and CD169 receptor or complement receptors (such as CR1 and CR3), PI3K, FcγR1, FcγR2A, FcγR2B2, FcγR2C, FcγR3A, BAH. Tyro3, Axl, Traf6, Syk, MyD88, Zap70, FcεR1, FcαR1, BAFF-R, DAP 12, NFAM1, MRC1, ItgB5, MERTK, ELMO, and CD79b.

In certain embodiments, the activating receptor-binding domain of the multi-specific molecule provided herein comprises an Fc domain or a variant thereof, which activates Fc receptors (FcRs) on macrophages, such as FcγRII.

In certain embodiments, the activating receptor-binding domain comprises the Fc domain of human IgG1 (hIgG1) or human IgG4 (hIgG4), heavy chain constant regions of which are shown below respectively:
a) Heavy chain constant region (hIgG4.S228P), SEQ ID NO: 155 (the sequence of CH1 region is in bold and wavy underlined (SEQ ID NO: 228), the sequence of hinge region is in italic (SEQ ID NO: 229), and the sequence of Fc region is underlined (SEQ ID NO: 230)):

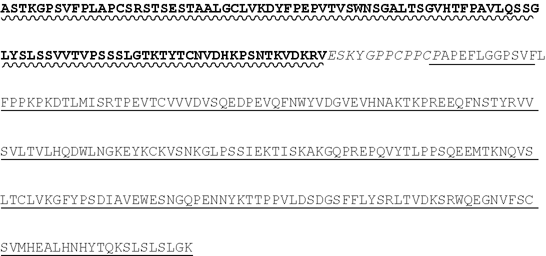

b) Heavy chain constant region (hIgG1), SEQ ID NO: 156 (the sequence of CH1 region is in bold and wavy underlined (SEQ ID NO: 231), the sequence of hinge region is in italic (SEQ ID NO: 232), and the sequence of Fc region is underlined (SEQ ID NO: 233)):

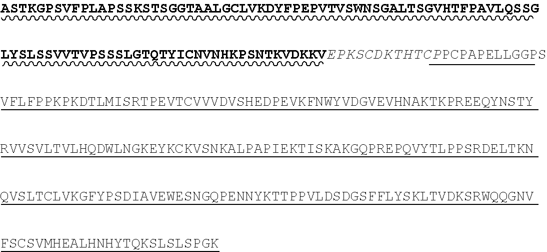

The multi-specific molecules provided herein can also include a human light chain constant region (CL), such as kappa chain and lamda chain, amino acid sequences of which are provided below:

Light chain constant region (kappa), SEQ ID NO: 157:

RTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPV
TKSENRGEC

Light chain constant region (Lambda), SEQ ID NO: 158:

GQPKAAPSVTLFPPSSEELQANKATLVCLISDFYPGAVTVAWKADSSPV
KAGVETTTPSKQSNNKYAASSYLSLTPEQWKSHRSYSCQVTHEGSTVEK
TVAPTECS

v. Configurations of the Multi-Specific Molecules

In certain embodiments, the engager provided herein is a recombinant protein comprising multiple binding domains described throughout the specification, wherein each of the multiple binding domains has specific binding affinity to SIRPalpha, activating receptor, or target antigen, and is connected with each other by one or more linkers. The one or more linkers may have cognate peptides that exhibit complementary binding with each other. For example, the SIRP-alpha binding domain of the engager provided herein is fused with the first of a pair of cognate peptides, the target antigen binding domain of the engager provided herein is fused with the second of the pairs of cognate peptides; as such, the SIRP-alpha binding domain and the target antigen binding domain can be connected by the pair of cognate peptides through the complementary binding between each of the pair of cognate peptides.

In certain embodiments, the pair of cognate peptides comprises two heavy chains of an antibody or any complementary portion thereof, a pair of light chain and heavy chain complementary to each other of an antibody or any complementary portion thereof, leucine zipper domains that exhibit complementary binding with each other (e.g., the zipper sequences within the binding regions of c-Fos and c-June proteins), or synthetic peptides designed to specifically bind to each other via synthetic connectors.

The SIRP-alpha binding domain, the activating receptor binding domain and the target antigen binding domain of the engager provided herein can also be connected via chemical binding, such as crosslinking (e.g., BS2G crosslinker (Bis[Sulfosuccmimidyl]glutarate), BS3 crosslinker (Bis[sulfosuccinimidyl] suberate), Sulfo-DSS, DST crosslinker (Disuccinimidyl tartrate), BMPS (N-(B-Maleimidopropyloxy) succinimide ester; MBS crosslinker (mMaleimidobenzoyl-N-hydroxysuccinimide ester); or PDPH crosslinker (3-[2-Pyridyldithio]propionylhydrazide)).

In certain embodiments, the multi-specific molecule provided herein is configured such that the SIRP-alpha binding domain and the activating receptor-binding domain are in close proximity to permit binding of the multi-specific molecule provided herein to both SIRP-alpha and the activating receptor co-expressed on the same immune effector cell. As used herein, the term “close proximity” between the SIRP-alpha binding domain and the activating receptor-binding domain refers to the relative position between the two domains from a three-dimensional perspective, which allows binding of the two domains on the same immune effector cell to SIRP-alpha and activating receptors respectively co-expressed on the immune effector cell. Specific binding of the SIRP-alpha binding domain and the activating receptor-binding domain to SIRP-alpha and the activating receptor respectively, which are co-expressed on the same immune effector cell can be detected by, for example, confocal microscopy, fluorescence resonance energy transfer (FRET), or single-molecule superresolution microscopy.

The target antigen binding domain of the multi-specific molecules provided herein may comprise an antibody domain or an antibody mimetic domain that binds to the target antigen. The term “antibody domain” and the term “antibody mimetic domain” are defined above.

In certain embodiments, the target antigen binding antibody domain (e.g., the tumor antigen binding antibody domain) is linked to N-terminus of the activating receptor-binding domain (e.g., Fc domain). In certain embodiments, the target antigen binding antibody domain (e.g., the tumor antigen binding antibody domain) comprises a Fab domain, optionally, the Fab domain comprises a heavy chain linked to one of the N-termini of the activating receptor-binding domain (e.g., Fc domain). In certain embodiments, the multi-specific molecule provided herein comprises two target antigen binding antibody domains, each of which comprises an Fab domain, optionally, each of the Fab domains comprises a heavy chain linked to each N-terminus of the activating receptor-binding domain (e.g., Fc domain), respectively.

In certain embodiments, the SIRP-alpha binding domain is linked to the activating receptor-binding domain (e.g., Fc domain) or to the target antigen binding antibody domain (e.g., the tumor antigen binding antibody domain).

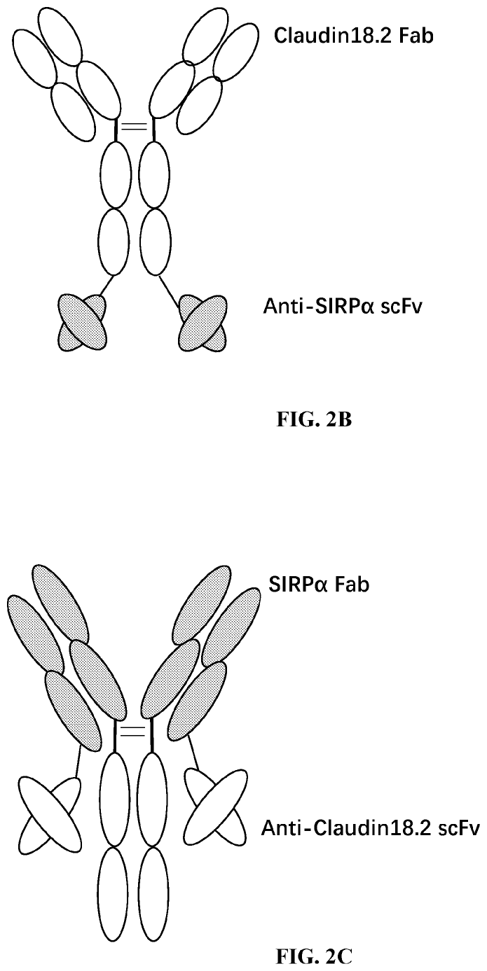

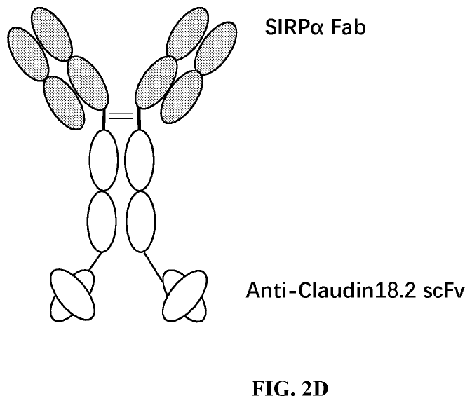

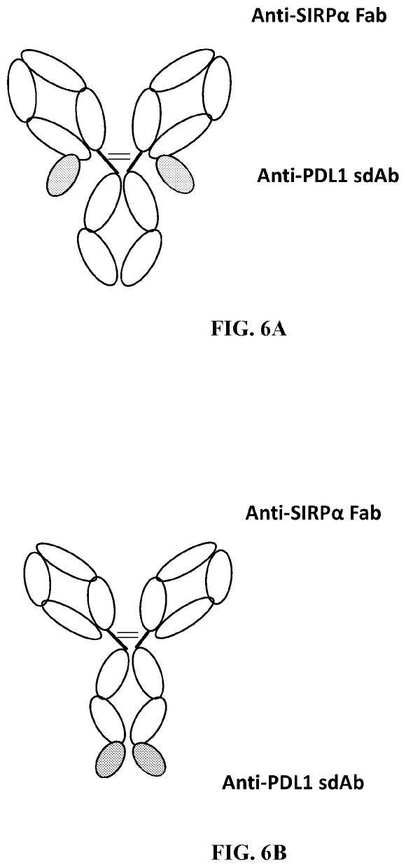

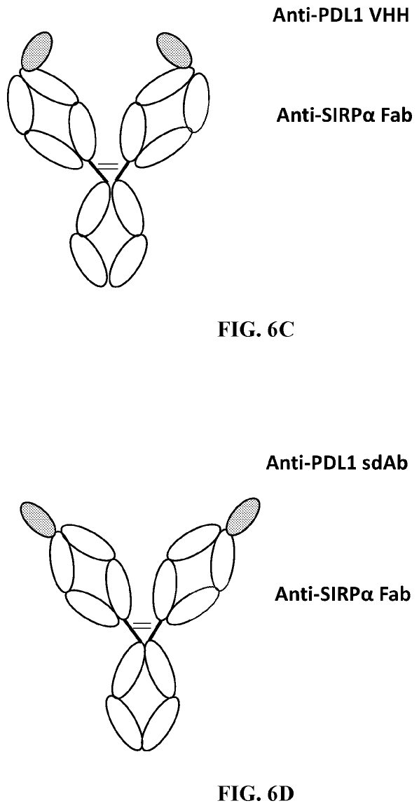

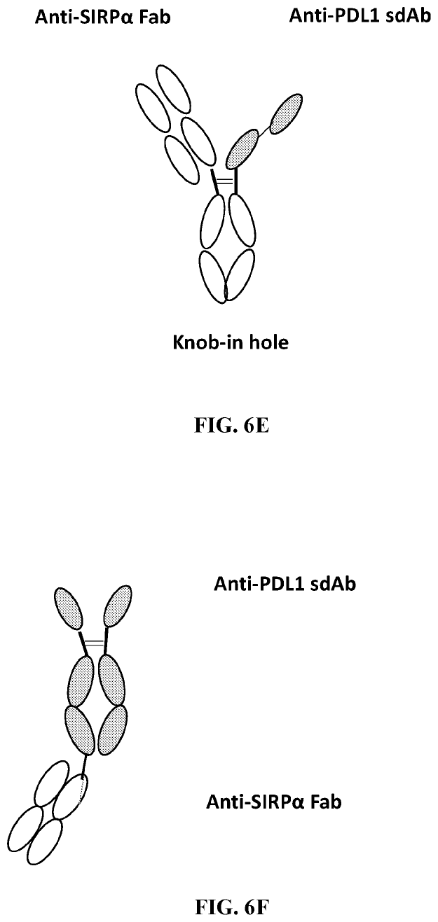

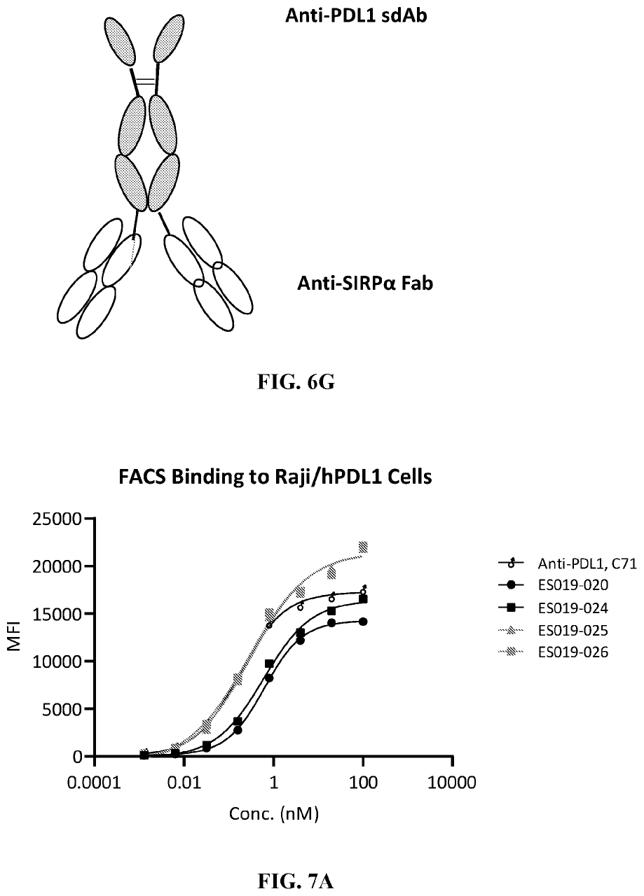

In certain embodiments, the SIRP-alpha binding domain is linked to C-terminus of the activating receptor-binding domain (e.g., Fc domain). In such embodiments, the multi-specific molecules provided herein comprises a target antigen binding antibody, wherein the SIRP-alpha binding domain is linked to C-terminus of the Fc region of the target antigen binding antibody. Illustrative embodiments are found, for example, at FIGS. 2B, 6F, and 6G.

In certain embodiments, both the target antigen binding antibody domain (e.g., the tumor antigen binding antibody domain) and the SIRP-alpha binding domain are linked to N-terminus of the activating receptor-binding domain (e.g., Fc domain), with the proviso that the SIRP-alpha binding domain and the target antigen binding antibody domain (e.g., the tumor antigen binding antibody domain) are not linked to the same N-terminus of the activating receptor-binding domain (e.g., Fc domain). Illustrative embodiments are found, for example, at FIG. 6E.

In certain embodiments, the SIRP-alpha binding domain is linked to the C-terminus of light chain of the target antigen binding domain (e.g., tumor antigen binding Fab domain). Illustrative embodiments are found, for example, at FIG. 2A.

Where the multi-specific molecule provided herein comprises two target antigen binding antibody domains, the SIRP-alpha binding domain can be linked to the C-terminus of light chain of the one of the two target antigen binding domains (e.g., tumor antigen binding Fab domains); the SIRP-alpha binding domains can also be linked to the C-terminus of light chain of both the two target antigen binding domains (e.g., tumor antigen binding Fab domains).

In certain other embodiments, the SIRP-alpha binding antibody domain is linked to N-terminus of the activating receptor-binding domain (e.g., Fc domain). In certain embodiments, the SIRP-alpha binding antibody domain comprises a Fab domain, optionally, the Fab domain comprises a heavy chain linked to one of the N-termini of the activating receptor-binding domain (e.g., Fc domain). In certain embodiments, the multi-specific molecules provided herein comprise two SIRP-alpha binding antibody domains, each of which comprises an Fab domain, optionally, each of the Fab domains comprises a heavy chain linked to each N-terminus of the activating receptor-binding domain (e.g., Fc domain), respectively. In such embodiments, the multi-specific molecules provided herein comprises a SIRP-alpha binding antibody. In certain of these embodiments, the target antigen binding domain (e.g., the tumor antigen binding domain) is linked to the activating receptor-binding domain (e.g., Fc domain) or to the SIRP-alpha binding antibody domain.

In certain embodiments, the multi-specific molecules provided herein comprises a SIRP-alpha binding antibody, wherein the target antigen binding domain is linked to C-terminus of the Fc region of the SIRP-alpha binding antibody (see, e.g. FIG. 2D, 6B), or linked to C-terminus of the light chain of the SIRP-alpha binding antibody (see, e.g. FIGS. 2C and 6A).

In certain embodiments, the target antigen binding domain (e.g., the tumor antigen binding domain) is linked to the C-terminus of the light chain of the SIRP-alpha binding Fab domain. Illustrative embodiments are found, for example, at FIG. 6A. In certain embodiments, the target antigen binding domain (e.g., the tumor antigen binding domain) is linked to the N-terminus of the heavy chain or the light chain of the SIRP-alpha binding Fab domain. Illustrative embodiments are found, for example, at FIGS. 6C and 6D.

As used herein, the term “linked to” refers to covalent or non-covalent interactions (e.g., hydrogen bonds, ionic bonds, van der Waals interactions, and hydrophobic bonds) between two components.

Configurations of the multi-specific molecules provided herein are summarized in Table 7 below.

TABLE 7
configurations of the multi-specific molecules provided herein

| | | | Antibody | | |
|---|---|---|---|---|---|
| | | | from which | | |
| | | | the binding | | |
| | Binding | | domain is | | Polypeptide chains |
| BiME | domain | Form | derived | Configuration | constituting the BiME |
| ES028- | Claudin18.2- | antibody | Derived from | The C terminus | 1^{st} Chain: |
| 001 (FIG. | binding domain | | hu28.H1L2 | of each of the | VL (hu28.H1L2)-CL- |
| 2A) | SIPRalpha- | scFv | Derived | light chain of | linker-scFv (C25); |
| | binding domain | | from C25 | anti- | 2^{nd} Chain: |
| | | | | claudin18.2 | VH (hu28.H1L2)-CH1- |
| | | | | antibody is | Fc |
| | | | | fused to the N | |
| | | | | terminus of | |
| | | | | each of the two | |
| | | | | anti-SIRPα | |
| | | | | scFv | |
| ES028- | Claudin18.2- | antibody | Derived from | The C terminus | 1^{st} Chain: |
| 005 (FIG. | binding domain | | hu28.H1L2 | of each of the | VL (hu28.H1L2)-CL; |
| 2B) | SIPRalpha- | scFv | Derived | heavy chain of | 2^{nd} Chain: |
| | binding domain | | from C25 | anti- | VH (hu28.H1L2)-CH1- |
| | | | | claudin18.2 | Fc-linker-scFv (C25) |
| | | | | antibody is | |
| | | | | fused to the N | |
| | | | | terminus of | |
| | | | | each of the two | |
| | | | | anti-SIRPα | |
| | | | | scFv | |
| ES028- | Claudin18.2- | scFv | Derived from | The C terminus | 1^{st} Chain: |
| 009 (FIG. | binding domain | | hu28.H1L2 | of each of the | VL (C25)-CL-linker- |
| 2C) | SIPRalpha- | antibody | Derived | light chain of | scFv (hu28.H1L2); |
| | binding domain | | from C25 | anti- SIRPα | 2^{nd} Chain: |
| | | | | antibody is | VH (C25)-CH1-Fc |
| | | | | fused to the N | |
| | | | | terminus of | |
| | | | | each of the two | |
| | | | | anti- | |
| | | | | Claudin18.2 | |
| | | | | scFv | |
| ES028- | Claudin18.2- | scFv | Derived from | The C terminus | 1^{st} Chain: |
| 013 (FIG. | binding domain | | hu28.H1L2 | of each of the | VL (C25)-CL; |
| 2D) | SIPRalpha- | antibody | Derived | heavy chain of | 2^{nd} Chain: |
| | binding domain | | from C25 | anti- SIRPα | VH (C25)-CH1-Fc- |
| | | | | antibody is | linker-scFv (hu28.H1L2) |
| | | | | fused to the N | |
| | | | | terminus of | |
| | | | | each of the two | |
| | | | | anti- | |
| | | | | Claudin18.2 | |
| | | | | scFv | |
| ES019- | PD-L1- | sdAb | Derived | The C terminus | 1^{st} Chain: |
| 020 (FIG. | binding domain | | from C71 | of each of the | VL (C25)-CL-linker- |
| 6A) | SIPRalpha- | antibody | Derived | light chains of | sdAb (C71); |
| | binding domain | | from C25 | anti- SIRPα | 2^{nd} Chain: |
| | | | | antibody is | VH (C25)-CH1-Fc |
| | | | | fused to the N | |
| | | | | terminus of | |
| | | | | each of the two | |
| | | | | anti-PDL1 | |
| | | | | sdAb | |
| ES019- | PD-L1- | sdAb | Derived | The C terminus | 1^{st} Chain: |
| 024 (FIG. | binding domain | | from C71 | of each of the | VL (C25)-CL; |
| 6B) | SIPRalpha- | antibody | Derived | heavy chain of | 2^{nd} Chain: |
| | binding domain | | from C25 | anti- SIRPα | VH (C25)-CH1-Fc- |
| | | | | antibody is | linker-sdAb (C71) |
| | | | | fused to the N | |
| | | | | terminus of | |
| | | | | each of the two | |
| | | | | anti-PDL1 | |
| | | | | sdAb | |
| ES019- | PD-L1- | sdAb | Derived | The N | 1^{st} Chain: |
| 025 (FIG. | binding domain | | from C71 | terminus of | VL (C25)-CL; |
| 6C) | SIPRalpha- | antibody | Derived | each of the | 2^{nd} Chain: |
| | binding domain | | from C25 | heavy chain of | sdAb (C71) -linker-VH |
| | | | | anti- SIRPα | (C25)-CH1-Fc |
| | | | | antibody is | |
| | | | | fused to the C | |
| | | | | terminus of | |
| | | | | each of the two | |
| | | | | anti-PDL1 | |
| | | | | sdAb | |
| ES019- | PD-L1- | sdAb | Derived | The N | 1^{st} Chain: |
| 026 (FIG. | binding domain | | from C71 | terminus of | sdAb (C71)-linker-VL |
| 6D) | SIPRalpha- | antibody | Derived | each of the | (C25)-CL; |
| | binding domain | | from C25 | light chains of | 2^{nd} Chain: |
| | | | | anti- SIRPα | VH (C25)-CH1-Fc |
| | | | | antibody is | |
| | | | | fused to the C | |
| | | | | terminus of | |
| | | | | each of the two | |
| | | | | anti-PDL1 | |
| | | | | sdAb | |
| ES019- | PD-L1- | sdAb | Derived | one | 1^{st} Chain: |
| 029 (FIG. | binding domain | | from C71 | asymmetric | sdAb (C71) -linker- |
| 6E) | SIPRalpha- | Asymmetric | Derived | antibody with | sdAb (C71) -linker -Fc; |
| | binding domain | antibody | from C25 | one Fab arm | 2^{nd} Chain: |
| | | with one | | targeting | VH (C25)-CH1-Fc; |
| | | Fab arm | | SIRPα, and the | 3^{rd} Chain: |
| | | | | other arm | VL (C25)-CL |
| | | | | containing two | |
| | | | | anti-PDL1 | |
| | | | | sdAb; the | |
| | | | | heterodimer is | |
| | | | | connected by | |
| | | | | knob-in hole | |
| | | | | mutations in Fc | |
| | | | | region | |
| ES019- | PD-L1- | sdAb | Derived | one | 1^{st} Chain: |
| 072 (FIG. | binding domain | | from C71 | asymmetric | sdAb (C71) -linker--Fc; |
| 6F) | SIPRalpha- | Fab | Derived | antibody with | 2^{nd} Chain: |
| | binding domain | | from C25 | the C terminus | sdAb (C71) -linker- Fc- |
| | | | | of the two anti- | VH (C25)-CH1; |
| | | | | PDL1 sdAb | 3^{rd} Chain: |
| | | | | fused to the N- | VL (C25)-CL |
| | | | | terminus of Fc | |
| | | | | and the N | |
| | | | | terminu sofone | |
| | | | | anti- SIRPα | |
| | | | | Fab fused to C- | |
| | | | | terminus of Fc; | |
| | | | | the | |
| | | | | heterodimer is | |
| | | | | connected by | |
| | | | | knob-in hole | |
| | | | | mutations in Fc | |
| | | | | region | |
| ES019- | PD-L1- | sdAb | Derived | one | 1^{st} Chain: |
| 073 (FIG. | binding domain | | from C71 | asymmetric | VL (C25)-CL; |
| 6G) | SIPRalpha- | Fab | Derived | antibody with | 2^{nd} Chain: |
| | binding domain | | from C25 | the C terminus | sdAb (C71) -linker-Fc- |
| | | | | of two anti- | VH (C25)-CH1 |
| | | | | PDL1 sdAb | |
| | | | | fused to N- | |
| | | | | terminus of Fc | |
| | | | | and the N- | |
| | | | | terminus of | |
| | | | | two anti-SIRPα | |
| | | | | Fabs fused to | |
| | | | | C-terminus of | |
| | | | | Fc | |
| ES019- | PD-L1- | sdAb | Derived | one | 1^{st} Chain: |
| 079 (FIG. | binding domain | | from C570 | asymmetric | VL (C25)-CL; |
| 6G) | SIPRalpha- | Fab | Derived | antibody with | 2^{nd} Chain: |
| | binding domain | | from C25 | the C terminus | sdAb (C570) -linker-Fc- |
| | | | | of two anti- | VH (C25)-CH1 |
| | | | | PDL1 | |
| | | | | sdAbfused to | |
| | | | | N-terminus of | |
| | | | | Fc and the N- | |
| | | | | terminus of | |
| | | | | two anti-SIRPα | |
| | | | | Fabs fused to | |
| | | | | C-terminus of | |
| | | | | Fc | |

### B. Multi-Specific Molecule

In certain embodiments, the multi-specific molecule provided herein comprises a SIRP-alpha binding domain provided herein, an activating receptor-binding domain provided herein comprising an Fc domain, and a target antigen binding domain provided herein.

The multi-specific molecule provided herein can be in any suitable format. Illustrative examples are provided as follows. In certain embodiments, the multi-specific molecule provided herein comprises a target antigen-binding antibody comprising two heavy chains and two light chains, wherein the C-terminus of each of the light chains is fused to an anti-SIRPα scFv (i.e. SIRPα binding domain). The target antigen-binding antibody comprises the target antigen binding domain and Fc domain. Illustrative example is shown in FIG. 2A.

In certain embodiments, the multi-specific molecule provided herein comprises a target antigen-binding antibody comprising two heavy chains and two light chains, wherein the C-terminus of each of the heavy chains is fused to an anti-SIRPα scFv (i.e. SIRPα binding domain). The target antigen-binding antibody comprises the target antigen binding domain and Fc domain. Illustrative example is shown in FIG. 2B.

In certain embodiments, the multi-specific molecule provided herein comprises an anti-SIRPα antibody comprising two heavy chains and two light chains, wherein the C-terminus of each of the light chains is fused to an scFv capable of binding to a target antigen (i.e. target antigen binding domain). The anti-SIRPα antibody comprises the SIRPα binding domain and Fc domain. Illustrative example is shown in FIG. 2C.

In certain embodiments, the multi-specific molecule provided herein comprises an anti-SIRPα antibody comprising two heavy chains and two light chains, wherein the C-terminus of each of the heavy chains is fused to an scFv capable of binding to a target antigen (i.e. target antigen binding domain). The anti-SIRPα antibody comprises the SIRPα binding domain and Fc domain. Illustrative example is shown in FIG. 2D.

In certain embodiments, the multi-specific molecule provided herein comprises an anti-SIRPα antibody comprising two heavy chains and two light chains, wherein the C-terminus of each of the light chains is fused to a single domain antibody (sdAb) capable of binding to a target antigen (i.e. target antigen binding domain). The anti-SIRPα antibody comprises the SIRPα binding domain and Fc domain. Illustrative example is shown in FIG. 6A.

In certain embodiments, the multi-specific molecule provided herein comprises an anti-SIRPα antibody comprising two heavy chains and two light chains, wherein the C-terminus of each of the heavy chains is fused to a single domain antibody (sdAb) capable of binding to a target antigen (i.e. target antigen binding domain). The anti-SIRPα antibody comprises the SIRPα binding domain and Fc domain. Illustrative example is shown in FIG. 6B.

In certain embodiments, the multi-specific molecule provided herein comprises an anti-SIRPα antibody comprising two heavy chains and two light chains, wherein the N-terminus of each of the heavy chains is fused to a single domain antibody (sdAb) capable of binding to a target antigen (i.e. target antigen binding domain). The anti-SIRPα antibody comprises the SIRPα binding domain and Fc domain. Illustrative example is shown in FIG. 6C.

In certain embodiments, the multi-specific molecule provided herein comprises an anti-SIRPα antibody comprising two heavy chains and two light chains, wherein the N-terminus of each of the light chains is fused to a single domain antibody (sdAb) capable of binding to a target antigen (i.e. target antigen binding domain). The anti-SIRPα antibody comprises the SIRPα binding domain and Fc domain. Illustrative example is shown in FIG. 6D.

In certain embodiments, the multi-specific molecule provided herein comprises an anti-SIRPα binding domain (e.g. Fab) and a single domain antibody (sdAb) capable of binding to a target antigen, respectively, each being fused to N terminus of a polypeptide chain of the Fc domain. Illustrative example is shown in FIG. 6E.

In certain embodiments, the multi-specific molecule provided herein comprises two heavy chains, each comprising a single domain antibody (sdAb) capable of binding to a target antigen fused to N terminus of a polypeptide chain of the Fc domain, and further comprises at least one anti-SIRPα binding domain fused to the C terminus of one of the polypeptide chains of the Fc domain. In certain embodiments, the multi-specific molecule provided herein comprises one anti-SIRPα binding domain fused to the C terminus of one of the polypeptide chains of the Fc domain. Illustrative example is shown in FIG. 6F.

In certain embodiments, the multi-specific molecule provided herein comprises two anti-SIRPα binding domains, each being fused to the C terminus of one of the polypeptide chains of the Fc domain. Illustrative example is shown in FIG. 6G.

In certain of these embodiments, the target binding domain is a Claudin18.2 binding domain or a PD-L1-binding domain.

In certain embodiments, the multi-specific molecule provided herein comprises an SIRPα-binding domain comprising one or more (e.g. 1, 2, 3, 4, 5, or 6) CDR sequences of SEQ ID NO: 23-64 (derived from C25, hu025.021, hu025.033, hu025.023, hu025.059, hu025.060, C15, C42, C59 and C73), a Claudin18.2-binding domain comprising one or more (e.g. 1, 2, 3, 4, 5, or 6) CDR sequences of SEQ ID NO: 77-106 and 225 (derived from hu26.H1L1, hu26.H1L2, hu26.H1L2 (S92A), hu26.H3L1, hu26.H3L2, hu28.H1L2, C10, C29 and C30), and/or a PD-L1-binding domain comprising one or more (e.g. 1, 2, 3, 4, 5, or 6) CDR sequences of SEQ ID NO: 119-154 and 223 (derived from C71, C71v38, C239, C492, C570, 570h3, C446, C2811, C1778, C1793, C2855, C2713 and C2719)

In certain embodiments, the SIRPα/Claudin18.2/PD-L1-binding domain comprises an antibody domain or an antibody mimetic domain. In certain embodiments, the antibody domain is selected from the group consisting of Fab, a VHH, a single chain Fv (scFv), diabody, a Fab′, a F(ab′)₂, a Fd, an Fv fragment, a disulfide stabilized Fv fragment (dsFv), a (dsFv)₂, a bispecific dsFv (dsFv-dsFv′), a disulfide stabilized diabody (ds diabody), F(ab)₂, an scFv dimer (bivalent diabody), a camelized single domain antibody (adAb), a nanobody, a Tetrabody, a domain antibody, or a bivalent domain antibody. In certain embodiments, the antibody mimetic domain is selected from the group consisting of an intrabody (e.g., fibronectin domain), a monobody, a linear peptide, Z domain of protein A (Affibody), gamma-B crystalline domain, ubiquitin domain, cystatin domain, Sac7d domain, triple helix coiled coil domain, lipocalins domain, A domains of a membrane receptor, Ankyrin repeat motif, SH3 domain of Fyn, Kunitz domain of a protease inhibitor, type III domain of fibronectin (Minibody), a DARPin domain, carbohydrae binding module 32-2.

In certain embodiments, the multi-specific molecule provided herein comprises
a) a SIRPα-binding domain comprising:
heavy chain CDR1 comprising the sequence of SEQ ID NO: 23, heavy chain CDR2 comprising the sequence of SEQ ID NO: 24, and heavy chain CDR3 comprising the sequence of SEQ ID NO: 25; and/or light chain CDR1 comprising the sequence of SEQ ID NO: 26, light chain CDR2 comprising the sequence of SEQ ID NO: 27, and light chain CDR3 comprising the sequence of SEQ ID NO: 28;
b) a Claudin18.2-binding domain comprising:
   i) heavy chain CDR1 comprising the sequence of SEQ ID NO: 83, heavy chain CDR2 comprising the sequence of SEQ ID NO: 84, and heavy chain CDR3 comprising the sequence of SEQ ID NO: 85, and/or light chain CDR1 comprising the sequence of SEQ ID NO: 86, light chain CDR2 comprising the sequence of SEQ ID NO: 87, and light chain CDR3 comprising the sequence of SEQ ID NO: 88, or
   ii) heavy chain CDR1 comprising the sequence of SEQ ID NO: 77, heavy chain CDR2 comprising the sequence of SEQ ID NO: 78, and heavy chain CDR3 comprising the sequence of SEQ ID NO: 79, and/or light chain CDR1 comprising the sequence of SEQ ID NO: 80, light chain CDR2 comprising the sequence of SEQ ID NO: 81, and light chain CDR3 comprising the sequence of SEQ ID NO: 223; and/or
c) a PD-L1-binding domain comprising:
   i) heavy chain CDR1 comprising the sequence of SEQ ID NO: 119, heavy chain CDR2 comprising the sequence of SEQ ID NO: 120, and heavy chain CDR3 comprising the sequence of SEQ ID NO: 121, or
   ii) heavy chain CDR1 comprising the sequence of SEQ ID NO: 131, heavy chain CDR2 comprising the sequence of SEQ ID NO: 132, and heavy chain CDR3 comprising the sequence of SEQ ID NO: 133.

In certain embodiments, the SIRPα-binding domain comprises a heavy chain variable region comprising the sequence selected from the group consisting of SEQ ID NOs: 1, 3, 5, 7, 9, 11, 13, 15, 17, 19 and 21, or a homologous sequence thereof having at least 80% sequence identity yet retaining specific binding affinity to SIRPα (e.g. human SIRPα), and/or a light chain variable region comprising the sequence selected from the group consisting of SEQ ID NOs: 2, 4, 6, 8, 10, 12, 14, 16, 18, 20 and 22, or a homologous sequence thereof having at least 80% sequence identity yet retaining specific binding affinity to SIRPα (e.g. human SIRPα).

In certain embodiments, the SIRPα-binding domain comprises an anti-SIRPα scFv. In certain embodiments, the scFv comprises, from N terminus to C terminus, a heavy chain variable region linked to a light chain variable region, optionally via a linker (e.g. a polypeptide linker). Alternatively, in certain embodiments, the scFv comprises, from N terminus to C terminus, a light chain variable region linked to a heavy chain variable region, optionally via a linker (e.g. a polypeptide linker). In certain embodiments, the anti-SIRPα scFv comprises the amino acid sequence of SEQ ID NO: SEQ ID NO: 226.

In certain embodiments, the Claudin18.2-binding domain comprises a heavy chain variable region comprising the sequence selected from the group consisting of SEQ ID NOs: 65, 68, 69, 71, 73 and 75, or a homologous sequence thereof having at least 80% sequence identity yet retaining specific binding affinity to Claudin18.2 (e.g. human Claudin18.2), and/or a light chain variable region comprising the sequence selected from the group consisting of SEQ ID NOs: 66, 67, 70, 72, 74, 76 and 224 or a homologous sequence thereof having at least 80% sequence identity yet retaining specific binding affinity to Claudin18.2 (e.g. human Claudin18.2).

In certain embodiments, the Claudin18.2-binding domain comprises an anti-Claudin18.2 scFv. In certain embodiments, the scFv comprises, from N terminus to C terminus, a heavy chain variable region linked to a light chain variable region, optionally via a linker (e.g. a polypeptide linker). Alternatively, in certain embodiments, the scFv comprises, from N terminus to C terminus, a light chain variable region linked to a heavy chain variable region, optionally via a linker (e.g. a polypeptide linker). In certain embodiments, the anti-Claudin18.2 scFv comprises the amino acid sequence of SEQ ID NO: 227.

In certain embodiments, the PD-L1-binding domain comprises a heavy chain variable region comprising the sequence selected from the group consisting of SEQ ID NOs: 107-118 and 223, or a homologous sequence thereof having at least 80% sequence identity yet retaining specific binding affinity to PD-L1 (e.g. human PD-L1).

Various techniques can be used for the production of such antigen-binding domains. Illustrative methods include, enzymatic digestion of intact antibodies (see, e.g., Morimoto et al., Journal of Biochemical and Biophysical Methods 24:107-117 (1992); and Brennan et al., Science, 229:81 (1985)), recombinant expression by host cells such as E. Coli (e.g. for Fab, Fv and ScFv antibody fragments), and screening from a phage display library as discussed above (e.g. for ScFv). Other techniques for the production of antibody fragments will be apparent to a skilled practitioner

In certain embodiments, the SIRPα-binding domain and/or the Claudin18.2-binding domain is or comprises a scFv or a Fab. Generation of scFv is described in, for example, WO 93/16185; U.S. Pat. Nos. 5,571,894; and 5,587,458. scFv may be fused to an effector protein at either the amino or the carboxyl terminus to provide for a fusion protein (see, for example, Antibody Engineering, ed. Borrebaeck). An scFv can comprise a VH linked directly or via a peptide linker to a VL. In certain embodiments, the VH can be at the N-terminus and the VL can be at the C terminus of the scFv. In certain embodiments, the VL can be at the N-terminus and the VH can be at the C terminus of the scFv.

In certain embodiments, the SIRPalpha-binding domain, the target antigen-binding domain (e.g., claudin18.2-binding domain/PD-L1-binding domain), and the activating receptor-binding domain are connected via a peptide linker. The peptide linker can comprise a single or repeated sequences composed of threonine/serine and glycine, such as TGGGG (SEQ ID NO: 183), GGGGS (SEQ ID NO: 184), GGGGSGGGGS (SEQ ID NO: 185), (Gly4Ser)3 (SEQ ID NO: 186) or SGGGG (SEQ ID NO: 187) or its tandem repeats (e.g. 2, 3, 4, or more repeats). In certain embodiments, the peptide linker comprises GGGGSGGGGSGGGGSGGGGS (SEQ ID NO: 34). In certain embodiments, the peptide linker comprises or is (Gly4Ser)3 (SEQ ID NO: 186).

### C. Characterization of the Multi-Specific Molecules

In some embodiments, the multi-specific molecules provided herein are capable of specifically binding to both human SIRPalpha and CD47-expressing cell surface marker (e.g., human Claudin18.2 or PD-L1 expressed on surface of CD47-expressing cells). The multi-specific molecules provided herein retain the specific binding affinity to both human SIRPalpha and human Claudin18.2/human PD-L1, in certain embodiments are at least comparable to, or even better than, the parent antibodies in that aspect.

Binding of multi-specific molecules can be represented by “half maximal effective concentration” (EC₅₀) value, which refers to the concentration of an antibody where 50% of its maximal effect (e.g., binding or inhibition etc.) is observed. The EC₅₀ value can be measured by methods known in the art, for example, sandwich assay such as ELISA, Western Blot, flow cytometry assay, and other binding assay.

Binding affinity of the antigen-binding domains provided herein can also be represented by K_{D} value, which represents the ratio of dissociation rate to association rate (k_{off}/kₒₙ) when the binding between the antigen and antigen-binding molecule reaches equilibrium. The antigen-binding affinity (e.g. K_{D}) can be appropriately determined using suitable methods known in the art, including, for example, flow cytometry assay. In some embodiments, binding of the antigen-binding domains to the antigen at different concentrations can be determined by flow cytometry, the determined mean fluorescence intensity (MFI) can be firstly plotted against concentration of the antigen-binding domains, K_{D} value can then be calculated by fitting the dependence of specific binding fluorescence intensity (Y) and the concentration of antibodies (X) into the one site saturation equation: Y=Bₘₐₓ*X/(K_{D}+X) using Prism version 5 (GraphPad Software, San Diego, CA), wherein Bₘax refers to the maximum specific binding of the tested antigen-binding domains to the antigen.

In certain embodiments, the multi-specific molecules provided herein specifically bind to human SIRPalpha with a binding affinity (K_{D}) of no more than: 25×10⁻⁸ M, 20×10⁻⁸ M, 15×10⁻⁸ M, 12×10⁻⁸ M, 10×10⁻⁸ M, 9×10⁻⁸ M, 8×10⁻⁸ M, 7×10⁻⁸ M, 6×10⁻⁸ M, 5×10⁻⁸ M, 4×10⁻⁸ M, 3×10⁻⁸ M, 2×10⁻⁸ M, or 1×10⁻⁸ M as measured by Octet assay.

In certain embodiments, the multi-specific molecules provided herein specifically bind to human claudin18.2 with a binding affinity (K_{D}) of no more than 1×10⁻¹² M as measured by Octet assay.

In certain embodiments, the multi-specific molecules provided herein specifically bind to human PD-L1 with a binding affinity (K_{D}) of no more than: 70×10⁻⁸ M, 65×10⁻⁸ M, 60×10⁻⁸ M, 55×10⁻⁸ M, 50×10⁻⁸ M, 45×10⁻⁸ M, 40×10⁻⁸ M, 35×10⁻⁸ M, 30×10⁻⁸ M, 25×10⁻⁸ M, 20×10⁻⁸ M, 15×10⁻⁸ M, 12×10⁻⁸ M, 10×10⁻⁸ M, 9×10⁻⁸ M, 8×10⁻⁸ M, 7×10⁻⁸ M, 6×10⁻⁸ M, 5×10⁻⁸ M, 4×10⁻⁸ M, 3×10⁻⁸ M, 2×10⁻⁸ M, or 1×10⁻⁸ M as measured by Octet assay.

The blocking effect of the multi-specific molecules provided herein on CD47 and SIRPalpha interaction or PD-1 and PD-L1 interaction can be measured by various techniques, such as luciferase reporter assay, competitive ELISA assay and competitive FACS assay, which can be expressed in IC50. IC50 for blocking CD47 and SIRPalpha interaction indicates the concentration of the multi-specific molecules provided herein at which the binding of CD47 to SIRPalpha is decreased by 50% in presence of the multi-specific molecules of the present disclosure. IC50 for blocking PD-1 and PD-L1 interaction indicates the concentration of the multi-specific molecules provided herein at which the binding of PD-1 to PD-L1 is decreased by 50% in presence of the the multi-specific molecules of the present disclosure. In certain embodiments, the IC50 of the multi-specific molecules provided herein for blocking PD-1 and PD-L1 interaction is comparable with that of the anti-PD-1 C71.

In certain embodiments, the SIRP-alpha binding domain provided herein has an IC50 value for blocking SIRP-alpha and CD47 interaction of no more than 6.0 nM, 5.0 nM, 4.0 nM, 3.80 nM, 3.60 nM, 3.0 nM, 2.90 nM, 2.88 nM, 2.86 nM, 2.80 nM, 2.78 nM, 2.76 nM, 2.74 nM, 2.70 nM, 2.64 nM, 2.40 nM, 2.20 nM, 2.0 nM, 1.0 nM, 0.4 nM, 0.3 nM, 0.2 nM, 0.18 nM, 0.16 nM, 0.14 nM, 0.12 nM as measured by competitive ELISA assay or competitive FACS assay. In certain embodiments, the SIRP-alpha binding domain provided herein has an IC50 value for blocking SHP-1 recruitment of no more than 6.0 nM, no more than 5.0 nM, no more than 4.0 nM, no more than 3.80 nM, no more than 3.60 nM, no more than 3.0 nM, no more than 2.90 nM, no more than 2.88 nM, no more than 2.86 nM, no more than 2.80 nM, no more than 2.78 nM, no more than 2.76 nM, no more than 2.74 nM, no more than 2.70 nM, no more than 2.64 nM, no more than 2.40 nM, no more than 2.20 nM, no more than 2.0 nM, no more than 1.0 nM, no more than 0.4 nM, no more than 0.3 nM, no more than 0.2 nM, no more than 0.18 nM, no more than 0.16 nM, no more than 0.14 nM, no more than 0.12 nM, no more than 0.10 nM, no more than 0.09 nM, no more than 0.08 nM or no more than 0.07 nM as measured by competitive ELISA assay or competitive FACS assay.

### D. Variants

The multi-specific molecules provided herein also encompass various variants thereof. In certain embodiments, the variants comprise one or more modifications or substitutions in one or more CDR sequences as provided in Tables 2, 4 and 6, one or more variable region sequences (but not in any of the CDR sequences) provided in Tables 1, 3 and 5, and/or the constant region (e.g. Fc region). Such variants retain specific binding affinity to SIRPα, Claudin18.2 and/or PD-L1 of their parent antibodies, but have one or more desirable properties conferred by the modification(s) or substitution(s). For example, the variants may have improved antigen-binding affinity, improved productivity, improved stability, improved glycosylation pattern, reduced risk of glycosylation, reduced deamination, reduced or depleted effector function(s), improved FcRn receptor binding, increased pharmacokinetic half-life, pH sensitivity, and/or compatibility to conjugation (e.g. one or more introduced cysteine residues).

The parent antibody sequence may be screened to identify suitable or preferred residues to be modified or substituted, using methods known in the art, for example “alanine scanning mutagenesis” (see, for example, Cunningham and Wells (1989) Science, 244:1081-1085). Briefly, target residues (e.g., charged residues such as Arg, Asp, His, Lys, and Glu) can be identified and replaced by a neutral or negatively charged amino acid (e.g., alanine or polyalanine), and the modified antibodies are produced and screened for the interested property. If substitution at a particular amino acid location demonstrates an interested functional change, then the position can be identified as a potential residue for modification or substitution. The potential residues may be further assessed by substituting with a different type of residue (e.g. cysteine residue, positively charged residue, etc.).

In certain embodiments, the SIRPα-binding domains, the Claudin18.2-binding domains, and/or the PD-L1 binding domains provided herein comprise one or more amino acid residue substitutions in one or more CDR sequences, and/or one or more FR sequences, and/or one or more variable region sequences. In certain embodiments, a variant comprises no more than 10, 9, 8, 7, 6, 5, 4, 3, 2, or 1 substitutions in the CDR sequences and/or FR sequences and/or one or more variable region sequences in total.

In certain embodiments, the SIRPα-binding domains comprise 1, 2, 3, 4, 5, or 6 CDR sequences having at least 80% (e.g. at least 85%, 88%, 90%, 91%, 92%, 93%, 94%, 95%, 96%, 97%, 98%, 99%) sequence identity to 1, 2, 3, 4, 5, or 6 sequences selected from SEQ ID NOs: 23-64 and 198, and in the meantime retain the binding affinity to SIRPα at a level similar to or even higher than its parent antibody.

In certain embodiments, the SIRPα-binding domains comprise one or more variable region sequences having at least 80% (e.g. at least 85%, 88%, 90%, 91%, 92%, 93%, 94%, 95%, 96%, 97%, 98%, 99%) sequence identity to one or more sequences selected from SEQ ID NOs: 1-22, and in the meantime retain the binding affinity to SIRPα at a level similar to or even higher than its parent antibody. In some embodiments, a total of 1 to 10 amino acids have been substituted, inserted, or deleted in a variable region sequence selected from SEQ ID NOs: 1-22. In some embodiments, the substitutions, insertions, or deletions occur in regions outside the CDRs (e.g., in the FRs).

In certain embodiments, the Claudin18.2-binding domains comprise 1, 2, 3, 4, 5, or 6 CDR sequences having at least 80% (e.g. at least 85%, 88%, 90%, 91%, 92%, 93%, 94%, 95%, 96%, 97%, 98%, 99%) sequence identity to 1, 2, 3, 4, 5, or 6 sequences selected from SEQ ID NOs: 77-106 and 225, and in the meantime retain the binding affinity to SIRPα at a level similar to or even higher than its parent antibody.

In certain embodiments, the Claudin18.2-binding domains comprise one or more variable region sequences having at least 80% (e.g. at least 85%, 88%, 90%, 91%, 92%, 93%, 94%, 95%, 96%, 97%, 98%, 99%) sequence identity to one or more sequences selected from SEQ ID NOs: 65-76 and 224, and in the meantime retain the binding affinity to Claudin18.2 at a level similar to or even higher than its parent antibody. In some embodiments, a total of 1 to 10 amino acids have been substituted, inserted, or deleted in a variable region sequence selected from SEQ ID NOs: 65-76 and 224. In some embodiments, the substitutions, insertions, or deletions occur in regions outside the CDRs (e.g., in the FRs).

In certain embodiments, the PD-L1-binding domains comprise 1, 2, or 3 CDR sequences having at least 80% (e.g. at least 85%, 88%, 90%, 91%, 92%, 93%, 94%, 95%, 96%, 97%, 98%, 99%) sequence identity to 1, 2, or 3 sequences selected from SEQ ID NOs:119-154, and in the meantime retain the binding affinity to PD-L1 at a level similar to or even higher than its parent antibody.

In certain embodiments, the PD-L1-binding domains comprise one or more variable region sequences having at least 80% (e.g. at least 85%, 88%, 90%, 91%, 92%, 93%, 94%, 95%, 96%, 97%, 98%, 99%) sequence identity to one or more sequences selected from SEQ ID NOs: 107-118 and 223, and in the meantime retain the binding affinity to PD-L1 at a level similar to or even higher than its parent antibody. In some embodiments, a total of 1 to 10 amino acids have been substituted, inserted, or deleted in a variable region sequence selected from SEQ ID NOs: 107-118 and 223. In some embodiments, the substitutions, insertions, or deletions occur in regions outside the CDRs (e.g., in the FRs).

i. Glycosylation Variant

The multi-specific molecules provided herein also encompass a glycosylation variant, which can be obtained to either increase or decrease the extent of glycosylation of the antigen-binding domains or the activating-receptor domain of the multi-specific molecules.

The multi-specific molecules provided herein may comprise one or more amino acid residues with a side chain to which a carbohydrate moiety (e.g. an oligosaccharide structure) can be attached. Glycosylation of antibodies antigen-binding domains is typically either N-linked or O-linked. N-linked refers to the attachment of the carbohydrate moiety to the side chain of an asparagine residue, for example, an asparagine residue in a tripeptide sequence such as asparagine-X-serine and asparagine-X-threonine, where X is any amino acid except proline. O-linked glycosylation refers to the attachment of one of the sugars N-aceylgalactosamine, galactose, or xylose to a hydroxyamino acid, most commonly to serine or threonine. Removal of a native glycosylation site can be conveniently accomplished, for example, by altering the amino acid sequence such that one of the above-described tripeptide sequences (for N-linked glycosylation sites) or serine or threonine residues (for O-linked glycosylation sites) present in the sequence in the is substituted. A new glycosylation site can be created in a similar way by introducing such a tripeptide sequence or serine or threonine residue.

ii. Cysteine-Engineered Variant

The multi-specific molecules provided herein also encompass a cysteine-engineered variant, which comprises one or more introduced free cysteine amino acid residues.

A free cysteine residue is one which is not part of a disulfide bridge. A cysteine-engineered variant is useful for conjugation with for example, a cytotoxic and/or imaging compound, a label, or a radioisoptype among others, at the site of the engineered cysteine, through for example a maleimide or haloacetyl. Methods for engineering antibody polypeptides to introduce free cysteine residues are known in the art, see, for example, WO2006/034488.

iii. Fc Variant

The multi-specific molecules provided herein also encompass an Fc variant, which comprises one or more amino acid residue modifications or substitutions at its Fc region and/or hinge region, for example, to provide for altered effector functions such as ADCC, ADCP and CDC. Methods of altering ADCC activity by antibody engineering have been described in the art, see for example, Shields R L. et al., J Biol Chem. 2001. 276(9): 6591-604; Idusogie E E. et al., J Immunol. 2000.164(8):4178-84; Steurer W. et al., J Immunol. 1995, 155(3): 1165-74; Idusogie E E. et al., J Immunol. 2001, 166(4): 2571-5; Lazar G A. et al., PNAS, 2006, 103(11): 4005-4010; Ryan M C. et al., Mol. Cancer Ther., 2007, 6: 3009-3018; Richards J O., et al., Mol Cancer Ther. 2008, 7(8): 2517-27; Shields R. L. et al, J. Biol. Chem, 2002, 277: 26733-26740; Shinkawa T. et al, J. Biol. Chem, 2003, 278: 3466-3473.

CDC activity of the antibodies provided herein can also be altered, for example, by improving or diminishing C1q binding and/or CDC (see, for example, WO99/51642; Duncan & Winter Nature 322:738-40 (1988); U.S. Pat. Nos. 5,648,260; 5,624,821); and WO94/29351 concerning other examples of Fc region variants. One or more amino acids selected from amino acid residues 329, 331 and 322 of the Fc region can be replaced with a different amino acid residue to alter C1q binding and/or reduced or abolished complement dependent cytotoxicity (CDC) (see, U.S. Pat. No. 6,194,551 by Idusogie et al). One or more amino acid substitution(s) can also be introduced to alter the ability of the antibody to fix complement (see PCT Publication WO 94/29351 by Bodmer et al.).

The term “Antibody-dependent cellular phagocytosis” and “ADCP” refer to a process by which antibody-coated cells or particles are internalized, either in whole or in part, by phagocytic immune cells (e.g., macrophages, neutrophils and dendritic cells) that bind to an immunoglobulin Fc region. Methods for altering the ADCP activity of antibodies by antibody engineering are known in the art, see for example, Kellner C et al., Transfus Med Hemother, (2017)44:327-336 and Chung A W et al., AIDS, (2014) 28:2523-2530. Examples of Fc variants are known in the art, see, for example, Wang et al., Protein Cell 2018, 9(1): 63-73 and Kang et al., Exp & Mol., Med. (2019) 51:138, which are incorporated herein to their entirety.

i) Fc Variant with Enhanced Effector Functions

In certain embodiments, the Fc variants provided herein has increased ADCC and/or increased affinity to an Fcγ receptor (e.g. FcγRI (CD64), FcγRII (CD32) and/or FcγRIII (CD16)) relative to a wildtype Fc (e.g. Fc of IgG1). In certain embodiments, an Fc variant comprises one or more amino acid substitution(s) at one or more of the following positions: 234, 235, 236, 238, 239, 240, 241, 243, 244, 245, 246, 247, 248, 249, 252, 254, 255, 256, 258, 260, 262, 263, 264, 265, 267, 268, 269, 270, 272, 274, 276, 278, 280, 283, 285, 286, 289, 290, 292, 293, 294, 295, 296, 298, 299, 300, 301, 303, 304, 305, 307, 309, 312, 313, 315, 320, 322, 324, 325, 326, 327, 329, 330, 331, 332, 333, 334, 335, 337, 338, 339, 340, 345, 360, 373, 376, 378, 382, 388, 389, 396, 398, 414, 416, 419, 430, 433, 434, 435, 436, 437, 438, 439 and 440 of the Fc region (see WO 00/42072 by Presta, WO2006/019447 by Lazar, and WO2016/196228, incorporated herein to its entirety), wherein the numbering of the residues in the Fc region is that of the EU index as in Kabat (see, Kabat E. A. et al., Sequences of Proteins of immunological Interest, 5th Ed. Public Health Service, National Institutes of Health, Bethesda, Md. (1991)). Exemplary substitutions for increased effector functions include, without limitation, 234Y, 235Q, 236A, 236W, 239D, 239E, 239M, 243L, 2471, 268D, 267E, 268D, 268E, 268F, 270E, 280H, 290S, 292P, 298A, 298D, 298V, 300L, 305I, 324T, 326A, 326D, 326W, 330L, 330M, 333S, 332D, 332E, 298A, 333A, 334A, 334E, 326A, 2471, 339D, 339Q, 345R, 280H, 290S, 298D, 298V, 243L, 292P, 300L, 396L, 305I, 396L, 430G, 440Y, or any combination thereof (such as 239D/332E, 239D/332E/330L, 236A/332E, 236A/239D/332E, 268F/324T, 267E/268F, 267E/324T, and 267E/268F/324T) (see, WO2016/196228; Richards et al. (2008) Mol. Cancer Therap. 7:2517; Moore et al. (2010) mAbs 2:181; and Strohl (2009) Current Opinion in Biotechnology 20:685-691).

Specific mutations at positions 256, 290, 298, 333, 334 and 339 were shown to improve binding to FcγRIII. Additionally, the following combination mutants were shown to improve FcγRIII binding: T256A/S298A, S298A/E333A, S298A/K224A, F243L/R292P/Y300L/V305I/P396L, S298A/E333A/K334A and L234Y/L235Q/G236W/S239M/H268D/D270E/S298A in one heavy chain and D270E/K326D/A330M/K334E in the opposing heavy chain (having enhanced FcγRIII binding and ADCC activity). Other Fc variants with strongly enhanced binding to FcγRIIIa include variant with S239D/I332E and S239D/I332E/A330L mutations, which showed the greatest increase in affinity for FcγRIIIa, a decrease in FcγRIIb binding, and strong cytotoxic activity, and variants with L235V, F243L, R292P, Y300L, V305I and P396L mutations, which exhibited enhancing FcγRIIIa and concomitantly enhanced ADCC activity. (see Lazar et a. (2006) Proc. Nat'l Acad Sci. (USA) 103:4005; Awan et al. (2010) Blood 115: 1204; Desjarlais & Lazar (2011) Exp. Cell Res, Stavenhagen et al. (2007) Cancer Res 67:8882). Modifications that increase binding to C1q can be introduced in order to enhance CDC activity. Exemplary modifications include, a K326 (e.g., K326W) and/or E333 modification in an IgG2, or a S267E/H268F/S324T modification, alone or in any combination, in an IgG1 (see Idusogie et al. (2001) J. Immunol. 166:2571, Moore et al. (2010) mAbs 2: 181). Other exemplary modifications include, K326W/E333S, S267E/H268F/S324T, and E345R/E430G/S440Y.

ii) Fc with Reduced Effector Functions

In certain embodiments, the Fe variants provided herein has reduced effector functions relative to a wildtype Fe (e.g. Fe of IgG1), and comprise one or more amino acid substitution(s) at a position selected from the group consisting of: 220, 226, 229, 233, 234, 235, 236, 237, 238, 267, 268, 269, 270, 297, 309, 318, 320, 322, 325, 328, 329, 330, and 331 of the Fc region (see, WO2016/196228; Richards et al. (2008) Mol. Cancer Therap. 7:2517; Moore et al. (2010) mAbs 2:181; and Strohl (2009) Current Opinion in Biotechnology 20:685-691), wherein the numbering of the residues in the Fc region is that of the EU index as in Kabat. Exemplary substitutions for reduced effector functions include, without limitation, 220S, 226S, 228P, 229S, 233P, 234V, 234G, 234A, 234F, 234A, 235A, 235G, 235E, 236E, 236R, 237A, 237K, 238S, 267R, 268A, 268Q, 269R, 297A, 297Q, 297G, 309L, 318A, 322A, 325L, 328R, 330S, 331S, or any combination thereof (see, WO2016/196228; and Strohl (2009) Current Opinion in Biotechnology 20:685-691).

In certain embodiments, the Fe variant provided herein is of IgG1 isotype and comprises one or more amino acid substitution(s) selected from the group consisting of: L234A, L234F, L234V, F234A, V234A, L235A, L235E, G237A, P238S, H268Q, H268A, N297A, N297Q, N297G, V309L, A330S, and P331S, or any combination thereof (such as L234A/L235A). In certain embodiments, the Fe variant provided herein is of IgG2 isotype, and comprises one or more amino acid substitution(s) selected from the group consisting of: H268Q, V309L, A330S, P331S, V234A, G237A, P238S, H268A, and any combination thereof. In certain embodiments, the Fe variant provided herein is of IgG4 isotype, and comprises one or more amino acid substitution(s) selected from the group consisting of: S228P, F234A, L235E, L235A, G237A, E318A, N297A, N297Q, N297G, and any combination thereof.

iii) Fc with Altered Binding to FcRn

In certain embodiments, the Fe variant comprises one or more amino acid substitution(s) that improves binding affinity to neonatal Fc receptor (FcRn) at pH 6.0 while retaining minimal binding at pH 7.4. Such a variant can have an extended pharmacokinetic half-life, as it binds to FcRn at acidic pH which allows it to escape from degradation in the lysosome and then be translocated and released out of the cell. Methods of engineering an antibody and antigen-binding fragment thereof to improve binding affinity with FcRn are well-known in the art, see, for example, Vaughn, D. et al, Structure, 6(1): 63-73, 1998; Kontermann, R. et al, Antibody Engineering, Volume 1, Chapter 27: Engineering of the Fc region for improved PK, published by Springer, 2010; Yeung, Y. et al, Cancer Research, 70: 3269-3277 (2010); Hinton, P. et al, J. Immunology, 176:346-356 (2006); Petkova et al. (2006) Int. Immunol. 18:1759, Ball Acqua et al. Journal of Immunology 2002, 169:5171-5180, Dall' Acqua W F. et al., J Biol Chem. 281:23514-23524 (2006); Zalevsky J, et al, Nat Biotechnol.; 28:157-159 (2010); WO 2009/086320; U.S. Pat. Nos. 6,277,375; 6,821,505; WO 97/34631; and WO 2002/060919.

Non-limiting examples of Fc modifications that may result in an increase in serum half-life of the antibody when administered include, e.g., substitution(s) at one or more positions selected from: 234 (e.g., with F), 235 (e.g., with Q), 238 (e.g., with D), 250 (e.g., with E or Q), 252 (e.g., with L/Y/F/W or T), 254 (e.g., with S or T), 256 (e.g., with S/R/Q/E/D or T); 259 (e.g., with I); 272 (e.g., with A), 305(e.g., with A), 307(e.g., with A or P), 308 (e.g., with F, C or P), 311(e.g., with A or R), 312 (e.g., with A), 322 (e.g., Q), 328 (e.g. E), 331 (e.g., with A), 378 (e.g., with A), 380 (e.g., with A), 382 (e.g., with A), 428 (e.g., with L or F), 432 (e.g., with C), 433 (e.g., with H/L/R/S/P/Q or K), 434 (e.g., with H/F or Y or S or A or W), 435 (e.g. with H), 436 (e.g., with L) and 437 (e.g., with C) (all positions by EU numbering) (see, WO2016049000A2; WO2020052692; WO2016196228). In some embodiments, the Fc variant comprises one or more amino acid substitution(s) selected from the group consisting of 234F, 235Q, 238D, 250Q, 252T, 252Y, 254T, 256E, 259I, 272A, 305A, 307A, 308F, 311A, 322Q, 328E, 331S, 380A, 428L, 432C, 433K, 433S, 434S, 434Y, 434F, 434W, 434A, 435H, 436L, 437C and any combination thereof. In some embodiments, the Fc modifications comprises one or pairs or groups of modifications selected from: a) a 428L (e.g., M428L) and 434S (e.g., N434S) substitution; a 428L, 259I (e.g., V259I), and 308F (e.g., V308F) substitution; b) a 433K (e.g., H433K) and 434 (e.g., N434Y or N434F) substitution; c) a 252Y, 254T, and 256E (e.g., M252Y, S254T, and T256E) substitution; d) a 250Q and 428L substitution (e.g., T250Q and M428L); e) a 307A, 380A and 434A substitution (e.g., T307A, E380A and N434A); f) a P238D and L328E substitution; g) a L234F, L235Q, K322Q, M252T, S254T and T256E substitution; and h) and a L432C, H433S, N434W, Y436L and T437C substitution.

In some embodiments, hybrid IgG isotypes may be used to increase FcRn binding and half-life of antibodies. A hybrid Ig can be generated from two or more isotypes. For example, an IgG1/IgG3 hybrid variant may be constructed by substituting IgG1 positions in the CH2 and/or CH3 region with the amino acids from IgG3 at positions where the two isotypes differ. In some embodiments, a hybrid Ig can comprises one or more modifications (e.g. substitutions) disclosed here.

### E. Conjugates

In some embodiments, the multi-specific molecules provided herein further comprise a conjugate moiety. The conjugate moiety can be linked to the multi-specific molecules. A conjugate moiety is a non-proteinaceous moiety that can be attached to the multi-specific molecules. It is contemplated that a variety of conjugate moieties may be linked to the multi-specific molecules provided herein (see, for example, “Conjugate Vaccines”, Contributions to Microbiology and Immunology, J. M. Cruse and R. E. Lewis, Jr. (eds.), Carger Press, New York, (1989)). These conjugate moieties may be linked to the multi-specific molecules by covalent binding, affinity binding, intercalation, coordinate binding, complexation, association, blending, or addition, among other methods.

In certain embodiments, the multi-specific molecules disclosed herein may be engineered to contain specific sites outside the epitope binding portion that may be utilized for binding to one or more conjugates. For example, such a site may include one or more reactive amino acid residues, such as for example cysteine or histidine residues, to facilitate covalent linkage to a conjugate.

In certain embodiments, the multi-specific molecules may be linked to a conjugate moiety indirectly, or through another conjugate moieties. For example, the multi-specific molecules may be conjugated to biotin, then indirectly conjugated to a second conjugate moiety that is conjugated to avidin. The conjugate moieties can be a clearance-modifying agent, a toxin (e.g., a chemotherapeutic agent), a detectable label (e.g., a radioactive isotope, a lanthanide, a luminescent label, a fluorescent label, or an enzyme-substrate label), or purification moiety.

A “toxin” can be any agent that is detrimental to cells or that can damage or kill cells. Examples of toxin include, without limitation, taxol, cytochalasin B, gramicidin D, ethidium bromide, emetine, mitomycin, etoposide, tenoposide, vincristine, MMAE, MMAF, DM1, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin and analogs thereof, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), anti-mitotic agents (e.g., vincristine and vinblastine), a topoisomerase inhibitor, and a tubulin-binders.

Examples of detectable label may include a fluorescent labels (e.g. fluorescein, rhodamine, dansyl, phycoerythrin, or Texas Red), enzyme-substrate labels (e.g. horseradish peroxidase, alkaline phosphatase, luceriferases, glucoamylase, lysozyme, saccharide oxidases or β-D-galactosidase), radioisotopes (e.g. ¹²³I, ¹²⁴I, ¹²⁵I, ¹³¹I, ³⁵S, ³H, ¹¹¹In, ¹¹²In, ¹⁴C, ⁶⁴Cu, ⁶⁷Cu, ⁸⁶Y, ⁸⁸Y, ⁹⁰Y, ¹⁷⁷Lu, ²¹¹At, ¹⁸⁶Re, ¹⁸⁸Re, ¹⁵³Sm, ²¹²Bi, and ³²P, other lanthanides), luminescent labels, chromophoric moiety, digoxigenin, biotin/avidin, a DNA molecule or gold for detection.

In certain embodiments, the conjugate moiety can be a clearance-modifying agent which helps increase half-life of the multi-specific molecule. Illustrative example include water-soluble polymers, such as PEG, carboxymethylcellulose, dextran, polyvinyl alcohol, polyvinyl pyrrolidone, copolymers of ethylene glycol/propylene glycol, and the like. The polymer may be of any molecular weight, and may be branched or unbranched. The number of polymers attached to the multi-specific molecules may vary, and if more than one polymer are attached, they can be the same or different molecules.

In certain embodiments, the conjugate moiety can be a purification moiety such as a magnetic bead.

In certain embodiments, the multi-specific molecule provided herein is used for a base for a conjugate.

### F. Polynucleotides and Recombinant Methods

The present disclosure provides isolated polynucleotides that encode the multi-specific molecules provided herein.

The term “nucleic acid” or “polynucleotide” as used herein refers to deoxyribonucleic acids (DNA) or ribonucleic acids (RNA) and polymers thereof in either single- or double-stranded form. Unless specifically limited, the term encompasses polynucleotides containing known analogues of natural nucleotides that have similar binding properties as the reference nucleic acid and are metabolized in a manner similar to naturally occurring nucleotides. Unless otherwise indicated, a particular polynucleotide sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (see Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

Many vectors are available. The vector components generally include, but are not limited to, one or more of the following: a signal sequence, an origin of replication, one or more marker genes, an enhancer element, a promoter (e.g. SV40, CMV, EF-1α), and a transcription termination sequence.

The present disclosure provides vectors (e.g., expression vectors) containing the nucleic acid sequence provided herein encoding the multi-specific molecule, at least one promoter (e.g., SV40, CMV, EF-1α) operably linked to the nucleic acid sequence, and at least one selection marker. Examples of vectors include, but are not limited to, retrovirus (including lentivirus), adenovirus, adeno-associated virus, herpesvirus (e.g., herpes simplex virus), poxvirus, baculovirus, papillomavirus, papovavirus (e.g., SV40), lambda phage, and M13 phage, plasmid pcDNA3.3, pMD18-T, pOptivec, pCMV, pEGFP, pIRES, pQD-Hyg-GSeu, pALTER, pBAD, pcDNA, pCal, pL, pET, pGEMEX, pGEX, pCI, pEGFT, pSV2, pFUSE, pVITRO, pVIVO, pMAL, pMONO, pSELECT, pUNO, pDUO, Psg5L, pBABE, pWPXL, pBI, p15TV-L, pPro18, pTD, pRS10, pLexA, pACT2.2, pCMV-SCRIPT®, pCDM8, pCDNA1.1/amp, pcDNA3.1, pRc/RSV, PCR 2.1, pEF-1, pFB, pSG5, pXT1, pCDEF3, pSVSPORT, pEF-Bos etc.

Vectors comprising the polynucleotide sequence encoding the multi-specific molecule can be introduced to a host cell for cloning or gene expression. Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas such as P. aeruginosa, and Streptomyces.

In addition to prokaryotes, eukaryotic microbes such as filamentous fungi or yeast are suitable cloning or expression hosts for the vectors provided. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated multi-specific molecule provided herein are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruiffly), and Bombyx mori have been identified. A variety of viral strains for transfection are publicly available, e.g., the L-1 variant of Autographa calfornica NPV and the Bm-5 strain of Bombyx mori NPV, and such viruses may be used as the virus herein according to the present invention, particularly for transfection of Spodoptera frugiperda cells. Plant cell cultures of cotton, corn, potato, soybean, petunia, tomato, and tobacco can also be utilized as hosts.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/−DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2). In some preferable embodiments, the host cell is 293F cell.

Host cells are transformed with the above-described expression or cloning vectors for production of the multi-specific molecules provided herein and cultured in conventional nutrient media modified as appropriate for inducing promoters, selecting transformants, or amplifying the genes encoding the desired sequences. In another embodiment, the multi-specific molecules provided herein may be produced by homologous recombination known in the art.

The host cells used to produce the multi-specific molecules provided herein may be cultured in a variety of media. Commercially available media such as Ham's F10 (Sigma), Minimal Essential Medium (MEM), (Sigma), RPMI-1640 (Sigma), and Dulbecco's Modified Eagle's Medium (DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

When using recombinant techniques, the multi-specific molecules can be produced intracellularly, in the periplasmic space, or directly secreted into the medium. If the antibody is produced intracellularly, as a first step, the particulate debris, either host cells or lysed fragments, is removed, for example, by centrifugation or ultrafiltration. Carter et al., Bio/Technology 10:163-167 (1992) describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the multi-specific molecules are secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The multi-specific molecules prepared from the cells can be purified using, for example, hydroxylapatite chromatography, gel electrophoresis, dialysis, DEAE-cellulose ion exchange chromatography, ammonium sulfate precipitation, salting out, and affinity chromatography, with affinity chromatography being the preferred purification technique.

In certain embodiments, Protein A immobilized on a solid phase is used for immunoaffinity purification of the multi-specific molecules. The suitability of protein A as an affinity ligand depends on the species and isotype of any immunoglobulin Fc domain that is present in the multi-specific molecules. Protein A can be used to purify antibodies that are based on human gamma1, gamma2, or gamma4 heavy chains (Lindmark et al., J. Immunol. Meth. 62:1-13 (1983)). Protein G is recommended for all mouse isotypes and for human gamma3 (Guss et al., EMBO J. 5:1567 1575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the multi-specific molecules comprises a CH3 domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody molecule of interest and contaminants may be subjected to low pH hydrophobic interaction chromatography using an elution buffer at a pH between about 2.5-4.5, preferably performed at low salt concentrations (e.g., from about 0-0.25M salt).

### G. Pharmaceutical Composition

The present disclosure further provides pharmaceutical compositions comprising the multi-specific molecules and one or more pharmaceutically acceptable carriers.

Pharmaceutical acceptable carriers for use in the pharmaceutical compositions disclosed herein may include, for example, pharmaceutically acceptable liquid, gel, or solid carriers, aqueous vehicles, nonaqueous vehicles, antimicrobial agents, isotonic agents, buffers, antioxidants, anesthetics, suspending/dispending agents, sequestering or chelating agents, diluents, adjuvants, excipients, or non-toxic auxiliary substances, other components known in the art, or various combinations thereof.

Suitable components may include, for example, antioxidants, fillers, binders, disintegrants, buffers, preservatives, lubricants, flavorings, thickeners, coloring agents, emulsifiers or stabilizers such as sugars and cyclodextrins. Suitable antioxidants may include, for example, methionine, ascorbic acid, EDTA, sodium thiosulfate, platinum, catalase, citric acid, cysteine, thioglycerol, thioglycolic acid, thiosorbitol, butylated hydroxanisol, butylated hydroxytoluene, and/or propyl gallate. As disclosed herein, inclusion of one or more antioxidants such as methionine in a composition comprising a multi-specific molecules and conjugates as provided herein decreases oxidation of the multi-specific molecules. This reduction in oxidation prevents or reduces loss of binding affinity, thereby improving antibody stability and maximizing shelf-life. Therefore, in certain embodiments compositions are provided that comprise one or more multi-specific molecules as disclosed herein and one or more antioxidants such as methionine. Further provided are methods for preventing oxidation of, extending the shelf-life of, and/or improving the efficacy of a multi-specific molecules as provided herein by mixing the multi-specific molecules with one or more antioxidants such as methionine.

To further illustrate, pharmaceutical acceptable carriers may include, for example, aqueous vehicles such as sodium chloride injection, Ringer's injection, isotonic dextrose injection, sterile water injection, or dextrose and lactated Ringer's injection, nonaqueous vehicles such as fixed oils of vegetable origin, cottonseed oil, corn oil, sesame oil, or peanut oil, antimicrobial agents at bacteriostatic or fungistatic concentrations, isotonic agents such as sodium chloride or dextrose, buffers such as phosphate or citrate buffers, antioxidants such as sodium bisulfate, local anesthetics such as procaine hydrochloride, suspending and dispersing agents such as sodium carboxymethylcelluose, hydroxypropyl methylcellulose, or polyvinylpyrrolidone, emulsifying agents such as Polysorbate 80 (TWEEN-80), sequestering or chelating agents such as EDTA (ethylenediaminetetraacetic acid) or EGTA (ethylene glycol tetraacetic acid), ethyl alcohol, polyethylene glycol, propylene glycol, sodium hydroxide, hydrochloric acid, citric acid, or lactic acid. Antimicrobial agents utilized as carriers may be added to pharmaceutical compositions in multiple-dose containers that include phenols or cresols, mercurials, benzyl alcohol, chlorobutanol, methyl and propyl p-hydroxybenzoic acid esters, thimerosal, benzalkonium chloride and benzethonium chloride. Suitable excipients may include, for example, water, saline, dextrose, glycerol, or ethanol. Suitable non-toxic auxiliary substances may include, for example, wetting or emulsifying agents, pH buffering agents, stabilizers, solubility enhancers, or agents such as sodium acetate, sorbitan monolaurate, triethanolamine oleate, or cyclodextrin.

The pharmaceutical compositions can be a liquid solution, suspension, emulsion, pill, capsule, tablet, sustained release formulation, or powder. Oral formulations can include standard carriers such as pharmaceutical grades of mannitol, lactose, starch, magnesium stearate, polyvinyl pyrollidone, sodium saccharine, cellulose, magnesium carbonate, etc.

In certain embodiments, the pharmaceutical compositions are formulated into an injectable composition. The injectable pharmaceutical compositions may be prepared in any conventional form, such as for example liquid solution, suspension, emulsion, or solid forms suitable for generating liquid solution, suspension, or emulsion. Preparations for injection may include sterile and/or non-pyretic solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use, and sterile and/or non-pyretic emulsions. The solutions may be either aqueous or nonaqueous.

In certain embodiments, unit-dose parenteral preparations are packaged in an ampoule, a vial or a syringe with a needle. All preparations for parenteral administration should be sterile and not pyretic, as is known and practiced in the art.

In certain embodiments, a sterile, lyophilized powder is prepared by dissolving a multi-specific molecules as disclosed herein in a suitable solvent. The solvent may contain an excipient which improves the stability or other pharmacological components of the powder or reconstituted solution, prepared from the powder. Excipients that may be used include, but are not limited to, water, dextrose, sorbital, fructose, corn syrup, xylitol, glycerin, glucose, sucrose or other suitable agent. The solvent may contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides a desirable formulation. In one embodiment, the resulting solution will be apportioned into vials for lyophilization. Each vial can contain a single dosage or multiple dosages of the multi-specific molecules or composition thereof. Overfilling vials with a small amount above that needed for a dose or set of doses (e.g., about 10%) is acceptable so as to facilitate accurate sample withdrawal and accurate dosing. The lyophilized powder can be stored under appropriate conditions, such as at about 4° C. to room temperature.

Reconstitution of a lyophilized powder with water for injection provides a formulation for use in parenteral administration. In one embodiment, for reconstitution the sterile and/or non-pyretic water or other liquid suitable carrier is added to lyophilized powder. The precise amount depends upon the selected therapy being given, and can be empirically determined.

### H. Methods of Use

In another aspect, the present disclosure provides methods of use for the multi-specific molecule provided herein. The multi-specific molecule provided herein comprises a SIRP-alpha binding domain provided herein, an activating receptor-binding domain provided herein, and a target antigen binding domain provided herein. The target antigen binding domain binds to a target antigen expressed on a target cell co-expressing the target antigen and CD47. The multi-specific molecule provided herein is capable of selectively inducing effector function of an immune effector cell in the presence of the target antigen, and the immune effector cell co-expresses SIRP-alpha and the activating receptor.

In one aspect, the present disclosure provides a method of inducing phagocytosis in vitro, comprising contacting a target cell with a SIRPα positive phagocytic cell sample in the presence of the multi-specific molecule provided herein, thereby inducing the phagocytosis of the target cell by the SIRPα positive phagocytic cell.

In one aspect, the present disclosure provides methods of inducing phagocytosis of a target cell in a subject, comprising administering to the subject the multi-specific molecule provided herein in a dose effective to induce phagocytosis of the target cell in the subject.

In one aspect, the present disclosure provides methods of redirecting tumor-associated monocytes or macrophages (TAMs) into anti-tumor macrophages so as to enhance phagocytosis of the cancer cells in a subject in need thereof, comprising administering to the subject a therapeutically effective amount of the multi-specific molecule or engagers provided herein. In addition, the engagers provided herein can also activate macrophages and engage the activated macrophages into the tumor microenvironment to exhibit phagocytotic effect on cancer cells.

In another aspect, the present disclosure also provides a method of increasing the level of M1 macrophage in a tumor microenvironment of a subject in need thereof, comprising administering to the subject a therapeutically effective amount of the multi-specific molecule or engagers provided herein. As used herein, the term “tumor microenvironment” refers to the tissue(s), cells and environment that surround the cancer cells or tumor cells. Tumor microenvironment can comprise stromal cells such as fibroblasts, pericytes, endothelial cells, adipose cells, and bone marrow mesenchymal stromal cells (MSCs). Tumor microenvironment can also comprise extracellular matrix associated with the cancer cells or associated with the stromal cells surrounding the cancer cells. Extracellular matrix is primarily composed of ground substance—a porous, hydrated gel, made mainly from proteoglycan aggregates—and connective tissue fibers. Experimentally, the tumor microenvironment for a particular tumor can be obtained, for example, by dissecting and isolating a tissue bearing the specific tumor.

The term “increase” or “increasing” with respect to the level of M1 macrophage, as used herein, refers to an elevated M1 macrophage count normalized to total macrophage count in a tumor microenvironment in presence of the multi-specific molecule or engagers provided herein as compared to a tumor microenvironment in absence of the multi-specific molecule or engagers provided herein. The level of M1 macrophages or the M1 macrophage count in a tumor microenvironment can be measured by conventional techniques known in the art, such as a FACS assay for the amount of macrophages with surface marker profiles of M1 phenotype (CD80^{high} and CD206^{mid} profile, see, Zhang M et al., Anti-CD47 Treatment Stimulates Phagocytosis of Glioblastoma by M1 and M2 Polarized Macrophages and Promotes M1 Polarized Macrophages In Vivo. PloS one 11, e0153550, 10.1371 journal.pone.0153550 (2016).). The level of M1 macrphages or the M1 macrophage count can also be measured by qPCR analysis of mRNA expression level (normalized to total mRNA expression level) of inducible nitric oxide synthase 1 (Nos1), which is known to be elevated in M1 macrophages (Zhang M et al., Anti-CD47 Treatment Stimulates Phagocytosis of Glioblastoma by M1 and M2 Polarized Macrophages and Promotes M1 Polarized Macrophages In Vivo. PloS one 11, e0153550, 10.1371 journal.pone.0153550 (2016).). Other methods that can count the number of macrophages with M1 phenotype, or that can determine the expression level of one or more characteristic markers for M1 macrophages, such as confomal fluorescence microscopy or western blot, are also within the contemplation of the present disclosure. In another aspect, the present disclosure also provides methods of treating a disease, disorder or condition that can be benefited from induced phagocytosis of a target cell in a subject, comprising administering to the subject a therapeutically effective amount of the multi-specific molecule provided herein.

In certain embodiments, the target cell co-expresses the target antigen and CD47. In some embodiments, the target cells comprise cancer cells, inflammatory cells, and/or chronically infected cells. In some embodiments, the target antigen is a tumor surface antigen, an inflammatory antigen, or an antigen of an infectious microorganism. In some embodiments, the target antigen can be tumor antigen (e.g., tumor associated antigens (TAA), tumor specific antigen (TSA), such as neoantigen), or antigens presented on infected cells (e.g., Hepatitis B surface antigen (HBsAg)).

In certain embodiments, the disorder or condition that can be benefited from induced phagocytosis of a target cell can include, for example, cancer (e.g. solid tumor, hematological malignancy), an inflammatory disease, an infectious disease (e.g. chronic infection), an autoimmune disease (e.g. multiple sclerosis), a neurologic disease, a brain injury, a nerve injury, a polycythemia, a hemochromatosis, a trauma, a septic shock, fibrosis, atherosclerosis, obesity, type II diabetes, a transplant dysfunction, and arthritis.

In another aspect, the present disclosure also provides methods of treating a target antigen related disease, disorder or condition in a subject, comprising administering to the subject a therapeutically effective amount of the multi-specific molecule provided herein. For example, if the target antigen comprises a tumor antigen, then the target antigen related disease can include tumor or cancer. For example, if the target antigen comprises an antigen presented on infected cells, then the target antigen related disease can include the related infectious disease. In certain embodiments, the target antigen comprises PD-L1. In certain embodiments, the target antigen comprises claudin 18.2.

In another aspect, the present disclosure also provides methods of treating a SIRPα related disease, disorder or condition in a subject, comprising administering to the subject a therapeutically effective amount of the multi-specific molecule provided herein.

In another aspect, the present disclosure also provides methods of treating a CD47 related disease, disorder or condition in a subject, comprising administering to the subject a therapeutically effective amount of the multi-specific molecule provided herein.

In some embodiments, the subject is human. In some embodiments, the subject is homozygous for SIRPα v1. In some embodiments, the subject is homozygous for SIRPα v2.

In some embodiments, the subject has been diagnosed with or is at risk for a disease, disorder or condition selected from the group consisting of cancer (e.g. solid tumor, hematological malignancy), an inflammatory disease, an infectious disease (e.g. chronic infection), an autoimmune disease (e.g. multiple sclerosis), a neurologic disease, a brain injury, a nerve injury, a polycythemia, a hemochromatosis, a trauma, a septic shock, fibrosis, atherosclerosis, obesity, type II diabetes, a transplant dysfunction, and arthritis. In preferred embodiments, the subject has been diagnosed with or is at risk for one or more solid tumors.

In certain embodiments, the condition or a disorder treatable by the methods provided herein can be immune related disease or disorder, tumors and cancers, autoimmune diseases, or infectious disease. In certain embodiments, the immune related disease or disorder is selected from the group consisting of systemic lupus erythematosus, acute respiratory distress syndrome (ARDS), vasculitis, myasthenia gravis, idiopathic pulmonary fibrosis, Crohn's Disease, asthma, rheumatoid arthritis, graft versus host disease, a spondyloarthropathy (e.g., ankylosing spondylitis, psoriatic arthritis, isolated acute enteropathic arthritis associated with inflammatory bowel disease, reactive arthritis, Behcet's syndrome, undifferentiated spondyloarthropathy, anterior uveitis, and juvenile idiopathic arthritis.), multiple sclerosis, endometriosis, glomerulonephritis, sepsis, diabetes, acute coronary syndrome, ischemic reperfusion, psoriasis, progressive systemic sclerosis, atherosclerosis, Sjogren's syndrome, scleroderma, or inflammatory autoimmune myositis.

In certain embodiments, the condition or a disorder treatable by the methods provided herein include tumors and cancers. In certain embodiments, the condition or a disorder treatable by the methods provided herein include solid tumor and hematologic malignancy. Examples of cancers and tumors include, non-small cell lung cancer, small cell lung cancer, renal cell cancer, colorectal cancer, ovarian cancer, breast cancer, pancreatic cancer, gastric carcinoma, bladder cancer, esophageal cancer, mesothelioma, melanoma, head and neck cancer, thyroid cancer, sarcoma, prostate cancer, glioblastoma, cervical cancer, thymic carcinoma, leukemia, lymphomas, myelomas, mycoses fungoids, merkel cell cancer, and other hematologic malignancies, such as classical Hodgkin lymphoma (CHL), primary mediastinal large B-cell lymphoma, T-cell/histiocyte-rich B-cell lymphoma, EBV-positive and -negative PTLD, and EBV-associated diffuse large B-cell lymphoma (DLBCL), plasmablastic lymphoma, extranodal NK/T-cell lymphoma, nasopharyngeal carcinoma, and HHV8-associated primary effusion lymphoma, Hodgkin's lymphoma, neoplasm of the central nervous system (CNS), such as primary CNS lymphoma, spinal axis tumor, brain stem glioma, anal cancer, appendix cancer, astrocytoma, basal cell carcinoma, gallbladder cancer, gastric cancer, lung cancer, bronchial cancer, bone cancer, liver and bile duct cancer, pancreatic cancer, breast cancer, liver cancer, ovarian cancer, testicle cancer, kidney cancer, renal pelvis and ureter cancer, salivary gland cancer, small intestine cancer, urethral cancer, bladder cancer, head and neck cancer, spine cancer, brain cancer, cervix cancer, uterine cancer, endometrial cancer, colon cancer, colorectal cancer, rectal cancer, esophageal cancer, gastrointestinal cancer, skin cancer, prostate cancer, pituitary cancer, vagina cancer, thyroid cancer, throat cancer, glioblastoma, melanoma, myelodysplastic syndrome, sarcoma, teratoma, chronic lymphocytic leukemia (CLL), chronic myeloid leukemia (CML), acute lymphocytic leukemia (ALL), acute myeloid leukemia (AML), Hodgkin lymphoma, non-Hodgkin lymphoma, multiple myeloma, T or B cell lymphoma, GI organ interstitialoma, soft tissue tumor, hepatocellular carcinoma, and adenocarcinoma, or the metastases thereof. In preferred embodiments, the condition or disorder treatable by the methods provided herein comprises one or more solid tumors.

In some embodiments, the cancer is a CD47-positive cancer. In some embodiments, the cancer is a CD47-positive and target antigen-positive cancer. In some embodiments, the subject to be treated has been identified as having a CD47-positive cancer, or a CD47-positive and target antigen-positive cancer. “CD47-positive” cancer as used herein refers to a cancer characterized in expressing CD47 in a cancer cell, or expressing CD47 in a cancer cell at a level significantly higher than that would have been expected of a normal cell. “Target antigen-positive” cancer as used herein refers to a cancer characterized in expressing the target antigen in a cancer cell, or expressing the target antigen in a cancer cell at a level significantly higher than that would have been expected of a normal cell.

The presence and/or amount of CD47 and the target antigen in an interested biological sample can be determined in a test biological sample from the subject using various suitable methods. For example, the test biological sample can be exposed to anti-CD47 antibody or anti-target antigen antibody or antigen-binding fragment thereof, which binds to and detects the expressed CD47 protein or the target antigen protein. Alternatively, CD47 or the target antigen protein can also be detected at nucleic acid expression level, using methods such as qPCR, reverse transcriptase PCR, microarray, SAGE, FISH, and the like. In some embodiments, the test sample is derived from a cancer cell or tissue, or tumor infiltrating immune cells. In certain embodiments, presence or up-regulated level of the CD47 or the target antigen protein in the test biological sample indicates likelihood of responsiveness. The term “up-regulated” as used herein, refers to an overall increase of no less than 10%, 15%, 20%, 25%, 30%, 35%, 40%, 45%, 50%, 55%, 60%, 65%, 70%, 75%, 80% or greater, in the expression level of CD47 or the target antigen protein in the test sample, as compared to the reference expression level of CD47 or the target antigen. The reference level can be the level of CD47 or the target antigen expression found in normal cells of the same tissue type, optionally normalized to expression level of another gene (e.g. a house keeping gene). Alternatively, the reference level can be the level of CD47 or the target antigen expression found in healthy subjects. The reference sample can be a control sample obtained from a healthy or non-diseased individual, or a healthy or non-diseased sample obtained from the same individual from whom the test sample is obtained. For example, the reference sample can be a non-diseased sample adjacent to or in the neighborhood of the test sample (e.g. tumor). In some embodiments, a reference is tested and/or determined substantially simultaneously with the testing or determination of interest. In some embodiments, a reference is a historical reference, optionally embodied in a tangible medium. Typically, as would be understood by the skilled person in the art, a reference is determined or characterized under comparable conditions or circumstances to those under assessment.

In certain embodiments, the tumors and cancers are metastatic, especially metastatic tumors expressing CD47.

In certain embodiments, the tumors and cancers are PD-L1 positive cancer. In certain embodiments, the PD-L1 positive cancer is selected from the group consisting of NSCLC, SCLC, Melanoma, Head and Neck Cancer, Hepatocellular carcinoma, MSI-H or dMMIR cancers, cervical cancer, breast cancer, gastric carcinoma, classical Hodgkin lymphoma, pancreatic cancer, urothelial cancer.

In certain embodiments, the tumors and cancers are claudin 18.2 positive cancer. In some embodiments, the claudin 18.2 positive cancer is an epithelial-cell derived cancer. In some embodiments, the cancer is gastric cancer, pancreatic cancer, lung cancer, esophagus cancer, ovarian cancer and the metastases thereof.

In certain embodiments, the condition or a disorder treatable by the methods provided herein include autoimmune diseases. Autoimmune diseases include, but are not limited to, Acquired Immunodeficiency Syndrome (AIDS, which is a viral disease with an autoimmune component), alopecia areata, ankylosing spondylitis, antiphospholipid syndrome, autoimmune Addison's disease, autoimmune diabetes, autoimmune hemolytic anemia, autoimmune hepatitis, autoimmune inner ear disease (AIED), autoimmune lymphoproliferative syndrome (ALPS), autoimmune thrombocytopenic purpura (ATP), Behcet's disease, cardiomyopathy, celiac sprue-dermatitis hepetiformis; chronic fatigue immune dysfunction syndrome (CFIDS), chronic inflammatory demyelinating polyneuropathy (CIPD), cicatricial pemphigold, cold agglutinin disease, crest syndrome, Crohn's disease, Degos' disease, dermatomyositis-juvenile, discoid lupus, essential mixed cryoglobulinemia, fibromyalgia-fibromyositis, Graves' disease, Guillain-Barre syndrome, Hashimoto's thyroiditis, idiopathic pulmonary fibrosis, idiopathic thrombocytopenia purpura (ITP), IgA nephropathy, insulin-dependent diabetes mellitus, juvenile chronic arthritis (Still's disease), juvenile rheumatoid arthritis, Meniere's disease, mixed connective tissue disease, multiple sclerosis, myasthenia gravis, pemacious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynaud's phenomena, Reiter's syndrome, rheumatic fever, rheumatoid arthritis, sarcoidosis, scleroderma (progressive systemic sclerosis (PSS), also known as systemic sclerosis (SS)), Sjogren's syndrome, stiff-man syndrome, systemic lupus erythematosus, Takayasu arteritis, temporal arteritis/giant cell arteritis, ulcerative colitis, uveitis, vitiligo and Wegener's granulomatosis.

In certain embodiments, the condition or a disorder treatable by the methods provided herein include infectious disease. Infectious disease include, for example, chronic viral infection, for example, fungus infection, parasite/protozoan infection or chronic viral infection, for example, malaria, coccidioiodmycosis immitis, histoplasmosis, onychomycosis, aspergilosis, blastomycosis, candidiasis albicans, paracoccidioiomycosis, microsporidiosis, Acanthamoeba keratitis, Amoebiasis, Ascariasis, Babesiosis, Balantidiasis, Baylisascariasis, Chagas disease, Clonorchiasis, Cochliomyia, Cryptosporidiosis, Diphyllobothriasis, Dracunculiasis, Echinococcosis, Elephantiasis, Enterobiasis, Fascioliasis, Fasciolopsiasis, Filariasis, Giardiasis, Gnathostomiasis, Hymenolepiasis, Isosporiasis, Katayama fever, Leishmaniasis, Lyme disease, Metagonimiasis, Myiasis, Onchocerciasis, Pediculosis, Scabies, Schistosomiasis, Sleeping sickness, Strongyloidiasis, Taeniasis, Toxocariasis, Toxoplasmosis, Trichinosis, Trichuriasis, Trypanosomiasis, helminth infection, infection of hepatitis B (HBV), hepatitis C (HCV), herpes virus, Epstein-Barr virus, HIV-1, HIV-2, cytomegalovirus, herpes simplex virus type I, herpes simplex virus type II, human papilloma virus, adenovirus, Kaposi West sarcoma associated herpes virus epidemics, thin ring virus (Torquetenovirus), human T lymphotrophic viruses I, human T lymphotrophic viruses II, varicella zoster, JC virus or BK virus.

The therapeutically effective amount of an multi-specific molecule as provided herein will depend on various factors known in the art, such as for example body weight, age, past medical history, present medications, state of health of the subject and potential for cross-reaction, allergies, sensitivities and adverse side-effects, as well as the administration route and extent of disease development. Dosages may be proportionally reduced or increased by one of ordinary skill in the art (e.g., physician or veterinarian) as indicated by these and other circumstances or requirements.

In certain embodiments, the multi-specific molecule as provided herein may be administered at a therapeutically effective dosage of about 0.01 mg/kg to about 100 mg/kg. In certain of these embodiments, the multi-specific molecule is administered at a dosage of about 50 mg/kg or less, and in certain of these embodiments the dosage is 10 mg/kg or less, 5 mg/kg or less, 3 mg/kg or less, 1 mg/kg or less, 0.5 mg/kg or less, or 0.1 mg/kg or less. In certain embodiments, the administration dosage may change over the course of treatment. For example, in certain embodiments the initial administration dosage may be higher than subsequent administration dosages. In certain embodiments, the administration dosage may vary over the course of treatment depending on the reaction of the subject.

Dosage regimens may be adjusted to provide the optimum desired response (e.g., a therapeutic response). For example, a single dose may be administered, or several divided doses may be administered over time.

The multi-specific molecule disclosed herein may be administered by any route known in the art, such as for example parenteral (e.g., subcutaneous, intraperitoneal, intravenous, including intravenous infusion, intramuscular, or intradermal injection) or non-parenteral (e.g., oral, intranasal, intraocular, sublingual, rectal, or topical) routes.

In some embodiments, the multi-specific molecules disclosed herein may be administered alone or in combination with one or more additional therapeutic means or agents. For example, the multi-specific molecules disclosed herein may be administered in combination with another therapeutic agent, for example, a chemotherapeutic agent or an anti-cancer drug.

In certain of these embodiments, a multi-specific molecule as disclosed herein that is administered in combination with one or more additional therapeutic agents may be administered simultaneously with the one or more additional therapeutic agents, and in certain of these embodiments the multi-specific molecule and the additional therapeutic agent(s) may be administered as part of the same pharmaceutical composition. However, a multi-specific molecule administered “in combination” with another therapeutic agent does not have to be administered simultaneously with or in the same composition as the agent. A multi-specific molecule administered prior to or after another agent is considered to be administered “in combination” with that agent as the phrase is used herein, even if the multi-specific molecule and second agent are administered via different routes. Where possible, additional therapeutic agents administered in combination with the multi-specific molecule disclosed herein are administered according to the schedule listed in the product information sheet of the additional therapeutic agent, or according to the Physicians' Desk Reference 2003 (Physicians' Desk Reference, 57th Ed; Medical Economics Company; ISBN: 1563634457; 57th edition (November 2002)) or protocols well known in the art.

The present disclosure further provides methods of using the multi-specific molecule thereof.

In some embodiments, the present disclosure also provides use of the multi-specific molecule provided herein in the manufacture of a medicament for treating a disease, disorder or condition that can be benefited from induced phagocytosis of a target cell in a subject.

The present disclosure is based on surprising discoveries of multi-specific molecules (e.g. multi-specific antibodies) of anti-SIRP-alpha antibodies or antigen-binding fragments thereof with particular properties, e.g., substantially or completely blocking interactions between SIRP-alpha and CD47; substantially or completely blocking SHP-1 recruitment downstream SIRP-alpha and CD47 interaction; not inducing phagocytosis of certain target cells when used alone, and/or capable of binding to an epitope outside the IgV domain of SIRPα. Such multi-specific molecules can be in a specific configuration as described above, and that the resulting multi-specific molecules exhibit unexpectedly high selectivity to induce immune responses (e.g., phagocytosis) against unwanted cells so as to eliminate the unwanted cells (e.g., cancerous cells and infected cells).

Accordingly, the present disclosure also provides use of such multi-specific molecules provided herein in the manufacture of a medicament for treating CD47-related conditions and disorders.

The following examples are provided to better illustrate the claimed invention and are not to be interpreted as limiting the scope of the invention. All specific compositions, materials, and methods described below, in whole or in part, fall within the scope of the present invention. These specific compositions, materials, and methods are not intended to limit the invention, but merely to illustrate specific embodiments falling within the scope of the invention. One skilled in the art may develop equivalent compositions, materials, and methods without the exercise of inventive capacity and without departing from the scope of the invention. It will be understood that many variations can be made in the procedures herein described while still remaining within the bounds of the present invention. It is the intention of the inventors that such variations are included within the scope of the invention.

### EXAMPLES

### Example 1: Anti-Claudin18.2/SIRPα Bispecific Antibody Construction and Expression

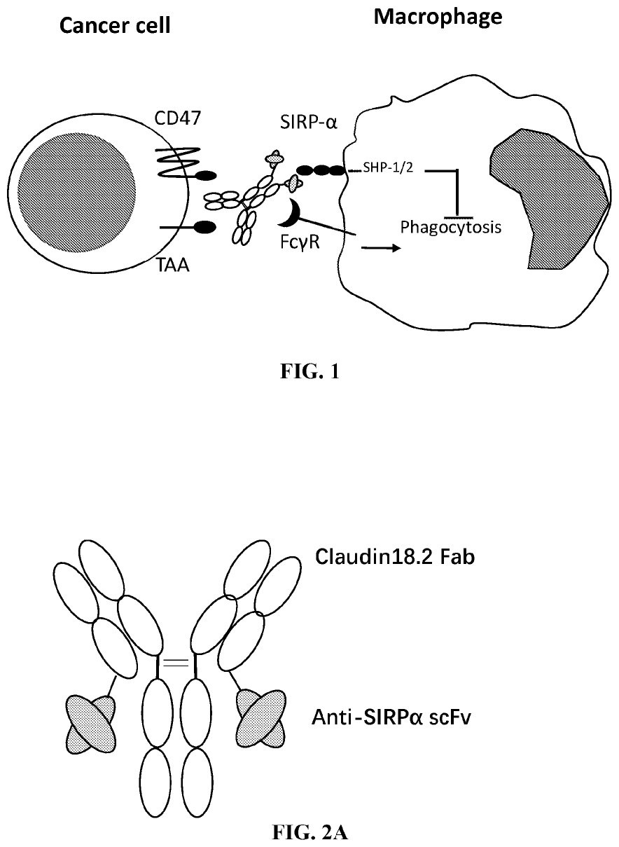

The principle of SIRPα based bispecific macrophage engager (BiME) antibodies is depicted in FIG. 1. The BiME can physically bridge macrophages to cancer cells for specific killing; blocking CD47-SIRPα interaction, removing SHP-1/2 inhibition and engaging Fc receptors on macrophages to activate phagocytosis.

Anti-Claudin18.2/SIRPα bispecific antibodies are constructed as an anti-Claudin18.2 antibody hu28.H1L2 fused with anti-SIRPα C25 single-chain variable fragment (scFv) at the C-terminus of heavy or light chain (FIG. 2A-B). A flexible (Gly4Ser)3 linker was genetically linked to the N-terminus of the anti-SIRPα scFv. Anti-Claudin18.2/SIRPα bispecific antibodies are constructed as an anti-SIRPα antibody fused with anti-Claudin18.2 single-chain variable fragment (scFv) at the C-terminus of heavy or light chain (FIG. 2C-D). A flexible (Gly4Ser)3 linker was genetically linked to the N-terminus of the anti-Claudin18.2 scFv.

The bispecific protein ES028-001 comprising one anti-Claudin18.2 antibody and two anti-SIRPα scFv at the C-terminus of light chain (FIG. 2A, see also, Table 7).

The bispecific protein ES028-005 comprising one anti-Claudin18.2 antibody and two anti-SIRPα scFv at the C-terminus of heavy chain (FIG. 2B, see also, Table 7).

The bispecific protein ES028-009 comprising one anti-SIRPα antibody and two anti-Claudin18.2 scFv at the C-terminus of light chain (FIG. 2C, see also, Table 7).

The bispecific protein ES028-013 comprising one anti-SIRPα antibody and two anti-Claudin18.2 scFv at the C-terminus of heavy chain (FIG. 2D, see also, Table 7).

For expression, the DNA encoding the light chain and the heavy chain in either the same expression vector or separate expression vectors were used to transfect HEK293 cell for transfection. The culture media were harvested and the fusion protein was purified by Protein A Sepharose column.

### Example 2 Anti-Claudin18.2/SIRPα Bispecific Antibody Binding Affinities

The Anti-Claudin18.2/SIRPα bispecific protein were characterized for binding affinity against human Claudin18.2 or SIRPα using Octet assay (ForeBio) according to manufacturer's manual, separately. Briefly, the antibodies were coupled on sensors and then the sensors were dipped into Claudin18.2 or SIRPα protein gradients (start at 200 nM, with 2-fold dilution and totally 8 doses). Their binding responses were measured in real-time and results were fit globally. The affinity data of the tested antibody are summarized in Table 8 and Table 9.

TABLE 8
Binding affinity of bispecific antibody
to human SIRPα as measured by Octet assay

| Sample | KD (M) | kon(1/Ms) | koff(1/s) |
|---|---|---|---|
| Anti- SIRPα, C25 | 2.12E−09 | 5.12E+05 | 1.09E−03 |
| ES028-001 | 1.51E−09 | 1.79E+05 | 2.71E−04 |
| ES028-005 | 1.41E−09 | 3.34E+05 | 4.70E−04 |
| ES028-009 | 2.44E−09 | 5.44E+05 | 1.33E−03 |

TABLE 9
Binding affinity of bispecific antibody to human
Claudin18.2 as measured by Octet assay

| Sample | KD (M) | kon(1/Ms) | koff(1/s) |
|---|---|---|---|
| Anti-Claudin18.2, | <1.0E−12 | 9.58E+04 | <1.0E−07 |
| hu28.H1L2 | | | |
| ES028-001 | <1.0E−12 | 1.05E+05 | <1.0E−07 |
| ES028-005 | <1.0E−12 | 1.06E+05 | <1.0E−07 |
| ES028-009 | <1.0E−12 | 2.11E+04 | <1.0E−07 |
| Anti-Claudin18.2, | 1.81E−09 | 3.45E+04 | 6.25E−05 |
| hu26.H1L2 (S92A) | | | |

### Example 3 Binding of Anti-Claudin18.2/SIRPα Bispecific Antibody to Claudin18.2 and SIRPα by FACS

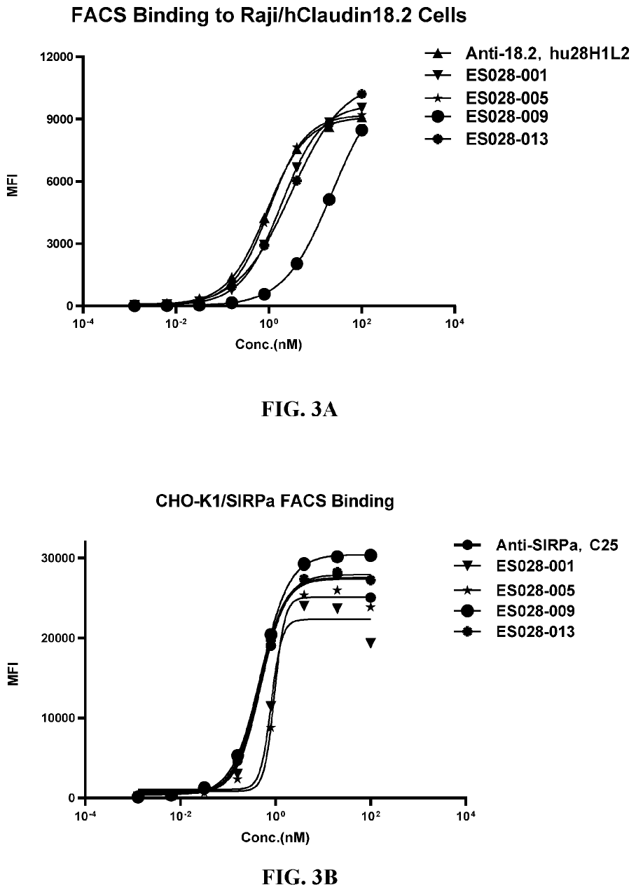

Approximately 100,000 Raji lymphoma cells overexpressing human Claudin18.2 were washed with wash buffer and incubated with 100 ul serial dilution of Claudin18.2/SIRPα bispecific protein for 30 minutes on ice. Cells were then washed twice with wash buffer and incubated with 100 ul of anti-human Fc-PE for 30 minutes on ice. Cells were then washed twice with wash buffer and analyzed on a FACS Canto II analyzer (BD Biosciences). As shown in FIG. 3A, the anti-Claudin18.2/SIRPα bispecific antibodies bound to Raji/hClaudin18.2 cell in a dose-dependent manner. The bispecific antibodies ES028-001, ES028-005, ES028-013 bound Raji/hClaudin18.2 similar to anti-Claudin18.2 monoclonal antibody hu28H1L2 whereas ES 028-009 bound Raji/hClaudin18.2 to a lesser extent relative to anti-Claudin18.2, hu28H1L2.

CHO-K1 cells overexpressing human SIRPα were washed with wash buffer and incubated with 100 ul serial dilution of Claudin18.2/SIRPα bispecific protein for 30 minutes on ice. Cells were then washed twice with wash buffer and incubated with 100 ul of anti-human Fc-PE for 30 minutes on ice. Cells were then washed twice with wash buffer and analyzed on a FACS Canto II analyzer (BD Biosciences). As shown in FIG. 3B, the anti-Claudin18.2/SIRPα bispecific antibodies bound to CHO-K1/SIRPα cell in a dose-dependent manner. The ES028-001 and ES028-005 bound CHO-K1/hSIRPα to a lesser extent relative to anti-SIRPα, C25.

### Example 4 Anti-Claudin18.2/SIRPα Bispecific Antibody Enhances In Vitro Macrophage Phagocytosis of Claudin18.2+ Cancer Cells

Mouse MC38 colon tumor cells expressing human CD47 and human Claudin18.2 were labeled with the fluorescent dye CFSE, and incubated with mouse bone marrow derived macrophages (BMDMs) prepared from C57BL6/hCD47/hSIRPα knock-in mice either in the presence of an isotype control, anti-Claudin18.2 antibody, anti-SIRPα antibody, the combination of anti-Claudin18.2 and anti-SIRPα antibody, anti-Claudin18.2/SIRPα bispecific antibody. After 2 hours, the macrophages were harvested, stained with a fluorescently labeled anti-mouse macrophage antibody, and analyzed by flow cytometry. CD11b+CFSE+ double positive events identify macrophages that have phagocytosed CFSE-labeled tumor cell. Each sample is represented by a different color. Phagocytic index is shown for three separate samples.

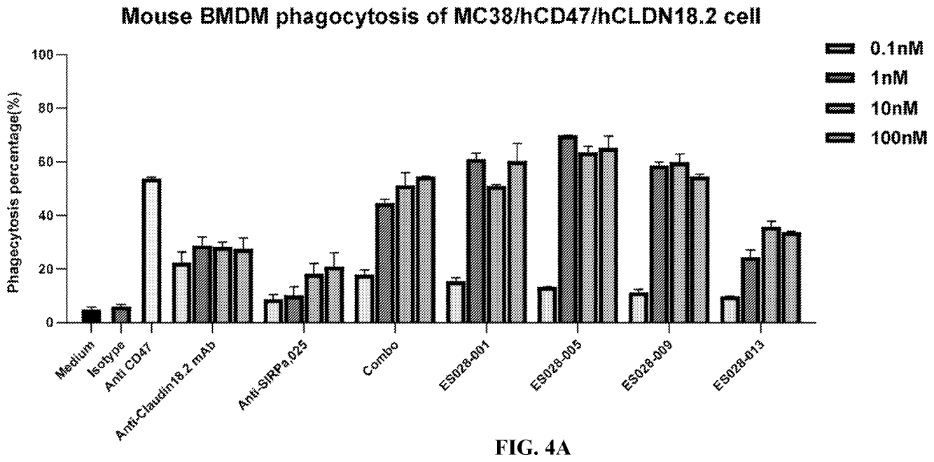

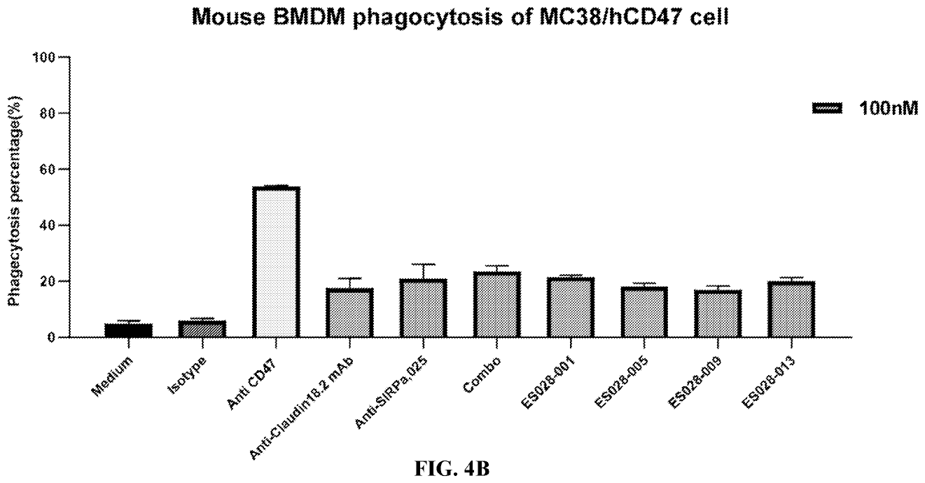

As shown in FIG. 4A, anti-Claudin18.2 antibody hu28H1L2 induced ˜25% phagocytosis by antibody dependent cellular phagocytosis (ADCP) and anti-SIRPα antibody C25 barely induce phagocytosis. The combination of anti-Claudin18.2 and anti-SIRPα antibody C25 significantly improve the phagocytosis. Anti-Claudin18.2/SIRPα bispecific antibody ES028-001, ES028-005, ES028-009 induce stronger phagocytosis in a dose dependent manner compared to combination and ES028-005 showing the best phagocytosis effect; ES028-013 didn't show synergistic phagocytosis effect. For MC38 cells that do not express Claudin18.2, the combination of anti-Claudin18.2 and anti-SIRPα antibody C25 do not significantly improve the phagocytosis against the Claudin18.2 non-expressing cells (FIG. 4B). Anti-Claudin18.2/SIRP bispecific antibodies or combination of anti-Claudin18.2 antibody and anti-SIRPalpha antibody do not induce improved phagocytosis on the cells that do not express Claudin18.2 either (FIG. 4B).

### Example 5 Anti-Claudin18.2/SIRPα Bispecific Antibody Active IgG1 Fe Activates FcγR-Dependent ADCP and Provides Stronger Potency than IgG4 or IgG1LALA Isotype

In another mouse BMDM phagocytosis assay of MC38/hCD47/hClaudin18.2, anti-Claudin18.2 antibody with IgG1, IgG4 and IgG1LALA different isotypes were tested monotreatment or combination with anti-SIRPα antibody. In similar, anti-Claudin18.2/SIRPα bispecific antibody ES028-001, ES028-005 and ES028-009 with IgG1, IgG4 and IgG1LALA were also tested for phagocytosis.

After co-culture for 2 hours, the macrophages were harvested, stained with a fluorescently labeled anti-human macrophage antibody, and analyzed by flow cytometry. CD11b+CFSE+ double positive events identify macrophages that have phagocytosed CFSE-labeled tumor cell. Each sample is represented by a different color. Phagocytic index is shown for three separate samples.

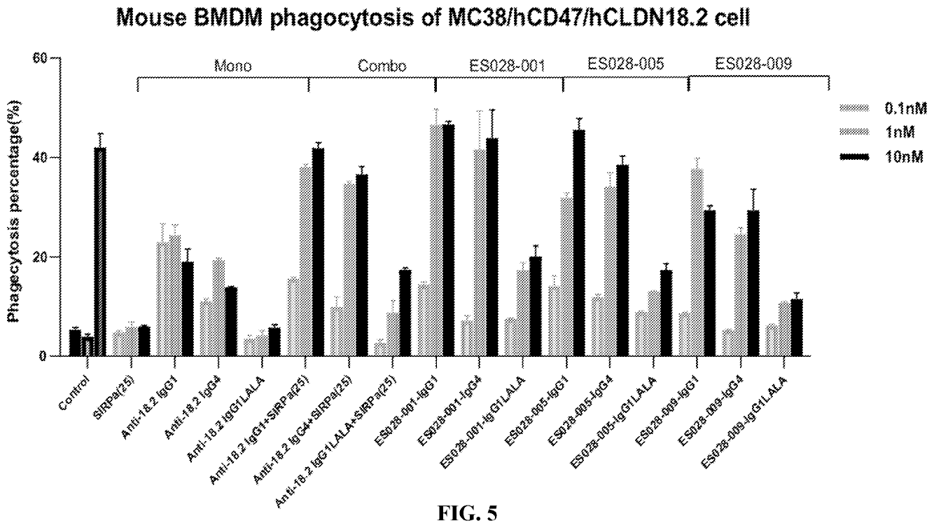

As shown in FIG. 5, anti-Claudin18.2 IgG1 showed the best phagocytosis compared to anti-Claudin18.2 IgG4 and anti-Claudin18.2 IgG1LALA mutant both in monotreatment and combination with anti-SIRPα antibody. The anti-Claudin18.2/SIRPα bispecific antibody ES028-001 IgG1, ES028-005 IgG1 and ES028-009 IgG1 induce stronger phagocytosis effect compared to ES028-001 IgG4, ES028-005 IgG4 and ES028-009 IgG4 whereas there is no phagocytosis efficacy for anti-Claudin18.2/SIRPα bispecific antibody ES028-001 IgG1LALA, ES028-005 IgG1LALA and ES028-009 IgG1LALA. The data exhibited that anti-Claudin18.2/SIRPα bispecific antibody IgG1 activates FcγR-dependent ADCP and provides stronger potency than IgG4 or IgG1LALA Isotype.

### Example 6 Anti-PDL1/SIRPα Bispecific Antibody Construction and Expression

Anti-PDL1/SIRPα bispecific antibodies are constructed as an anti-SIRPα antibody C25 fused with anti-PDL1 single domain antibody (sdAb) C71 or C570 at the N-terminus or C-terminus of heavy or light chain (FIG. 6A-D, see also, Table 7). The Anti-PDL1/SIRPα bispecific antibodies can also be constructed anti-SIRPα Fab in one arm and two copied of anti-PDL1 sdAb in another arm linked by knob-in hole (KiH) in Fc region (FIG. 6E, see also, Table 7). The Anti-PDL1/SIRPα bispecific antibodies can also be constructed as anti-PDL1 sdAb in N-terminus of Fc and anti-SIRPα Fab fused to C-terminus of Fe (FIG. 6F-G, see also, Table 7). A flexible (Gly4Ser)3 linker was genetically linked to the N-terminus of the anti-SIRPα Fab.

The bispecific protein ES019-020 comprising one anti-SIRPα antibody and two anti-PDL1 sdAb at the C-terminus of light chain (FIG. 6A, see also, Table 7).

The bispecific protein ES019-024 comprising one anti-SIRPα antibody and two anti-PDL1 sdAb at the C-terminus of heavy chain (FIG. 6B, see also, Table 7).

The bispecific protein ES019-025 comprising one anti-SIRPα antibody and two anti-PDL1 sdAb at the N-terminus of heavy chain (FIG. 6C, see also, Table 7).

The bispecific protein ES019-026 comprising one anti-SIRPα antibody and two anti-PDL1 sdAb at the N-terminus of light chain (FIG. 6D, see also, Table 7).

The bispecific protein ES019-029 comprising one anti-SIRPα Fab arm and two anti-PDL1 sdAb arm heterodimer by knob-in hole mutations in Fc region (FIG. 6E, see also, Table 7).

The bispecific protein ES019-072 comprising two anti-PDL1 sdAbs in N-terminus of Fc and one copy of anti-SIRPα Fab in C-terminus of Fc region. The asymmetric heterodimer is formed by knob-in hole mutations in Fc region (FIG. 6F, see also, Table 7).

The bispecific protein ES019-073 or ES019-079 comprising two anti-PDL1 sdAbs in N-terminus of Fc and two anti-SIRPα Fabs in C-terminus of Fc region (FIG. 6G, see also, Table 7).

### Example 7: Anti-PDL1/SIRPα Bispecific Antibody Binding Affinities

The Anti-PDL1/SIRPα bispecific protein were characterized for binding affinity against human PDL1 using Octet assay (ForeBio) according to manufacturer's manual, separately. Briefly, the antibodies were coupled on sensors and then the sensors were dipped into PDL1 protein gradients (start at 200 nM, with 2-fold dilution and totally 8 doses). Their binding responses were measured in real-time and results were fit globally. The affinity data of the tested antibody are summarized in Table 9.1.

TABLE 9.1
Binding affinity of bispecific antibody
to human PDL1 as measured by Octet assay

| Sample | KD (M) | kon(1/Ms) | koff(1/s) |
|---|---|---|---|
| Anti-PDL1, C71 | 5.31E−09 | 9.42E+05 | 5.01E−03 |
| Anti-PDL1, C570 | 3.74E−10 | 8.19E+05 | 3.06E−04 |
| Anti-PDL1, 570h3 | 1.98E−10 | 4.70E+05 | 9.29E−05 |
| ES019-020 | 5.54E−09 | 9.18E+05 | 5.09E−03 |
| ES019-024 | 6.56E−09 | 9.20E+05 | 6.03E−03 |
| ES019-025 | 3.81E−09 | 1.58E+06 | 6.04E−03 |
| ES019-026 | 4.10E−09 | 1.56E+06 | 6.39E−03 |
| ES019-029 | 1.23E−09 | 1.11E+06 | 1.36E−03 |
| ES019-079 | 4.85E−11 | 5.88E+05 | 2.85E−05 |

### Example 8: Binding of Anti-PDL1/SIRPα Bispecific Antibody to PDL1 and SIRPα by FACS

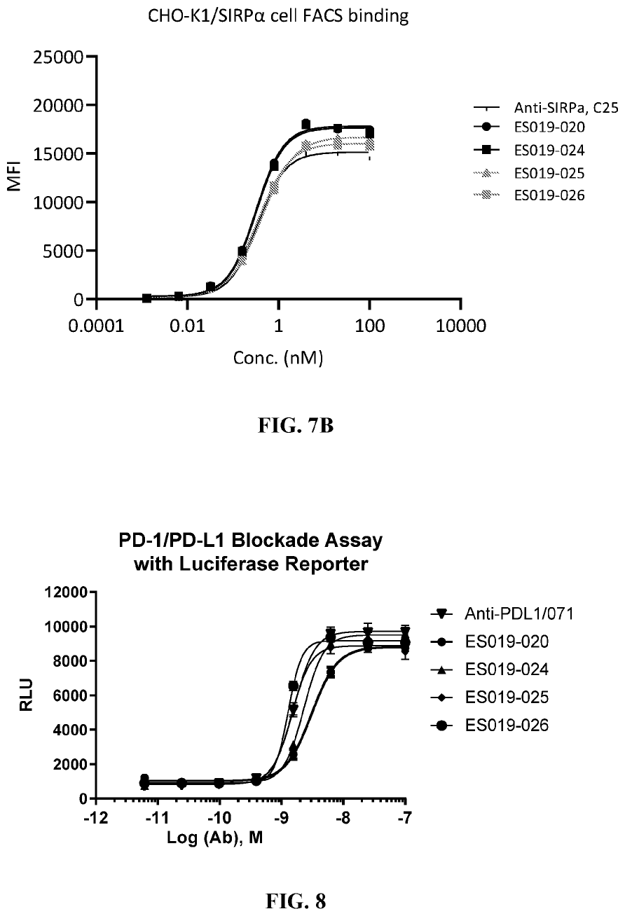

Approximately 100,000 Raji cells overexpressing human PDL1 were washed with wash buffer and incubated with 100 ul serial dilution of PDL1/SIRPα bispecific protein for 30 minutes on ice. Cells were then washed twice with wash buffer and incubated with 100 ul of anti-human Fc-PE for 30 minutes on ice. Cells were then washed twice with wash buffer and analyzed on a FACS Canto II analyzer (BD Biosciences). As shown in FIG. 7A, the anti-PDL1/SIRPα bispecific antibodies bound to Raji/hPDL1 cell in a dose-dependent manner. The bispecific antibodies ES019-025, ES019-026 bound Raji/hPDL1 similar to anti-PDL1 monoclonal antibody C71 whereas ES019-020, ES019-024 bound Raji/hPDL1 to a lesser extent relative to anti-PDL1, C71.

CHO-K1 cells overexpressing human SIRPα were washed with wash buffer and incubated with 100 ul serial dilution of PDL1/SIRPα bispecific protein for 30 minutes on ice. Cells were then washed twice with wash buffer and incubated with 100 ul of anti-human Fc-PE for 30 minutes on ice. Cells were then washed twice with wash buffer and analyzed on a FACS Canto II analyzer (BD Biosciences). As shown in FIG. 7B, the anti-PDL1/SIRPα bispecific antibodies bound to CHO-K1/SIRPα cell in a dose-dependent manner. The ES019-020, ES019-024, ES019-025 and ES019-026 bound CHO-K1/hSIRPα similar to anti-SIRPα, C25.

### Example 9: PDL1 Blocking Activity of Anti-PDL1/SIRPα Bispecific Antibody by Jurkat/PD1 Reporter Cell Assay

The PD1-PDL1 blockade reporter assays were performed using the PD1 blockade reporter assay developed by GenScript that uses a two cell system based on CHO cells that expressed PDL1 and anti-CD3 scFv and a Jurkat/NFAT-RE Reporter Cell Line overexpressing PD1. Cells growing in logarithmic phase were harvested and resuspended in RPMI 1640 20 containing 1% heat inactivated FBS) at a concentration of 2×106 cells/ml CHO and 4×106 cells/ml for Jurkat/PD1 NFAT-RE cells. Next, test antibodies in assay medium (RPMI 1640 containing 1% FBS) in a serial dilution were added to each well. Plates were incubated for 6 hours at 37° C., 5% C02, in 95% relative humidity. 40 ul of luciferase (Bio-Glo Luciferase Assay System) was added the next day and the amount of luciferase activity was measured using BioTek Multi-Mode Microplate Reader.

As shown in FIG. 8, the bispecific antibodies ES019-024, ES019-025, ES019-026 activate Jurkat/PD1 reporter cell similar to anti-PDL1 monoclonal antibody C71 whereas ES019-020 activate Jurkat/PD1 reporter cell to a lesser extent relative to anti-PDL1, C71.

### Example 10: Anti-PDL1/SIRPα Bispecific Antibody Enhances In Vitro Macrophage Phagocytosis of PDL1+ Cancer Cells

Human K562 leukemia tumor cells that lacks of human PDL1 expression, or K562 expressing human PDL1 were labeled with the fluorescent dye CFSE, and incubated with human macrophage-colony stimulating factor (M-CSF) treated monocyte derived macrophage either in the presence of an isotype control, anti-PDL1 antibody, anti-SIRPα antibody, the combination of anti-PDL1 and anti-SIRPα antibody, anti-PDL1/SIRPα bispecific antibody. After 2 hours, the macrophages were harvested, stained with a fluorescently labeled anti-human macrophage antibody, and analyzed by flow cytometry. CD11b+CFSE+ double positive events identify macrophages that have phagocytosed CFSE-labeled tumor cell. Each sample is represented by a different color. Phagocytic index is shown for three separate samples.

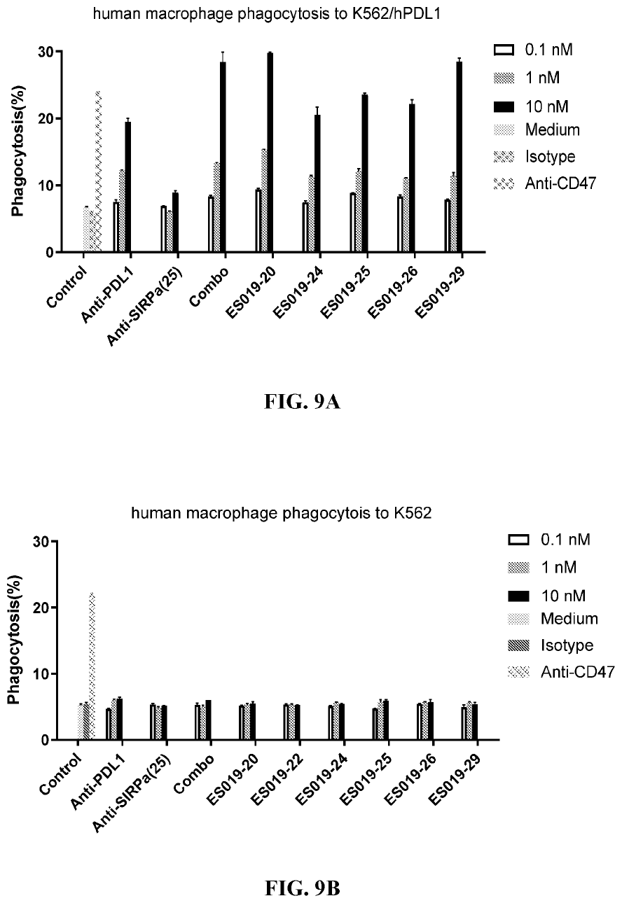

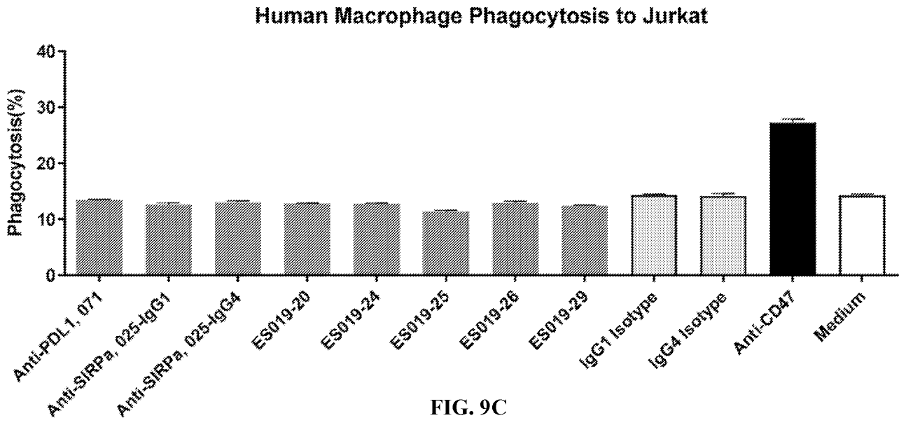

As shown in FIG. 9A, anti-PDL1 antibody C71 induced ˜20% phagocytosis by antibody dependent cellular phagocytosis (ADCP) and anti-SIRPα antibody C25 barely induce phagocytosis. The combination of anti-PDL1 and anti-SIRPα antibody C25 significantly improve the phagocytosis. Anti-PDL1/SIRPα bispecific antibody ES019-020, ES019-029 induce strong phagocytosis in a dose dependent manner similar to combination; ES019-024, ES019-025 and ES019-026 didn't show synergistic phagocytosis effect.

As shown in FIG. 9B, anti-PDL1 antibody C71 monotherapy or combination with anti-SIRPα antibody C25 barely induce phagocytosis against K562 parental cell that lacks of PDL1 expression. Anti-PDL1/SIRPα bispecific antibody ES019-020, ES019-024, ES019-025, ES019-026, ES019-029 didn't show phagocytosis effect against K562 cell (PDL1 negative).

As shown in FIG. 9C, anti-PDL1 antibody C71 monotherapy or combination with anti-SIRPα antibody C25 barely induce phagocytosis against human Jurkat T cell line that express human SIRPγ. Anti-PDL1/SIRPα bispecific antibody ES019-020, ES019-024, ES019-025, ES019-026, ES019-029 didn't show phagocytosis effect against Jurkat T cell (SIRPγ positive).

### Example 11: Anti-Claudin18.2/SIRPα Bispecific Antibody Enhances In Vitro Macrophage Phagocytosis of PDL1+ Cancer Cells

Human SIRPα/CD47 double KI mice were inoculated with hCD47/hCLDN18.2 overexpression MC38 cells. Mice were grouped according to tumor volume into 7 groups when the mean tumor volume reached ˜70-100 mm3. Mice were i.p. dosed with the same molar concentration of 10 mpk isotype antibody, 10 mpk CLDN18.2 mAb, 10 mpk SIRPα mAb, 10 mpk CLDN18.2 mAb plus 10 mpk SIRPα mAb or 14mpk ES028-001, ES028-005, ES009. Dosing schedule was BIW for 5 doses. Tumor volume were measured twice per week. 3 days post the 5th dosing, mice were sacrificed and tumors were weighted. Statistics were carried out by 2-way anova comparing the mean tumor volume of different treatment groups to that of isotype control group.

Relative tumor inhibition rate, TGI (%) was calculated as below:

$$TGI \% = ( 1 - T / C ) \times 100 \% . ( T and C are the relative tumor volume ( RTV ) or tumor weight ( TW ) of the treatment group and the control group at a specific time point , respectively ) .$$

$$T / C \% = TRTV / CRTV \times 100 \% ( TRTV : mean RTV of the treatment group ; CRTV : mean RTV of the vehicle control group ;$$

$$RTV = Vt / V 0 , V 0 is the tumor volume of the animal at the time of grouping , Vt is the tumor volume of animal after treatment ) ;$$

T/C can also be calculated based tumor weight as below:

$$T / C \% = TTW / CTW \times 100 \% ( TTW : mean tumor weight of the treatment group at the end ; \\ CTW : mean tumor weight of the vehicle control group at the end ) .$$

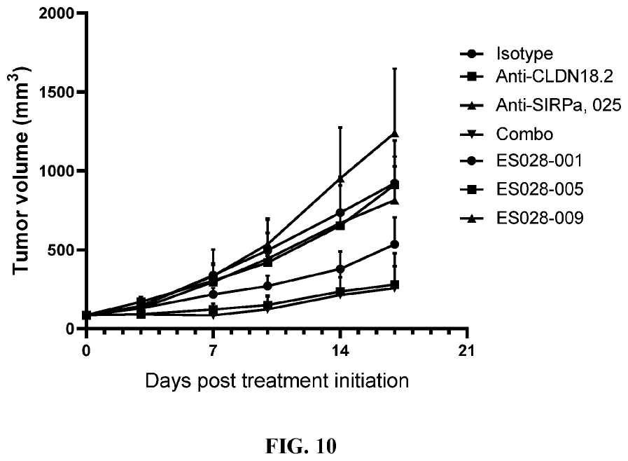

As shown in FIG. 10, anti-Claudin18.2 antibody hu28H1L2 and anti-SIRPα antibody C25 monotherapy do not inhibit tumor growth similar to isotype treatment. The combination of anti-Claudin18.2 and anti-SIRPα antibody C25 significantly reduce the growth of tumors. Anti-Claudin18.2/SIRPα bispecific antibody ES028-005, induce strong tumor growth inhibition similar to combination; ES028-001 show weaker tumor inhibition and ES028-009 didn't inhibit tumor growth.

### Example 12: Anti-SIRPα Complete Blocking Antibody Based BiME Provides Best Synergistic Phagocytosis Effect with Selectivity Against Tumor Cell

### Characterization of Chimeric Antibodies

### 1.1 Binding Specificity Detection

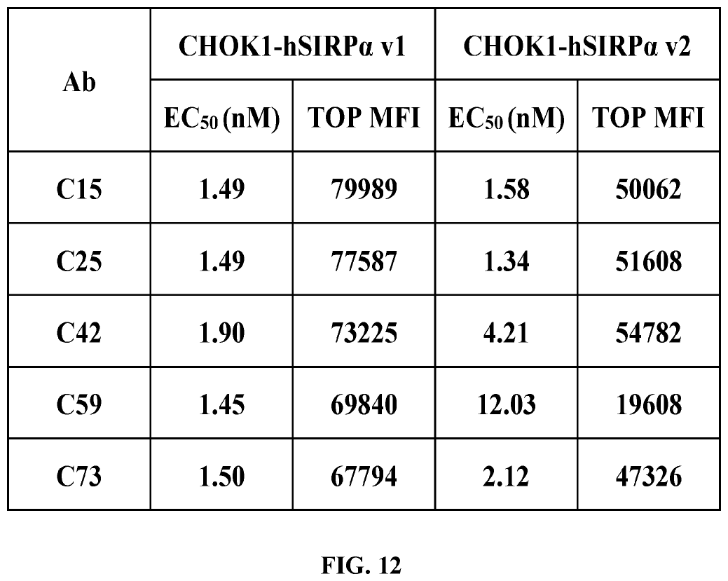

Binding activity of the purified chimeric antibodies against human SIRPα variants was detected by FACS assay using CHOK1 cells or 293F cells stably expressing human SIRPα v1 and CHOK1 cells stably expressing human SIRPα v2. As shown in FIG. 12, all antibodies as tested strongly bind to cell surface human SIRPα v1 and cell surface human SIRPα v2. EC₅₀ and top signal calculated using GraphPad Prism9.0.

### 1.2 CD47/SIRPα Interaction Blocking Activity Detection

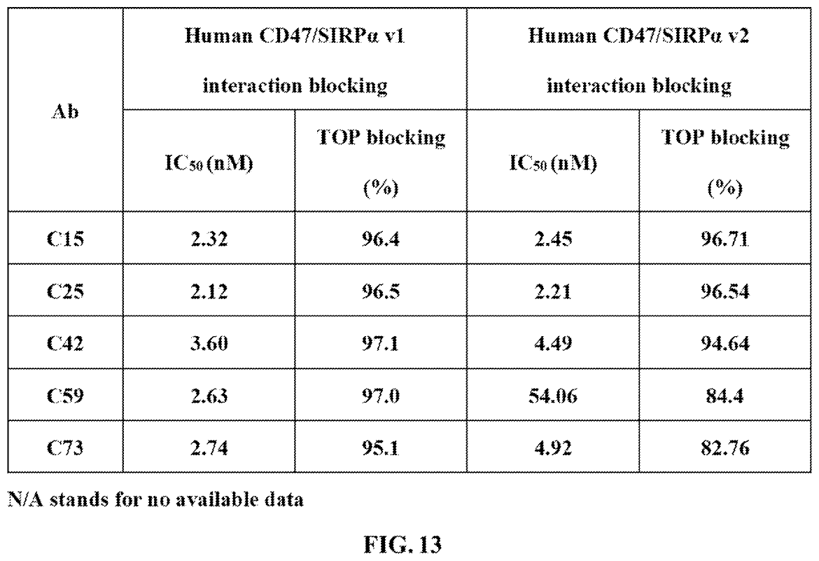

Competitive ELISA assay was used to determine whether the purified chimeric antibodies can block CD47 and SIRPα interaction. Briefly, antibody and mFc tagged human CD47 ECD recombinant protein were co-incubated with ELISA microplate coated human SIRPα v1 ECD or human SIRPα v2 ECD recombinant protein. The concentration of soluble ECD of CD47 is 25 nM, and the concentration of soluble ECD of SIRPα is 20 nM. After washing, HRP labeled anti-mouse Fc 2^{nd} antibody (Sigma) was added and incubated at 37° C. for 1 hour. Then, 100 μl/well of TMB solution (Biotechnology) was added. After incubation for 15 minutes at room temperature, the reaction was stopped by the addition of 50 μl of 1N HCl. OD 450 nm was read. Blocking ratio was determined by blockade of human SIRPα ECD recombinant protein binding to ELISA microplate coated human CD47 ECD recombinant protein. IC₅₀ and top blocking percentage calculated using GraphPad Prism9.0 are summarized in FIG. 13. All antibodies as tested can block interaction between human CD47 and different human SIRPα variants.

### 1.2.1 Epitope Analysis

Competitive ELISA assay was used for epitope binning of purified chimeric antibodies. Briefly excessive competitor antibody and mFc tagged human SIRPα v1 ECD recombinant protein were co-incubated with ELISA microplate coated antibody. After washing, HRP labeled anti-mouse Fc 2^{nd} antibody (Sigma) was added and incubated at 37° C. for 1 hour. Then, 100 μl/well of TMB solution (Biotechnology) was added. After incubation for 15 minutes at room temperature, the reaction was stopped by the addition of 50 μl of 1N HCl. OD 450 nm was read. Competition ratio was calculated. The antibodies that can compete each other for binding to SIRPα may have related binding epitopes. As shown in Table B, 025c didn't show competitive binding to human SIRPα with 042c, 073c and hu1H9G4, indicating that it may bind to a distinct epitope. Competition between 042c, 073c and hu1H9G4 is not bidirectional, indicating that their binding epitopes may be related but not completely identical.

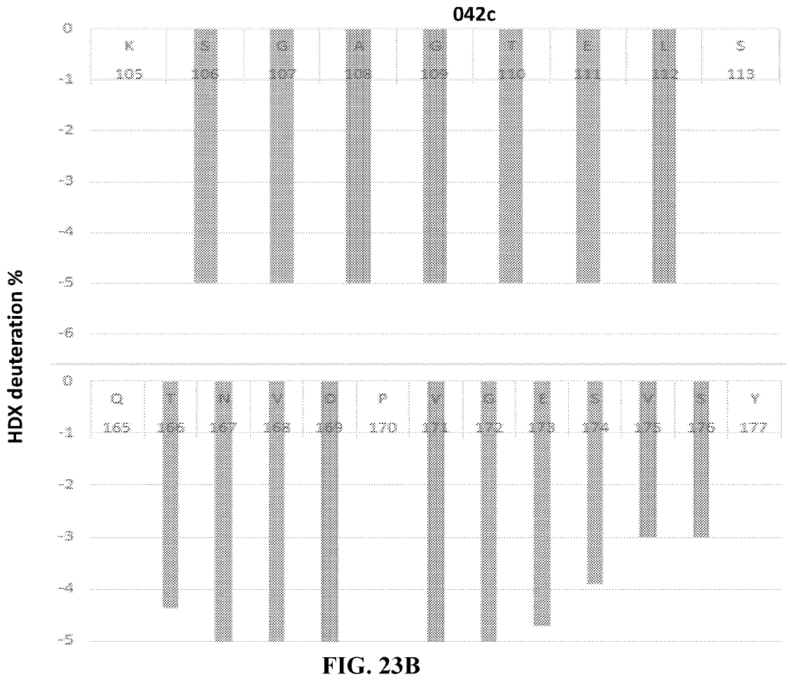

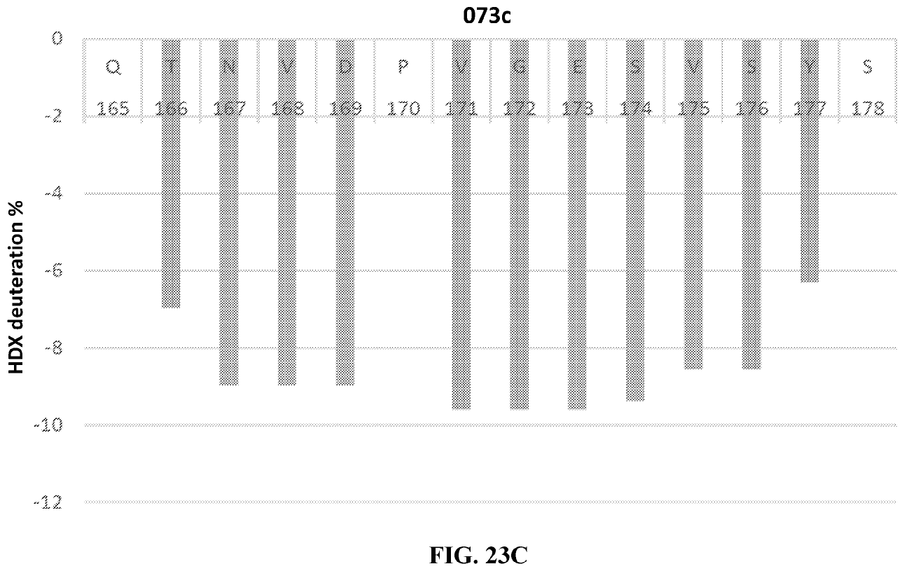

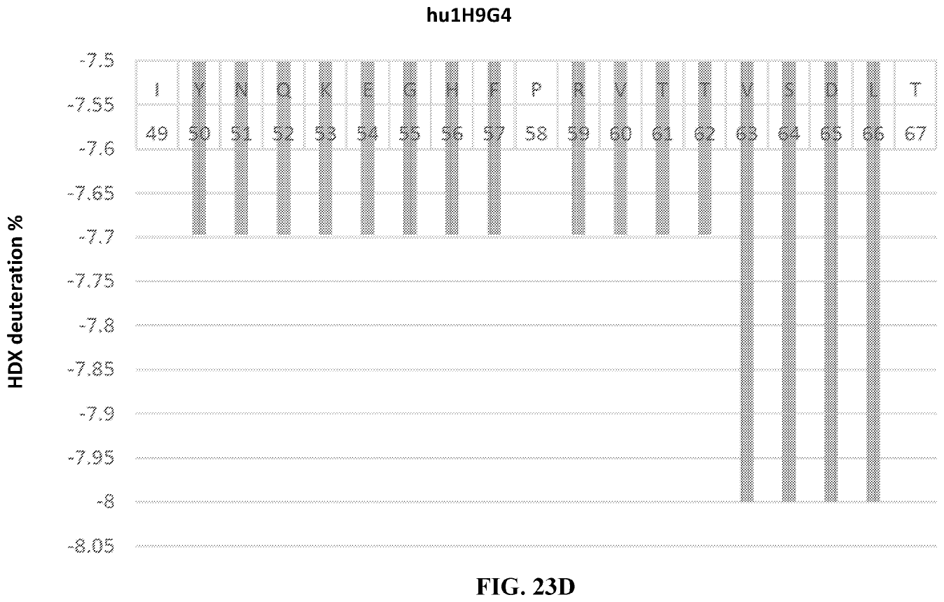

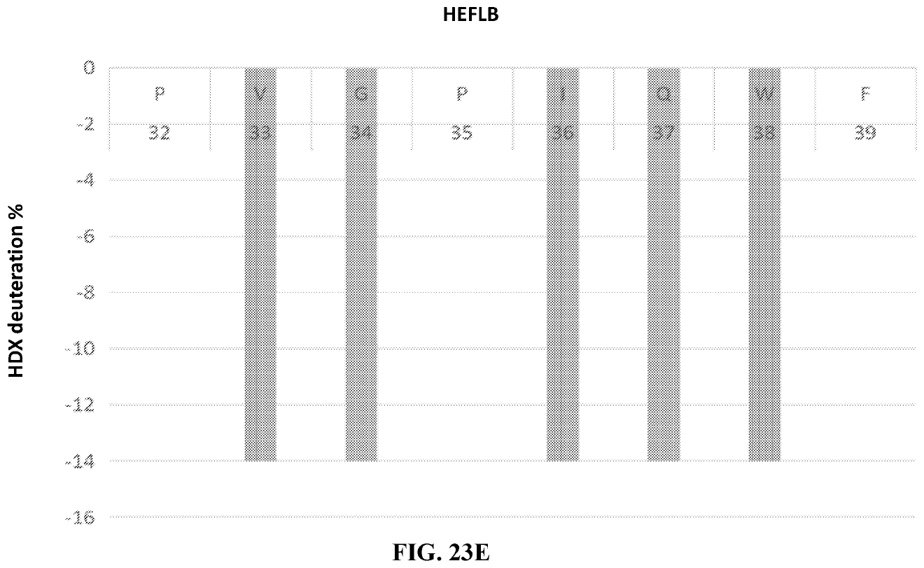

Epitope mapping of 025c, 042c, 073c, HEFLB and hu1H9G4 were further carried out using hydrogen deuterium exchange mass spectrometry (HDX-MS). As shown in FIG. 23A, 025c binding resulted in less hydrogen deuterium exchange ratio of the region of YNQKEGHFPRVTTVSDL (SEQ ID NO: 218) of His tagged human SIRPα v1 ECD, indicating these amino acids may be critical for 025c to bind. As shown in FIG. 23B, 042c binding resulted in less hydrogen deuterium exchange ratio of 2 regions of SGAGTEL (SEQ ID NO: 219) and TNVDPVGESVS (SEQ ID NO: 220) of His tagged human SIRPα v1 ECD, indicating these amino acids may be critical for 042c to bind. As shown in FIG. 23C, 073c binding resulted in less hydrogen deuterium exchange ratio of the region of TNVDPVGESVSY (SEQ ID NO: 221) of His tagged human SIRPα v1 ECD, indicating these amino acids may be critical for 073c to bind. In particular, these 3 regions are not located in IgV domain of SIRPα ECD where CD47 binds to, indicating 042c and 073c may work as allosteric antibody to block interaction of CD47 and SIRPα or blocking activity of 042c and 073c is steric hindrance effect.

As shown in FIG. 23D, hu1H9G4 binding resulted in less hydrogen deuterium exchange ratio of the region of YNQKEGHFPRVTTVSDL (SEQ ID NO: 218) of His tagged human SIRPα v1 ECD, indicating these amino acids may be critical for hu1H9G4 to bind. As shown in FIG. 23E, HEFLB binding resulted in less hydrogen deuterium exchange ratio of the region of VGPIQW (SEQ ID NO: 222) of his tagged human SIRPα v1 ECD, indicating these amino acids may be critical for HEFLB to bind.

Taking competitive ELISA data and HDX-MS data together, it is concluded 025c, 042c and 073c may have distinct binding epitopes, which are also different from reference antibodies of hu1H9G4 and HEFLB. In addition, the results show that the epitopes of 025c, 042c and 073c may be outside the IgV domain. The IgV domain is responsible for the binding of the extracellular Ig-domain of CD47. The sequence information of the reference antibodies are shown in Table A below.

TABLE A
Variable region amino acid sequences of HEFLB and hu1H9G4

| Antibody | VH | VL |
|---|---|---|
| HEFLB | SEQ ID NO: 214 | SEQ ID NO: 215 |
| | EVQLVQSGAEVKKPGESLRISC | DVVMTQSPLSLPVTLGQPAS |
| | KASGYSFTSYWVHWVRQMPG | ISCRSSQSLVHSYGNTYLYW |
| | KGLEWMGNIDPSDSDTHYSPSF | FQQRPGQSPRLLIYRVSNRFS |
| | QGHVTLSVDKSISTAYLQLSSL | GVPDRFSGSGSGTDFTLKISR |
| | KASDTAMYYCVRGGTGTLAYF | VEAEDVGVYYCFQGTHVPY |
| | AYWGQGTLVTVSS | TFGGGTKVEIK |
| hu1H9G4 | SEQ ID NO: 216 | SEQ ID NO: 217 |
| | QVQLVQSGAEVKKPGASVKVS | DIQMTQSPSSLSASVGDRVTI |
| | CKASGYTFTSYWITWVKQAPG | TCRASENIYSYLAWYQQKP |
| | QGLEWIGDIYPGSGSTNHIEKF | GKAPKLLIYTAKTLAEGVPS |
| | KSKATLTVDTSISTAYMELSRL | RFSGSGSGTDFTLTISSLQPE |
| | RSDDTAVYYCATGYGSSYGYF | DFATYYCQHQYGPPFTFGQ |
| | DYWGQGTLVTVSS | GTKLEIK |

TABLE B
anti-SIRPα chimeric antibodies epitope binning summary
% Competition

| | Coating mAbs | | |
|---|---|---|---|
| Competitors | 025c | 042c | hu1H9G4 |
| 025c | 93.5 | 19.1 | 15.3 |
| 042c | 8.9 | 93.9 | 29.8 |
| hu1H9G4 | 14.5 | 82.6 | 90.5 |
| 073c | 9.1 | 93.6 | 37.7 |

### 1.3 SHP-1 Recruitment Assay

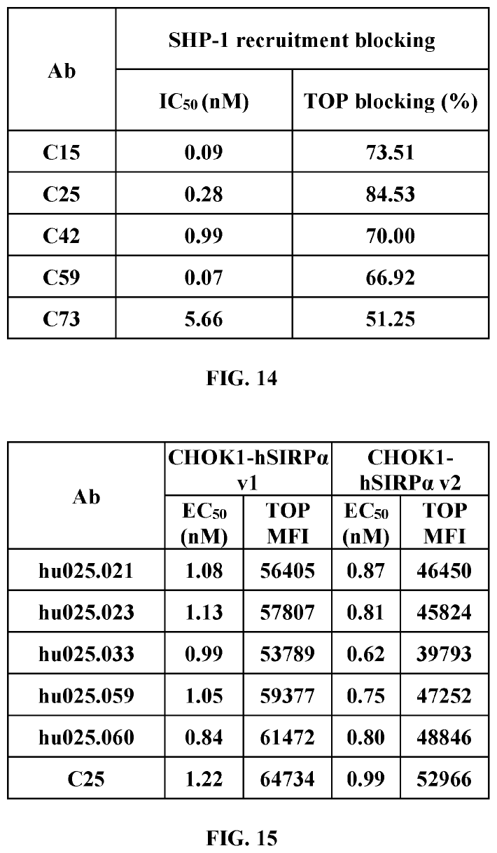

The efficacy of the purified chimeric antibodies to block CD47/SIRPα mediated “don't eat me” signaling was assessed by cell-based SHP-1 recruitment assay. Full length human SIRPα v1 was engineered with a small beta-gal fragment (ED) fused to its C-terminal, and the SH2-domain of SHP-1 was engineered with the complementing beta-gal fragment (EA). These constructs were stably expressed in K562 cells. Ligand engagement, through co-culture with human CD47 expressing cells, results in phosphorylation of SIRPα-ED fusion protein, leading to the recruitment of SHP-1-EA which forces to create an active beta-gal enzyme. This active enzyme hydrolyzes substrate to create chemiluminescence as a measure of reporter activity. IC₅₀ and top blocking percentage were calculated using GraphPad Prism9.0. As summarized in FIG. 14, all antibodies as tested can disrupt CD47/SIRPα mediated “don't eat me” signaling at different levels.

### 1.4 In Vitro Phagocytosis Assay

The function efficacy of the purified chimeric antibodies was assessed by a flow cytometry based phagocytosis assay. Briefly, M0 nonpolarized or M1 polarized human monocyte derived macrophages with different SIRPA genotypes were co-cultured with CellTrace Violet (Life Technologies) labeled CD47 expressing cancer cells in the presence of antibodies as tested. Phagocytosis was assayed by determining the percentage of macrophages positive for cell trace violet dye. For nonpolarized macrophages, peripheral blood mononuclear cells were seeded into 10 cm tissue culture plates in 1640 supplemented with 10% FBS and 50 ng/ml M-CSF for seven to nine days. Adherent cells were harvested as M0 nonpolarized macrophages. For M1 polarized macrophages, peripheral blood mononuclear cells were seeded into 10 cm tissue culture plates in 1640 supplemented with 10% FBS and 50 ng/ml GM-CSF for 5 days. 50 ug/ml IFNγ and 100 ug/ml LPS were added for additional two to four days culture. Adherent cells were harvested as M1 polarized macrophages.

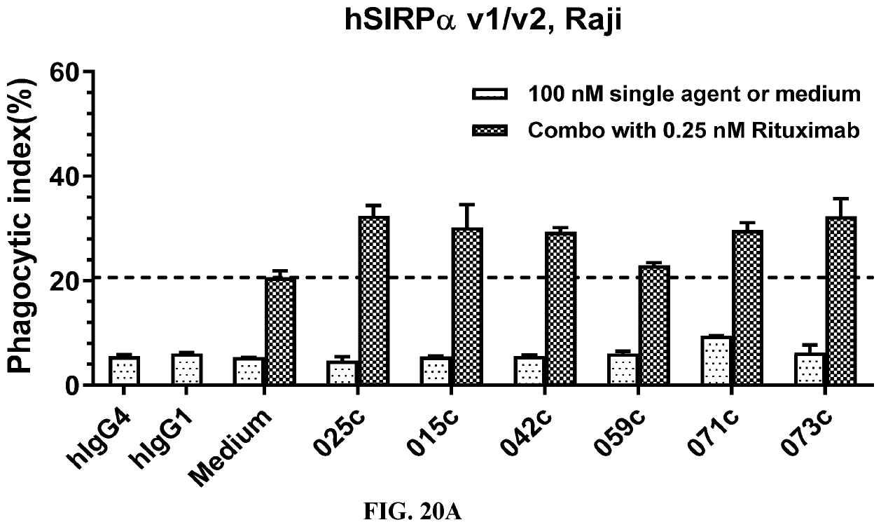

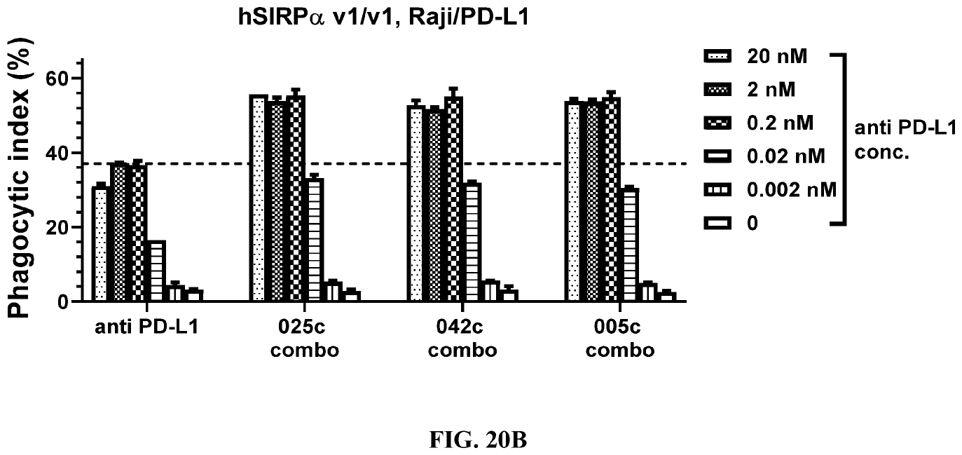

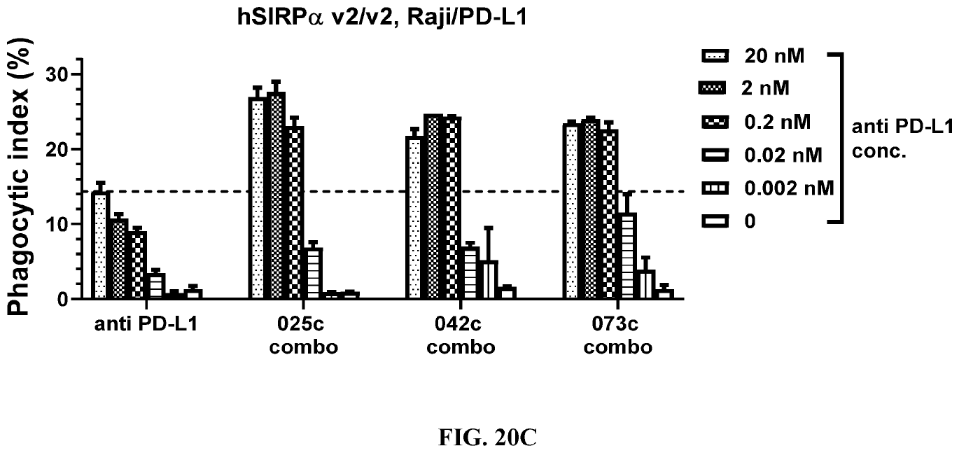

As shown in FIG. 20A, 015c, 025c, 042c, 059c and 073c showed no single agent activity to enhance tumor cell uptake of Raji cells by M0 macrophages obtained from SIRPA heterozygous v1/v2 individual. However, in the presence of Rituximab (an anti-CD20 antibody), other than 059c that has weaker activity to block interaction between human CD47 and human SIRPα v2, all the other purified chimeric antibodies as tested potentiated macrophage mediated antibody dependent cellular phagocytosis (ADCP) of Raji cells.

Combination of SIRPα antibody and PD-L1 antibody was tested in phagocytosis assay using M0 macrophages obtained from SIRPA homozygous v1/v1 (FIG. 20B) and v2/v2 (FIG. 20C) individuals. In the presence of PD-L1 antibody, 025c, 042c and 073c effectively potentiated macrophage mediated ADCP of Raji cells stably expressing PD-L1.

### Characterization of Humanized Antibodies

### 1.5 Binding Specificity Detection

All the humanized antibodies as tested were confirmed to retain the similar activity as the parental antibody of C25 to bind to SIRP family members. EC₅₀ and top signal calculated using GraphPad Prism9.0 are summarized in FIG. 15. Accordingly, it can be expected that all the humanized antibodies (e.g., hu025.021, hu025.033, hu025.023, hu025.059, hu025.060, hu26.H1L1, hu26.H1L2, hu26.H1L2 (S92A), C71, C71v38, C239, C492, C570, 570h3, C446, C2811, C1778, C1793, C2855, C2713 and C2719) provided herein have similar activity as the corresponding parental antibodies respectively.

### 1.5.1 Affinity Detection

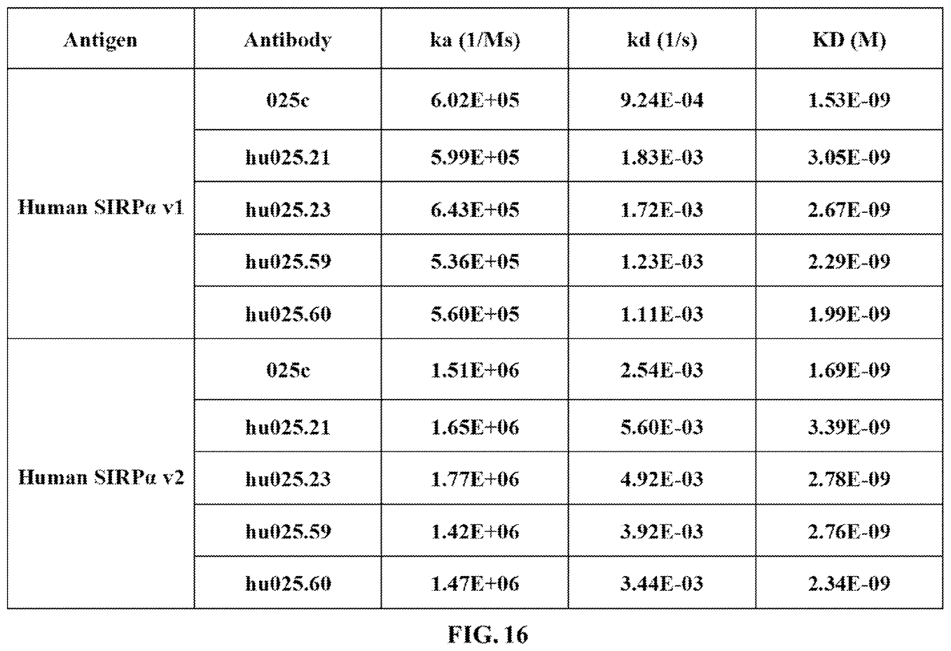

The humanized antibodies were characterized for binding affinity against human SIRPα v1, human SIRPα v2 using Surface Plasmon Resonance technology (Biacore system). The association and dissociation curves were fit with 1:1 binding model, and the Ka/Kd/KD values for each antibody were calculated. The affinity data of Ka/Kd/KD values for each antibody are summarized in FIG. 16.

### 1.7 CD47/SIRPα Interaction Blocking Activity Detection

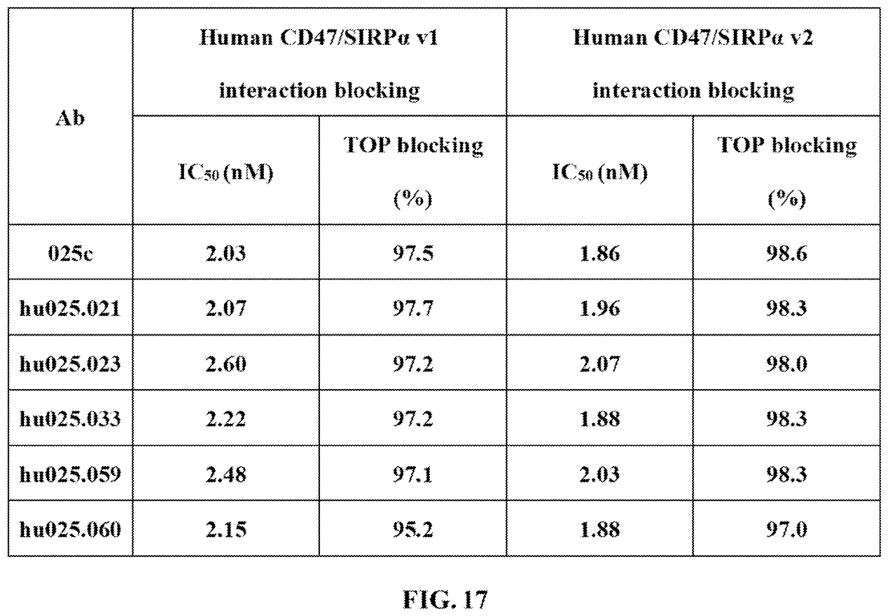

The humanized antibodies were tested for the ability to block CD47 and SIRPα interaction with competitive ELISA assay (refer to methods described above). As shown in FIG. 17, all the humanized antibodies as tested were confirmed to retain the similar activity as parental antibody of C25 to block interaction between human CD47 and different human SIRPα variants. IC₅₀ and top blocking percentage calculated using GraphPad Prism9.0 are summarized in FIG. 17.

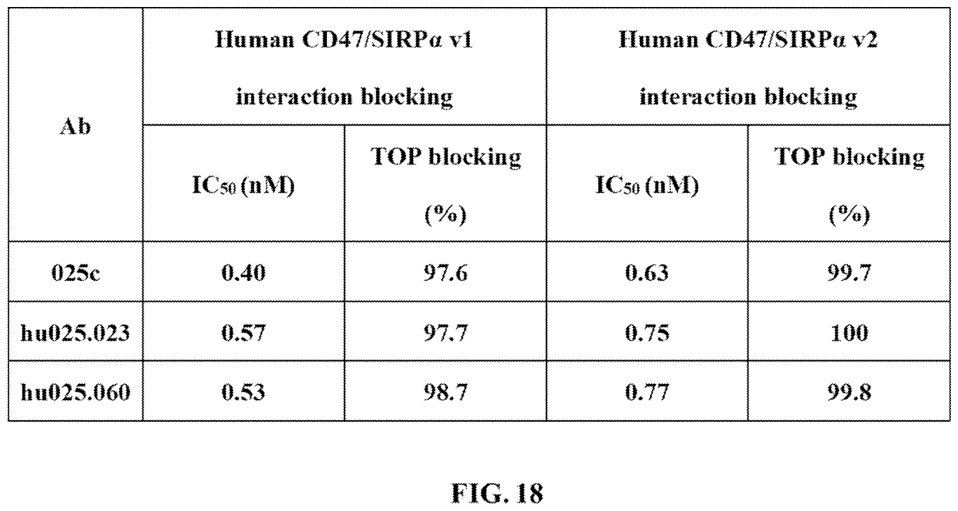

Competitive FACS assay was also set up to further compare the blocking activity of the humanized antibodies and some known anti-SIRPα antibodies. Briefly antibody and mFc tagged human CD47 ECD recombinant protein were co-incubated with CHOK1 cells stably expressing human SIRPα v1 or human SIRPα v2. After washing, dye labeled anti-mouse Fc 2^{nd} antibody (Sigma) was added and incubated at 37° C. for 1 hour. Fluorescence intensity was detected. Blocking ratio was determined by blockade of human CD47 ECD recombinant protein binding to SIRPα expressed CHOK1 cells. IC₅₀ and top blocking percentage calculated using GraphPad Prism9.0 are summarized in FIG. 18.

### 1.8 SIP-1 Recruitment Assay

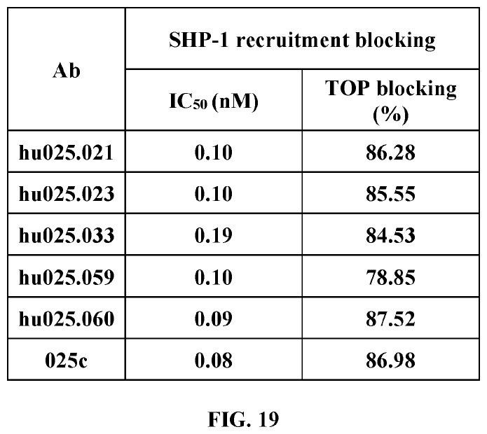

The efficacy of the humanized antibodies to block CD47/SIRPα mediated “don't eat me” signaling was assessed by cell-based SHP-1 recruitment assay (refer to methods described above). All the humanized antibodies as tested were confirmed to retain the similar activity as the parental antibody of C25 to block CD47 engagement resulted SHP-1 recruitment to SIRPα intracellular tail. IC₅₀ and top blocking percentage calculated using GraphPad Prism9.0 are summarized in FIG. 19.

### 1.9 In Vitro Phagocytosis Assay

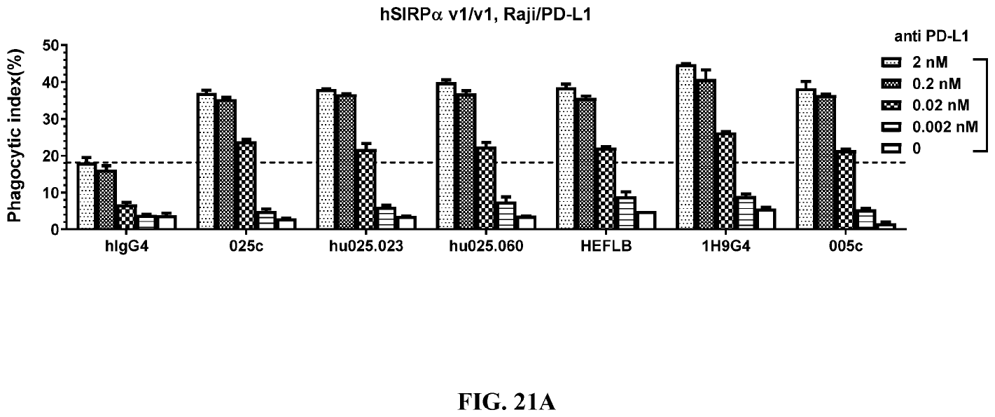

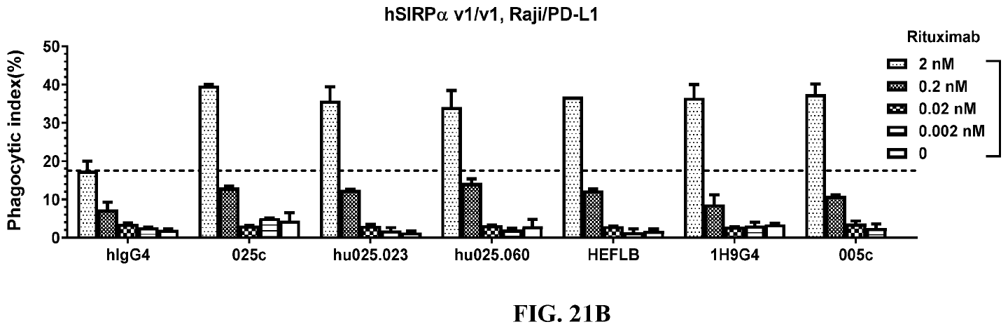

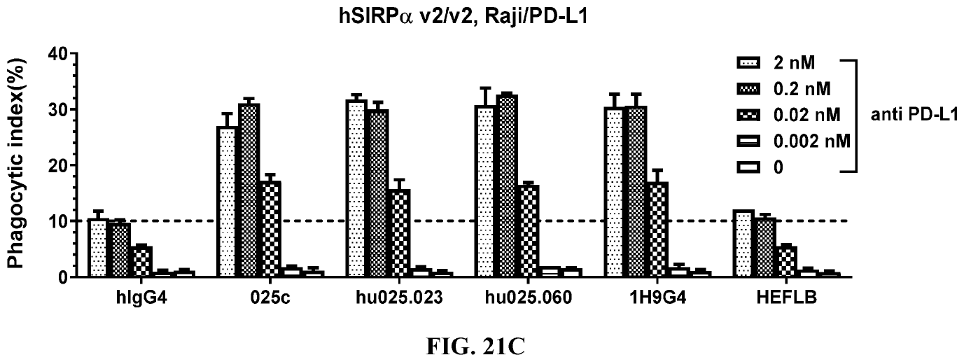

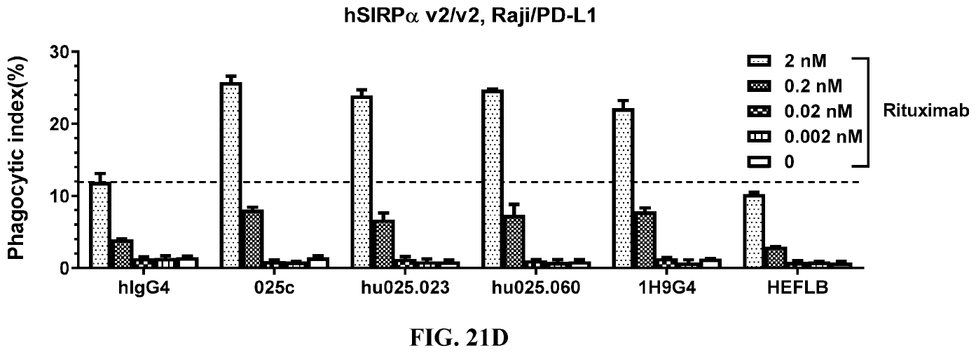

For in vitro function validation, combination of SIRPα antibody and PD-L1 antibody or Rituximab were tested in phagocytosis assay using M0 macrophages obtained from SIRPA homozygous v1/v1 (FIG. 21A and FIG. 21B) and v2/v2 (FIG. 21C and FIG. 21D) individuals. All the humanized antibodies as tested were confirmed to retain the similar activity as the parental antibody of 025c to potentiate macrophage mediated ADCP of Raji cells stably expressing PD-L1 in the presence of PD-L1 antibody or Rituximab.

2. Categorizing of the Anti-SIRPα Antibodies into Complete Blockers, Partial Blockers and Non-Blockers

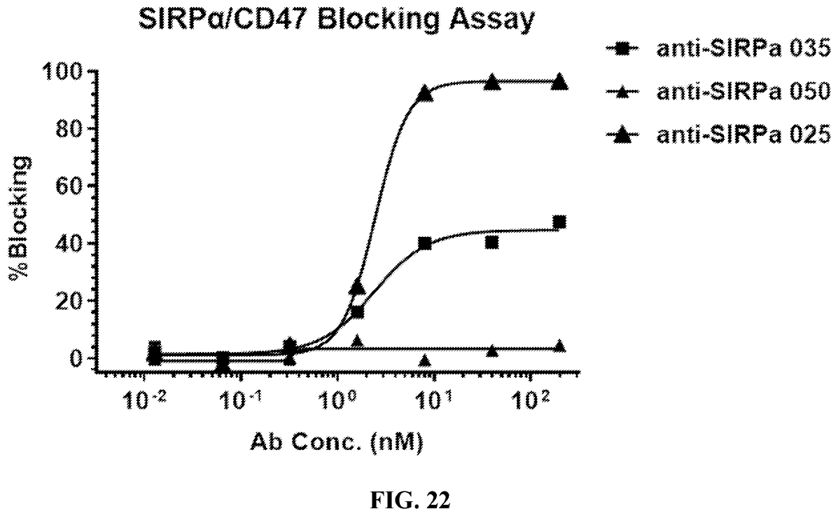

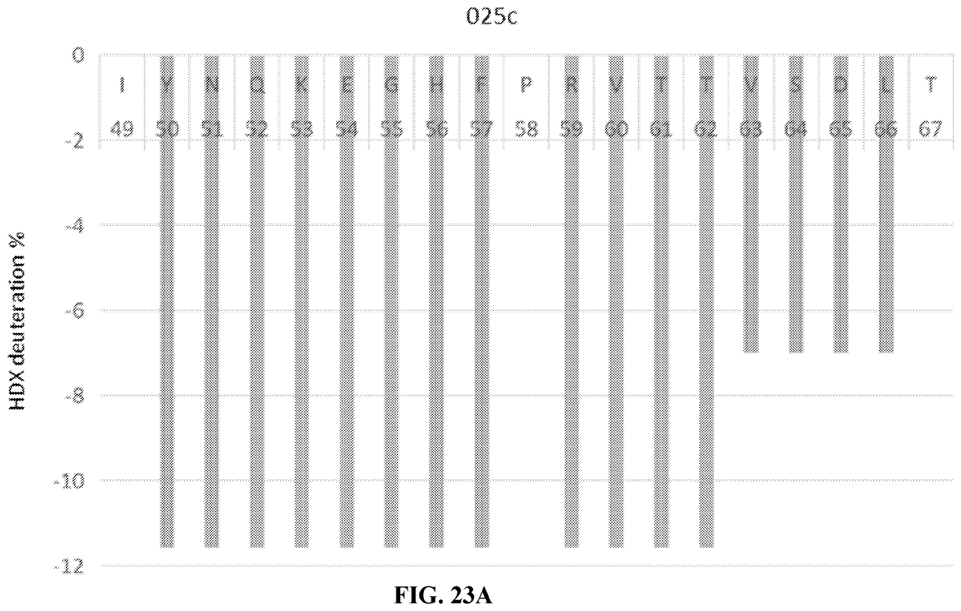

Competitive ELISA assay was used to determine whether the purified chimeric antibodies can block CD47 and SIRPα interaction. Briefly, antibody and mFc tagged human CD47 ECD recombinant protein were co-incubated with ELISA microplate coated human SIRPα v1 ECD recombinant protein. After washing, HRP labeled anti-mouse Fc 2nd antibody (Sigma) was added and incubated at 37° C. for 1 hour. Then, 100 μl/well of TMB solution (Biotechnology) was added. After incubation for 15 minutes at room temperature, the reaction was stopped by the addition of 50 μl of 1N HCl. OD 450 nm was read. Blocking ratio was determined by blockade of human SIRPα ECD recombinant protein binding to ELISA microplate coated human CD47 ECD recombinant protein. FIG. 22 shows that 025 or C25 has a maximal blocking percentage of more than 90%, which is defined as a complete blocker, that 050 or C50 has a maximal blocking percentage of close to zero, which is defined as a non-blocker, and that 035 or C35 has a maximal blocking percentage in between, which is defined as a partial blocker (Table 10).

TABLE 10
CD47 and SIRPα Blocking Properties of
Different Anti- SIRPα Clones

| CD47- SIRPα Interaction | |
|---|---|
| Blocking Activity | Anti- SIRPα Clone |
| Complete blocker | C25, C15, C42, C59, C73 |
| Partial blocker | C35 |
| Non-blocker | C50 |

### 3. Combination Therapy and Bi-Specific Molecules Using Complete Blockers C25, C15, C42, C59 and C73, Partial Blocker C35 and Non-Blocker C50

Different CD47 and SIRPα blocking antibodies are tested in human Raji lymphoma cells that lacks of human PDL1 expression, or Raji expressing human PDL1 were labeled with the fluorescent dye CFSE, and incubated with human macrophage-colony stimulating factor (M-CSF) treated monocyte derived macrophage either in the presence of an isotype control, anti-PDL1 antibody, anti-SIRPα antibody C25, C35 C50, the combination of anti-PDL1 and anti-SIRPα antibody or C25, C35, C50 based anti-PDL1/SIRPα bispecific antibody. After 2 hours, the macrophages were harvested, stained with a fluorescently labeled anti-human macrophage antibody, and analyzed by flow cytometry. CD11b+CFSE+ double positive events identify macrophages that have phagocytosed CFSE-labeled tumor cell. Each sample is represented by a different color. Phagocytic index is shown for three separate samples.

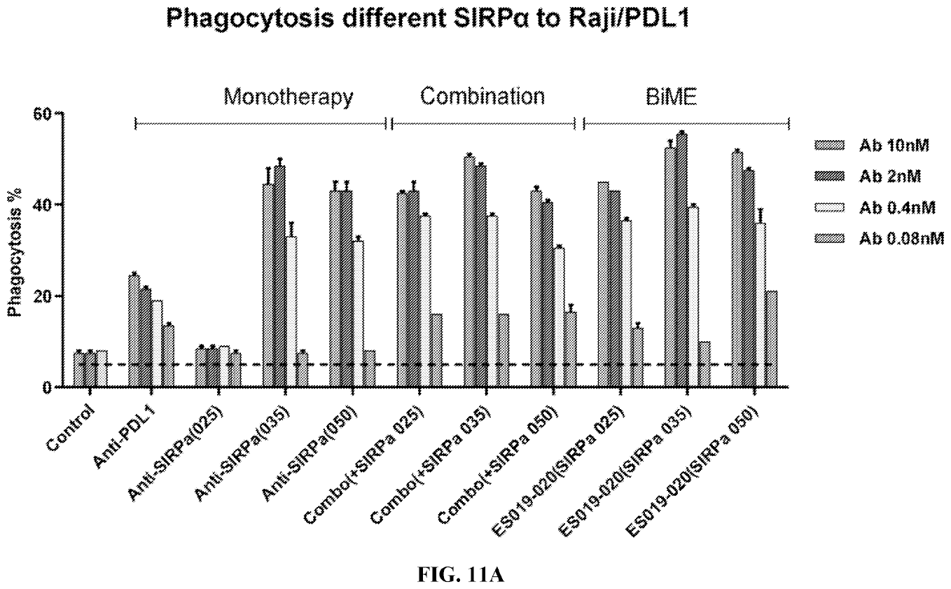

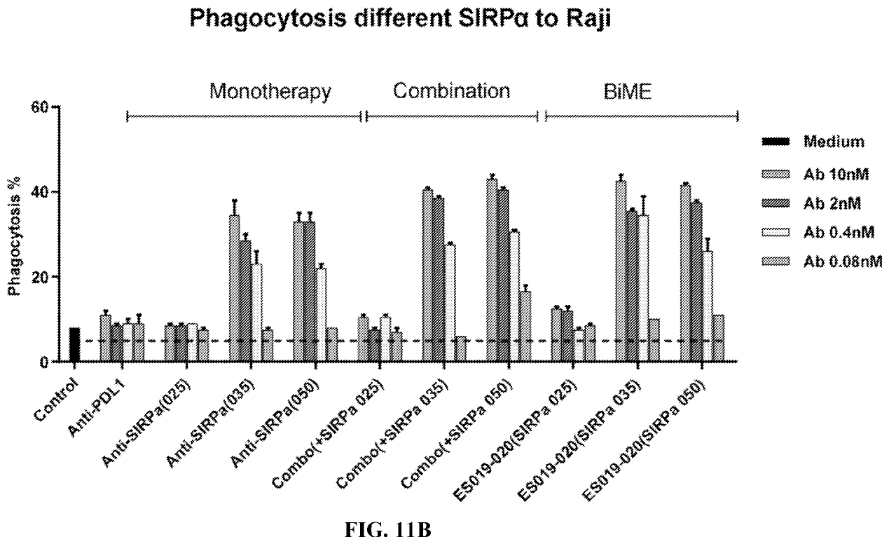

As shown in FIG. 11A, anti-SIRPα partial blocking antibody C35 and non-blocking antibody C50 induced ˜40% phagocytosis with mono-treatment against Raji/PDL1 cell; anti-SIRPα complete antibody C25 monotreatment barely induce phagocytosis. The combination of anti-PDL1 and anti-SIRPα antibody C25 or C25 based BiME significantly improve the phagocytosis. The combination of anti-PDL1 and anti-SIRPα antibody C35, C50 or based BiME didn't show synergistic phagocytosis effect.

In the phagocytosis assay against Raji (PDL1 negative) cell (FIG. 11B), anti-SIRPα partial blocking antibody C35 and non-blocking antibody C50 monotreatment can still induce phagocytosis against Raji(PDL1 negative) cell; anti-SIRPα complete antibody C25 monotreatment barely induce phagocytosis. The combination of anti-PDL1 and anti-SIRPα antibody C25 or C25 based PDL1/SIRPα bispecific antibody didn't induce the phagocytosis because the lack of PDL1 expression on tumor cell. The combination of anti-PDL1 and anti-SIRPα antibody C35, or C50 based BiME showed similar phagocytosis effect to C35 or C50 antibody monotreatment.

The result showed that anti-SIRPα antibody C35 or C50 monotreatment, combo or bispecific antibody can induce tumor cell phagocytosis independent of target antigen expression compared to C25 antibody. The property of anti-SIRPα antibody C25 based bispecific antibody provides a more specific and safe property.

The experimental data was summarized in the Table 11 below.

TABLE 11
Summary of the experimental results from Examples 10 and 12

| Tested cell | | | | |
|---|---|---|---|---|
| lines | Monotherapy | Combo | BiME | Example |
| K562 leukemia | C25: barely induce | C25 + C71: no | C25 + C71: no | 10 |
| tumor cells | phagocytosis | synergistic | synergistic | |
| (−human PDL1) | anti-PDL1 antibody | effect | effect | |
| | C71: barely induce | | | |
| | phagocytosis | | | |
| K562 leukemia | anti-PDL1 antibody | C25 + C71: | C25 + C71: | 10 |
| tumor cells | C71: ~20% | synergistic | synergistic | |
| (+human PDL1) | phagocytosis; | effect | effect | |
| | C25: barely induce | | | |
| | phagocytosis | | | |
| human Raji | C25: barely induce | C25 + C71: no | C25 + C71: no | 12 |
| lymphoma cells | phagocytosis | synergistic | synergistic | |
| (−human | anti-PDL1 antibody | effect | effect | |
| PDL1) | C71: barely induce | | | |
| | phagocytosis | | | |
| | C35 and C50: | C35 + C71 or | C35 + C71, or | |
| | induced ~40% | C50 + C71: | C50 + C71: | |
| | phagocytosis | similar to | similar to | |
| | | monotherapy | monotherapy | |
| human Raji | C25: barely induce | C25: | C25: | 12 |
| lymphoma cells | phagocytosis | synergistic | synergistic | |
| (+human | | effect | effect | |
| PDL1) | C35 or C50: | C35 + C71; or | C35 + C71; or | |
| | induced ~40% | C50 + C71: | C50 + C71: | |
| | phagocytosis | similar to | similar to | |
| | anti-PDL1 antibody | monotherapy | monotherapy | |
| | C71: induced ~20% | | | |
| | phagocytosis | | | |
| Mouse MC38 | anti-Claudin18.2 | C25 + | C25 + | 4 |
| colon tumor | antibody hu28H1L2: | hu28H1L2: | hu28H1L2: | |
| cells expressing | induced ~25% | synergistic | stronger | |
| human CD47 | phagocytosis | effect | synergistic | |
| and human | anti-SIRPα antibody | | effect | |
| Claudin18.2 | C25 barely induce | | | |
| | phagocytosis | | | |

As shown in Example 4, the combination of anti-Claudin18.2 and anti-SIRPα antibody C25 or C25 based BiME also significantly improve the phagocytosis selectively against cells expressing Claudin18.2 over non-Claudin18.2 expressing cells.

In addition, as shown in FIG. 20A and FIG. 21A, the combination of C15, C42, C59 or C73 and an antibody targeting the target antigen (e.g., PD-L1, Claudin18.2) is expected to have selective phagocytosis against cells expressing a target antigen (e.g., PD-L1, Claudin18.2) over target antigen non-expressing cells. Complete blockers C15, C42, C59 and C73 can also be used to make the multi-specific molecules provided in a form, such as, ES028-001, ES028-005, ES028-009, ES028-013, ES019-020, ES019-024, ES019-025, ES019-026, ES019-029, ES019-072, ES019-073 and ES019-079, and can be expected to have similar selective phagocytosis against cells expressing a target antigen (e.g., PD-L1, Claudin18.2) over target antigen non-expressing cells.

Although macrophages are exemplified throughout the specification, the multi-specific molecules, compositions and methods described here are applicable to cells of a myeloid cell lineage, such as a dendritic cell. Minor optimizations and changes are envisioned on a cell to cell basis as is known to one of skill in the art and is contemplated within the scope of the present disclosure.

What is claimed is:

1. A multi-specific molecule comprising:
(a) a SIRP-alpha binding domain,
(b) an activating receptor-binding domain, and
(c) a target antigen binding domain that binds to a target antigen expressed on a target cell co-expressing the target antigen and CD47,
wherein the multi-specific molecule selectively induces effector function of an immune effector cell in the presence of the target antigen, and wherein the immune effector cell co-expresses SIRP-alpha and the activating receptor.

2. The multi-specific molecule of claim 1, wherein the target cell co-expressing the target antigen and CD47 is a cancer cell, an infected cell, or a disease cell of interest for elimination by the effector function of the immune effector cell.

3. The multi-specific molecule of claim 1, wherein the multi-specific molecule induces minimal effector function of the immune effector cell in the absence of the target antigen.

4. The multi-specific molecule of any one of the preceding claims, wherein the effector function induced by the multi-specific molecule in the absence of the target antigen is no more than 10% of that induced in the presence of the target antigen.

5. The multi-specific molecule of any one of the preceding claims, wherein the immune effector cell is a myeloid cell, optionally, the immune effector cell is a macrophage cell, monocytes, neutrophils, eosinophil, phagocyte or basophil, optionally the immune effector cell is macrophage cell.

6. The multi-specific molecule of any one of the preceding claims, wherein the effector function comprises phagocytosis of the cell co-expressing the antigen and CD47 by the immune effector cell.

7. The multi-specific molecule of any one of the preceding claims, wherein the activating receptor is fragment crystallizable γ receptors (FcγRs), TREM2, lectin, scavenger receptor A1 (SRA1), MARCO, CD36, CD163, CD68, CD205, CD206, FcDR1, CD207, CD209, RAGE, CD14, CD64, F4/80, CD64, CD32a, CD16a, CD89, CD19, CD28, CSFR, PDGFR, MSR1, SCARA3, COLEC12, SCARA5, SCARB1, SCARB2, dectin 1, RAGE (SR-E1), LRP1, LRP2, ASGP, SR-PSOX, CXCL16, OLR1, SCARF1, SCARF2, CXCL16, STAB1, STAB2, SRCRB4D, SSC5D, CCR2, CX3CR1, CSF1R, Tie2, HuCRIg(L), and CD169 receptor or complement receptors (such as CR1 and CR3), PI3K, FcγR1, FcγR2A, FcγR2B2, FcγR2C, FcγR3A, BAH. Tyro3, Axl, Traf6, Syk, MyD88, Zap70, FcεR1, FcαR1, BAFF-R, DAP 12, NFAM1, MRC1, ItgB5, MERTK, ELMO, and CD79b; optionally, the activating receptor is FcγR.

8. The multi-specific molecule of any one of the preceding claims, wherein the activating receptor-binding domain comprises an Fc domain, optionally the Fc domain is derived from IgG1 or IgG4.

9. The multi-specific molecule of any one of the preceding claims, wherein the SIRP-alpha binding domain is capable of substantially blocking interaction between SIRP-alpha and CD47.

10. The multi-specific molecule of any one of the preceding claims, wherein the SIRP-alpha binding domain is capable of completely blocking interaction between SIRP-alpha and CD47.

11. The multi-specific molecule of any one of the preceding claims, wherein the SIRP-alpha binding domain is capable of substantially blocking SHP-1 recruitment mediated by interaction between SIRP-alpha and CD47.

12. The multi-specific molecule of any one of the preceding claims, wherein the SIRP-alpha binding domain is capable of completely blocking SHP-1 recruitment mediated by interaction between SIRP-alpha and CD47.

13. The multi-specific molecule of any one of the preceding claims, wherein the SIRP-alpha binding domain has minimal intrinsic activity to induce the effector function of the immune effector cell.

14. The multi-specific molecule of any one of the preceding claims, wherein the SIRP-alpha binding domain and the activating receptor-binding domain are in close proximity to permit binding of the multi-specific molecule to both SIRP-alpha and the activating receptor co-expressed on the same immune effector cell.

15. The multi-specific molecule of any of the preceding claims, wherein the SIRP-alpha binding domain and/or the target antigen binding domain comprises an antibody domain or an antibody mimetic domain; optionally the antibody mimetic domain comprises a fibronectin domain, Z domain of protein A (Affibody), gamma-B crystalline domain, ubiquitin domain, cystatin domain, Sac7d domain, triple helix coiled coil domain, lipocalins domain, A domains of a membrane receptor, Ankyrin repeat motif, SH3 domain of Fyn, Kunitz domain of a protease inhibitor, type III domain of fibronectin (Minibody), carbohydrae binding module 32-2.

16. The multi-specific molecule of claim 15, wherein the antibody domain comprises a Fab, a VHH, a single chain Fv (scFv), diabody, a Fab′, a F(ab′)₂, a Fd, an Fv fragment, a disulfide stabilized Fv fragment (dsFv), a (dsFv)₂, a bispecific dsFv (dsFv-dsFv′), a disulfide stabilized diabody (ds diabody), F(ab)₂, an scFv dimer (bivalent diabody), a camelized single domain antibody, a nanobody, a Tetrabody, a domain antibody, or a bivalent domain antibody.

17. The multi-specific molecule of any of the preceding claims, which comprises a multi-specific antibody comprising a target antigen binding antibody domain, a SIRP-a binding antibody domain, and an Fc domain.

18. The multi-specific molecule of claim 17, wherein the target antigen binding antibody domain is linked to N-terminus of the Fc domain.

19. The multi-specific molecule of claim 18, wherein the target antigen binding antibody domain comprises a Fab domain, optionally, the Fab domain comprises a heavy chain linked to one of the N-termini of the Fc domain.

20. The multi-specific molecule of claim 18, wherein the multi-specific molecule comprises two target antigen binding antibody domains, each of which comprises an Fab domain, optionally, each of the Fab domains comprises a heavy chain linked to each N-terminus of the Fc domain, respectively.

21. The multi-specific molecule of any of claims 18-20, wherein the SIRP-alpha binding domain is linked to the Fc domain or to the target antigen binding antibody domain.

22. The multi-specific molecule of claim 21, wherein the SIRP-alpha binding domain is linked to C-terminus of the Fc domain.

23. The multi-specific molecule of claim 21, wherein the SIRP-alpha binding domain is linked to N-terminus of the Fc domain, with the proviso that the SIRP-alpha binding domain and the target antigen binding antibody domain are not linked to the same N-terminus of the Fc domain.

24. The multi-specific molecule of claim 21, wherein the SIRP-alpha binding domain is linked to the C-terminus of light chain of the target antigen binding Fab domain.

25. The multi-specific molecule of claim 17, wherein the SIRP-alpha binding antibody domain is linked to N-terminus of the Fc domain.

26. The multi-specific molecule of claim 25, wherein the SIRP-alpha binding antibody domain comprises a Fab domain, optionally, the Fab domain comprises a heavy chain linked to one of the N-termini of the Fc domain.

27. The multi-specific molecule of claim 25, wherein the antibody comprise two SIRP-alpha binding antibody domains, each of which comprises an Fab domain, optionally, each of the Fab domains comprises a heavy chain linked to each N-terminus of the Fc domain, respectively.

28. The multi-specific molecule of any of claims 25-27, wherein the target antigen binding domain is linked to the Fc domain or to the SIRP-alpha binding antibody domain.

29. The multi-specific molecule of claim 28, wherein the target antigen binding domain is linked to the N-terminus of the Fc domain, with the provisio that the target antigen binding domain and the SIRP-alpha binding domain are not linked to the same N-terminus of the Fc domain.

30. The multi-specific molecule of claim 28, wherein the target antigen binding domain is linked to the C-terminus of the light chain of the SIRP-alpha binding Fab domain.

31. The multi-specific molecule of any of the preceding claims, wherein the target antigen comprises a tumor surface antigen.

32. The multi-specific molecule of claim 31, wherein the tumor surface antigen is PD-L1, claudin 18.2, BCMA, CD19, CD20, CD22, CD24, CD25, CD30, CD33, CD38, CD44, CD52, CD56, CD70, CD96, CD97, CD99, CD123, EGFR, HER2, HER3, CD117, C-Met, EGFR, EGFRvIII, ERBB3, ERBB4, VEGFR1, VEGFR2, ROR1, PTHR2, B7-H1(PD-L1), B7-H2, B7-H3, B7-H4, B7-H5, B7-H6, B7-H7, Trop-2, GPC-3, EPCAM, DLL-3, Nectin-4, Claudin6, Muc-1, PSMA, GD3, FAP, CEA, or EphA2.

33. The multi-specific molecule of any of the preceding claims, wherein the SIRP-alpha binding domain comprises:
a) a HCDR1 comprising the sequence of X₁YYMH (SEQ ID NO: 161), a HCDR2 comprising the sequence of RIDPEDX₂EX₃KYAPKFQG (SEQ ID NO: 162), and a HCDR3 comprising the sequence of GX₁₅X₄X₅Y (SEQ ID NO: 163); and/or a LCDR1 comprising the sequence of SASSSVSSSYLY (SEQ ID NO: 26), a LCDR2 comprising the sequence of STSNLAS (SEQ ID NO: 27), and a LCDR3 comprising the sequence of X₆QWSSYPYT (SEQ ID NO: 164); or
b) a HCDR1 comprising the sequence of TYGMS (SEQ ID NO: 35), a HCDR2 comprising the sequence of WINTYSGVX₇TX₈ADDFKG (SEQ ID NO: 165), and a HCDR3 comprising the sequence of DPHX₉YGX₁₀SPAWFX₁₁Y (SEQ ID NO: 166); and/or a LCDR1 comprising the sequence of X₁₂ASQX₁₃VGIX₁₄VA (SEQ ID NO: 188), a LCDR2 comprising the sequence of SASNRYT (SEQ ID NO: 39), and a LCDR3 comprising the sequence of QQYSX₁₆YPX₁₇T (SEQ ID NO: 189); or
c) a HCDR1 comprising the sequence of EYVLS (SEQ ID NO: 41), a HCDR2 comprising the sequence of EIYPGTITTYYNEKFKG (SEQ ID NO: 42), and a HCDR3 comprising the sequence of FYDYDGGWFAY (SEQ ID NO: 43); and/or a LCDR1 comprising the sequence of SASSSVSSSDLH (SEQ ID NO: 44), a LCDR2 comprising the sequence of GTSNLAS (SEQ ID NO: 45), and a LCDR3 comprising the sequence of QQWSGYPWT (SEQ ID NO: 46), wherein X₁ is A or D; X₂ is G or A; X₃ is T or S; X₄ is L or Y; X₅ is E or A; X₆ is Y or H; X₇ is S or P; X₈ is Y or C; X₉ is Y or S; X₁₀ is N or S; X₁₁ is P or V; X₁₂ is E or K; X₁₃ is N or I; X₁₄ is S or A; X₁₅ is S or absent; X₁₆ is S or A; X₁₇ is F or L.

34. The multi-specific molecule of any of the preceding claims, wherein the SIRP-alpha binding domain comprises:
a) a HCDR1 comprising the sequence of SEQ ID NO: 23, a HCDR2 comprising the sequence of SEQ ID NO: 24 or SEQ ID NO: 198, and a HCDR3 comprising the sequence of SEQ ID NO: 25; and/or a LCDR1 comprising the sequence of SEQ ID NO: 26, a LCDR2 comprising the sequence of SEQ ID NO: 27, and a LCDR3 comprising the sequence of SEQ ID NO: 28; or
b) a HCDR1 comprising the sequence of SEQ ID NO: 29, a HCDR2 comprising the sequence of SEQ ID NO: 30, and a HCDR3 comprising the sequence of SEQ ID NO: 31; and/or a LCDR1 comprising the sequence of SEQ ID NO: 32, a LCDR2 comprising the sequence of SEQ ID NO: 33, and a LCDR3 comprising the sequence of SEQ ID NO: 34; or
c) a HCDR1 comprising the sequence of SEQ ID NO: 35, a HCDR2 comprising the sequence of SEQ ID NO: 36, and a HCDR3 comprising the sequence of SEQ ID NO: 37; and/or a LCDR1 comprising the sequence of SEQ ID NO: 38, a LCDR2 comprising the sequence of SEQ ID NO: 39, and a LCDR3 comprising the sequence of SEQ ID NO: 40; or
d) a HCDR1 comprising the sequence of SEQ ID NO: 47, a HCDR2 comprising the sequence of SEQ ID NOs: 48, and a HCDR3 comprising the sequence of SEQ ID NOs: 49; and/or a LCDR1 comprising the sequence of SEQ ID NOs:
50, a LCDR2 comprising the sequence of SEQ ID NOs: 51, and a LCDR3 comprising the sequence of SEQ ID NOs: 52.

35. The multi-specific molecule of any of the preceding claims, wherein the SIRP-alpha binding domain comprises the same HCDRs and LCDRs as anti-SIRP-alpha antibody selected from the group consisting of C25, C15, C42, C59 and C73, wherein:
a) the C25 comprises a heavy chain variable region comprising the sequence of SEQ ID NO: 1, and/or a light chain variable region comprising the sequence of SEQ ID NO: 2,
b) the C15 comprises a heavy chain variable region comprising the sequence of SEQ ID NO: 11, and/or a light chain variable region comprising the sequence of SEQ ID NO: 12,
c) the C42 comprises a heavy chain variable region comprising the sequence of SEQ ID NO: 13, and/or a light chain variable region comprising the sequence of SEQ ID NO: 14,
d) the C59 comprises a heavy chain variable region comprising the sequence of SEQ ID NO: 15, and/or a light chain variable region comprising the sequence of SEQ ID NO: 16, and
e) the C73 comprises a heavy chain variable region comprising the sequence of SEQ ID NO: 17, and/or a light chain variable region comprising the sequence of SEQ ID NO: 18.

36. The multi-specific molecule of any of the preceding claims, wherein the SIRP-alpha binding domain further comprises one or more of heavy chain HFR1, HFR2, HFR3 and HFR4, and/or one or more of light chain LFR1, LFR2, LFR3 and LFR4, wherein:
i) the HFR1 comprises EVQLVQSGAEVKKPGATVKISCKX₂₀SGFNIK (SEQ ID NO: 190) or a homologous sequence of at least 80% sequence identity thereof, and/or
j) the HFR2 comprises WVQQAPGKGLEWIG (SEQ ID NO: 191) or a homologous sequence of at least 80% sequence identity thereof, and/or
k) the HFR3 sequence comprises RVTITADTSTX₂₁TAYMELSSLRSEDTAVYYCDR (SEQ ID NO: 192) or a homologous sequence of at least 80% sequence identity thereof, and/or
l) the HFR4 comprises WGQGTLVTVSS (SEQ ID NO: 193) or a homologous sequence of at least 80% sequence identity thereof, and/or
m) the LFR1 comprises EIVLTQSPATLSLSPGERATLSC (SEQ ID NO: 194) or a homologous sequence of at least 80% sequence identity thereof, and/or
n) the LFR2 comprises WYQQKPGQAPKLWIY (SEQ ID NO: 195) or a homologous sequence of at least 80% sequence identity thereof, and/or
o) the LFR3 comprises GIPARFSGSGSGTDX₂₂TLTISSLEPEDFAVYYC (SEQ ID NO: 196) or a homologous sequence of at least 80% sequence identity thereof, and/or
p) the LFR4 comprises FGQGTKLEIK (SEQ ID NO: 197) or a homologous sequence of at least 80% sequence identity thereof,
wherein X₂₀ is A or V; X₂₁ is N or D; X₂₂ is Y or F.

37. The multi-specific molecule of any of the preceding claims, wherein the SIRP-alpha binding domain comprises
a) the heavy chain variable region comprises the sequence of SEQ ID NO: 1 and/or the light chain variable region comprises the sequence of SEQ ID NO: 2; or
b) the heavy chain variable region comprises the sequence of SEQ ID NO: 3 and/or the light chain variable region comprises the sequence of SEQ ID NO: 4; or
c) the heavy chain variable region comprises the sequence of SEQ ID NO: 5 and/or the light chain variable region comprises the sequence of SEQ ID NO: 6; or
d) the heavy chain variable region comprises the sequence of SEQ ID NO: 7 and/or the light chain variable region comprises the sequence of SEQ ID NO: 8; or
e) the heavy chain variable region comprises the sequence of SEQ ID NO: 9 and/or the light chain variable region comprises the sequence of SEQ ID NO: 10; or
f) the heavy chain variable region comprises the sequence of SEQ ID NO: 11 and/or the light chain variable region comprises the sequence of SEQ ID NO: 12; or
g) the heavy chain variable region comprises the sequence of SEQ ID NO: 13 and/or the light chain variable region comprises the sequence of SEQ ID NO: 14; or
h) the heavy chain variable region comprises the sequence of SEQ ID NO: 15 and/or the light chain variable region comprises the sequence of SEQ ID NO: 16; or
i) the heavy chain variable region comprises the sequence of SEQ ID NO: 17 and/or the light chain variable region comprises the sequence of SEQ ID NO: 18 or
j) the heavy chain variable region comprises the sequence of SEQ ID NO: 159 and/or the light chain variable region comprises the sequence of SEQ ID NO: 160.

38. The multi-specific molecule of any of the preceding claims, wherein the target antigen binding domain comprises a claudin 18.2 binding domain.

39. The multi-specific molecule of any of the preceding claims, wherein the claudin 18.2 binding domain comprises:
a) a HCDR1 comprising the sequence of SEQ ID NO: 77, a HCDR2 comprising the sequence of SEQ ID NO: 78, and a HCDR3 comprising the sequence of SEQ ID NO: 79; and/or a LCDR1 comprising the sequence of SEQ ID NO: 80, a LCDR2 comprising the sequence of SEQ ID NO: 81, and a LCDR3 comprising the sequence of SEQ ID NO: 82 or SEQ ID NO: 225; or
b) a HCDR1 comprising the sequence of SEQ ID NO: 83, a HCDR2 comprising the sequence of SEQ ID NO: 84, and a HCDR3 comprising the sequence of SEQ ID NO: 85; and/or a LCDR1 comprising the sequence of SEQ ID NO: 86, a LCDR2 comprising the sequence of SEQ ID NO: 87, and a LCDR3 comprising the sequence of SEQ ID NO: 88; or
c) a HCDR1 comprising the sequence of SEQ ID NO: 89, a HCDR2 comprising the sequence of SEQ ID NO: 90, and a HCDR3 comprising the sequence of SEQ ID NO: 91; and/or a LCDR1 comprising the sequence of SEQ ID NO: 92, a LCDR2 comprising the sequence of SEQ ID NO: 93, and a LCDR3 comprising the sequence of SEQ ID NO: 94; or
d) a HCDR1 comprising the sequence of SEQ ID NO: 95, a HCDR2 comprising the sequence of SEQ ID NO: 96, and a HCDR3 comprising the sequence of SEQ ID NO: 97; and/or a LCDR1 comprising the sequence of SEQ ID NO: 98, a LCDR2 comprising the sequence of SEQ ID NO: 99, and a LCDR3 comprising the sequence of SEQ ID NO: 100; or
e) a HCDR1 comprising the sequence of SEQ ID NO: 101, a HCDR2 comprising the sequence of SEQ ID NO: 102, and a HCDR3 comprising the sequence of SEQ ID NO: 103; and/or a LCDR1 comprising the sequence of SEQ ID NO: 104, a LCDR2 comprising the sequence of SEQ ID NO: 105, and a LCDR3 comprising the sequence of SEQ ID NO: 106.

40. The multi-specific molecule of any of the preceding claims, wherein the claudin 18.2 binding domain comprises the same HCDRs and LCDRs as anti-claudin 18.2 antibody selected from the group consisting of hu26.H1L1, hu26.H1L2 (S92A), hu28.H1L2, C10, C29 and C30,
a) wherein the hu26.H1L1 comprises a heavy chain variable region comprising the sequence of SEQ ID NO: 65, and/or a light chain variable region comprising the sequence of SEQ ID NO: 66,
b) wherein the hu26.H1L2 (S92A) comprises a heavy chain variable region comprising the sequence of SEQ ID NO: 65, and/or a light chain variable region comprising the sequence of SEQ ID NO: 224
c) the hu28.H1L2 comprises a heavy chain variable region comprising the sequence of SEQ ID NO: 69, and/or a light chain variable region comprising the sequence of SEQ ID NO: 70,
d) the C10 comprises a heavy chain variable region comprising the sequence of SEQ ID NO: 71, and/or a light chain variable region comprising the sequence of SEQ ID NO: 72,
e) the C29 comprises a heavy chain variable region comprising the sequence of SEQ ID NO: 73, and/or a light chain variable region comprising the sequence of SEQ ID NO: 74, and
f) the C30 comprises a heavy chain variable region comprising the sequence of SEQ ID NO: 75, and/or a light chain variable region comprising the sequence of SEQ ID NO: 76.

41. The multi-specific molecule of any of the preceding claims, wherein the claudin 18.2 binding domain further comprises one or more of heavy chain HFR1, HFR2, HFR3 and HFR4, and/or one or more of light chain LFR1, LFR2, LFR3 and LFR4, wherein:
a) the HFR1 comprises an amino acid sequence selected from the group consisting of EVQLLESGGGLVQPGGSLRLSCAASGFTLS (SEQ ID NO: 167) and QVQLVQSGAEVKKPGASVKVSCKASGYTFT (SEQ ID NO: 168) or a homologous sequence of at least 80% sequence identity thereof,
b) the HFR2 comprises an amino acid sequence selected from the group consisting of WVRQAPGKGLEWVX₁₈ (SEQ ID NO: 169) and WVRQAPGQGLEWMG (SEQ ID NO: 170) or a homologous sequence of at least 80% sequence identity thereof,
c) the HFR3 sequence comprises an amino acid sequence selected from the group consisting of RFTISRDNSKNTLYLQMNSLRAEDTAVYYCAX₂₃ (SEQ ID NO: 171) and RVTMTRDTSTSTVYMELSSLRSEDTAVYYCAR (SEQ ID NO: 172) or a homologous sequence of at least 80% sequence identity thereof,
d) the HFR4 comprises WGQGTLVTVSS (SEQ ID NO: 173) or a homologous sequence of at least 80% sequence identity thereof,
e) the LFR1 comprises an amino acid sequence selected from the group consisting of DIQLTQSPSFLSASVGDRVTITC (SEQ ID NO: 174) and DIVMTQSPDSLAVSLGERATINC (SEQ ID NO: 175) or a homologous sequence of at least 80% sequence identity thereof,
f) the LFR2 comprises an amino acid sequence selected from the group consisting of WYQQKPGX₂₆X₂₇PKX₁₉LIY (SEQ ID NO: 176) or a homologous sequence of at least 80% sequence identity thereof,
g) the LFR3 comprises an amino acid sequence selected from the group consisting of GVPSRFSGSGSGTEX₂₄TLTISSLQPEDFATYYC (SEQ ID NO: 178) and GVPDRFSGSGSGTDFTLTISSLQAEDVAVYHC (SEQ ID NO: 179) or a homologous sequence of at least 80% sequence identity thereof, and
h) the LFR4 comprises FGX₂₅GTKLEIK (SEQ ID NO: 180) or a homologous sequence of at least 80% sequence identity thereof,
wherein X₁₈ is S or A, X₁₉ is L or A, X₂₃ is T or K, X₂₄ is Y or F, X₂₅ is Q or G, X₂₆ is Q or K, X₂₇ is P or A.

42. The multi-specific molecule of any of the preceding claims, wherein the claudin 18.2 binding domain comprises:
a) the heavy chain variable region comprises the sequence of SEQ ID NO: 65 or 68, and/or the light chain variable region comprises the sequence of SEQ ID NO: 66 or 67 or 224; or
b) the heavy chain variable region comprises the sequence of SEQ ID NO: 69 and/or the light chain variable region comprises the sequence of SEQ ID NO: 70; or
c) the heavy chain variable region comprises the sequence of SEQ ID NO: 71 and/or the light chain variable region comprises the sequence of SEQ ID NO: 72; or
d) the heavy chain variable region comprises the sequence of SEQ ID NO: 73 and/or the light chain variable region comprises the sequence of SEQ ID NO: 74; or
e) the heavy chain variable region comprises the sequence of SEQ ID NO: 75 and/or the light chain variable region comprises the sequence of SEQ ID NO: 76.

43. The multi-specific molecule of any of the preceding claims, wherein the target antigen binding domain comprises a PD-L1 binding domain.

44. The multi-specific molecule of any of the preceding claims, wherein the PD-L1 binding domain comprises:
a) a HCDR1 comprising the sequence of SEQ ID NO: 119, a HCDR2 comprising the sequence of SEQ ID NO: 120, and a HCDR3 comprising the sequence of SEQ ID NO: 121; or
b) a HCDR1 comprising the sequence of SEQ ID NO: 122, a HCDR2 comprising the sequence of SEQ ID NO: 123, and a HCDR3 comprising the sequence of SEQ ID NO: 124; or
c) a HCDR1 comprising the sequence of SEQ ID NO: 125, a HCDR2 comprising the sequence of SEQ ID NO: 126, and a HCDR3 comprising the sequence of SEQ ID NO: 127; or
d) a HCDR1 comprising the sequence of SEQ ID NO: 128, a HCDR2 comprising the sequence of SEQ ID NO: 129, and a HCDR3 comprising the sequence of SEQ ID NO: 130; or
e) a HCDR1 comprising the sequence of SEQ ID NO: 131, a HCDR2 comprising the sequence of SEQ ID NO: 132, and a HCDR3 comprising the sequence of SEQ ID NO: 133; or
f) a HCDR1 comprising the sequence of SEQ ID NO: 134, a HCDR2 comprising the sequence of SEQ ID NO: 135, and a HCDR3 comprising the sequence of SEQ ID NO: 136; or
g) a HCDR1 comprising the sequence of SEQ ID NO: 137, a HCDR2 comprising the sequence of SEQ ID NO: 138, and a HCDR3 comprising the sequence of SEQ ID NO: 139; or
h) a HCDR1 comprising the sequence of SEQ ID NO: 140, a HCDR2 comprising the sequence of SEQ ID NO: 141, and a HCDR3 comprising the sequence of SEQ ID NO: 142; or
i) a HCDR1 comprising the sequence of SEQ ID NO: 143, a HCDR2 comprising the sequence of SEQ ID NO: 144, and a HCDR3 comprising the sequence of SEQ ID NO: 145; or
j) a HCDR1 comprising the sequence of SEQ ID NO: 146, a HCDR2 comprising the sequence of SEQ ID NO: 147, and a HCDR3 comprising the sequence of SEQ ID NO: 148; or
k) a HCDR1 comprising the sequence of SEQ ID NO: 149, a HCDR2 comprising the sequence of SEQ ID NO: 150, and a HCDR3 comprising the sequence of SEQ ID NO: 151; or
a HCDR1 comprising the sequence of SEQ ID NO: 152, a HCDR2 comprising the sequence of SEQ ID NO: 153, and a HCDR3 comprising the sequence of SEQ ID NO: 154.

45. The multi-specific molecule of any of the preceding claims, wherein the PD-L1 binding domain comprises the same HCDRs as anti-PD-L1 antibody selected from the group consisting of C71, C71v38, C239, C492, C570, 570h3, C446, C2811, C1778, C1793, C2855, C2713 and C2719,
a) wherein the C71 comprises a heavy chain variable region comprising the sequence of SEQ ID NO: 107,
b) the C71v38 comprises a heavy chain variable region comprising the sequence of SEQ ID NO: 108,
c) the C239 comprises a heavy chain variable region comprising the sequence of SEQ ID NO: 109,
d) the C492 comprises a heavy chain variable region comprising the sequence of SEQ ID NO: 110,
e) the C570 comprises a heavy chain variable region comprising the sequence of SEQ ID NO: 111,
f) the 570h3 comprises a heavy chain variable region comprising the sequence of SEQ ID NO: 223,
g) the C446 comprises a heavy chain variable region comprising the sequence of SEQ ID NO: 112,
h) the C2811 comprises a heavy chain variable region comprising the sequence of SEQ ID NO: 113,
i) the C1778 comprises a heavy chain variable region comprising the sequence of SEQ ID NO: 114,
j) the C1793 comprises a heavy chain variable region comprising the sequence of SEQ ID NO: 115,
k) the C2855 comprises a heavy chain variable region comprising the sequence of SEQ ID NO: 116,
l) the C2713 comprises a heavy chain variable region comprising the sequence of SEQ ID NO: 117, and
m) the C2719 comprises a heavy chain variable region comprising the sequence of SEQ ID NO: 118.

46. The multi-specific molecule of any of the preceding claims, wherein the PD-L1 binding domain comprises: the heavy chain variable region comprises the sequence selected from the group consisting of SEQ ID NOs: 107-118 and 223.

47. The multi-specific molecule of any of claims 30-39, wherein
a) the SIRP-alpha binding domain further comprises one or more amino acid residue substitutions or modifications yet retains specific binding to human SIRPα; and/or
b) the claudin 18.2 binding domain further comprises one or more amino acid residue substitutions or modifications yet retains specific binding to claudin 18.2, and/or
c) the PD-L1 binding domain further comprises one or more amino acid residue substitutions or modifications yet retains specific binding to PD-L1.

48. The multi-specific molecule of any of the preceding claims, wherein at least one of the substitutions or modifications is in one or more of the CDR sequences, and/or in one or more of the non-CDR sequences of the heavy chain variable region or light chain variable region.

49. The multi-specific molecule of any of the preceding claims, which is humanized.

50. The multi-specific molecule of any of the preceding claims, which is linked to one or more conjugate moieties.

51. The multi-specific molecule of any of the preceding claims, wherein the conjugate moiety comprises a clearance-modifying agent, a chemotherapeutic agent, a toxin, a radioactive isotope, a lanthanide, a luminescent label, a fluorescent label, an enzyme-substrate label, a DNA-alkylator, a topoisomerase inhibitor, a tubulin-binder, a purification moiety, or other anticancer drugs.

52. A pharmaceutical composition comprising the multi-specific molecule of any one of the preceding claims, and one or more pharmaceutically acceptable carriers.

53. An isolated polynucleotide encoding the multi-specific molecule of any one of the preceding claims.

54. A vector comprising the isolated polynucleotide of claim 53.

55. A host cell comprising the vector of claim 54.

56. A kit comprising the multi-specific molecule of any one of claims 1-51 and/or the pharmaceutical composition of claim 52, and a second therapeutic agent.

57. A method of expressing the multi-specific molecule of any one of claims 1-51, comprising culturing the host cell of claim 55 under the condition at which the vector of claim 54 is expressed.

58. A method of treating a disease, disorder or condition that can be benefited from induced phagocytosis of a target cell in a subject, comprising administering to the subject a therapeutically effective amount of the multi-specific molecule of any one of claims 1-51.

59. A method of treating a target antigen related disease, disorder or condition in a subject, comprising administering to the subject a therapeutically effective amount of the multi-specific molecule of any one of claims 1-51.

60. A method of treating a SIRPα related disease, disorder or condition in a subject, comprising administering to the subject a therapeutically effective amount of the multi-specific molecule of any one of claims 1-51.

61. A method of treating a CD47 related disease, disorder or condition in a subject, comprising administering to the subject a therapeutically effective amount of the multi-specific molecule of any one of claims 1-51.

62. The method of any of claims 58-61, wherein the subject is human.

63. The method of claim 62, wherein the subject has been diagnosed with or is at risk for a disease, disorder or condition selected from the group consisting of immune related disease or disorder, tumors and cancers, autoimmune diseases, and infectious disease.

64. The method of claim 63, the immune related disease or disorder is selected from the group consisting of systemic lupus erythematosus, acute respiratory distress syndrome (ARDS), vasculitis, myasthenia gravis, idiopathic pulmonary fibrosis, Crohn's Disease, asthma, rheumatoid arthritis, graft versus host disease, a spondyloarthropathy (e.g., ankylosing spondylitis, psoriatic arthritis, isolated acute enteropathic arthritis associated with inflammatory bowel disease, reactive arthritis, Behcet's syndrome, undifferentiated spondyloarthropathy, anterior uveitis, and juvenile idiopathic arthritis.), multiple sclerosis, endometriosis, glomerulonephritis, sepsis, diabetes, acute coronary syndrome, ischemic reperfusion, psoriasis, progressive systemic sclerosis, atherosclerosis, Sjogren's syndrome, scleroderma, or inflammatory autoimmune myositis.

65. The method of claim 63, wherein the tumors and cancers are solid tumor or hematologic malignancy, optionally selected from the group consisting of non-small cell lung cancer, small cell lung cancer, renal cell cancer, colorectal cancer, ovarian cancer, breast cancer, pancreatic cancer, gastric carcinoma, bladder cancer, esophageal cancer, mesothelioma, melanoma, head and neck cancer, thyroid cancer, sarcoma, prostate cancer, glioblastoma, cervical cancer, thymic carcinoma, leukemia, lymphomas, myelomas, mycoses fungoids, merkel cell cancer, and other hematologic malignancies, such as classical Hodgkin lymphoma (CHL), primary mediastinal large B-cell lymphoma, T-cell/histiocyte-rich B-cell lymphoma, EBV-positive and -negative PTLD, and EBV-associated diffuse large B-cell lymphoma (DLBCL), plasmablastic lymphoma, extranodal NK/T-cell lymphoma, nasopharyngeal carcinoma, and HHV8-associated primary effusion lymphoma, Hodgkin's lymphoma, neoplasm of the central nervous system (CNS), such as primary CNS lymphoma, spinal axis tumor, brain stem glioma, anal cancer, appendix cancer, astrocytoma, basal cell carcinoma, gallbladder cancer, gastric cancer, lung cancer, bronchial cancer, bone cancer, liver and bile duct cancer, pancreatic cancer, breast cancer, liver cancer, ovarian cancer, testicle cancer, kidney cancer, renal pelvis and ureter cancer, salivary gland cancer, small intestine cancer, urethral cancer, bladder cancer, head and neck cancer, spine cancer, brain cancer, cervix cancer, uterine cancer, endometrial cancer, colon cancer, colorectal cancer, rectal cancer, esophageal cancer, gastrointestinal cancer, skin cancer, prostate cancer, pituitary cancer, vagina cancer, thyroid cancer, throat cancer, glioblastoma, melanoma, myelodysplastic syndrome, sarcoma, teratoma, chronic lymphocytic leukemia (CLL), chronic myeloid leukemia (CML), acute lymphocytic leukemia (ALL), acute myeloid leukemia (AML), Hodgkin lymphoma, non-Hodgkin lymphoma, multiple myeloma, T or B cell lymphoma, GI organ interstitialoma, soft tissue tumor, hepatocellular carcinoma, and adenocarcinoma, or the metastases thereof.

66. The method of any one of claims 58-65, wherein the administration is via oral, nasal, intravenous, subcutaneous, sublingual, or intramuscular administration.

67. The method of any one of claims 58-65, further comprising administering a therapeutically effective amount of a second therapeutic agent.

68. The method of claim 67, wherein the second therapeutic agent is selected from the group consisting of a chemotherapeutic agent, an anti-cancer drug, a radiation therapy agent, an immunotherapy agent, an anti-angiogenesis agent, a targeted therapy agent, a cellular therapy agent, a gene therapy agent, a hormonal therapy agent, an antiviral agent, an antibiotic, an analgesics, an antioxidant, a metal chelator, and cytokines.

69. Use of the multi-specific molecule of any one of claims 1-51 and/or the pharmaceutical composition of claim 52 in the manufacture of a medicament for treating, preventing or alleviating a disease, disorder or condition that can be benefited from induced phagocytosis of a target cell in a subject.

70. A method of inducing phagocytosis of a target cell in a subject, comprising administering to the subject the multi-specific molecule of any one of claims 1-51 and/or the pharmaceutical composition of claim 52 in a dose effective to induce phagocytosis of the target cell.

71. The method of claim 70, wherein the subject is human.

72. The method of claim 70 or 71, wherein the subject has been diagnosed with or is at risk for a disease, disorder or condition selected from the group consisting of immune related disease or disorder, tumors and cancers, autoimmune diseases, and infectious disease.

73. A method of inducing phagocytosis of a target cell in vitro, comprising contacting the target cell with a SIRPα positive phagocytic cell sample in the presence of the multi-specific molecule of any one of claims 1-51 and/or the pharmaceutical composition of claim 52, thereby inducing the phagocytosis of the target cell by the SIRPα positive phagocytic cell.

74. The method of claim 73, wherein the target cell is a cell expressing the target antigen.

75. A method of inducing elimination of a target cell co-expressing a target antigen and CD47 by phagocytosis, comprising contacting the target cell with the multi-specific molecule of any of the claims 1-51 in presence of a phagocytic immune cell.

76. A method of inducing phagocytic effect selectively against a target cell co-expressing a target antigen and CD47 over a cell that does not express the target antigen in a subject, comprising administering to the subject a therapeutically effective amount of the multi-specific molecule of any one of claims 1-51.

77. A method of increasing the level of M1 macrophage in a tumor microenvironment of a subject in need thereof, comprising administering to the subject a therapeutically effective amount of the multi-specific molecule of any one of claims 1-51.
